(19) United States
(12) Patent Application Publication
(10) Pub. No.: US 2024/0284692 A1
(43) Pub. Date: Aug. 22, 2024

(54) HETEROCYCLIC COMPOUND, ORGANIC LIGHT EMITTING DEVICE COMPRISING THE SAME, AND COMPOSITION FOR ORGANIC MATERIAL LAYER OF ORGANIC LIGHT EMITTING DEVICE

(71) Applicant: LT MATERIALS CO., LTD., Yongin-si, Gyeonggi-do (KR)

(72) Inventors: Gi Back LEE, Yongin-si (KR); Yu Jin HEO, Yongin-si (KR); Jun Tae MO, Yongin-si (KR); Dong Jun KIM, Yongin-si (KR)

(73) Assignee: LT MATERIALS CO., LTD., Yongin-si, Gyeonggi-do (KR)

(21) Appl. No.: 18/564,049

(22) PCT Filed: Jul. 13, 2022
(86) PCT No.: PCT/KR2022/010219

§ 371 Date: Nov. 24, 2023

(30) Foreign Application Priority Data
Sep. 1, 2021 (KR) 10-2021-0116531

(51) Int. Cl. H10K 50/11; C09K 11/06; H10K 85/60

(52) U.S. Cl. CPC H10K 50/11; C09K 11/06; H10K 85/615; H10K 85/633; H10K 85/636; H10K 85/654; H10K 85/6572; H10K 85/6574; H10K 85/6576; C09K 2211/1048; C09K 2211/1051; C09K 2211/1062; C09K 2211/1066

(57) ABSTRACT

The present specification relates to a heterocyclic compound represented by Chemical Formula 1, an organic light emitting device comprising the same, and a composition for an organic material layer of an organic light emitting device.

### CROSS-REFERENCE TO RELATED APPLICATION (S)

This application claims priority to Korean Patent Application No. 10-2021-0116531, filed on Sep. 1, 2021, the disclosure of which is incorporated herein by reference in its entirety.

### TECHNICAL FIELD

The present invention relates to a heterocyclic compound, an organic light emitting device comprising the same, and a composition for an organic material layer of an organic light emitting device.

### DESCRIPTION OF THE RELATED ART

An organic light emitting device is one type of self-emissive display devices, and has advantages of having a wide viewing angle and a high response speed as well as having an excellent contrast.

An organic light emitting device has a structure of disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### SUMMARY

An object of the present disclosure is to provide a heterocyclic compound, an organic light emitting device comprising the same, and a composition for an organic material layer of an organic light emitting device.

One embodiment of the present disclosure provides a heterocyclic compound represented by the following Chemical Formula 1:

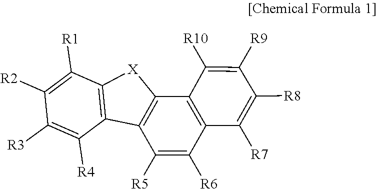

in Chemical Formula 1,
X is O or S,
R1 to R10 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 1-1; and a group represented by the following Chemical Formula 1-2, and
at least one of R5 and R6 is the group represented by the following Chemical Formula 1-1, and at least one of R7 to R10 is the group represented by the following Chemical Formula 1-2,

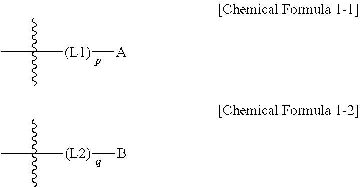

in Chemical Formulae 1-1 and 1-2,
L1 and L2 are the same as or different from each other, and are each independently a single bond; a substituted or unsubstituted C6 to C60 arylene group; or a substituted or unsubstituted C2 to C60 heteroarylene group,
A and B are different from each other, and each independently selected from the group consisting of NA101A102; and groups represented by the following Chemical Formula 1-3 and the following Chemical Formula 1-4, and any one of A and B is the group represented by the following Chemical Formula 1-3,
A101 and A102 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or group; a substituted or unsubstituted C2 to C60 alkynyl unsubstituted C1 to C60 alkoxy group; a substituted or unsubstituted C6 to C60 aryl group; and a substituted or unsubstituted C2 to C60 heteroaryl group, and
p and q are the same as or different from each other, and are each independently an integer of 0 to 3,

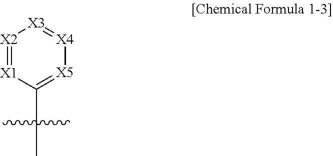

in Chemical Formula 1-3,
X1 is N or CRa,
X2 is N or CRb,
X3 is N or CRc,
X4 is N or CRd,
X5 is N or CRe,
at least one of X1 to X5 is N, and
Ra to Re are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heterocycle,

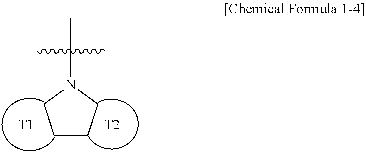

in Chemical Formula 1-4,
T1 ring and T2 ring are the same as or different from each other, and are each independently a substituted or unsubstituted C6 to C60 aromatic ring; or a substituted or unsubstituted C2 to C60 hetero aromatic ring.

In addition, one embodiment of the present disclosure provides an organic light emitting device comprising: a first electrode; a second electrode provided to face the first electrode; and one or more organic material layers provided between the first electrode and the second electrode, wherein one or more layers of the organic material layers comprise the heterocyclic compound represented by Chemical Formula 1.

In addition, one embodiment of the present disclosure provides an organic light emitting device, wherein the organic material layer further comprises a heterocyclic compound represented by the following Chemical Formula 2-1 or the following Chemical Formula 2-2:

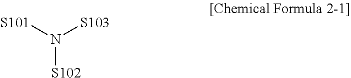

in Chemical Formula 2-1,
S101 to S103 are the same as or different from each other, and are each independently a substituted or unsubstituted C6 to C60 aryl group; or a substituted or unsubstituted C2 to C60 heteroaryl group,

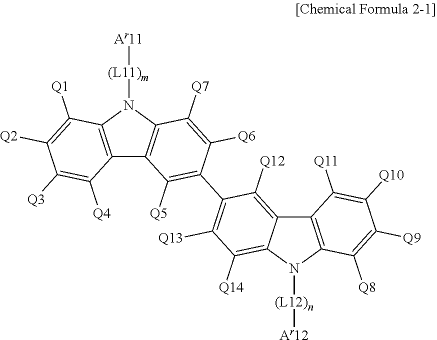

in Chemical Formula 2-2,
L11 and L12 are the same as or different from each other, and are each independently a single bond; a substituted or unsubstituted C6 to C60 arylene group; or a substituted or unsubstituted C2 to C60 heteroarylene group,
Ar11 and Ar12 are the same as or different from each other, and are each independently a substituted or unsubstituted C6 to C60 aryl group; or a substituted or unsubstituted C2 to C60 heteroaryl group,
Q1 to Q14 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group, and
m and n are the same as or different from each other, and are each independently an integer of 0 to 3.

In addition, one embodiment of the present disclosure provides a composition for an organic material layer of an organic light emitting device, the composition comprising the heterocyclic compound represented by Chemical Formula 1 and the heterocyclic compound represented by Chemical Formula 2-1 or Chemical Formula 2-2.

A heterocyclic compound according to one embodiment can be used as a material of an organic material layer of an organic light emitting device. The compound is capable of performing roles of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a light emitting layer material of an organic light emitting device.

Specifically, the heterocyclic compound can be used as a light emitting material either alone or as a mixture with a P-type host, and can be used as a host material or a dopant material of a light emitting layer. Using the compound represented by Chemical Formula 1 in an organic material layer is capable of lowering a driving voltage, enhancing light emission efficiency and enhancing lifetime properties in an organic light emitting device.

More specifically, using the heterocyclic compound according to one embodiment as a light emitting layer is capable of strengthening hole transport and electron transport properties, enhancing a hole transfer ability through adjusting a band gap and a triplet energy level (T₁ level) value, lowering a driving voltage of an organic light emitting device by increasing molecular stability, enhancing light efficiency, and enhancing lifetime properties of an organic light emitting device by enhanced thermal stability of the compound.

### BRIEF DESCRIPTION OF THE DRAWINGS

FIGS. 1 to 3 are views schematically showing a stacked structure of an organic light emitting device according to one embodiment of the present disclosure, respectively.

### DESCRIPTION OF SPECIFIC EMBODIMENTS

Hereinafter, the present disclosure will be described in more detail.

In the present specification, a term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, the term “substituted or unsubstituted” means that it is substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine or is unsubstituted, or being substituted with a substituent in which two or more substituents selected from among the substituents exemplified above are linked or being unsubstituted.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to and more specifically from 2 to 20.

In the present specification, an alkoxy group may be linear, branched or cyclic. The number of carbon atoms of the alkoxy group is not particularly limited, but is preferably from 1 to 20. Specific examples thereof may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, a phosphine oxide group is represented by —P(═O) T101T102, and T101 and T102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenyl phosphine oxide group, a dinaphthyl phosphine oxide group and the like, but are not limited thereto.

In the present specification, a silyl group is a substituent including Si and having the Si atom directly linked as a radical, and is represented by —SiT101T102T103. T101 to T103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

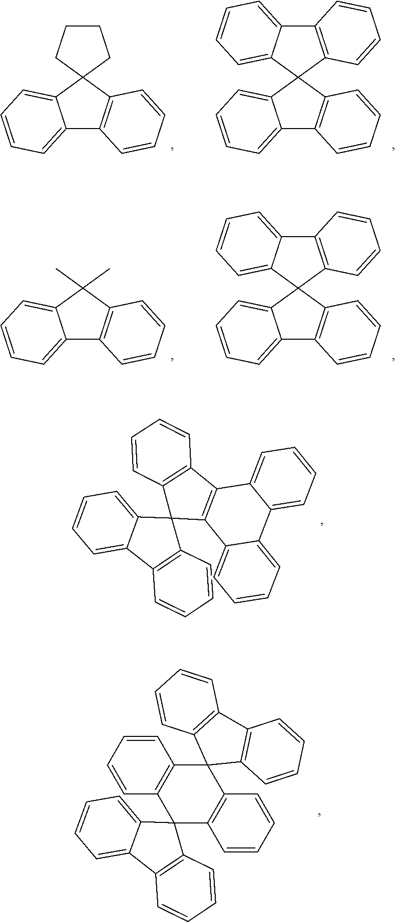

and the like may be included, however, the structure is not limited thereto.

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindenyl group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi (dibenzosilole) group, a dihydrophenazinyl group, a phenoxazinyl group, a phenanthridyl group, a thienyl group, an indolo[2,3-a] carbazolyl group, an indolo[2,3-b] carbazolyl group, an indolinyl group, a 10,11-dihydro-dibenzo[b, f]azepinyl group, a 9,10-dihydroacridinyl group, a phenanthrazinyl group, a phenothiathiazinyl group, a phthalazinyl group, a naphthylidinyl group, a phenanthrolinyl group, a benzo[c] [1,2,5] thiadiazolyl group, a 5,10-dihydrodibenzo[b, e] [1, 4]azasilinyl group, a pyrazolo[1,5-c] quinazolinyl group, a pyrido[1,2-b] indazolyl group, a pyrido[1,2-a]imidazo[1,2-e]indolinyl group, a 5,11-dihydroindeno[1,2-b] carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, an arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

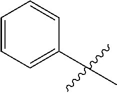 [SMILES: *c1ccccc1]

means that the total number of substituents that the phenyl group may have is 5 (T₁ in the formula), and the number of deuterium among these is 1 (T2 in the formula). In other words, having a deuterium content of 20% in a phenyl group may be represented by the following structural formulae.

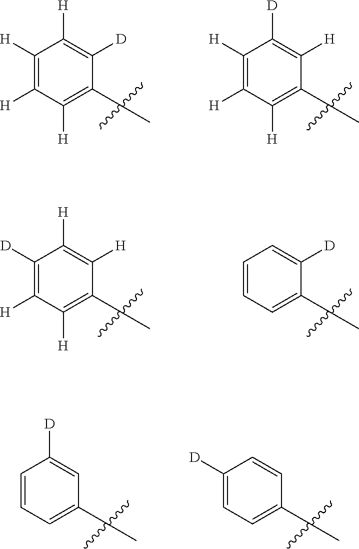 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present disclosure provides a heterocyclic compound represented by the following Chemical Formula 1:

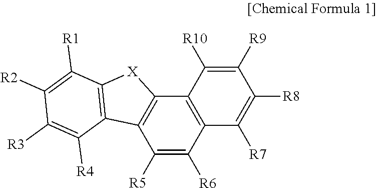

in Chemical Formula 1,
X is O or S,
R1 to R10 are the same as or different from each other, and are each independently selected from the group consisting of hydrogen; deuterium; halogen; a cyano group; a substituted or unsubstituted C1 to C60 a substituted or alkyl group; unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 1-1; and a group represented by the following Chemical Formula 1-2, and
at least one of R5 and R6 is the group represented by the following Chemical Formula 1-1, and at least one of R7 to R10 is the group represented by the following Chemical Formula 1-2,

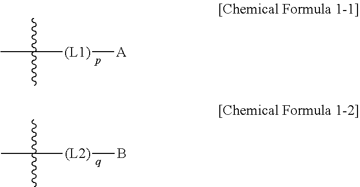

in Chemical Formulae 1-1 and 1-2,
L1 and L2 are the same as or different from each other, and are each independently a single bond; a substituted or unsubstituted C6 to C60 arylene group; or a substituted or unsubstituted C2 to C60 heteroarylene group,
A and B are different from each other, and are each independently selected from the group consisting of NA101A102; and groups represented by the following Chemical Formula 1-3 and the following Chemical Formula 1-4, and any one of A and B is the group represented by the following Chemical Formula 1-3,
A101 and A102 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, and
p and q are the same as or different from each other, and are each independently an integer of 0 to 3,

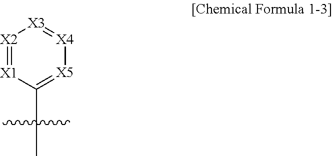

in Chemical Formula 1-3,
X1 is N or CRa,
X2 is N or CRb,
X3 is N or CRc,
X4 is N or CRd,
X5 is N or CRe,
at least one of X1 to X5 is N, and
Ra to Re are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heterocycle,

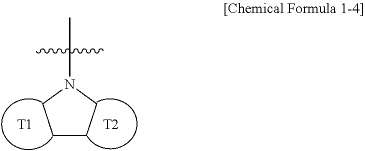

in Chemical Formula 1-4,
T1 ring and T2 ring are the same as or different from each other, and each independently a substituted or unsubstituted C6 to C60 aromatic ring; or a substituted or unsubstituted C2 to C60 hetero aromatic ring.

In one embodiment of the present disclosure, R1 to R10 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl a substituted or group; unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; the group represented by Chemical Formula 1-1; or the group represented by Chemical Formula 1-2, and at least one of R5 and R6 may be the group represented by Chemical Formula 1-1 and at least one of R7 to R10 may be the group represented by Chemical Formula 1-2.

In another embodiment of the present disclosure, R1 to R10 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; the group represented by Chemical Formula 1-1; or the group represented by Chemical Formula 1-2, and at least one of R5 and R6 may be the group represented by Chemical Formula 1-1 and at least one of R7 to R10 may be the group represented by Chemical Formula 1-2.

In one embodiment of the present disclosure, in Chemical Formula 1-1 and Chemical Formula 1-2, A and B are different from each other, and may be each independently selected from the group consisting of NA101A102; and the groups represented by Chemical Formula 1-3 and Chemical Formula 1-4, and any one of A and B may be the group represented by Chemical Formula 1-3.

In another embodiment of the present disclosure, in Chemical Formula 1-1 and Chemical Formula 1-2, A may be NA101A102; or the group represented by Chemical Formula 1-4, and B may be the group represented by Chemical Formula 1-3.

In another embodiment of the present disclosure, in Chemical Formula 1-1 and Chemical Formula 1-2, A may be the group represented by Chemical Formula 1-3, and B may be NA101A102; or the group represented by Chemical Formula 1-4.

In another embodiment of the present disclosure, in Chemical Formula 1-1 and Chemical Formula 1-2, A101 and A102 are the same as or different from each other, and may be each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C6 to C40 aryl group; and a substituted or unsubstituted C2 to C40 heteroaryl group.

In another embodiment of the present disclosure, in Chemical Formula 1-1 and Chemical Formula 1-2, A101 and A102 are the same as or different from each other, and may be each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C6 to C30 aryl group; and a substituted or unsubstituted C2 to C30 heteroaryl group.

In another embodiment of the present disclosure, in Chemical Formula 1-1 and Chemical Formula 1-2, L1 and L2 are the same as or different each other, and may be each independently a single bond; a substituted or unsubstituted C6 to C30 arylene group; or a substituted or unsubstituted C2 to C30 heteroarylene group.

In another embodiment of the present disclosure, in Chemical Formula 1-1 and Chemical Formula 1-2, L1 and L2 are the same as or different from each other, and may be each independently a single bond; a substituted or unsubstituted C6 to C20 arylene group; or a substituted or unsubstituted C2 to C20 heteroarylene group.

In another embodiment of the present disclosure, in Chemical Formula 1-1 and Chemical Formula 1-2, L1 and L2 are the same as or different from each other, and may be each independently a single bond; or a substituted or unsubstituted C6 to C20 arylene group.

In another embodiment of the present disclosure, in Chemical Formula 1-1 and Chemical Formula 1-2, L1 and L2 are the same as or different from each other, and may be each independently a single bond; or a substituted or unsubstituted phenylene group.

Specific examples of L1 and L2 are shown below, however, L1 and L2 are not limited to these examples.

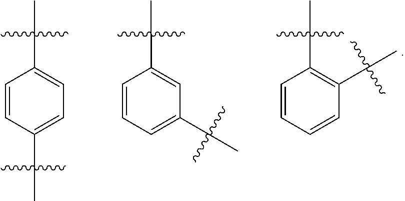 [SMILES: *c1ccc(*)cc1 ; *c1cccc(*)c1 ; *c1ccccc1*]

In one embodiment of the present disclosure, in Chemical Formula 1-3, X1 is N or CRa, X2 is N or CRb, X3 is N or CRc, X4 is N or CRd, X5 is N or CRe, and two or more of X1 to X5 may be N.

In another embodiment of the present disclosure, in Chemical Formula 1-3, Ra to Re are the same as or different from each other, and are each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C6 to C30 aryl group; and a substituted or unsubstituted C2 to C30 heteroaryl group, or two or more groups adjacent to each other may bond to each other to form a substituted or unsubstituted C6 to C30 aromatic hydrocarbon ring; or a substituted or unsubstituted C2 to C30 heteroring.

In another embodiment of the present disclosure, in Chemical Formula 1-3, Ra to Re are the same as or different from each other, and each independently selected from the group consisting of hydrogen; deuterium; halogen; a cyano group; a substituted or unsubstituted C to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, the group represented by Chemical Formula 1-3 may be any one of the following Chemical Formulae 1-3-1, 1-3-2 and 1-3-3, but is not limited to these examples:

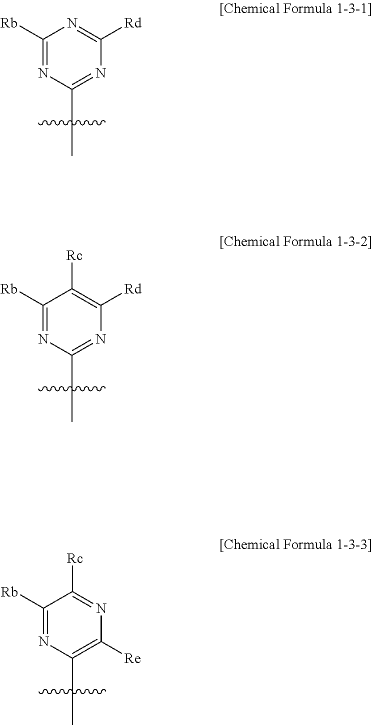

in Chemical Formulae 1-3-1, 1-3-2 and 1-3-3,
Rb to Re are the same as defined in Chemical Formula 1-3.

In one embodiment of the present disclosure, in Chemical Formula 1-4, T1 ring and T2 ring are the same as or different from each other, and may be each independently a substituted or unsubstituted C6 to C30 aromatic ring; or a substituted or unsubstituted C2 to C30 hetero aromatic ring.

In another embodiment of the present disclosure, in Chemical Formula 1-4, T1 ring and T2 ring are the same as or different from each other, and may be each independently a substituted or unsubstituted C6 to C20 aromatic ring; or a substituted or unsubstituted C2 to C20 hetero aromatic ring.

In one embodiment of the present disclosure, the group represented by Chemical Formula 1-4 may be any one of the following Chemical Formulae 1-4-1, 1-4-2 and 1-4-3, but is not limited to these examples:

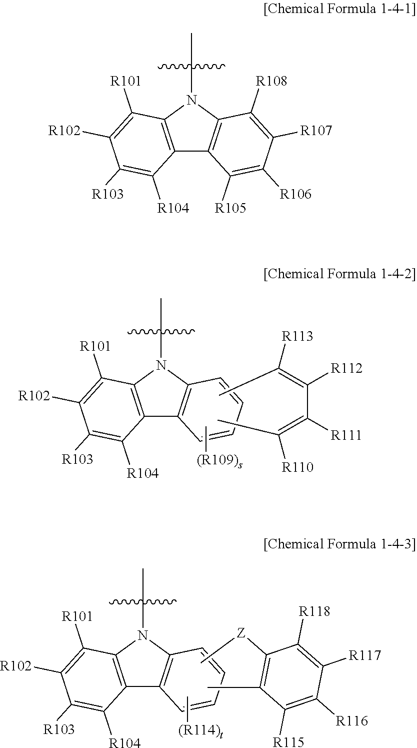

in Chemical Formulae 1-4-1, 1-4-2 and 1-4-3,
R101 to R118 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic ring; or a substituted or unsubstituted C2 to C60 heteroring,
Z is NR120, O or S,
R120 is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, and
s and t are the same as or different from each other, and are each independently an integer of 1 or 2.

In another embodiment of the present disclosure, in Chemical Formulae 1-4-1, 1-4-2 and 1-4-3, R101 to R118 are the same as or different from each other, and are each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and a substituted or unsubstituted C2 to C30 heteroaryl group, or two or more groups adjacent to each other may bond to each other to form a substituted or unsubstituted C6 to C30 aromatic ring; or a substituted or unsubstituted C2 to C30 heteroring.

In another embodiment of the present disclosure, in Chemical Formulae 1-4-1, 1-4-2 and 1-4-3, R101 to R118 are the same as or different from each other, and each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic ring; or a substituted or unsubstituted C2 to C20 heteroring.

In another embodiment of the present disclosure, in Chemical Formula 1-4-3, Z may be NR120, O or S, and R120 may be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C6 to C30 aryl group; and a substituted or unsubstituted C2 to C30 heteroaryl group.

In another embodiment of the present disclosure, in Chemical Formula 1-4-3, Z may be NR120, O or S, and R120 may be selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C6 to C20 aryl group; and a substituted or unsubstituted C2 to C20 heteroaryl group.

In one embodiment of the present disclosure, the group represented by Chemical Formula 1-4-2 may be any one of the following Chemical Formulae 1-4-2-a, 1-4-2-b and 1-4-2-c, but is not limited to these examples:

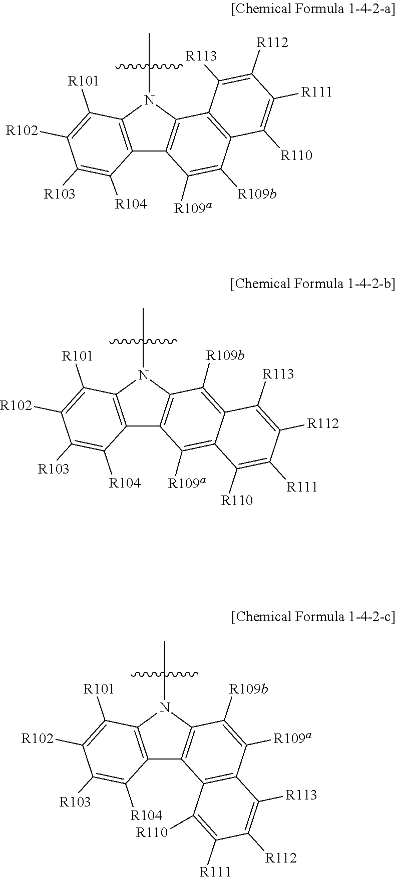

in Chemical Formulae 1-4-2-a, 1-4-2-b and 1-4-2-c,
R109a and R109b are the same as defined in R109, and
R101 to R104 and R109 to R113 are the same as defined in Chemical Formula 1-4-2.

In one embodiment of the present disclosure, the group represented by Chemical Formula 1-4-3 may be any one of the following Chemical Formulae 1-4-3-a to 1-4-3-f, but is not limited to these examples:

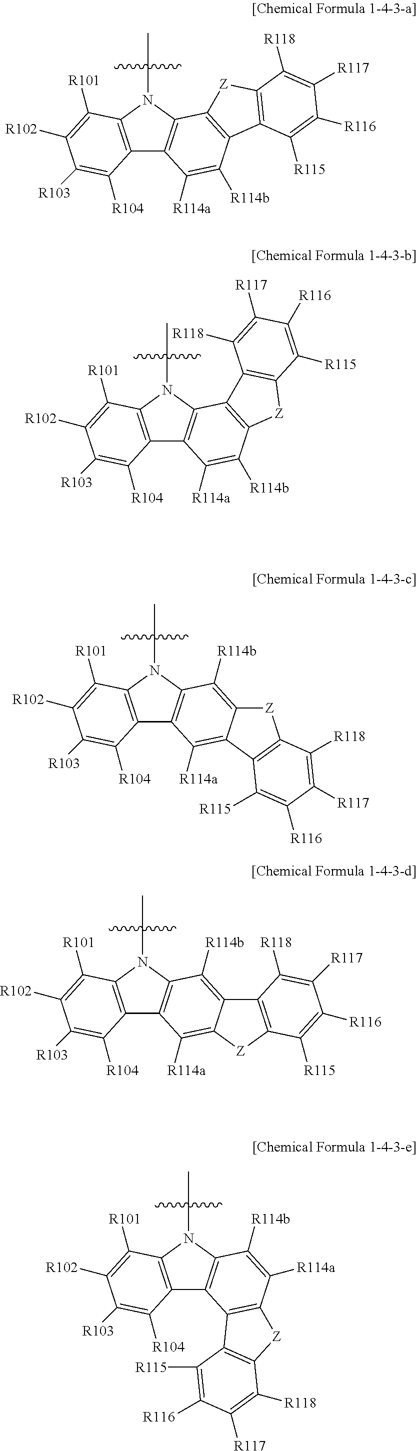

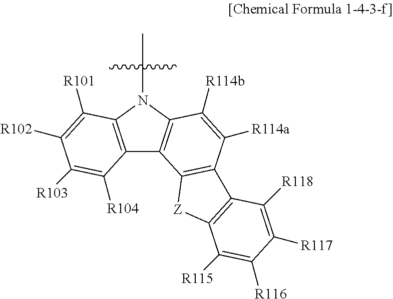

in Chemical Formulae 1-4-3-a to 1-4-3-f,
R114a and R114b are the same as defined in R114, and
R101 to R104 and R114 to R118 are the same as defined in Chemical Formula 1-4-3.

In one embodiment of the present disclosure, heterocyclic compound represented by Chemical Formula 1 does not include deuterium as a substituent, or the content of deuterium based on the total number of hydrogen atoms and deuterium atoms may be 1% to 100%.

In another embodiment of the present disclosure, the heterocyclic compound represented by Chemical Formula 1 does not include deuterium as a substituent, or the content of deuterium based on the total number of hydrogen atoms and deuterium atoms may be 10% to 90%.

In another embodiment of the present disclosure, the heterocyclic compound represented by Chemical Formula 1 does not include deuterium as a substituent, or the content of deuterium based on the total number of hydrogen atoms and deuterium atoms may be 20% to 80%.

In another embodiment of the present disclosure, the heterocyclic compound represented by Chemical Formula 1 does not include deuterium as a substituent, or the content of deuterium based on the total number of hydrogen atoms and deuterium atoms may be 30% to 70%.

For example, the heterocyclic compound represented by Chemical Formula 1 does not include deuterium as a substituent, or the content of deuterium may be more than 08, 1% or more, 5% or more, 10% or more, 15% or more, 20% or more, 25% or more, 30% or more, 35% or more, 40% or more, 45% or more or 50% or more, and 100% or less, 95% or less, 90% or less, 85% or less, 80% or less, 75% or less, 70% or less, 65% or less or 60% or less based on the total number of hydrogen atoms and deuterium atoms.

In one embodiment of the present disclosure, the heterocyclic compound represented by Chemical Formula 1 may be any one selected from the group consisting of the following compounds.

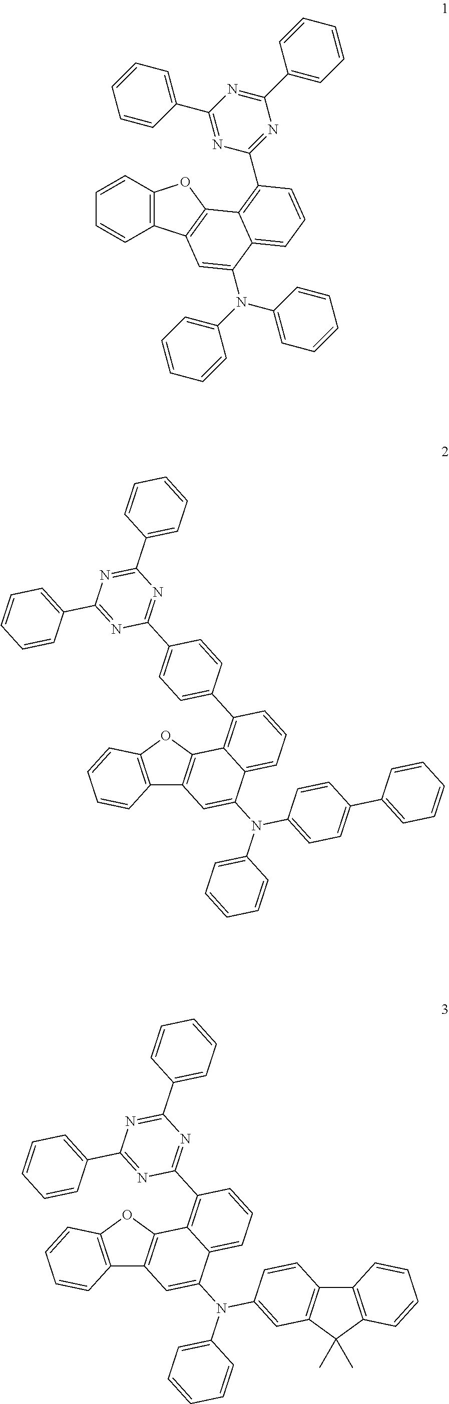 [SMILES: c1ccc(-c2nc(-c3ccccc3)nc(-c3cccc4c(N(c5ccccc5)c5ccccc5)cc5c6ccccc6oc5c34)n2)cc1 ; c1ccc(-c2ccc(N(c3ccccc3)c3cc4c5ccccc5oc4c4c(-c5ccc(-c6nc(-c7ccccc7)nc(-c7ccccc7)n6)cc5)cccc34)cc2)cc1 ; CC1(C)c2ccccc2-c2ccc(N(c3ccccc3)c3cc4c5ccccc5oc4c4c(-c5nc(-c6ccccc6)nc(-c6ccccc6)n5)cccc34)cc21]

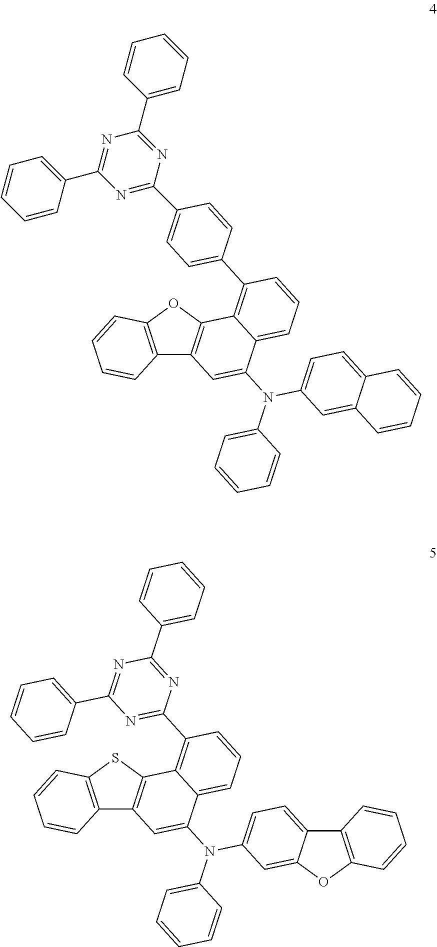 [SMILES: c1ccc(-c2nc(-c3ccccc3)nc(-c3ccc(-c4cccc5c(N(c6ccccc6)c6ccc7ccccc7c6)cc6c7ccccc7oc6c45)cc3)n2)cc1 ; c1ccc(-c2nc(-c3ccccc3)nc(-c3cccc4c(N(c5ccccc5)c5ccc6c(c5)oc5ccccc56)cc5c6ccccc6sc5c34)n2)cc1]

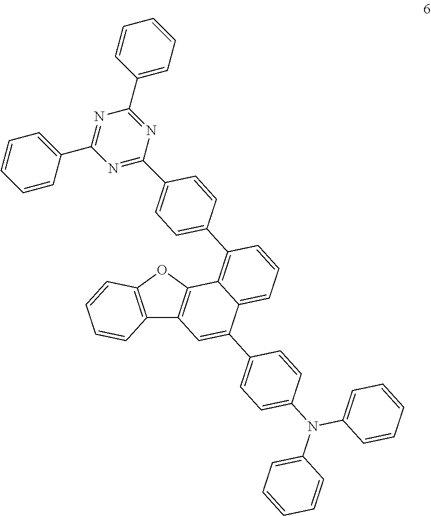 [SMILES: c1ccc(-c2nc(-c3ccccc3)nc(-c3ccc(-c4cccc5c(-c6ccc(N(c7ccccc7)c7ccccc7)cc6)cc6c7ccccc7oc6c45)cc3)n2)cc1]

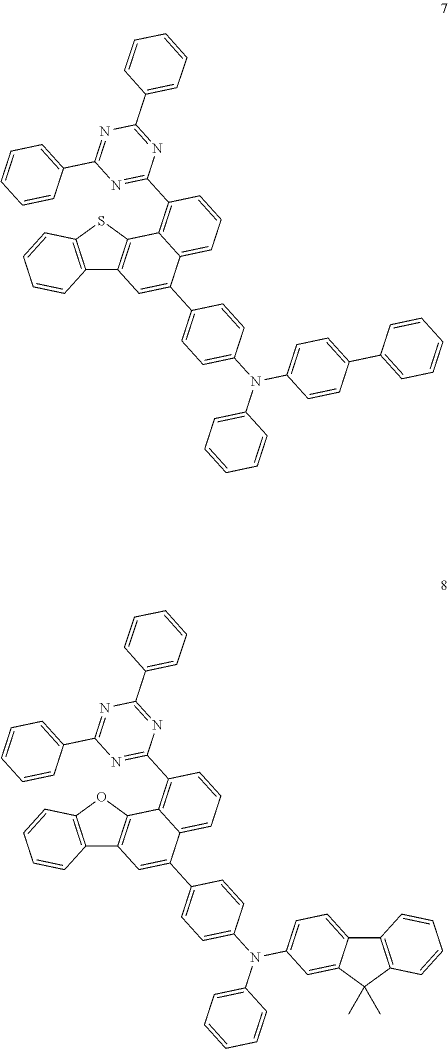 [SMILES: c1ccc(-c2ccc(N(c3ccccc3)c3ccc(-c4cc5c6ccccc6sc5c5c(-c6nc(-c7ccccc7)nc(-c7ccccc7)n6)cccc45)cc3)cc2)cc1 ; CC1(C)c2ccccc2-c2ccc(N(c3ccccc3)c3ccc(-c4cc5c6ccccc6oc5c5c(-c6nc(-c7ccccc7)nc(-c7ccccc7)n6)cccc45)cc3)cc21]

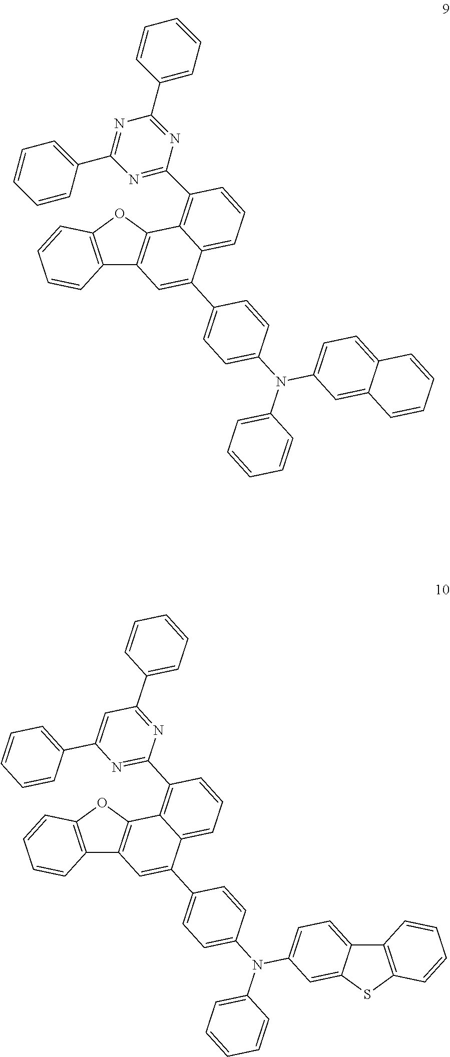 [SMILES: c1ccc(-c2nc(-c3ccccc3)nc(-c3cccc4c(-c5ccc(N(c6ccccc6)c6ccc7ccccc7c6)cc5)cc5c6ccccc6oc5c34)n2)cc1 ; c1ccc(-c2cc(-c3ccccc3)nc(-c3cccc4c(-c5ccc(N(c6ccccc6)c6ccc7c(c6)sc6ccccc67)cc5)cc5c6ccccc6oc5c34)n2)cc1]

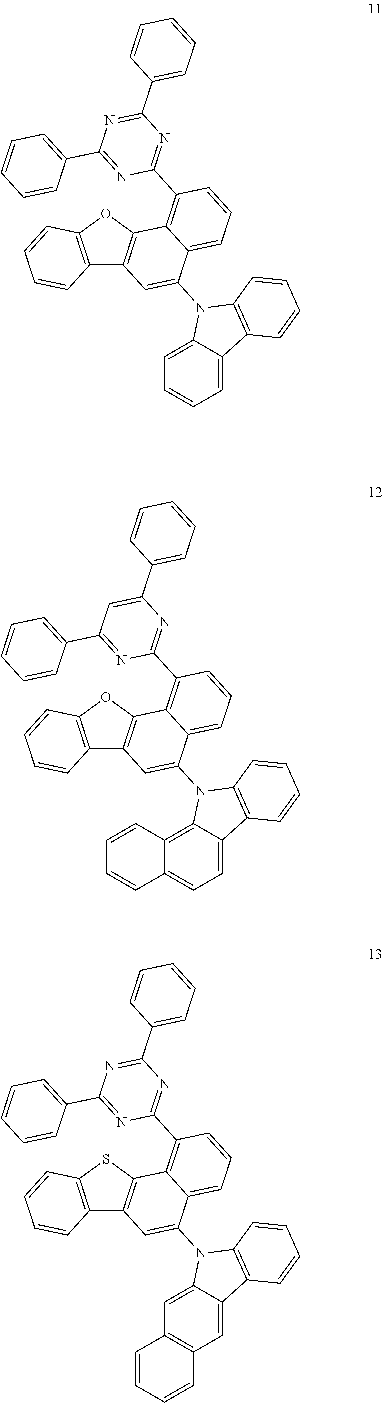 [SMILES: c1ccc(-c2nc(-c3ccccc3)nc(-c3cccc4c(-n5c6ccccc6c6ccccc65)cc5c6ccccc6oc5c34)n2)cc1 ; c1ccc(-c2cc(-c3ccccc3)nc(-c3cccc4c(-n5c6ccccc6c6ccc7ccccc7c65)cc5c6ccccc6oc5c34)n2)cc1 ; c1ccc(-c2nc(-c3ccccc3)nc(-c3cccc4c(-n5c6ccccc6c6cc7ccccc7cc65)cc5c6ccccc6sc5c34)n2)cc1]

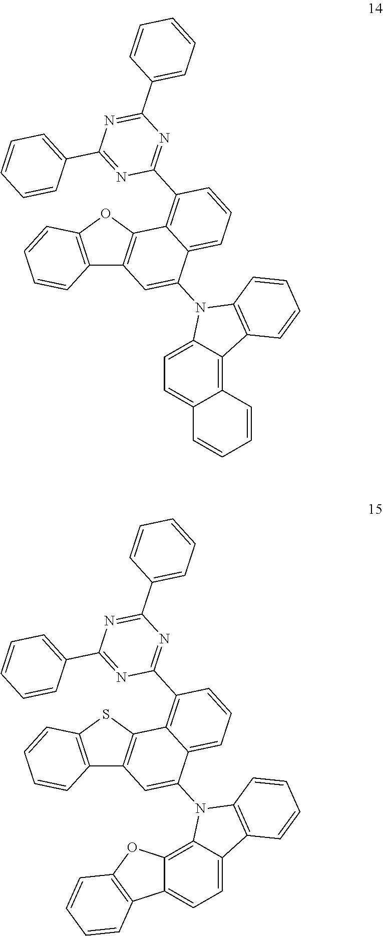 [SMILES: c1ccc(-c2nc(-c3ccccc3)nc(-c3cccc4c(-n5c6ccccc6c6c7ccccc7ccc65)cc5c6ccccc6oc5c34)n2)cc1 ; c1ccc(-c2nc(-c3ccccc3)nc(-c3cccc4c(-n5c6ccccc6c6ccc7c8ccccc8oc7c65)cc5c6ccccc6sc5c34)n2)cc1]

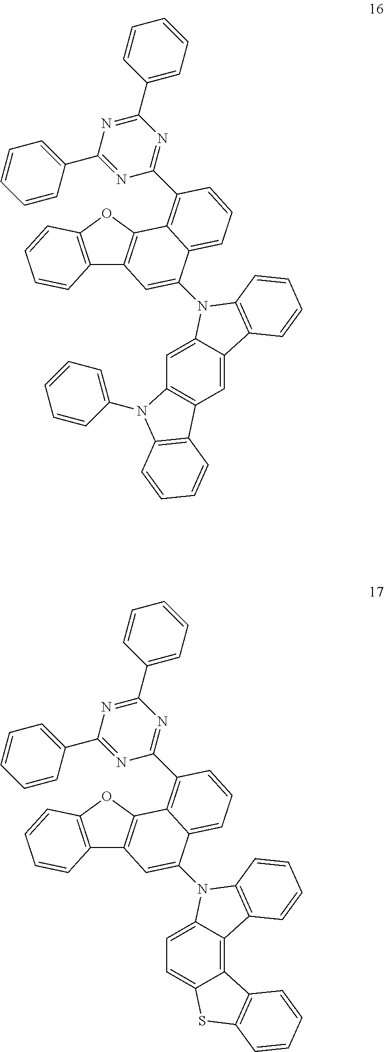 [SMILES: c1ccc(-c2nc(-c3ccccc3)nc(-c3cccc4c(-n5c6ccccc6c6cc7c8ccccc8n(-c8ccccc8)c7cc65)cc5c6ccccc6oc5c34)n2)cc1 ; c1ccc(-c2nc(-c3ccccc3)nc(-c3cccc4c(-n5c6ccccc6c6c7c(ccc65)sc5ccccc57)cc5c6ccccc6oc5c34)n2)cc1]

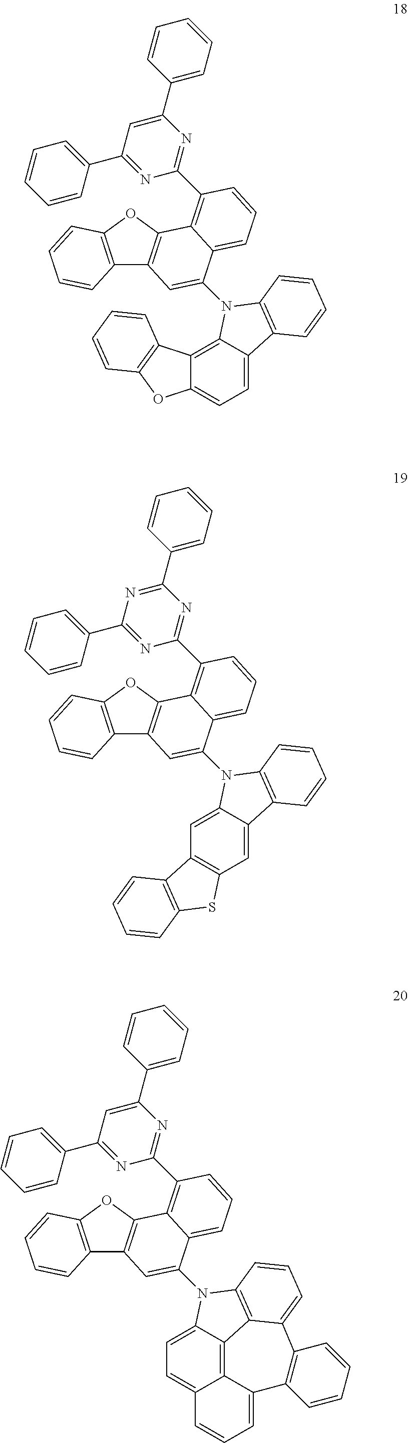 [SMILES: c1ccc(-c2cc(-c3ccccc3)nc(-c3cccc4c(-n5c6ccccc6c6ccc7oc8ccccc8c7c65)cc5c6ccccc6oc5c34)n2)cc1 ; c1ccc(-c2nc(-c3ccccc3)nc(-c3cccc4c(-n5c6ccccc6c6cc7sc8ccccc8c7cc65)cc5c6ccccc6oc5c34)n2)cc1 ; c1ccc(-c2cc(-c3ccccc3)nc(-c3cccc4c(-n5c6cccc7c6c6c8c(cccc8ccc65)-c5ccccc5-7)cc5c6ccccc6oc5c34)n2)cc1]

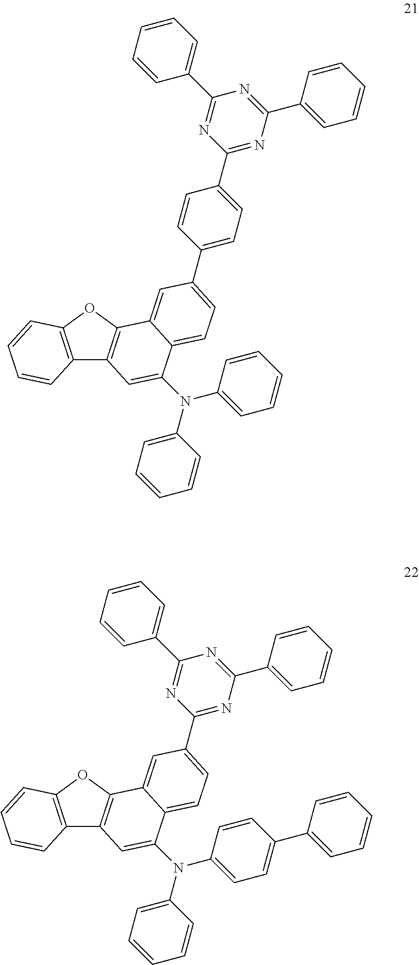 [SMILES: c1ccc(-c2nc(-c3ccccc3)nc(-c3ccc(-c4ccc5c(N(c6ccccc6)c6ccccc6)cc6c7ccccc7oc6c5c4)cc3)n2)cc1 ; c1ccc(-c2ccc(N(c3ccccc3)c3cc4c5ccccc5oc4c4cc(-c5nc(-c6ccccc6)nc(-c6ccccc6)n5)ccc34)cc2)cc1]

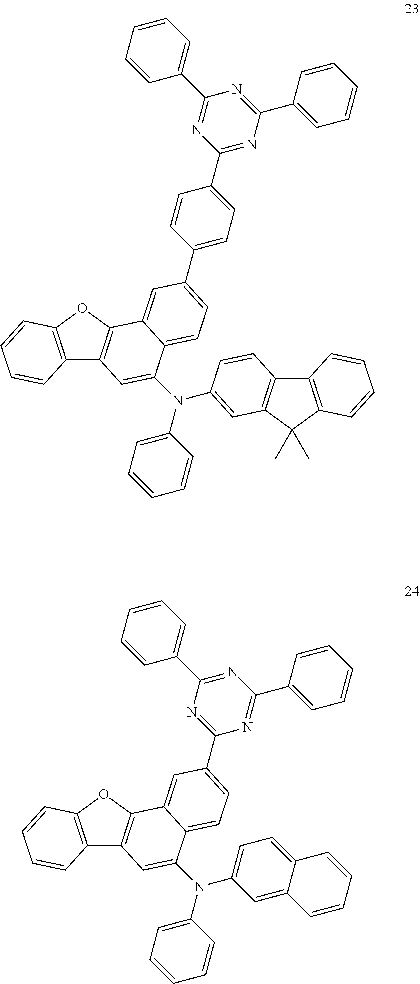 [SMILES: CC1(C)c2ccccc2-c2ccc(N(c3ccccc3)c3cc4c5ccccc5oc4c4cc(-c5ccc(-c6nc(-c7ccccc7)nc(-c7ccccc7)n6)cc5)ccc34)cc21 ; c1ccc(-c2nc(-c3ccccc3)nc(-c3ccc4c(N(c5ccccc5)c5ccc6ccccc6c5)cc5c6ccccc6oc5c4c3)n2)cc1]

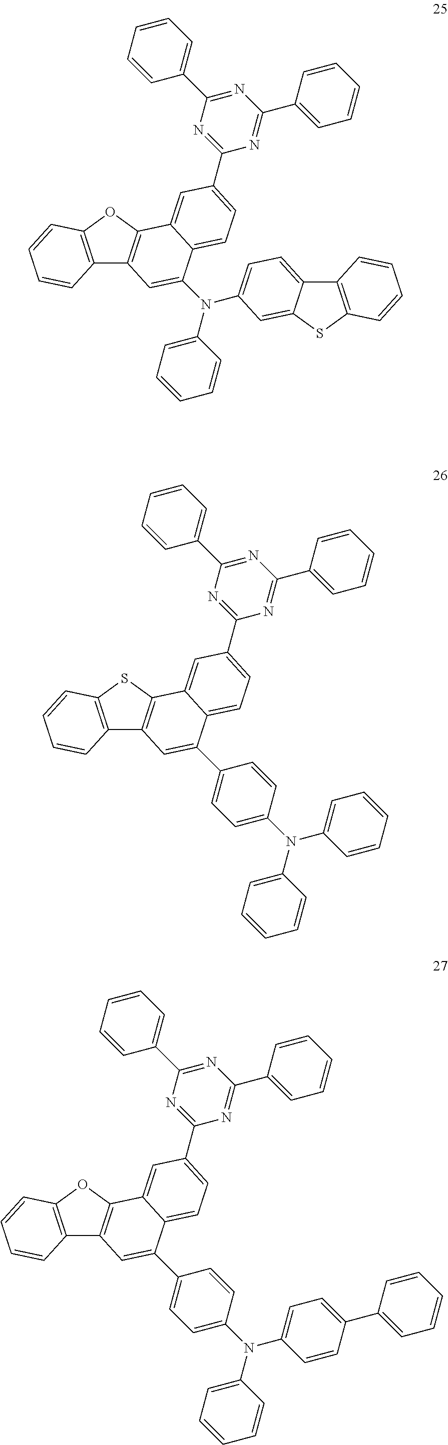 [SMILES: c1ccc(-c2nc(-c3ccccc3)nc(-c3ccc4c(N(c5ccccc5)c5ccc6c(c5)sc5ccccc56)cc5c6ccccc6oc5c4c3)n2)cc1 ; c1ccc(-c2nc(-c3ccccc3)nc(-c3ccc4c(-c5ccc(N(c6ccccc6)c6ccccc6)cc5)cc5c6ccccc6sc5c4c3)n2)cc1 ; c1ccc(-c2ccc(N(c3ccccc3)c3ccc(-c4cc5c6ccccc6oc5c5cc(-c6nc(-c7ccccc7)nc(-c7ccccc7)n6)ccc45)cc3)cc2)cc1]

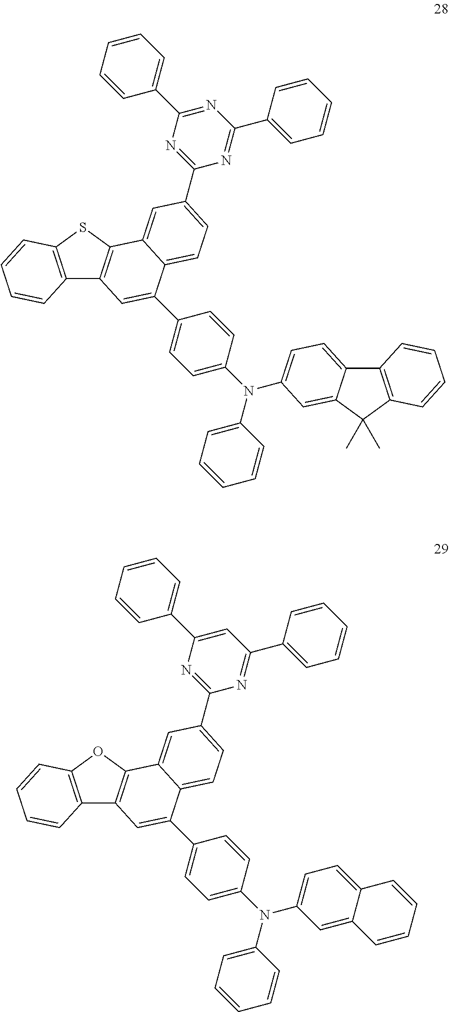 [SMILES: CC1(C)c2ccccc2-c2ccc(N(c3ccccc3)c3ccc(-c4cc5c6ccccc6sc5c5cc(-c6nc(-c7ccccc7)nc(-c7ccccc7)n6)ccc45)cc3)cc21 ; c1ccc(-c2cc(-c3ccccc3)nc(-c3ccc4c(-c5ccc(N(c6ccccc6)c6ccc7ccccc7c6)cc5)cc5c6ccccc6oc5c4c3)n2)cc1]

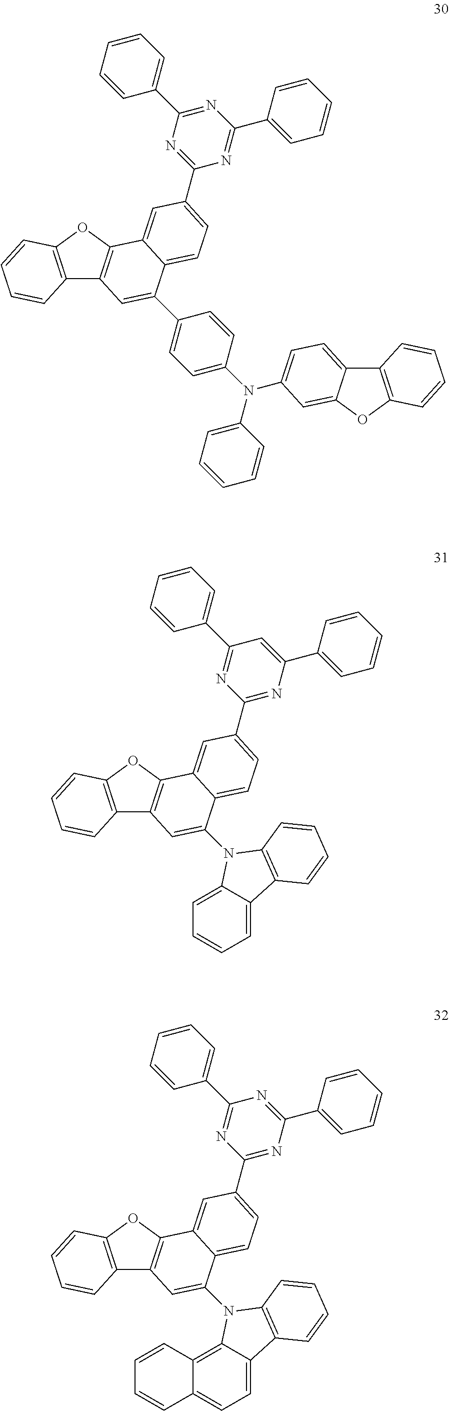 [SMILES: c1ccc(-c2nc(-c3ccccc3)nc(-c3ccc4c(-c5ccc(N(c6ccccc6)c6ccc7c(c6)oc6ccccc67)cc5)cc5c6ccccc6oc5c4c3)n2)cc1 ; c1ccc(-c2cc(-c3ccccc3)nc(-c3ccc4c(-n5c6ccccc6c6ccccc65)cc5c6ccccc6oc5c4c3)n2)cc1 ; c1ccc(-c2nc(-c3ccccc3)nc(-c3ccc4c(-n5c6ccccc6c6ccc7ccccc7c65)cc5c6ccccc6oc5c4c3)n2)cc1]

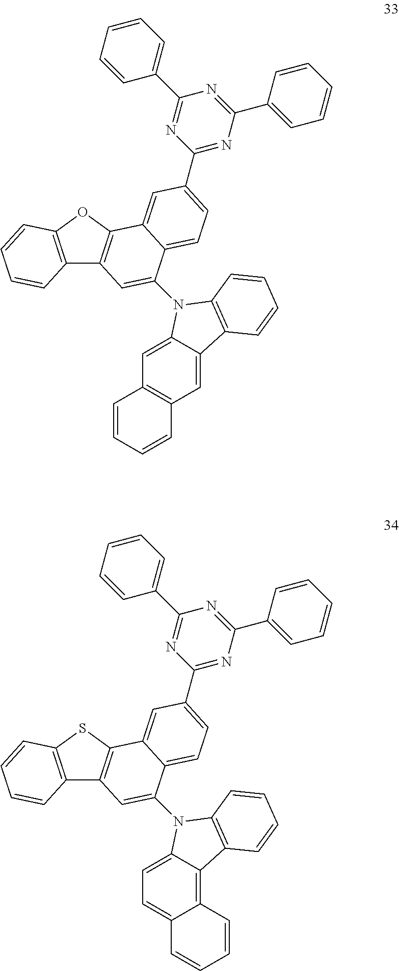 [SMILES: c1ccc(-c2nc(-c3ccccc3)nc(-c3ccc4c(-n5c6ccccc6c6cc7ccccc7cc65)cc5c6ccccc6oc5c4c3)n2)cc1 ; c1ccc(-c2nc(-c3ccccc3)nc(-c3ccc4c(-n5c6ccccc6c6c7ccccc7ccc65)cc5c6ccccc6sc5c4c3)n2)cc1]

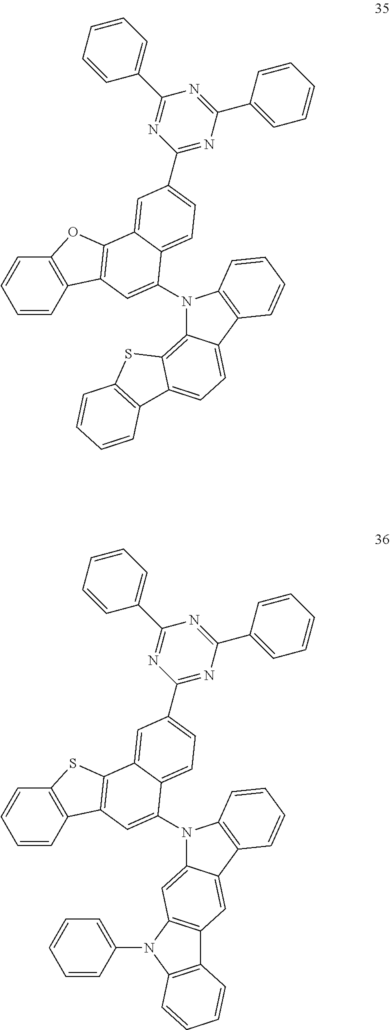 [SMILES: c1ccc(-c2nc(-c3ccccc3)nc(-c3ccc4c(-n5c6ccccc6c6ccc7c8ccccc8sc7c65)cc5c6ccccc6oc5c4c3)n2)cc1 ; c1ccc(-c2nc(-c3ccccc3)nc(-c3ccc4c(-n5c6ccccc6c6cc7c8ccccc8n(-c8ccccc8)c7cc65)cc5c6ccccc6sc5c4c3)n2)cc1]

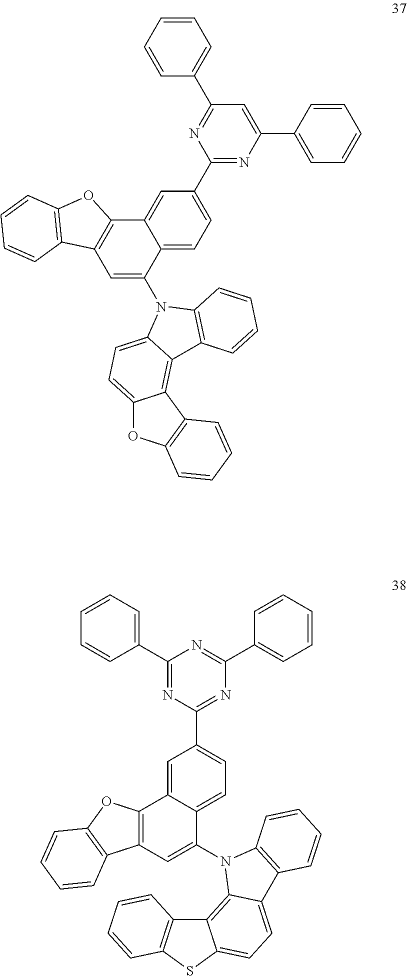 [SMILES: c1ccc(-c2cc(-c3ccccc3)nc(-c3ccc4c(-n5c6ccccc6c6c7c(ccc65)oc5ccccc57)cc5c6ccccc6oc5c4c3)n2)cc1 ; c1ccc(-c2nc(-c3ccccc3)nc(-c3ccc4c(-n5c6ccccc6c6ccc7sc8ccccc8c7c65)cc5c6ccccc6oc5c4c3)n2)cc1]

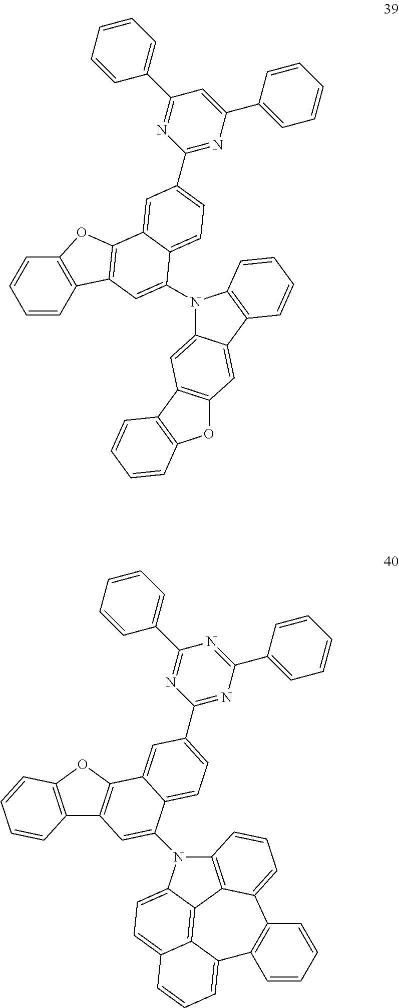 [SMILES: c1ccc(-c2cc(-c3ccccc3)nc(-c3ccc4c(-n5c6ccccc6c6cc7oc8ccccc8c7cc65)cc5c6ccccc6oc5c4c3)n2)cc1 ; c1ccc(-c2nc(-c3ccccc3)nc(-c3ccc4c(-n5c6cccc7c6c6c8c(cccc8ccc65)-c5ccccc5-7)cc5c6ccccc6oc5c4c3)n2)cc1]

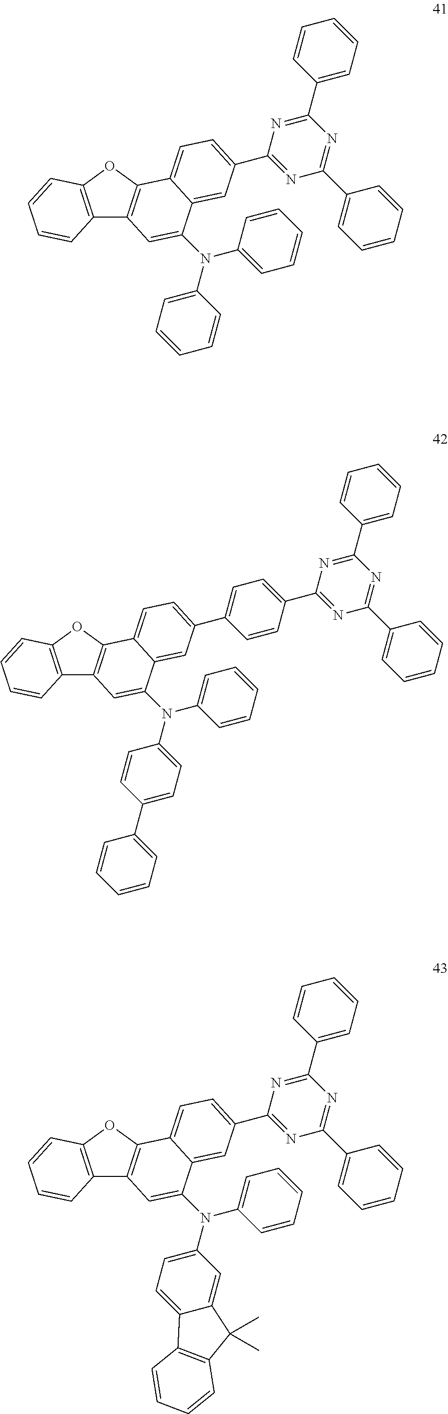 [SMILES: c1ccc(-c2nc(-c3ccccc3)nc(-c3ccc4c(c3)c(N(c3ccccc3)c3ccccc3)cc3c5ccccc5oc43)n2)cc1 ; c1ccc(-c2ccc(N(c3ccccc3)c3cc4c5ccccc5oc4c4ccc(-c5ccc(-c6nc(-c7ccccc7)nc(-c7ccccc7)n6)cc5)cc34)cc2)cc1 ; CC1(C)c2ccccc2-c2ccc(N(c3ccccc3)c3cc4c5ccccc5oc4c4ccc(-c5nc(-c6ccccc6)nc(-c6ccccc6)n5)cc34)cc21]

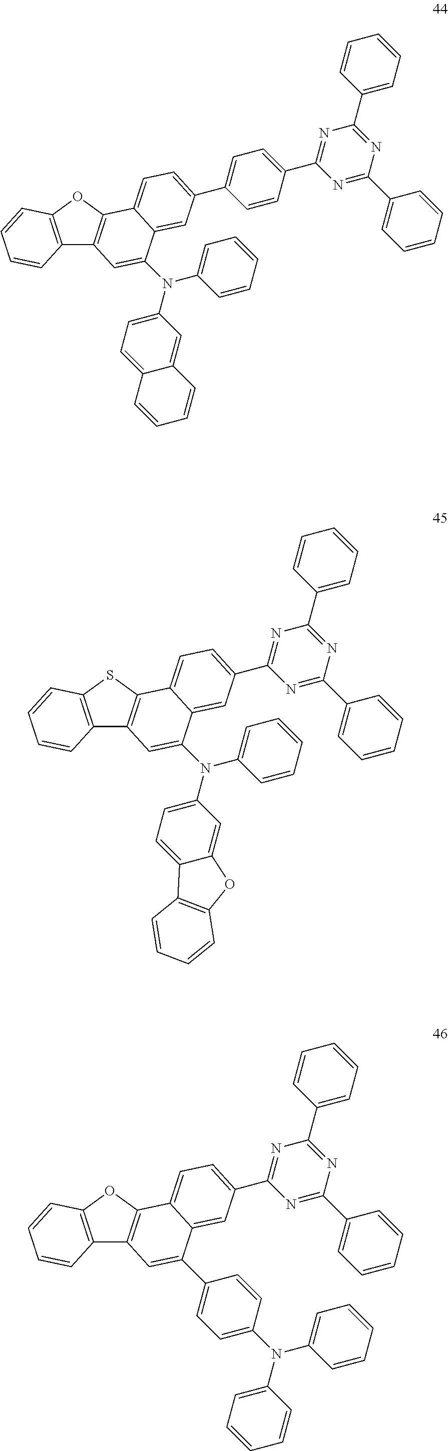 [SMILES: c1ccc(-c2nc(-c3ccccc3)nc(-c3ccc(-c4ccc5c(c4)c(N(c4ccccc4)c4ccc6ccccc6c4)cc4c6ccccc6oc54)cc3)n2)cc1 ; c1ccc(-c2nc(-c3ccccc3)nc(-c3ccc4c(c3)c(N(c3ccccc3)c3ccc5c(c3)oc3ccccc35)cc3c5ccccc5sc43)n2)cc1 ; c1ccc(-c2nc(-c3ccccc3)nc(-c3ccc4c(c3)c(-c3ccc(N(c5ccccc5)c5ccccc5)cc3)cc3c5ccccc5oc43)n2)cc1]

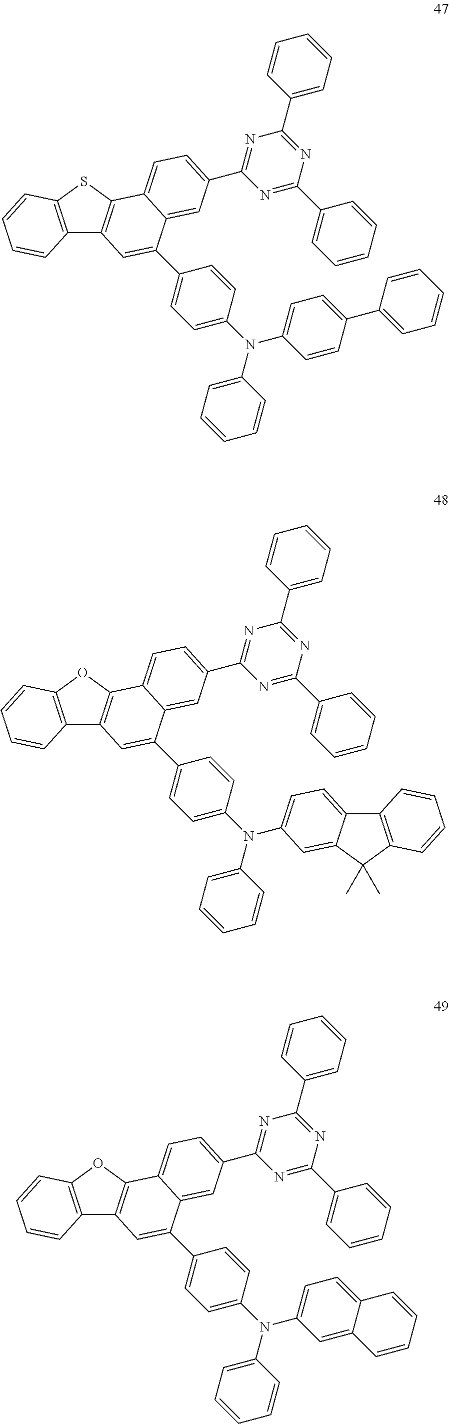 [SMILES: c1ccc(-c2ccc(N(c3ccccc3)c3ccc(-c4cc5c6ccccc6sc5c5ccc(-c6nc(-c7ccccc7)nc(-c7ccccc7)n6)cc45)cc3)cc2)cc1 ; CC1(C)c2ccccc2-c2ccc(N(c3ccccc3)c3ccc(-c4cc5c6ccccc6oc5c5ccc(-c6nc(-c7ccccc7)nc(-c7ccccc7)n6)cc45)cc3)cc21 ; c1ccc(-c2nc(-c3ccccc3)nc(-c3ccc4c(c3)c(-c3ccc(N(c5ccccc5)c5ccc6ccccc6c5)cc3)cc3c5ccccc5oc43)n2)cc1]

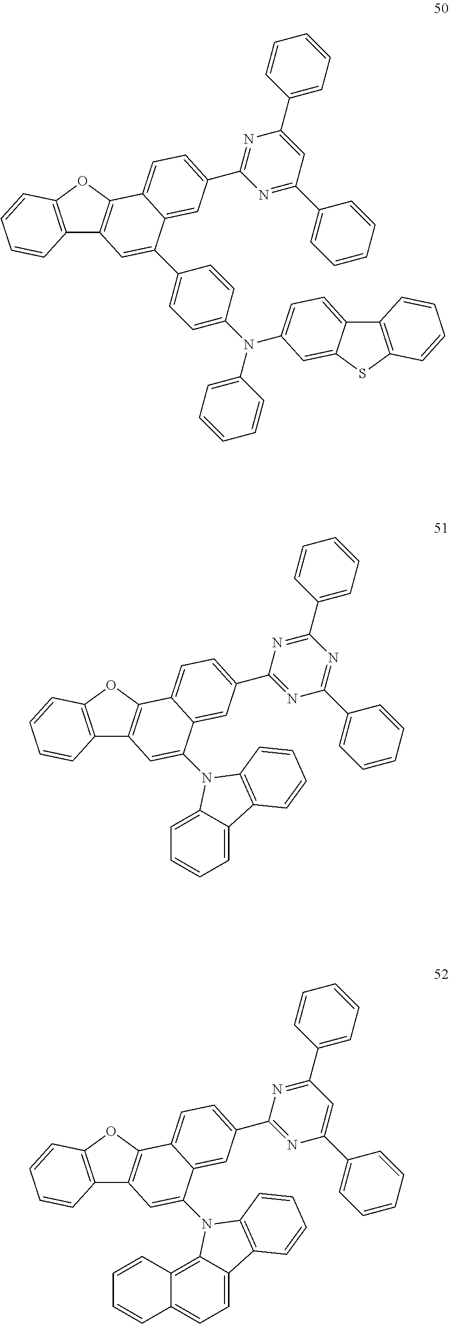 [SMILES: c1ccc(-c2cc(-c3ccccc3)nc(-c3ccc4c(c3)c(-c3ccc(N(c5ccccc5)c5ccc6c(c5)sc5ccccc56)cc3)cc3c5ccccc5oc43)n2)cc1 ; c1ccc(-c2nc(-c3ccccc3)nc(-c3ccc4c(c3)c(-n3c5ccccc5c5ccccc53)cc3c5ccccc5oc43)n2)cc1 ; c1ccc(-c2cc(-c3ccccc3)nc(-c3ccc4c(c3)c(-n3c5ccccc5c5ccc6ccccc6c53)cc3c5ccccc5oc43)n2)cc1]

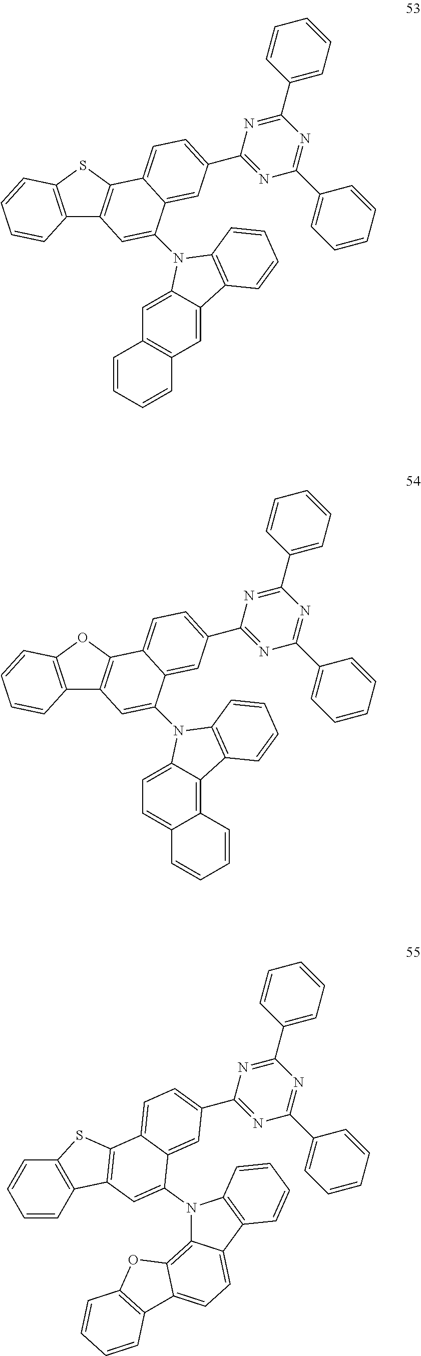 [SMILES: c1ccc(-c2nc(-c3ccccc3)nc(-c3ccc4c(c3)c(-n3c5ccccc5c5cc6ccccc6cc53)cc3c5ccccc5sc43)n2)cc1 ; c1ccc(-c2nc(-c3ccccc3)nc(-c3ccc4c(c3)c(-n3c5ccccc5c5c6ccccc6ccc53)cc3c5ccccc5oc43)n2)cc1 ; c1ccc(-c2nc(-c3ccccc3)nc(-c3ccc4c(c3)c(-n3c5ccccc5c5ccc6c7ccccc7oc6c53)cc3c5ccccc5sc43)n2)cc1]

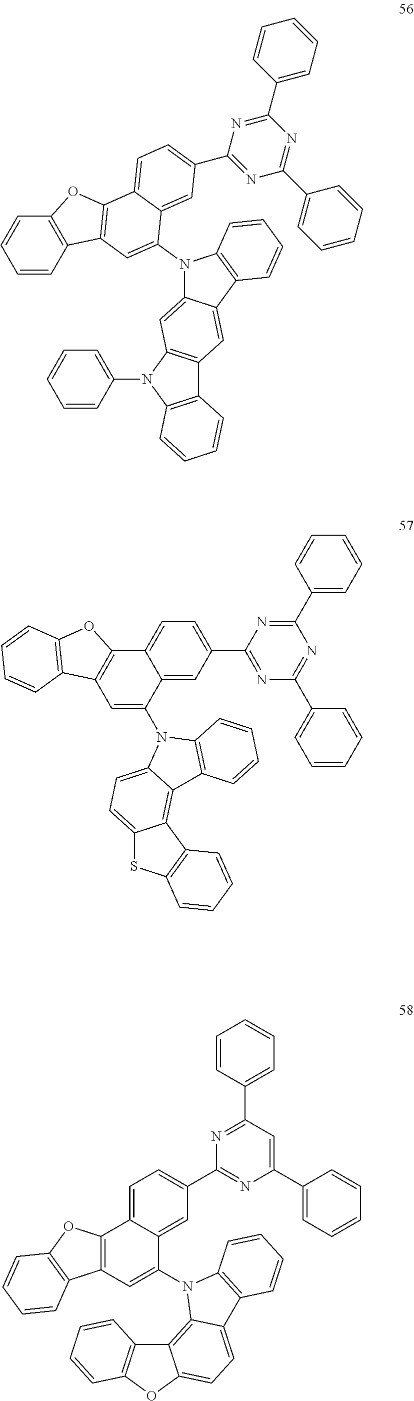 [SMILES: c1ccc(-c2nc(-c3ccccc3)nc(-c3ccc4c(c3)c(-n3c5ccccc5c5cc6c7ccccc7n(-c7ccccc7)c6cc53)cc3c5ccccc5oc43)n2)cc1 ; c1ccc(-c2nc(-c3ccccc3)nc(-c3ccc4c(c3)c(-n3c5ccccc5c5c6c(ccc53)sc3ccccc36)cc3c5ccccc5oc43)n2)cc1 ; c1ccc(-c2cc(-c3ccccc3)nc(-c3ccc4c(c3)c(-n3c5ccccc5c5ccc6oc7ccccc7c6c53)cc3c5ccccc5oc43)n2)cc1]

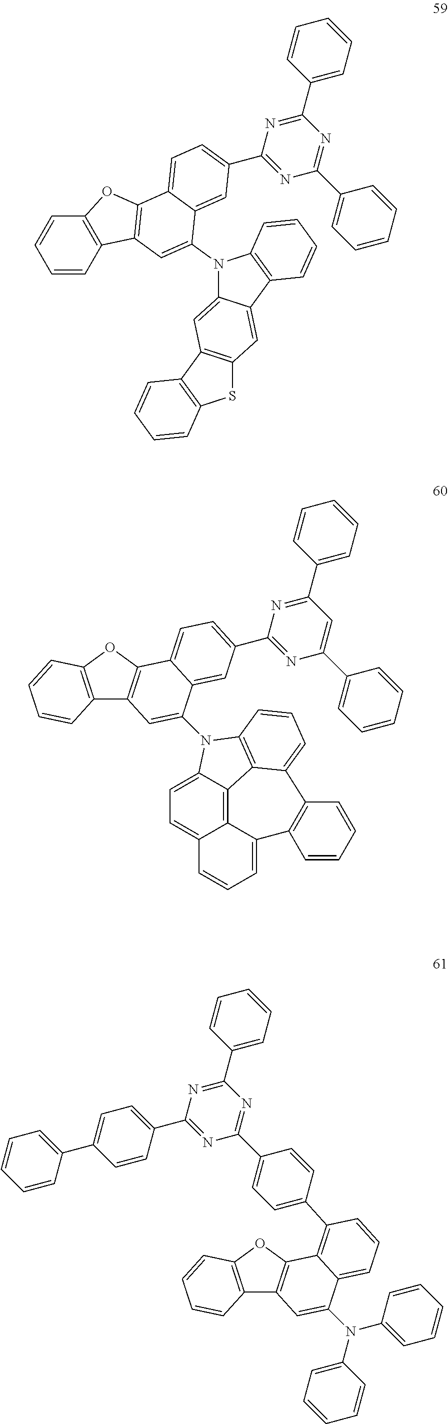 [SMILES: c1ccc(-c2nc(-c3ccccc3)nc(-c3ccc4c(c3)c(-n3c5ccccc5c5cc6sc7ccccc7c6cc53)cc3c5ccccc5oc43)n2)cc1 ; c1ccc(-c2cc(-c3ccccc3)nc(-c3ccc4c(c3)c(-n3c5cccc6c5c5c7c(cccc7ccc53)-c3ccccc3-6)cc3c5ccccc5oc43)n2)cc1 ; c1ccc(-c2ccc(-c3nc(-c4ccccc4)nc(-c4ccc(-c5cccc6c(N(c7ccccc7)c7ccccc7)cc7c8ccccc8oc7c56)cc4)n3)cc2)cc1]

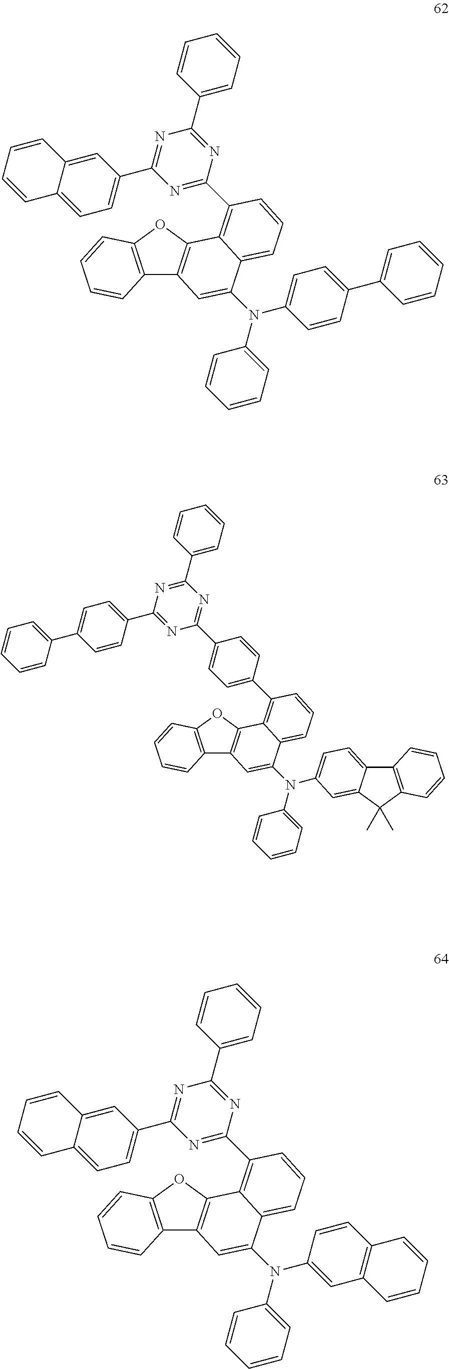 [SMILES: c1ccc(-c2ccc(N(c3ccccc3)c3cc4c5ccccc5oc4c4c(-c5nc(-c6ccccc6)nc(-c6ccc7ccccc7c6)n5)cccc34)cc2)cc1 ; CC1(C)c2ccccc2-c2ccc(N(c3ccccc3)c3cc4c5ccccc5oc4c4c(-c5ccc(-c6nc(-c7ccccc7)nc(-c7ccc(-c8ccccc8)cc7)n6)cc5)cccc34)cc21 ; c1ccc(-c2nc(-c3ccc4ccccc4c3)nc(-c3cccc4c(N(c5ccccc5)c5ccc6ccccc6c5)cc5c6ccccc6oc5c34)n2)cc1]

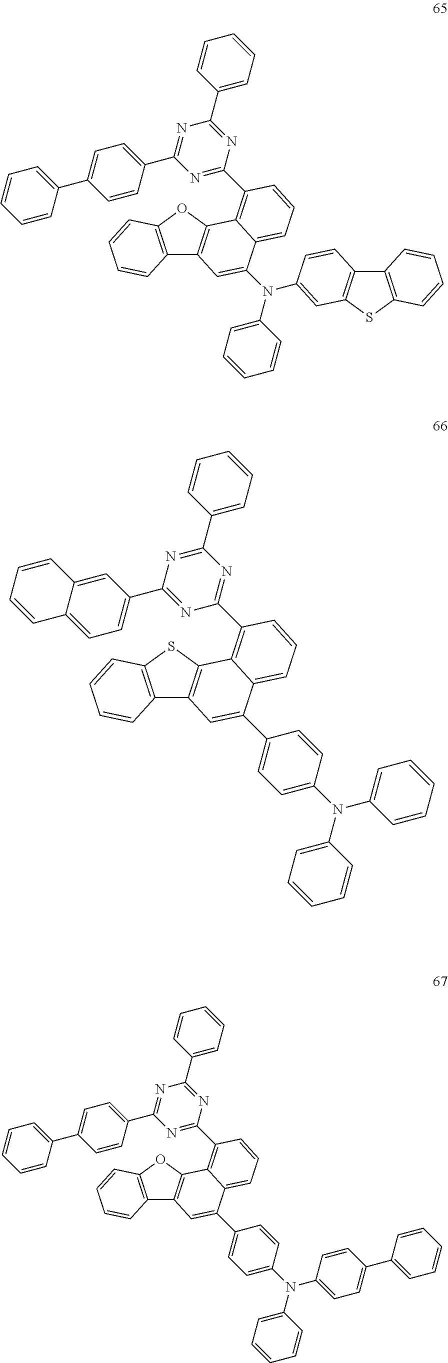 [SMILES: c1ccc(-c2ccc(-c3nc(-c4ccccc4)nc(-c4cccc5c(N(c6ccccc6)c6ccc7c(c6)sc6ccccc67)cc6c7ccccc7oc6c45)n3)cc2)cc1 ; c1ccc(-c2nc(-c3ccc4ccccc4c3)nc(-c3cccc4c(-c5ccc(N(c6ccccc6)c6ccccc6)cc5)cc5c6ccccc6sc5c34)n2)cc1 ; c1ccc(-c2ccc(-c3nc(-c4ccccc4)nc(-c4cccc5c(-c6ccc(N(c7ccccc7)c7ccc(-c8ccccc8)cc7)cc6)cc6c7ccccc7oc6c45)n3)cc2)cc1]

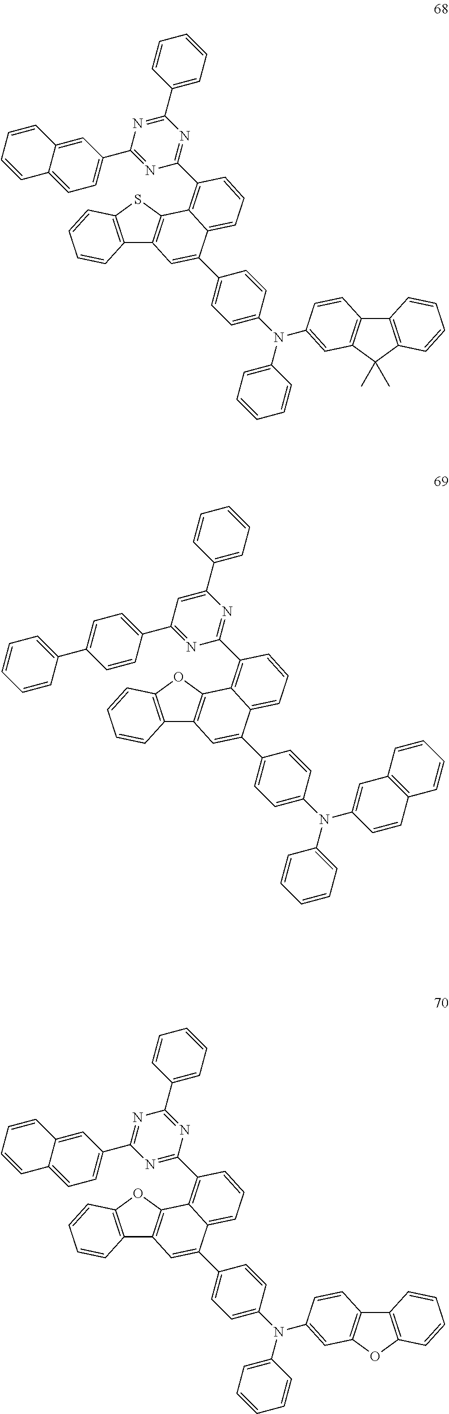 [SMILES: CC1(C)c2ccccc2-c2ccc(N(c3ccccc3)c3ccc(-c4cc5c6ccccc6sc5c5c(-c6nc(-c7ccccc7)nc(-c7ccc8ccccc8c7)n6)cccc45)cc3)cc21 ; c1ccc(-c2ccc(-c3cc(-c4ccccc4)nc(-c4cccc5c(-c6ccc(N(c7ccccc7)c7ccc8ccccc8c7)cc6)cc6c7ccccc7oc6c45)n3)cc2)cc1 ; c1ccc(-c2nc(-c3ccc4ccccc4c3)nc(-c3cccc4c(-c5ccc(N(c6ccccc6)c6ccc7c(c6)oc6ccccc67)cc5)cc5c6ccccc6oc5c34)n2)cc1]

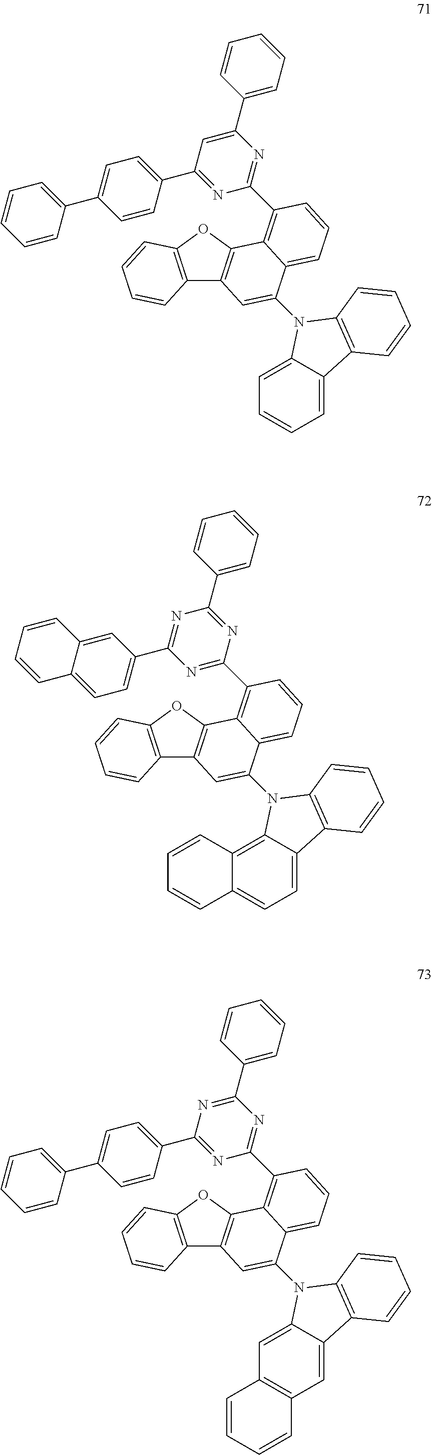 [SMILES: c1ccc(-c2ccc(-c3cc(-c4ccccc4)nc(-c4cccc5c(-n6c7ccccc7c7ccccc76)cc6c7ccccc7oc6c45)n3)cc2)cc1 ; c1ccc(-c2nc(-c3ccc4ccccc4c3)nc(-c3cccc4c(-n5c6ccccc6c6ccc7ccccc7c65)cc5c6ccccc6oc5c34)n2)cc1 ; c1ccc(-c2ccc(-c3nc(-c4ccccc4)nc(-c4cccc5c(-n6c7ccccc7c7cc8ccccc8cc76)cc6c7ccccc7oc6c45)n3)cc2)cc1]

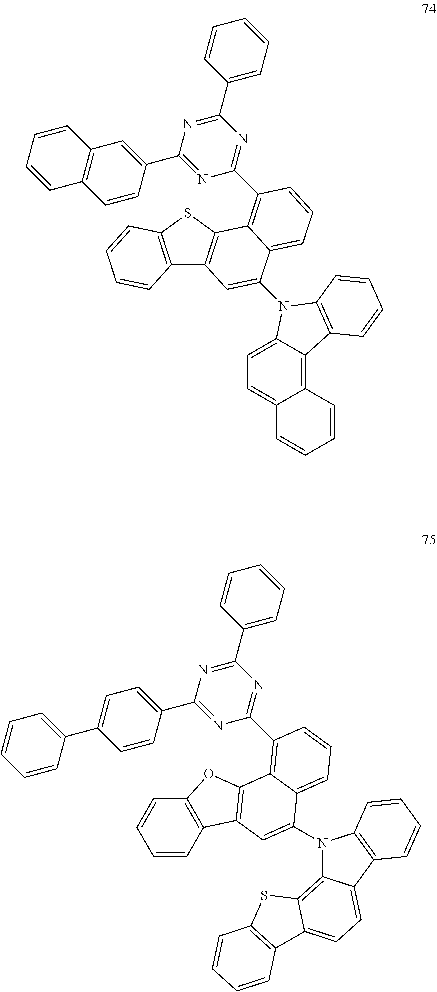 [SMILES: c1ccc(-c2nc(-c3ccc4ccccc4c3)nc(-c3cccc4c(-n5c6ccccc6c6c7ccccc7ccc65)cc5c6ccccc6sc5c34)n2)cc1 ; c1ccc(-c2ccc(-c3nc(-c4ccccc4)nc(-c4cccc5c(-n6c7ccccc7c7ccc8c9ccccc9sc8c76)cc6c7ccccc7oc6c45)n3)cc2)cc1]

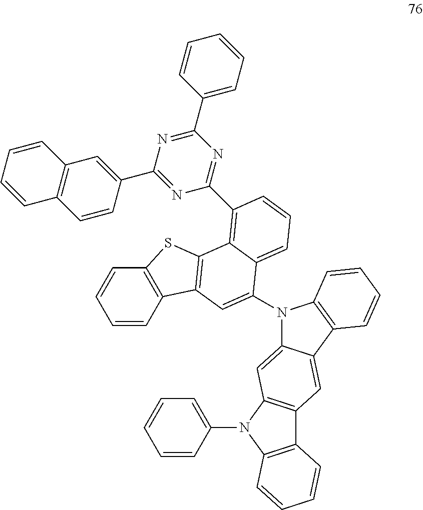 [SMILES: c1ccc(-c2nc(-c3ccc4ccccc4c3)nc(-c3cccc4c(-n5c6ccccc6c6cc7c8ccccc8n(-c8ccccc8)c7cc65)cc5c6ccccc6sc5c34)n2)cc1]

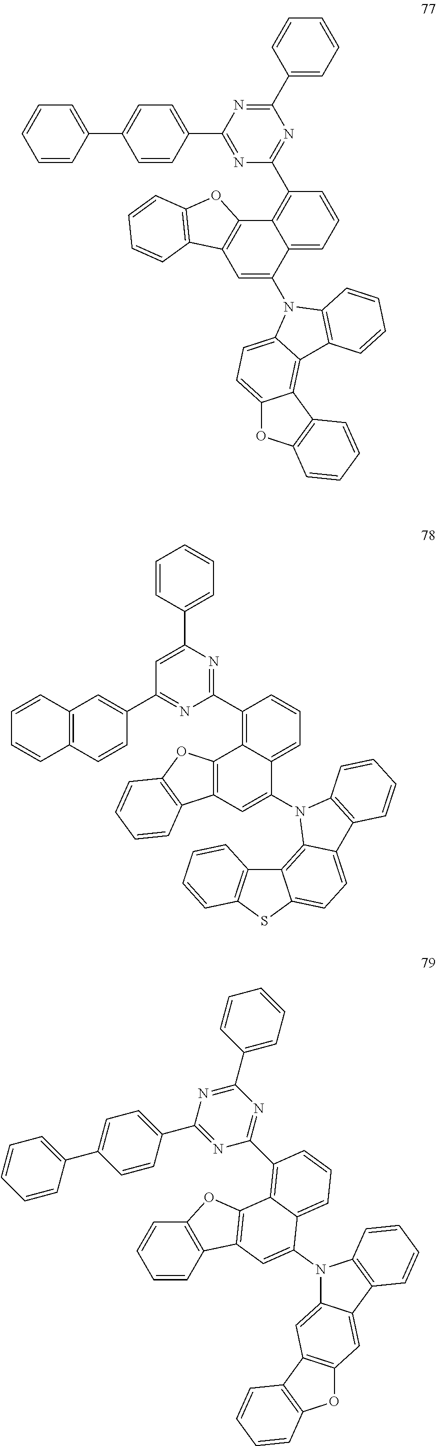 [SMILES: c1ccc(-c2ccc(-c3nc(-c4ccccc4)nc(-c4cccc5c(-n6c7ccccc7c7c8c(ccc76)oc6ccccc68)cc6c7ccccc7oc6c45)n3)cc2)cc1 ; c1ccc(-c2cc(-c3ccc4ccccc4c3)nc(-c3cccc4c(-n5c6ccccc6c6ccc7sc8ccccc8c7c65)cc5c6ccccc6oc5c34)n2)cc1 ; c1ccc(-c2ccc(-c3nc(-c4ccccc4)nc(-c4cccc5c(-n6c7ccccc7c7cc8oc9ccccc9c8cc76)cc6c7ccccc7oc6c45)n3)cc2)cc1]

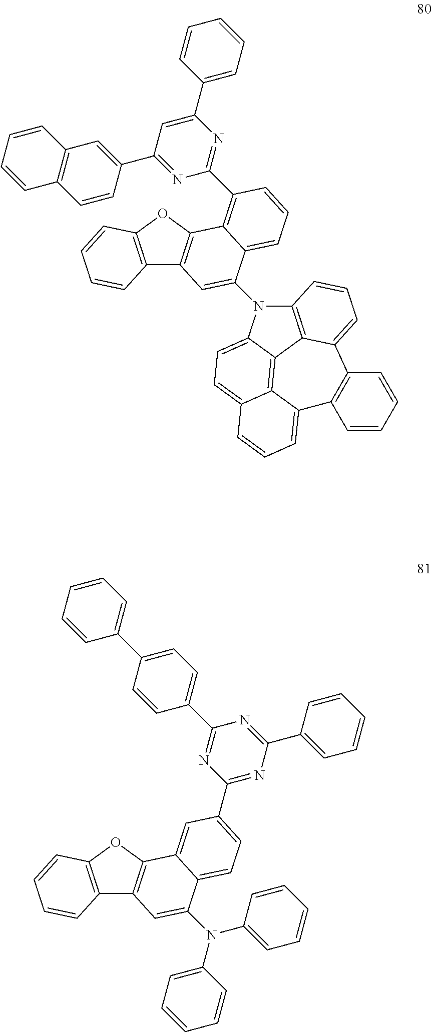 [SMILES: c1ccc(-c2cc(-c3ccc4ccccc4c3)nc(-c3cccc4c(-n5c6cccc7c6c6c8c(cccc8ccc65)-c5ccccc5-7)cc5c6ccccc6oc5c34)n2)cc1 ; c1ccc(-c2ccc(-c3nc(-c4ccccc4)nc(-c4ccc5c(N(c6ccccc6)c6ccccc6)cc6c7ccccc7oc6c5c4)n3)cc2)cc1]

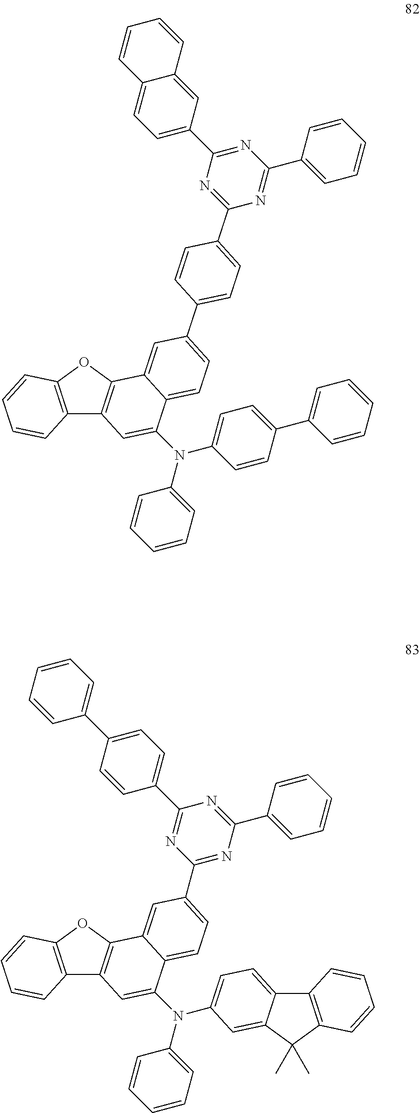 [SMILES: c1ccc(-c2ccc(N(c3ccccc3)c3cc4c5ccccc5oc4c4cc(-c5ccc(-c6nc(-c7ccccc7)nc(-c7ccc8ccccc8c7)n6)cc5)ccc34)cc2)cc1 ; CC1(C)c2ccccc2-c2ccc(N(c3ccccc3)c3cc4c5ccccc5oc4c4cc(-c5nc(-c6ccccc6)nc(-c6ccc(-c7ccccc7)cc6)n5)ccc34)cc21]

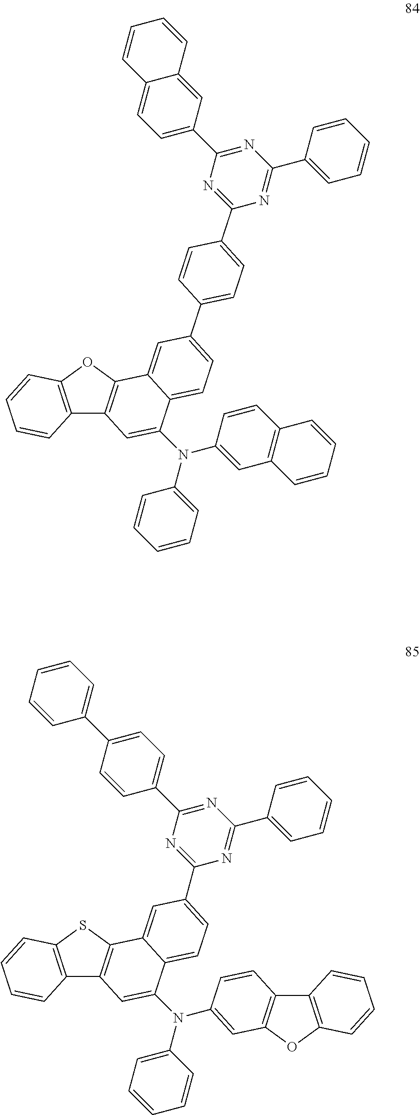 [SMILES: c1ccc(-c2nc(-c3ccc(-c4ccc5c(N(c6ccccc6)c6ccc7ccccc7c6)cc6c7ccccc7oc6c5c4)cc3)nc(-c3ccc4ccccc4c3)n2)cc1 ; c1ccc(-c2ccc(-c3nc(-c4ccccc4)nc(-c4ccc5c(N(c6ccccc6)c6ccc7c(c6)oc6ccccc67)cc6c7ccccc7sc6c5c4)n3)cc2)cc1]

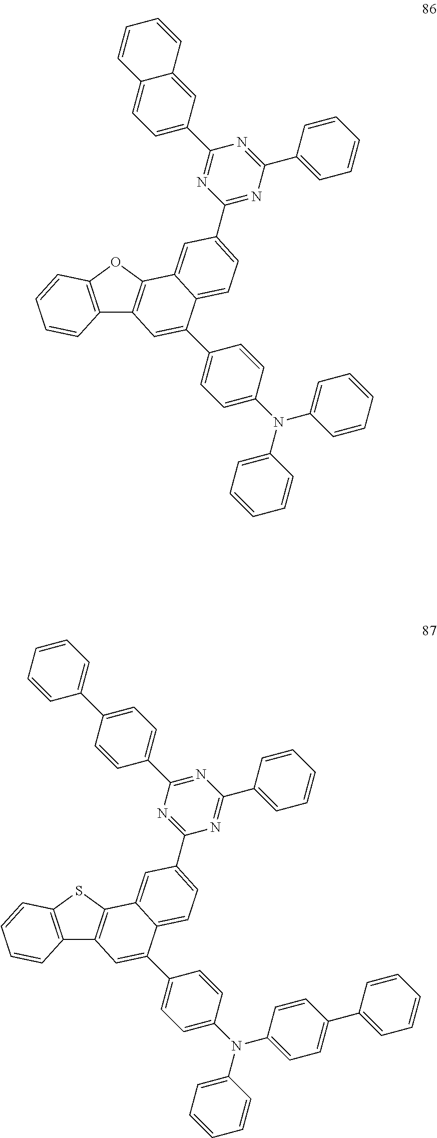 [SMILES: c1ccc(-c2nc(-c3ccc4ccccc4c3)nc(-c3ccc4c(-c5ccc(N(c6ccccc6)c6ccccc6)cc5)cc5c6ccccc6oc5c4c3)n2)cc1 ; c1ccc(-c2ccc(-c3nc(-c4ccccc4)nc(-c4ccc5c(-c6ccc(N(c7ccccc7)c7ccc(-c8ccccc8)cc7)cc6)cc6c7ccccc7sc6c5c4)n3)cc2)cc1]

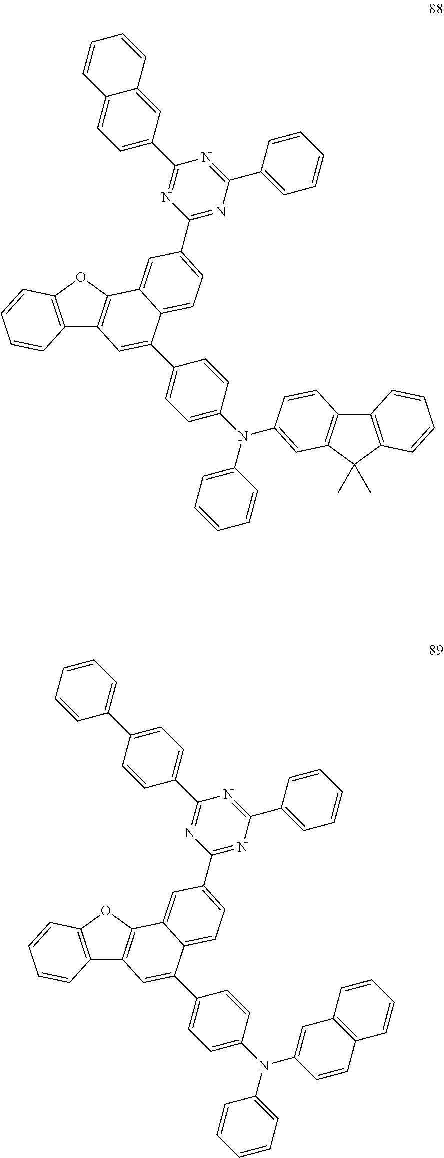 [SMILES: CC1(C)c2ccccc2-c2ccc(N(c3ccccc3)c3ccc(-c4cc5c6ccccc6oc5c5cc(-c6nc(-c7ccccc7)nc(-c7ccc8ccccc8c7)n6)ccc45)cc3)cc21 ; c1ccc(-c2ccc(-c3nc(-c4ccccc4)nc(-c4ccc5c(-c6ccc(N(c7ccccc7)c7ccc8ccccc8c7)cc6)cc6c7ccccc7oc6c5c4)n3)cc2)cc1]

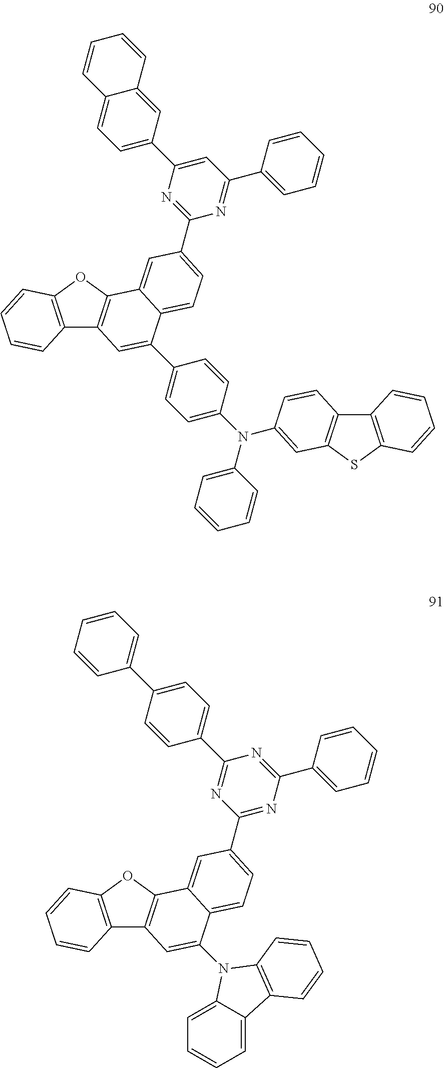 [SMILES: c1ccc(-c2cc(-c3ccc4ccccc4c3)nc(-c3ccc4c(-c5ccc(N(c6ccccc6)c6ccc7c(c6)sc6ccccc67)cc5)cc5c6ccccc6oc5c4c3)n2)cc1 ; c1ccc(-c2ccc(-c3nc(-c4ccccc4)nc(-c4ccc5c(-n6c7ccccc7c7ccccc76)cc6c7ccccc7oc6c5c4)n3)cc2)cc1]

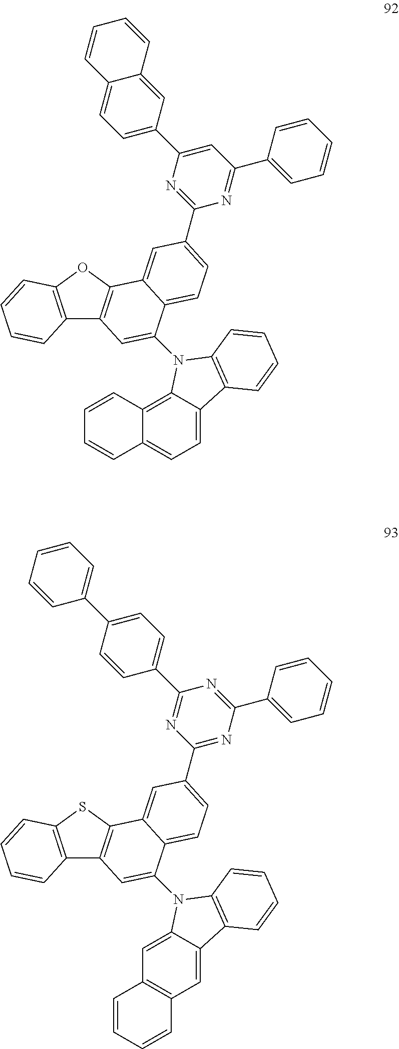 [SMILES: c1ccc(-c2cc(-c3ccc4ccccc4c3)nc(-c3ccc4c(-n5c6ccccc6c6ccc7ccccc7c65)cc5c6ccccc6oc5c4c3)n2)cc1 ; c1ccc(-c2ccc(-c3nc(-c4ccccc4)nc(-c4ccc5c(-n6c7ccccc7c7cc8ccccc8cc76)cc6c7ccccc7sc6c5c4)n3)cc2)cc1]

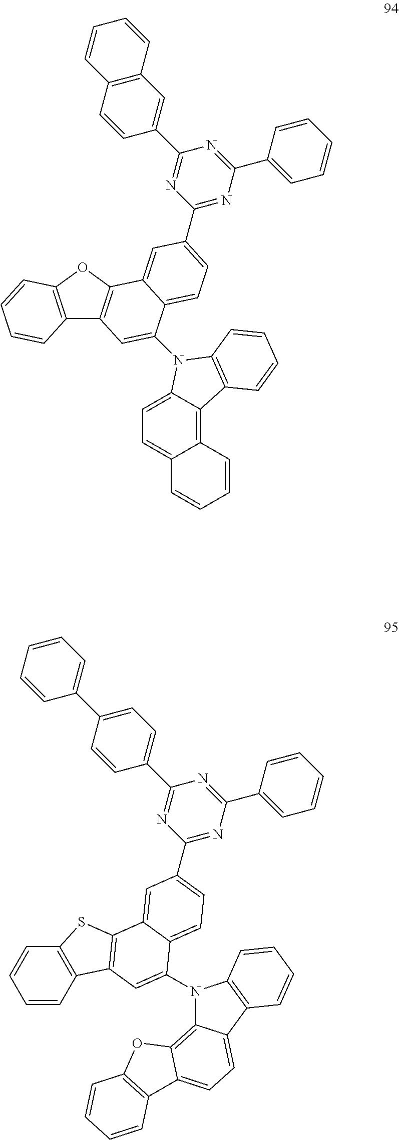 [SMILES: c1ccc(-c2nc(-c3ccc4ccccc4c3)nc(-c3ccc4c(-n5c6ccccc6c6c7ccccc7ccc65)cc5c6ccccc6oc5c4c3)n2)cc1 ; c1ccc(-c2ccc(-c3nc(-c4ccccc4)nc(-c4ccc5c(-n6c7ccccc7c7ccc8c9ccccc9oc8c76)cc6c7ccccc7sc6c5c4)n3)cc2)cc1]

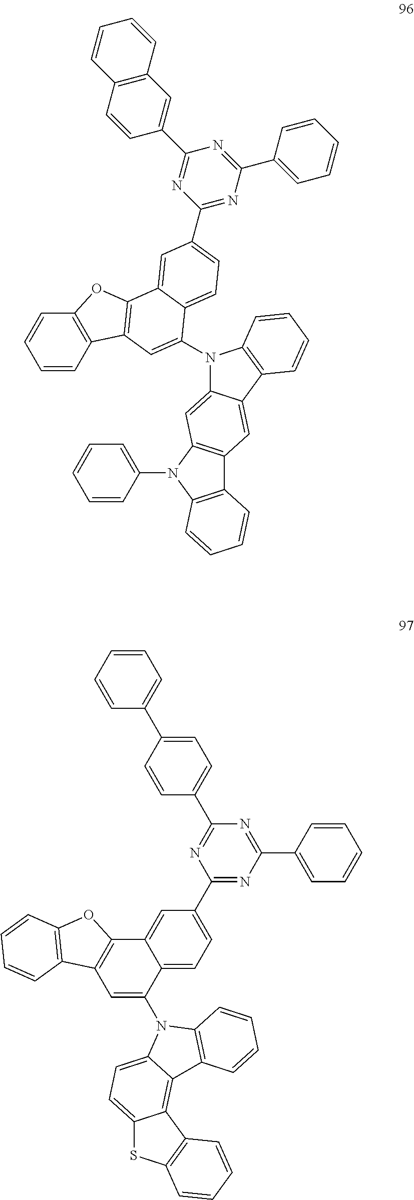 [SMILES: c1ccc(-c2nc(-c3ccc4ccccc4c3)nc(-c3ccc4c(-n5c6ccccc6c6cc7c8ccccc8n(-c8ccccc8)c7cc65)cc5c6ccccc6oc5c4c3)n2)cc1 ; c1ccc(-c2ccc(-c3nc(-c4ccccc4)nc(-c4ccc5c(-n6c7ccccc7c7c8c(ccc76)sc6ccccc68)cc6c7ccccc7oc6c5c4)n3)cc2)cc1]

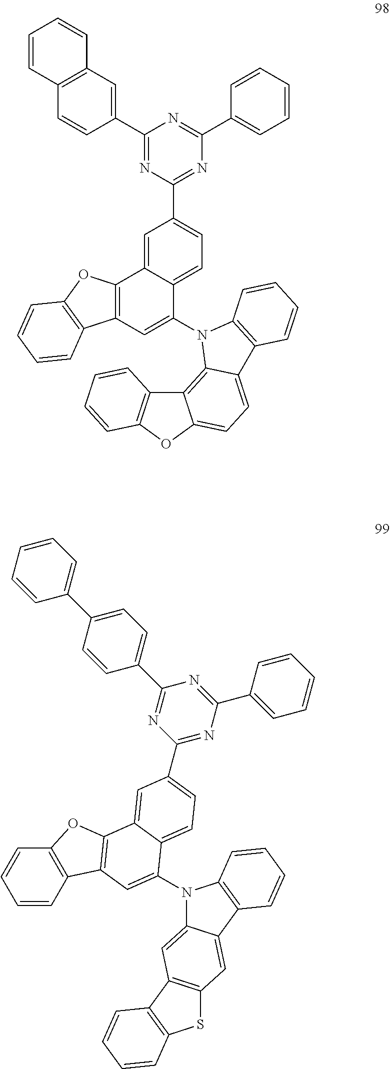 [SMILES: c1ccc(-c2nc(-c3ccc4ccccc4c3)nc(-c3ccc4c(-n5c6ccccc6c6ccc7oc8ccccc8c7c65)cc5c6ccccc6oc5c4c3)n2)cc1 ; c1ccc(-c2ccc(-c3nc(-c4ccccc4)nc(-c4ccc5c(-n6c7ccccc7c7cc8sc9ccccc9c8cc76)cc6c7ccccc7oc6c5c4)n3)cc2)cc1]

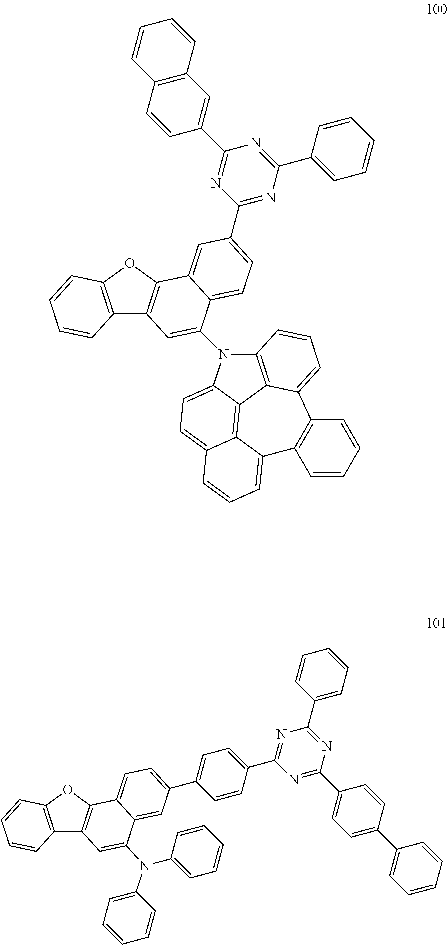 [SMILES: c1ccc(-c2nc(-c3ccc4ccccc4c3)nc(-c3ccc4c(-n5c6cccc7c6c6c8c(cccc8ccc65)-c5ccccc5-7)cc5c6ccccc6oc5c4c3)n2)cc1 ; c1ccc(-c2ccc(-c3nc(-c4ccccc4)nc(-c4ccc(-c5ccc6c(c5)c(N(c5ccccc5)c5ccccc5)cc5c7ccccc7oc65)cc4)n3)cc2)cc1]

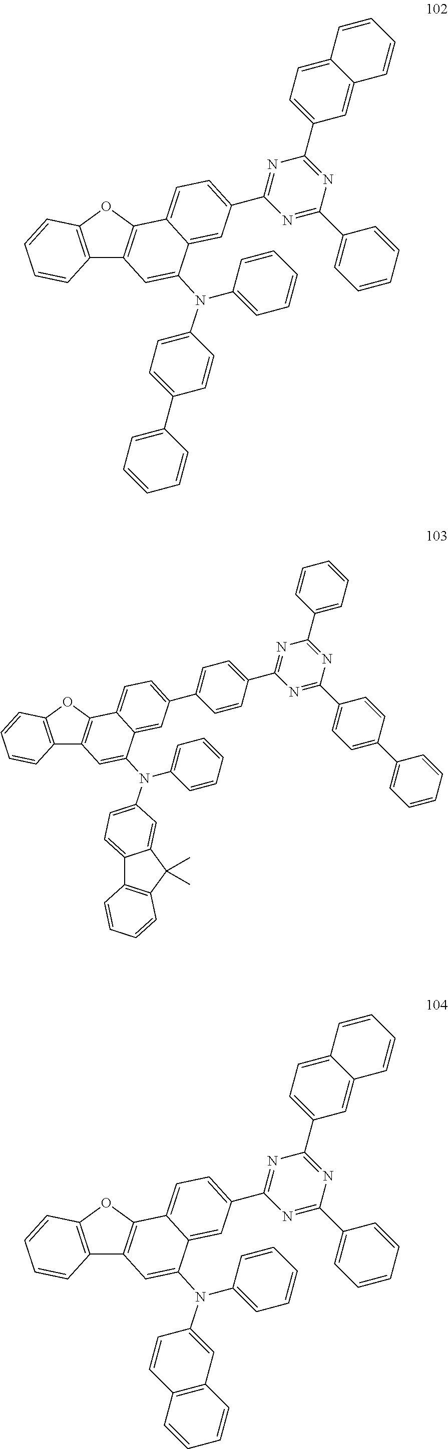 [SMILES: c1ccc(-c2ccc(N(c3ccccc3)c3cc4c5ccccc5oc4c4ccc(-c5nc(-c6ccccc6)nc(-c6ccc7ccccc7c6)n5)cc34)cc2)cc1 ; CC1(C)c2ccccc2-c2ccc(N(c3ccccc3)c3cc4c5ccccc5oc4c4ccc(-c5ccc(-c6nc(-c7ccccc7)nc(-c7ccc(-c8ccccc8)cc7)n6)cc5)cc34)cc21 ; c1ccc(-c2nc(-c3ccc4ccccc4c3)nc(-c3ccc4c(c3)c(N(c3ccccc3)c3ccc5ccccc5c3)cc3c5ccccc5oc43)n2)cc1]

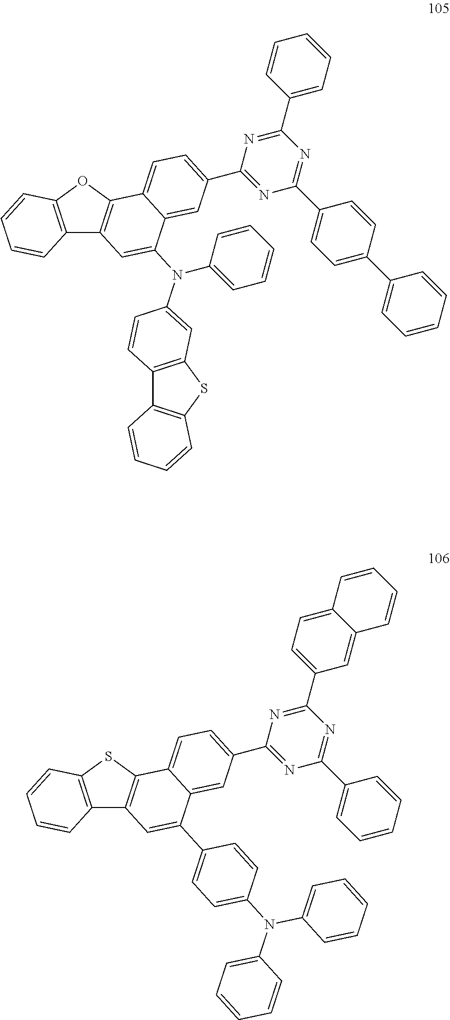 [SMILES: c1ccc(-c2ccc(-c3nc(-c4ccccc4)nc(-c4ccc5c(c4)c(N(c4ccccc4)c4ccc6c(c4)sc4ccccc46)cc4c6ccccc6oc54)n3)cc2)cc1 ; c1ccc(-c2nc(-c3ccc4ccccc4c3)nc(-c3ccc4c(c3)c(-c3ccc(N(c5ccccc5)c5ccccc5)cc3)cc3c5ccccc5sc43)n2)cc1]

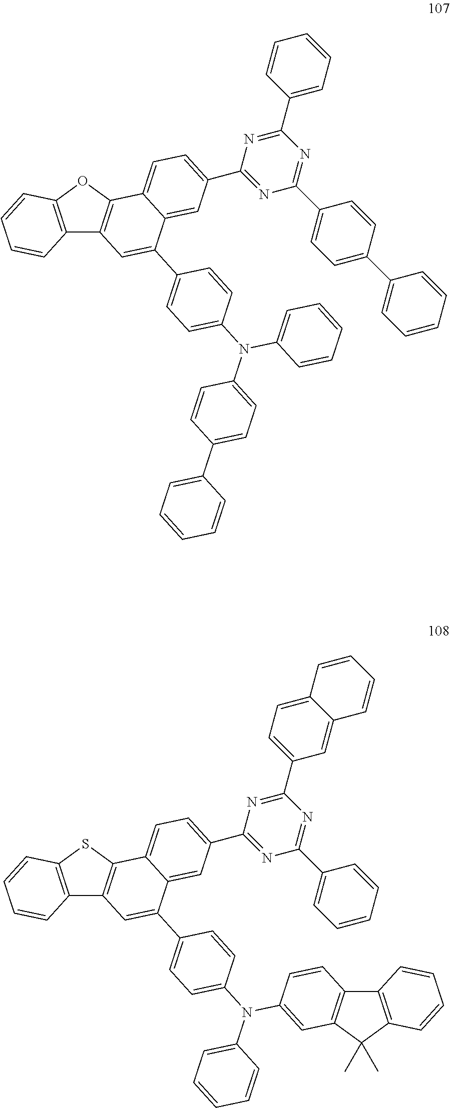 [SMILES: c1ccc(-c2ccc(-c3nc(-c4ccccc4)nc(-c4ccc5c(c4)c(-c4ccc(N(c6ccccc6)c6ccc(-c7ccccc7)cc6)cc4)cc4c6ccccc6oc54)n3)cc2)cc1 ; CC1(C)c2ccccc2-c2ccc(N(c3ccccc3)c3ccc(-c4cc5c6ccccc6sc5c5ccc(-c6nc(-c7ccccc7)nc(-c7ccc8ccccc8c7)n6)cc45)cc3)cc21]

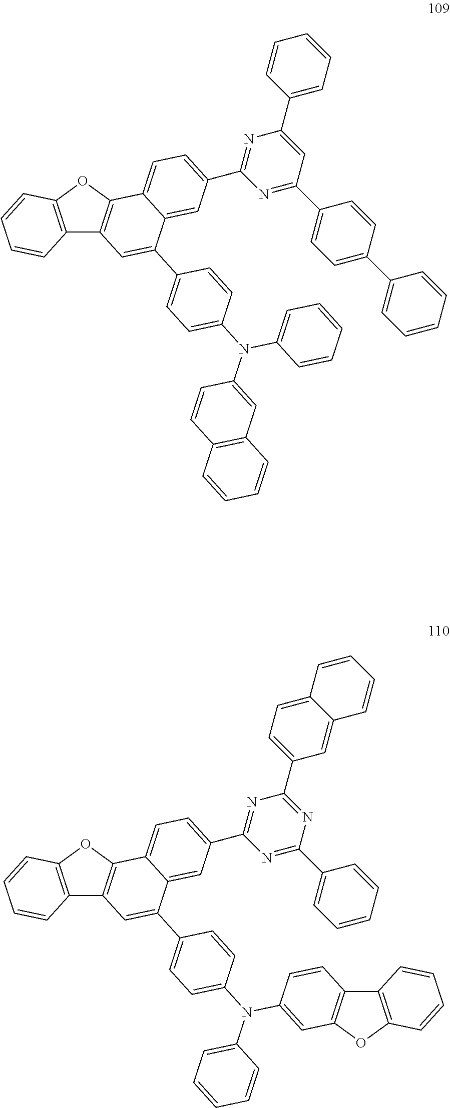 [SMILES: c1ccc(-c2ccc(-c3cc(-c4ccccc4)nc(-c4ccc5c(c4)c(-c4ccc(N(c6ccccc6)c6ccc7ccccc7c6)cc4)cc4c6ccccc6oc54)n3)cc2)cc1 ; c1ccc(-c2nc(-c3ccc4ccccc4c3)nc(-c3ccc4c(c3)c(-c3ccc(N(c5ccccc5)c5ccc6c(c5)oc5ccccc56)cc3)cc3c5ccccc5oc43)n2)cc1]

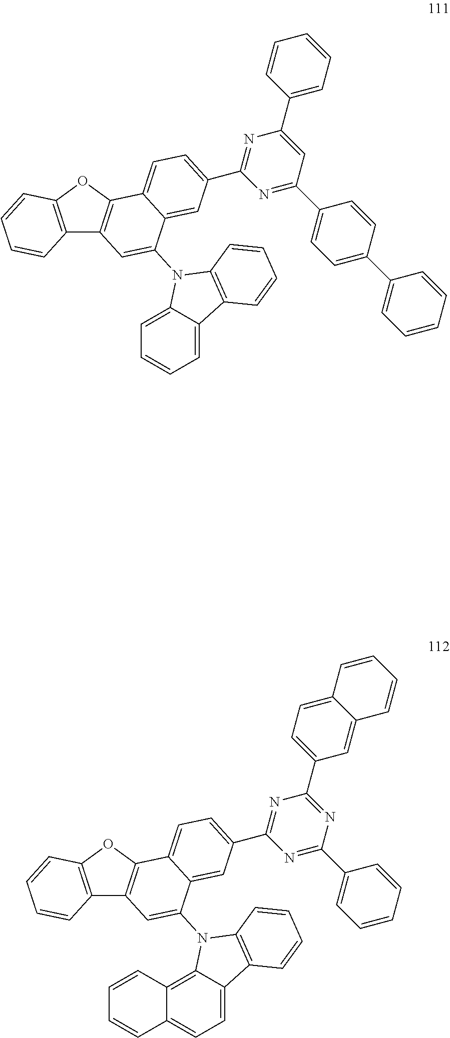 [SMILES: c1ccc(-c2ccc(-c3cc(-c4ccccc4)nc(-c4ccc5c(c4)c(-n4c6ccccc6c6ccccc64)cc4c6ccccc6oc54)n3)cc2)cc1 ; c1ccc(-c2nc(-c3ccc4ccccc4c3)nc(-c3ccc4c(c3)c(-n3c5ccccc5c5ccc6ccccc6c53)cc3c5ccccc5oc43)n2)cc1]

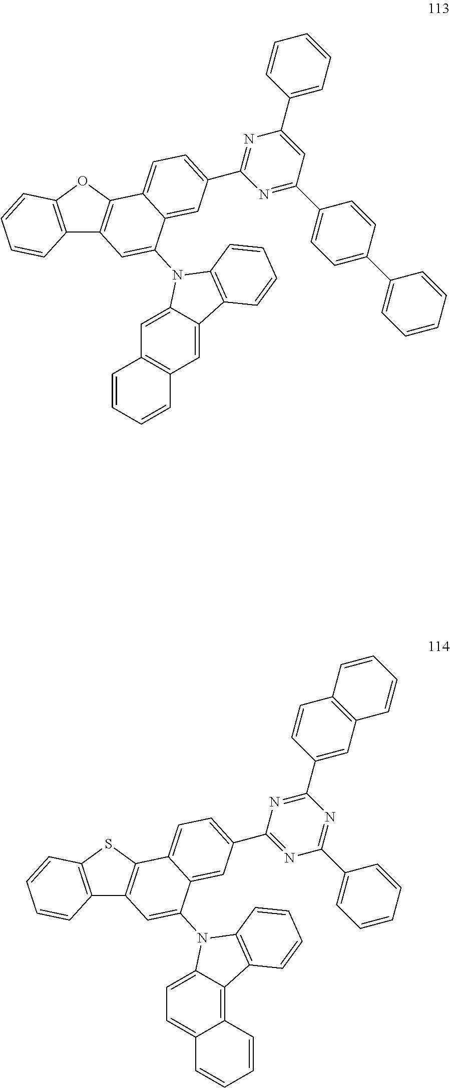 [SMILES: c1ccc(-c2ccc(-c3cc(-c4ccccc4)nc(-c4ccc5c(c4)c(-n4c6ccccc6c6cc7ccccc7cc64)cc4c6ccccc6oc54)n3)cc2)cc1 ; c1ccc(-c2nc(-c3ccc4ccccc4c3)nc(-c3ccc4c(c3)c(-n3c5ccccc5c5c6ccccc6ccc53)cc3c5ccccc5sc43)n2)cc1]

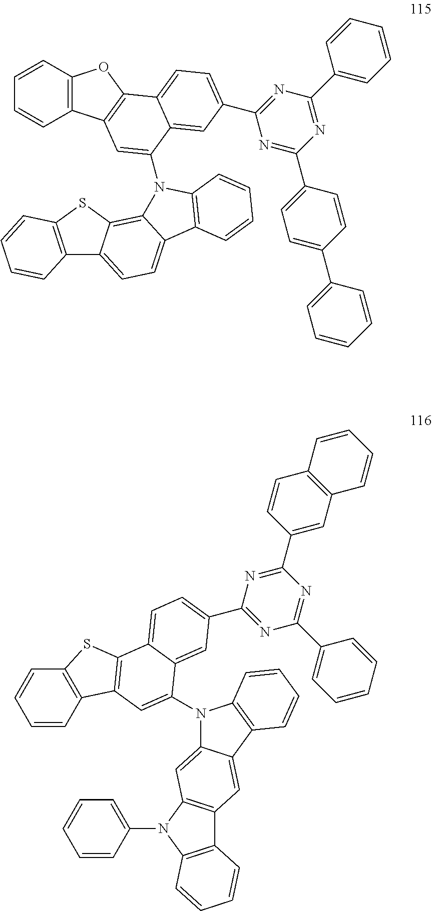 [SMILES: c1ccc(-c2ccc(-c3nc(-c4ccccc4)nc(-c4ccc5c(c4)c(-n4c6ccccc6c6ccc7c8ccccc8sc7c64)cc4c6ccccc6oc54)n3)cc2)cc1 ; c1ccc(-c2nc(-c3ccc4ccccc4c3)nc(-c3ccc4c(c3)c(-n3c5ccccc5c5cc6c7ccccc7n(-c7ccccc7)c6cc53)cc3c5ccccc5sc43)n2)cc1]

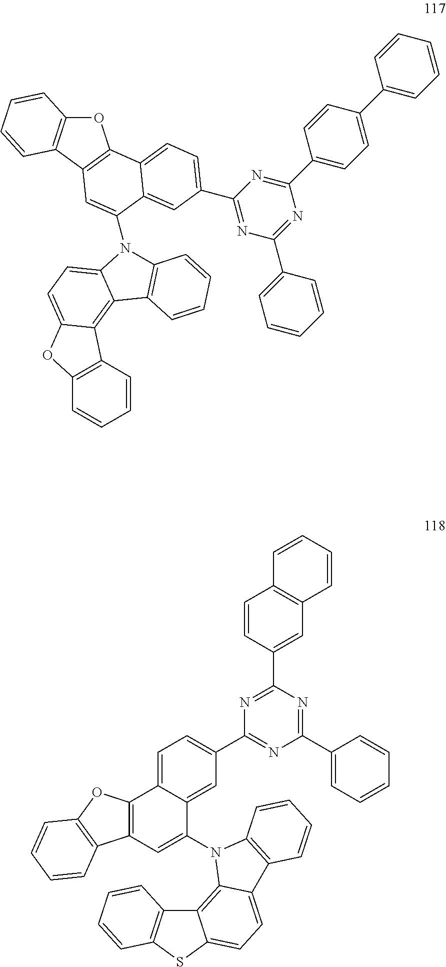 [SMILES: c1ccc(-c2ccc(-c3nc(-c4ccccc4)nc(-c4ccc5c(c4)c(-n4c6ccccc6c6c7c(ccc64)oc4ccccc47)cc4c6ccccc6oc54)n3)cc2)cc1 ; c1ccc(-c2nc(-c3ccc4ccccc4c3)nc(-c3ccc4c(c3)c(-n3c5ccccc5c5ccc6sc7ccccc7c6c53)cc3c5ccccc5oc43)n2)cc1]

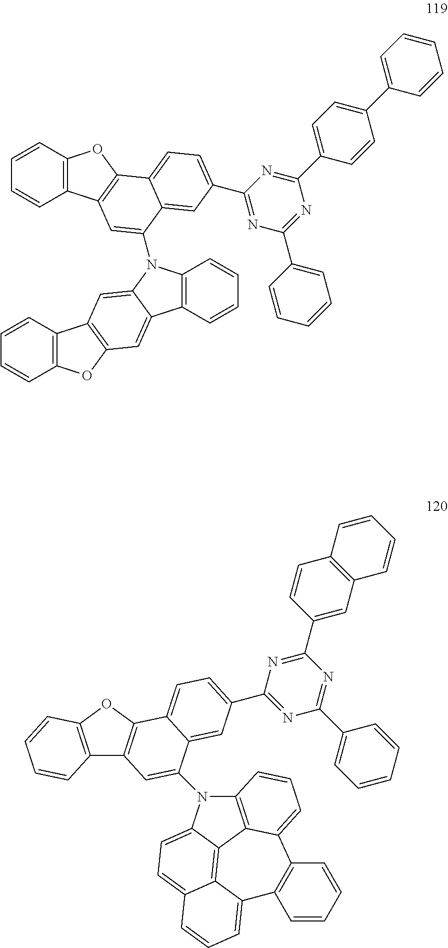 [SMILES: c1ccc(-c2ccc(-c3nc(-c4ccccc4)nc(-c4ccc5c(c4)c(-n4c6ccccc6c6cc7oc8ccccc8c7cc64)cc4c6ccccc6oc54)n3)cc2)cc1 ; c1ccc(-c2nc(-c3ccc4ccccc4c3)nc(-c3ccc4c(c3)c(-n3c5cccc6c5c5c7c(cccc7ccc53)-c3ccccc3-6)cc3c5ccccc5oc43)n2)cc1]

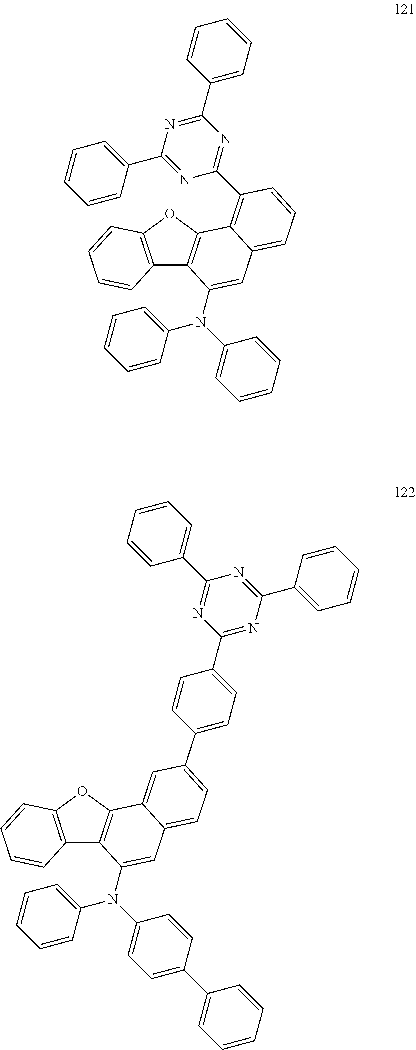 [SMILES: c1ccc(-c2nc(-c3ccccc3)nc(-c3cccc4cc(N(c5ccccc5)c5ccccc5)c5c6ccccc6oc5c34)n2)cc1 ; c1ccc(-c2ccc(N(c3ccccc3)c3cc4ccc(-c5ccc(-c6nc(-c7ccccc7)nc(-c7ccccc7)n6)cc5)cc4c4oc5ccccc5c34)cc2)cc1]

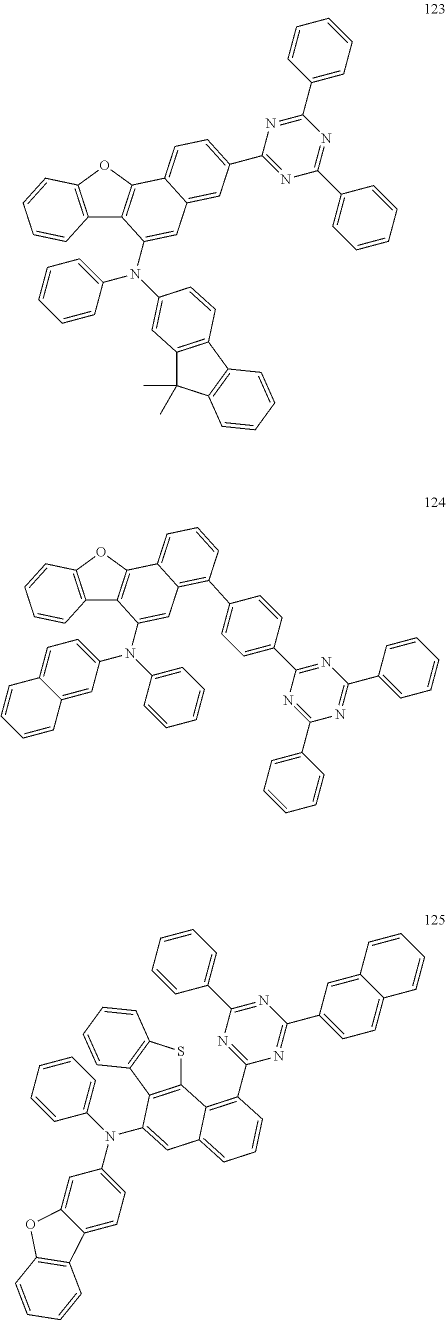 [SMILES: CC1(C)c2ccccc2-c2ccc(N(c3ccccc3)c3cc4cc(-c5nc(-c6ccccc6)nc(-c6ccccc6)n5)ccc4c4oc5ccccc5c34)cc21 ; c1ccc(-c2nc(-c3ccccc3)nc(-c3ccc(-c4cccc5c4cc(N(c4ccccc4)c4ccc6ccccc6c4)c4c6ccccc6oc54)cc3)n2)cc1 ; c1ccc(-c2nc(-c3ccc4ccccc4c3)nc(-c3cccc4cc(N(c5ccccc5)c5ccc6c(c5)oc5ccccc56)c5c6ccccc6sc5c34)n2)cc1]

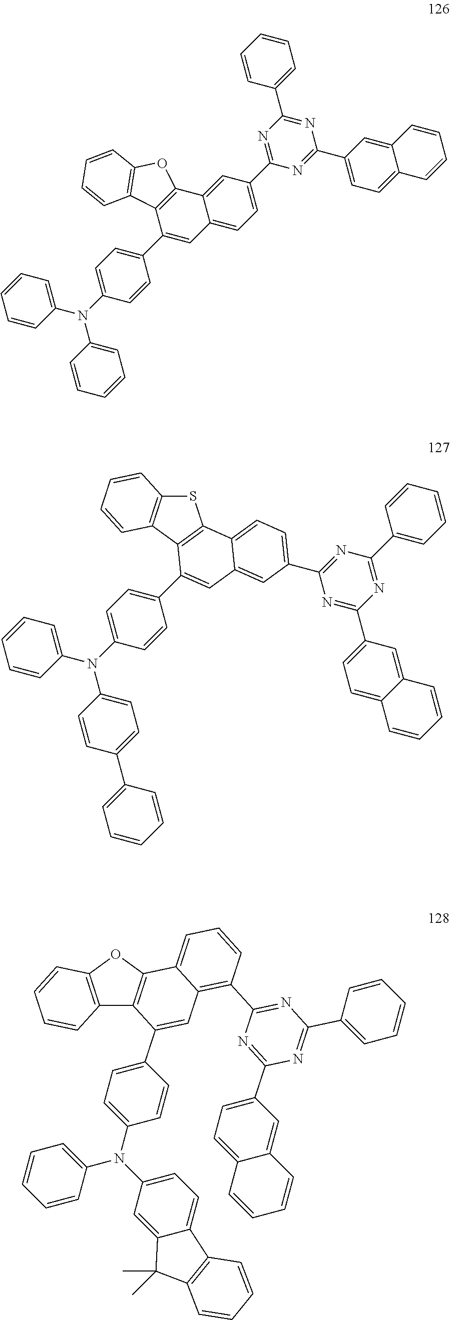 [SMILES: c1ccc(-c2nc(-c3ccc4ccccc4c3)nc(-c3ccc4cc(-c5ccc(N(c6ccccc6)c6ccccc6)cc5)c5c6ccccc6oc5c4c3)n2)cc1 ; c1ccc(-c2ccc(N(c3ccccc3)c3ccc(-c4cc5cc(-c6nc(-c7ccccc7)nc(-c7ccc8ccccc8c7)n6)ccc5c5sc6ccccc6c45)cc3)cc2)cc1 ; CC1(C)c2ccccc2-c2ccc(N(c3ccccc3)c3ccc(-c4cc5c(-c6nc(-c7ccccc7)nc(-c7ccc8ccccc8c7)n6)cccc5c5oc6ccccc6c45)cc3)cc21]

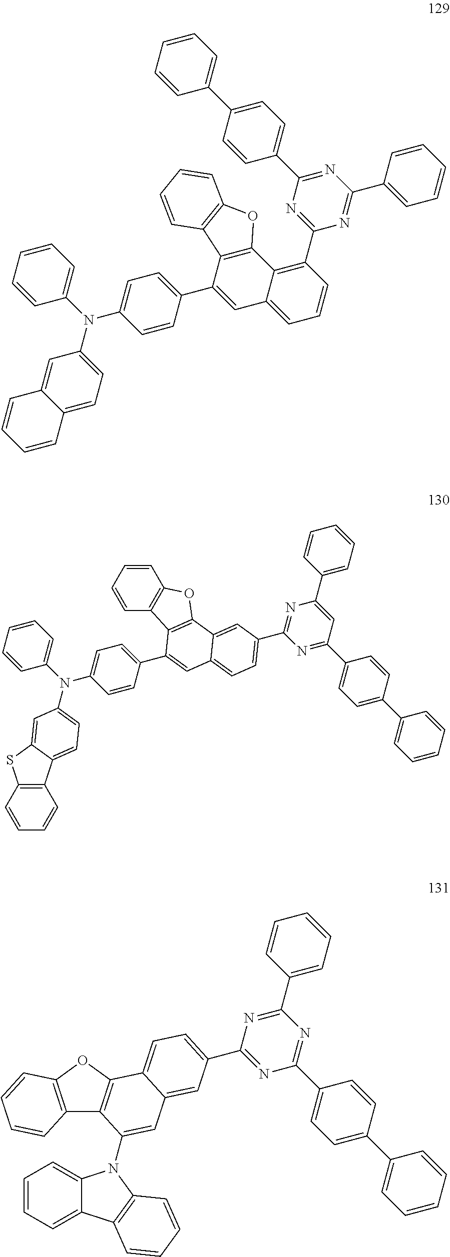 [SMILES: c1ccc(-c2ccc(-c3nc(-c4ccccc4)nc(-c4cccc5cc(-c6ccc(N(c7ccccc7)c7ccc8ccccc8c7)cc6)c6c7ccccc7oc6c45)n3)cc2)cc1 ; c1ccc(-c2ccc(-c3cc(-c4ccccc4)nc(-c4ccc5cc(-c6ccc(N(c7ccccc7)c7ccc8c(c7)sc7ccccc78)cc6)c6c7ccccc7oc6c5c4)n3)cc2)cc1 ; c1ccc(-c2ccc(-c3nc(-c4ccccc4)nc(-c4ccc5c(c4)cc(-n4c6ccccc6c6ccccc64)c4c6ccccc6oc54)n3)cc2)cc1]

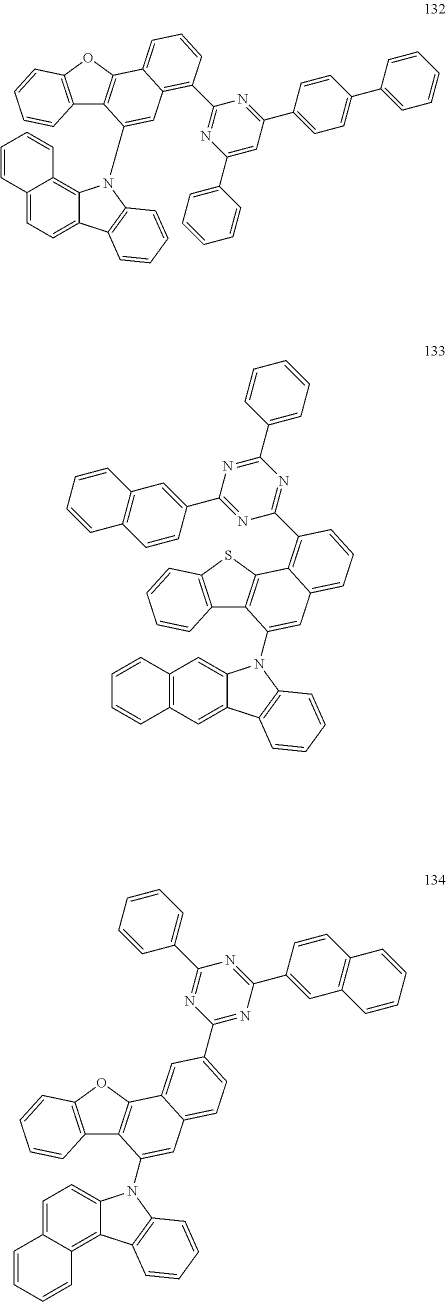 [SMILES: c1ccc(-c2ccc(-c3cc(-c4ccccc4)nc(-c4cccc5c4cc(-n4c6ccccc6c6ccc7ccccc7c64)c4c6ccccc6oc54)n3)cc2)cc1 ; c1ccc(-c2nc(-c3ccc4ccccc4c3)nc(-c3cccc4cc(-n5c6ccccc6c6cc7ccccc7cc65)c5c6ccccc6sc5c34)n2)cc1 ; c1ccc(-c2nc(-c3ccc4ccccc4c3)nc(-c3ccc4cc(-n5c6ccccc6c6c7ccccc7ccc65)c5c6ccccc6oc5c4c3)n2)cc1]

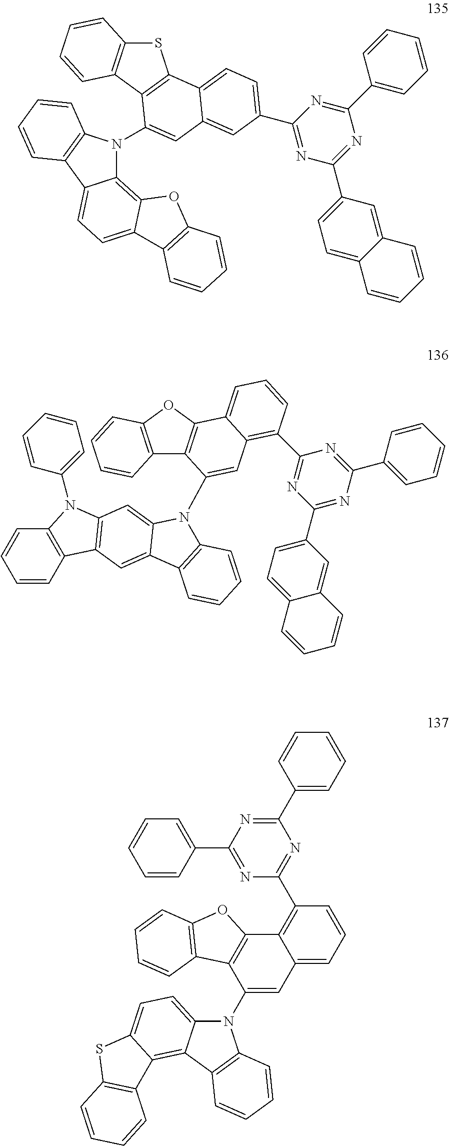 [SMILES: c1ccc(-c2nc(-c3ccc4ccccc4c3)nc(-c3ccc4c(c3)cc(-n3c5ccccc5c5ccc6c7ccccc7oc6c53)c3c5ccccc5sc43)n2)cc1 ; c1ccc(-c2nc(-c3ccc4ccccc4c3)nc(-c3cccc4c3cc(-n3c5ccccc5c5cc6c7ccccc7n(-c7ccccc7)c6cc53)c3c5ccccc5oc43)n2)cc1 ; c1ccc(-c2nc(-c3ccccc3)nc(-c3cccc4cc(-n5c6ccccc6c6c7c(ccc65)sc5ccccc57)c5c6ccccc6oc5c34)n2)cc1]

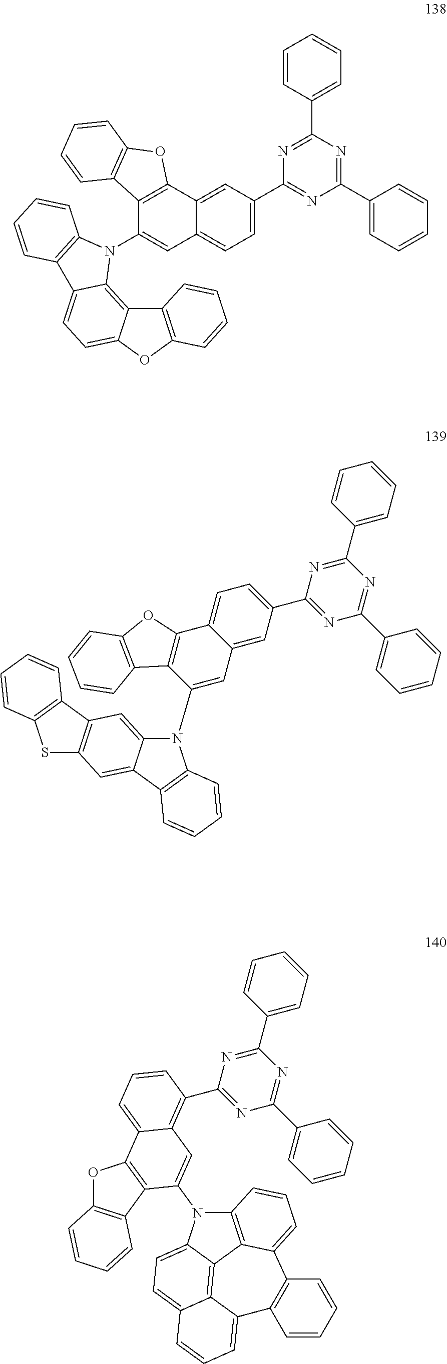 [SMILES: c1ccc(-c2nc(-c3ccccc3)nc(-c3ccc4cc(-n5c6ccccc6c6ccc7oc8ccccc8c7c65)c5c6ccccc6oc5c4c3)n2)cc1 ; c1ccc(-c2nc(-c3ccccc3)nc(-c3ccc4c(c3)cc(-n3c5ccccc5c5cc6sc7ccccc7c6cc53)c3c5ccccc5oc43)n2)cc1 ; c1ccc(-c2nc(-c3ccccc3)nc(-c3cccc4c3cc(-n3c5cccc6c5c5c7c(cccc7ccc53)-c3ccccc3-6)c3c5ccccc5oc43)n2)cc1]

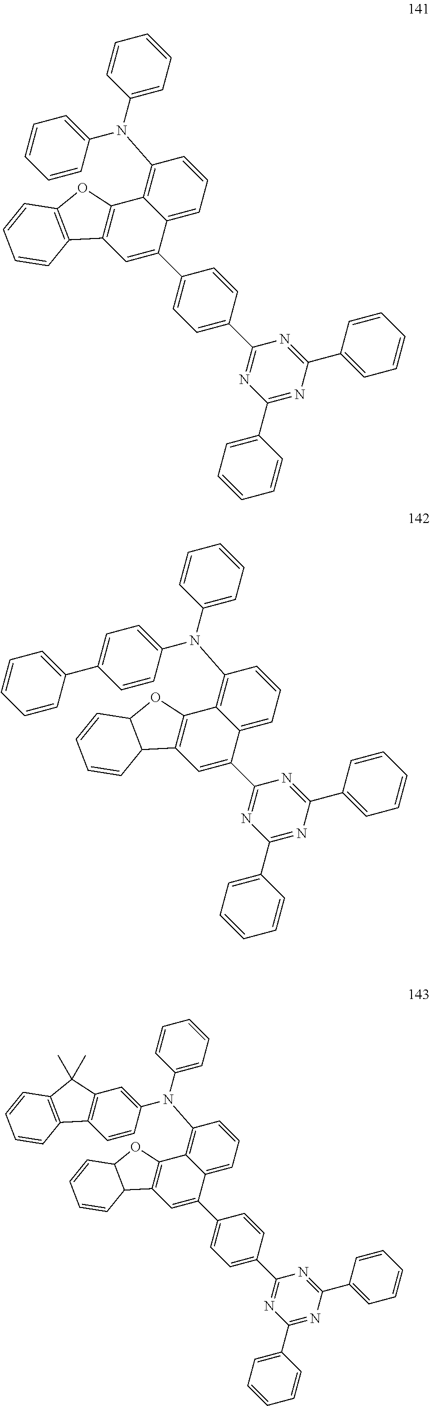 [SMILES: c1ccc(-c2nc(-c3ccccc3)nc(-c3ccc(-c4cc5c6ccccc6oc5c5c(N(c6ccccc6)c6ccccc6)cccc45)cc3)n2)cc1 ; C1=CC2Oc3c(cc(-c4nc(-c5ccccc5)nc(-c5ccccc5)n4)c4cccc(N(c5ccccc5)c5ccc(-c6ccccc6)cc5)c34)C2C=C1 ; CC1(C)c2ccccc2-c2ccc(N(c3ccccc3)c3cccc4c(-c5ccc(-c6nc(-c7ccccc7)nc(-c7ccccc7)n6)cc5)cc5c(c34)OC3C=CC=CC53)cc21]

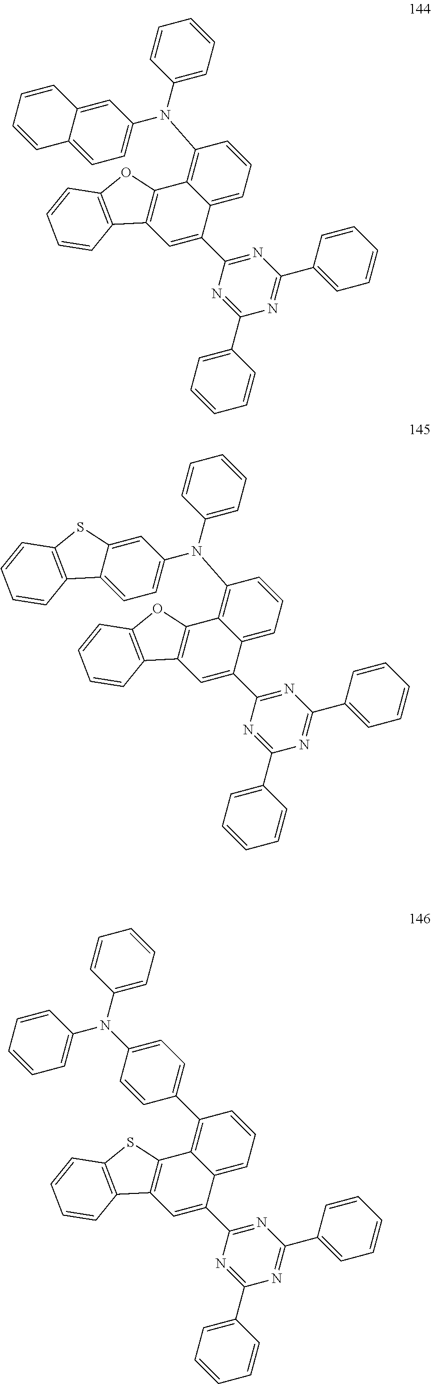

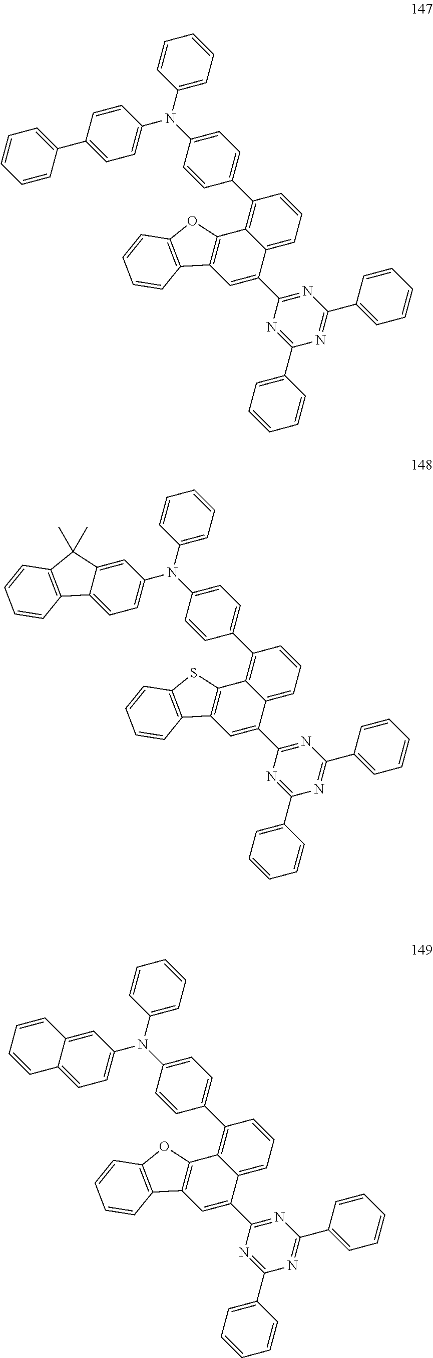 [SMILES: c1ccc(-c2ccc(N(c3ccccc3)c3ccc(-c4cccc5c(-c6nc(-c7ccccc7)nc(-c7ccccc7)n6)cc6c7ccccc7oc6c45)cc3)cc2)cc1 ; CC1(C)c2ccccc2-c2ccc(N(c3ccccc3)c3ccc(-c4cccc5c(-c6nc(-c7ccccc7)nc(-c7ccccc7)n6)cc6c7ccccc7sc6c45)cc3)cc21 ; c1ccc(-c2nc(-c3ccccc3)nc(-c3cc4c5ccccc5oc4c4c(-c5ccc(N(c6ccccc6)c6ccc7ccccc7c6)cc5)cccc34)n2)cc1]

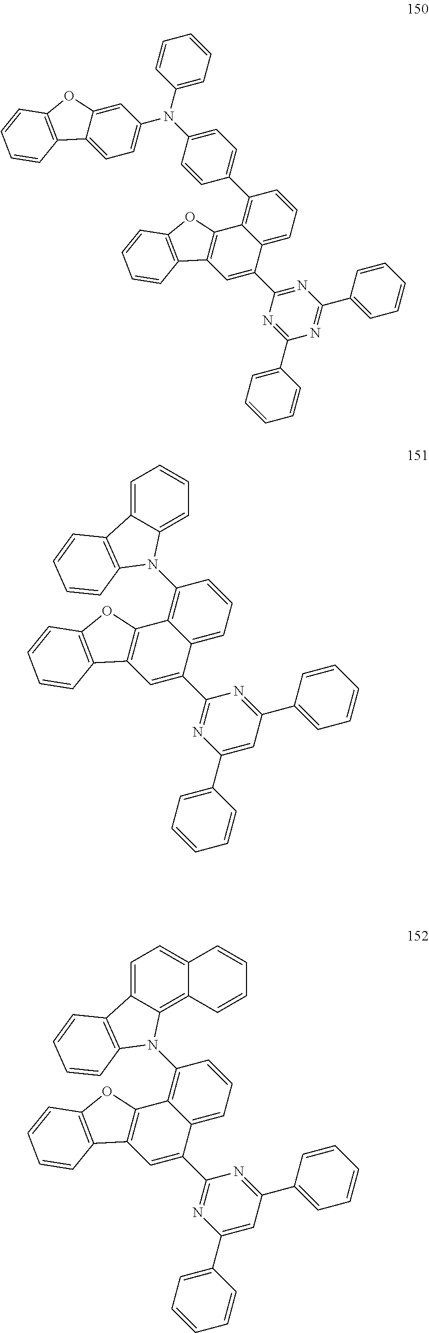 [SMILES: c1ccc(-c2nc(-c3ccccc3)nc(-c3cc4c5ccccc5oc4c4c(-c5ccc(N(c6ccccc6)c6ccc7c(c6)oc6ccccc67)cc5)cccc34)n2)cc1 ; c1ccc(-c2cc(-c3ccccc3)nc(-c3cc4c5ccccc5oc4c4c(-n5c6ccccc6c6ccccc65)cccc34)n2)cc1 ; c1ccc(-c2cc(-c3ccccc3)nc(-c3cc4c5ccccc5oc4c4c(-n5c6ccccc6c6ccc7ccccc7c65)cccc34)n2)cc1]

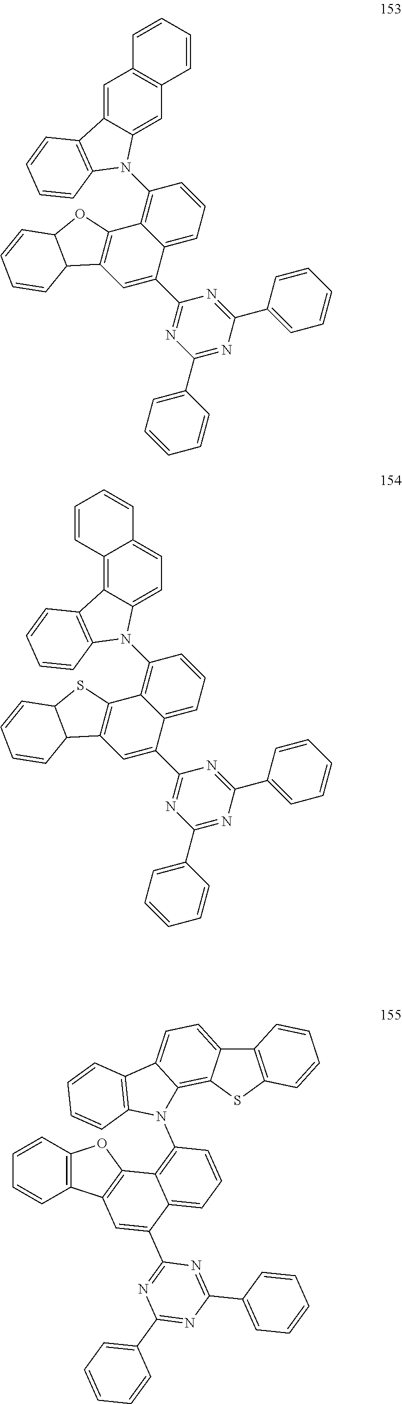 [SMILES: C1=CC2Oc3c(cc(-c4nc(-c5ccccc5)nc(-c5ccccc5)n4)c4cccc(-n5c6ccccc6c6cc7ccccc7cc65)c34)C2C=C1 ; C1=CC2Sc3c(cc(-c4nc(-c5ccccc5)nc(-c5ccccc5)n4)c4cccc(-n5c6ccccc6c6c7ccccc7ccc65)c34)C2C=C1 ; c1ccc(-c2nc(-c3ccccc3)nc(-c3cc4c5ccccc5oc4c4c(-n5c6ccccc6c6ccc7c8ccccc8sc7c65)cccc34)n2)cc1]

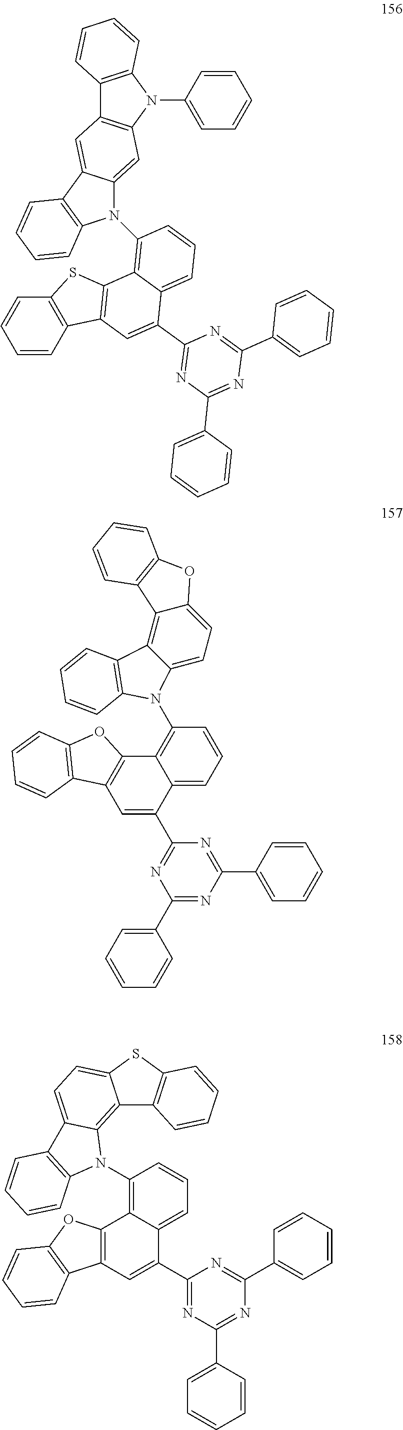 [SMILES: c1ccc(-c2nc(-c3ccccc3)nc(-c3cc4c5ccccc5sc4c4c(-n5c6ccccc6c6cc7c8ccccc8n(-c8ccccc8)c7cc65)cccc34)n2)cc1 ; c1ccc(-c2nc(-c3ccccc3)nc(-c3cc4c5ccccc5oc4c4c(-n5c6ccccc6c6c7c(ccc65)oc5ccccc57)cccc34)n2)cc1 ; c1ccc(-c2nc(-c3ccccc3)nc(-c3cc4c5ccccc5oc4c4c(-n5c6ccccc6c6ccc7sc8ccccc8c7c65)cccc34)n2)cc1]

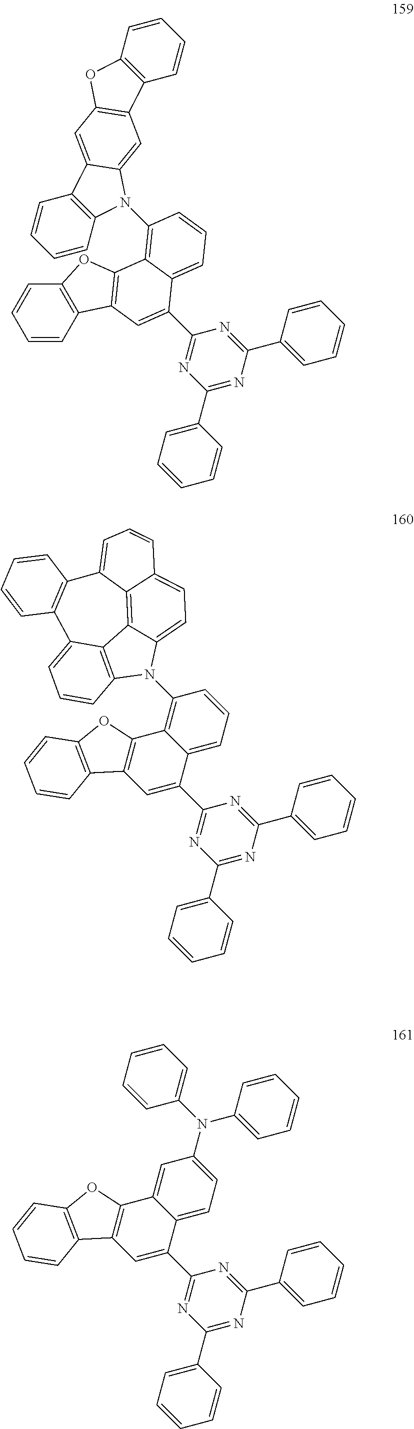 [SMILES: c1ccc(-c2nc(-c3ccccc3)nc(-c3cc4c5ccccc5oc4c4c(-n5c6ccccc6c6cc7oc8ccccc8c7cc65)cccc34)n2)cc1 ; c1ccc(-c2nc(-c3ccccc3)nc(-c3cc4c5ccccc5oc4c4c(-n5c6cccc7c6c6c8c(cccc8ccc65)-c5ccccc5-7)cccc34)n2)cc1 ; c1ccc(-c2nc(-c3ccccc3)nc(-c3cc4c5ccccc5oc4c4cc(N(c5ccccc5)c5ccccc5)ccc34)n2)cc1]

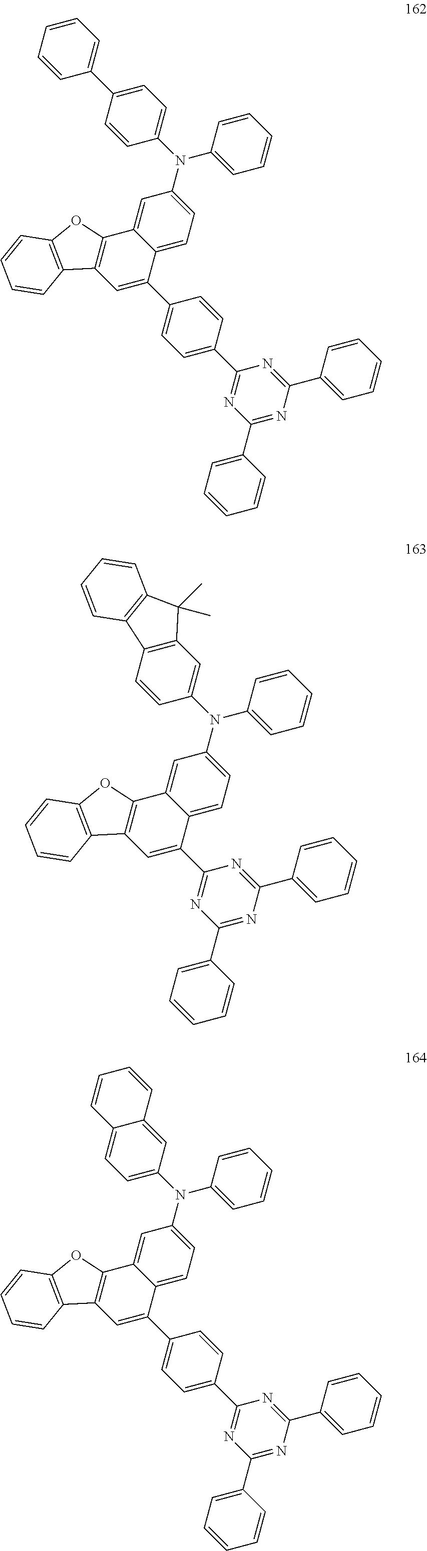 [SMILES: c1ccc(-c2ccc(N(c3ccccc3)c3ccc4c(-c5ccc(-c6nc(-c7ccccc7)nc(-c7ccccc7)n6)cc5)cc5c6ccccc6oc5c4c3)cc2)cc1 ; CC1(C)c2ccccc2-c2ccc(N(c3ccccc3)c3ccc4c(-c5nc(-c6ccccc6)nc(-c6ccccc6)n5)cc5c6ccccc6oc5c4c3)cc21 ; c1ccc(-c2nc(-c3ccccc3)nc(-c3ccc(-c4cc5c6ccccc6oc5c5cc(N(c6ccccc6)c6ccc7ccccc7c6)ccc45)cc3)n2)cc1]

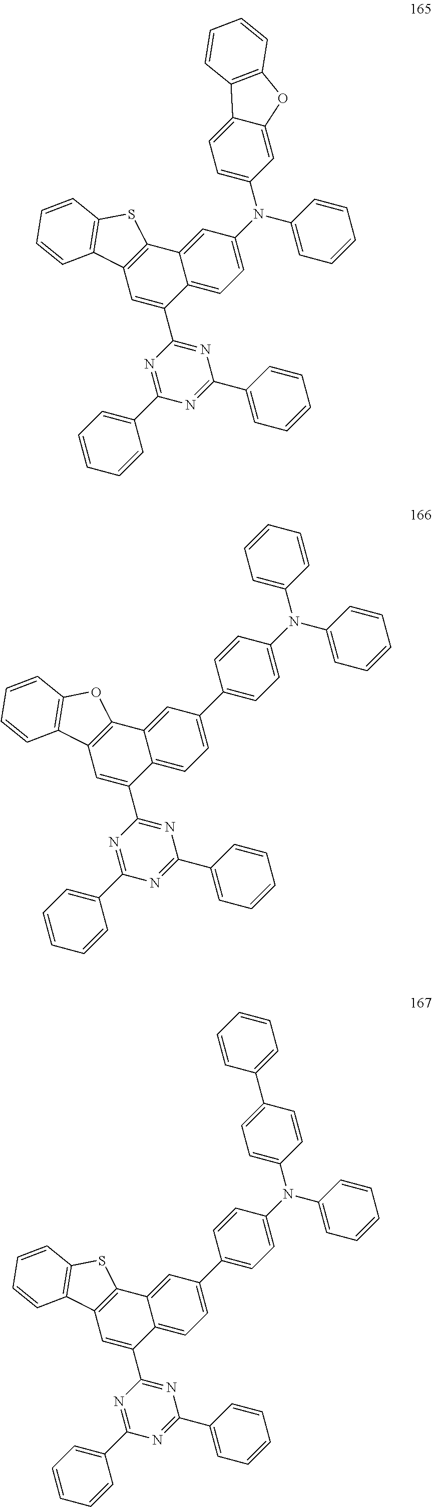 [SMILES: c1ccc(-c2nc(-c3ccccc3)nc(-c3cc4c5ccccc5sc4c4cc(N(c5ccccc5)c5ccc6c(c5)oc5ccccc56)ccc34)n2)cc1 ; c1ccc(-c2nc(-c3ccccc3)nc(-c3cc4c5ccccc5oc4c4cc(-c5ccc(N(c6ccccc6)c6ccccc6)cc5)ccc34)n2)cc1 ; c1ccc(-c2ccc(N(c3ccccc3)c3ccc(-c4ccc5c(-c6nc(-c7ccccc7)nc(-c7ccccc7)n6)cc6c7ccccc7sc6c5c4)cc3)cc2)cc1]

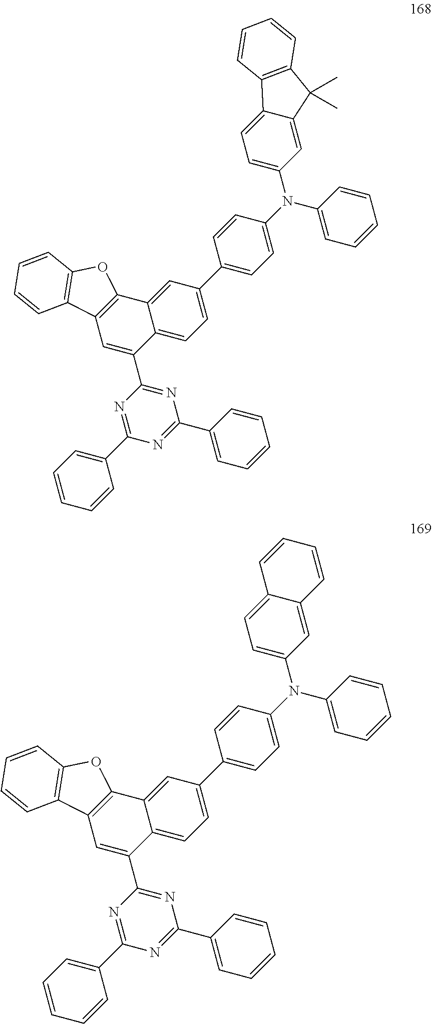 [SMILES: CC1(C)c2ccccc2-c2ccc(N(c3ccccc3)c3ccc(-c4ccc5c(-c6nc(-c7ccccc7)nc(-c7ccccc7)n6)cc6c7ccccc7oc6c5c4)cc3)cc21 ; c1ccc(-c2nc(-c3ccccc3)nc(-c3cc4c5ccccc5oc4c4cc(-c5ccc(N(c6ccccc6)c6ccc7ccccc7c6)cc5)ccc34)n2)cc1]

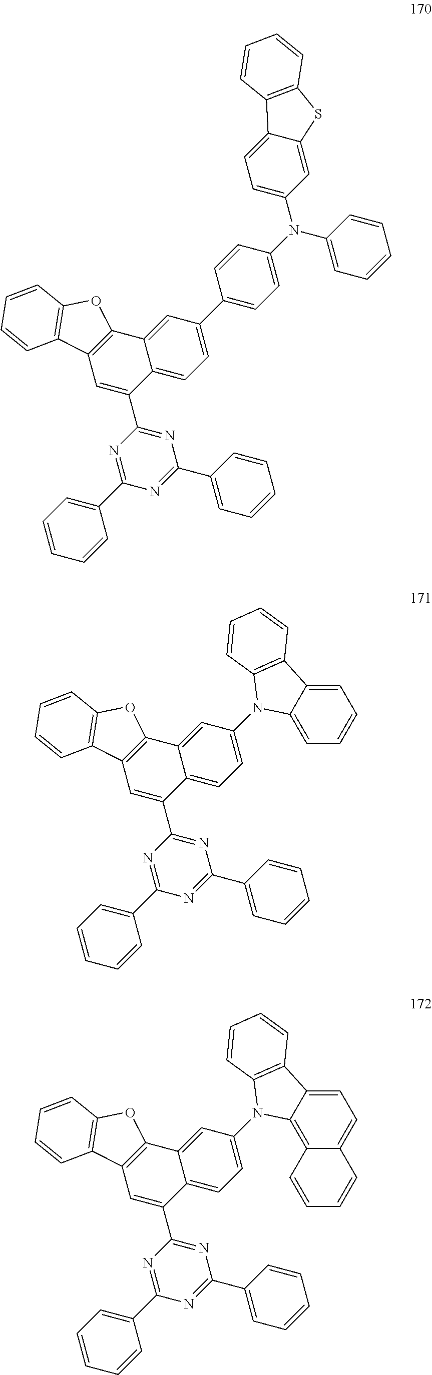 [SMILES: c1ccc(-c2nc(-c3ccccc3)nc(-c3cc4c5ccccc5oc4c4cc(-c5ccc(N(c6ccccc6)c6ccc7c(c6)sc6ccccc67)cc5)ccc34)n2)cc1 ; c1ccc(-c2nc(-c3ccccc3)nc(-c3cc4c5ccccc5oc4c4cc(-n5c6ccccc6c6ccccc65)ccc34)n2)cc1 ; c1ccc(-c2nc(-c3ccccc3)nc(-c3cc4c5ccccc5oc4c4cc(-n5c6ccccc6c6ccc7ccccc7c65)ccc34)n2)cc1]

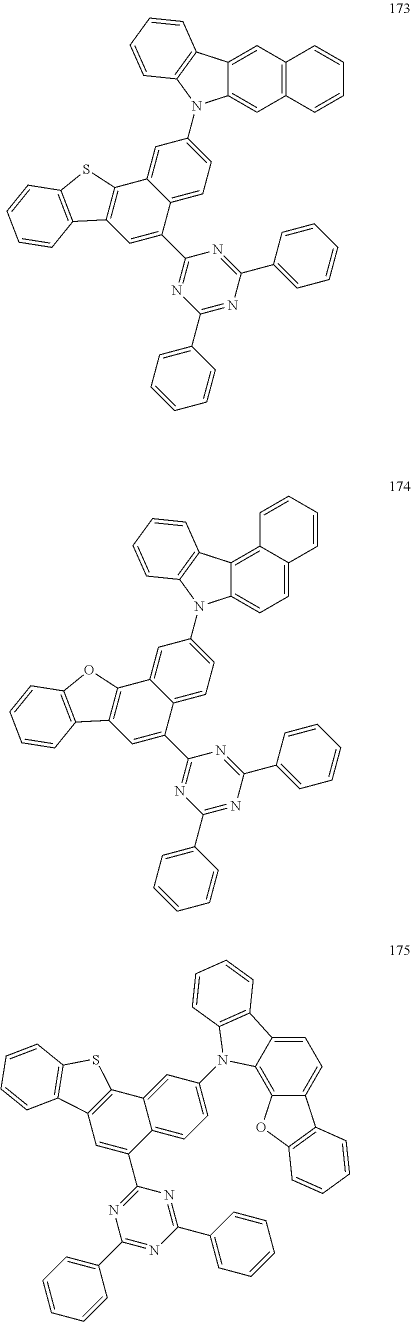 [SMILES: c1ccc(-c2nc(-c3ccccc3)nc(-c3cc4c5ccccc5sc4c4cc(-n5c6ccccc6c6cc7ccccc7cc65)ccc34)n2)cc1 ; c1ccc(-c2nc(-c3ccccc3)nc(-c3cc4c5ccccc5oc4c4cc(-n5c6ccccc6c6c7ccccc7ccc65)ccc34)n2)cc1 ; c1ccc(-c2nc(-c3ccccc3)nc(-c3cc4c5ccccc5sc4c4cc(-n5c6ccccc6c6ccc7c8ccccc8oc7c65)ccc34)n2)cc1]

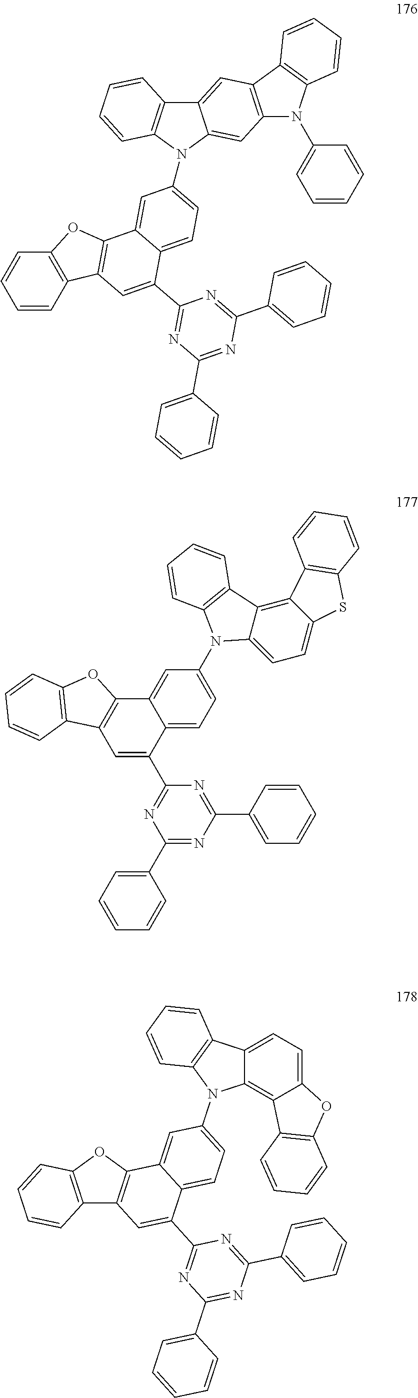 [SMILES: c1ccc(-c2nc(-c3ccccc3)nc(-c3cc4c5ccccc5oc4c4cc(-n5c6ccccc6c6cc7c8ccccc8n(-c8ccccc8)c7cc65)ccc34)n2)cc1 ; c1ccc(-c2nc(-c3ccccc3)nc(-c3cc4c5ccccc5oc4c4cc(-n5c6ccccc6c6c7c(ccc65)sc5ccccc57)ccc34)n2)cc1 ; c1ccc(-c2nc(-c3ccccc3)nc(-c3cc4c5ccccc5oc4c4cc(-n5c6ccccc6c6ccc7oc8ccccc8c7c65)ccc34)n2)cc1]

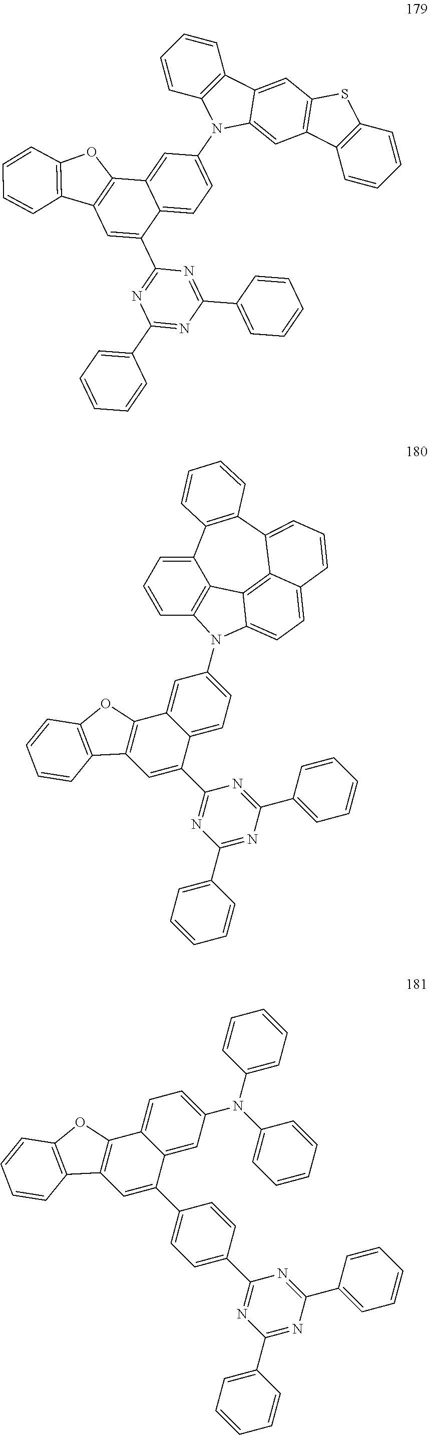 [SMILES: c1ccc(-c2nc(-c3ccccc3)nc(-c3cc4c5ccccc5oc4c4cc(-n5c6ccccc6c6cc7sc8ccccc8c7cc65)ccc34)n2)cc1 ; c1ccc(-c2nc(-c3ccccc3)nc(-c3cc4c5ccccc5oc4c4cc(-n5c6cccc7c6c6c8c(cccc8ccc65)-c5ccccc5-7)ccc34)n2)cc1 ; c1ccc(-c2nc(-c3ccccc3)nc(-c3ccc(-c4cc5c6ccccc6oc5c5ccc(N(c6ccccc6)c6ccccc6)cc45)cc3)n2)cc1]

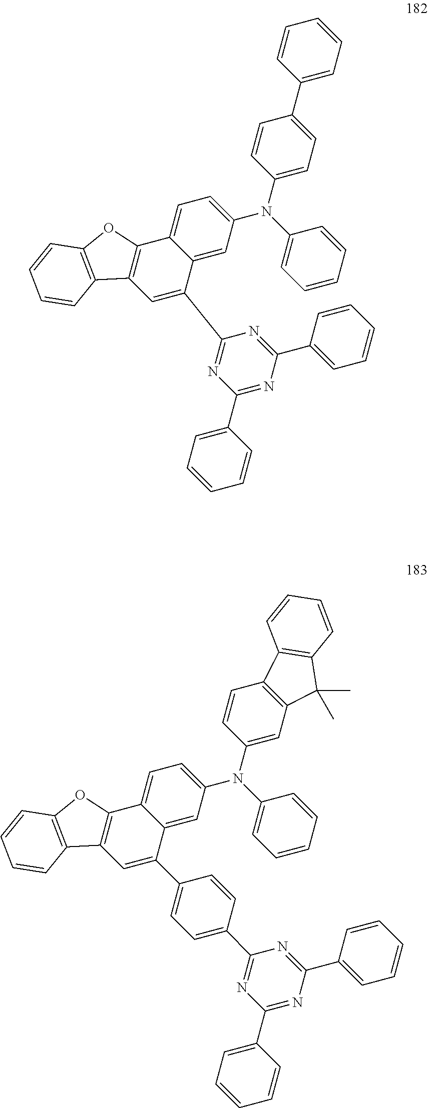 [SMILES: c1ccc(-c2ccc(N(c3ccccc3)c3ccc4c(c3)c(-c3nc(-c5ccccc5)nc(-c5ccccc5)n3)cc3c5ccccc5oc43)cc2)cc1 ; CC1(C)c2ccccc2-c2ccc(N(c3ccccc3)c3ccc4c(c3)c(-c3ccc(-c5nc(-c6ccccc6)nc(-c6ccccc6)n5)cc3)cc3c5ccccc5oc43)cc21]

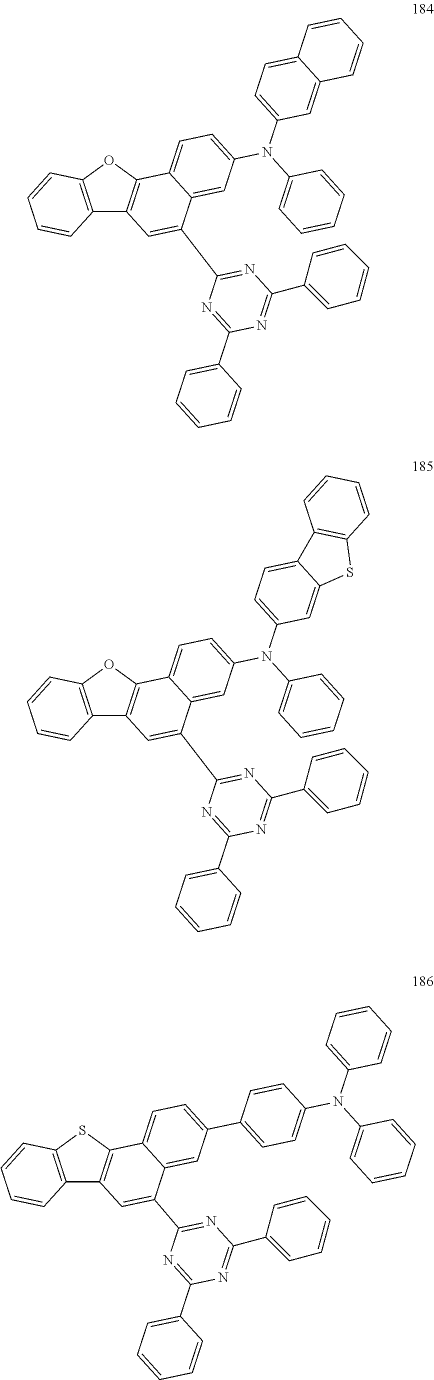 [SMILES: c1ccc(-c2nc(-c3ccccc3)nc(-c3cc4c5ccccc5oc4c4ccc(N(c5ccccc5)c5ccc6ccccc6c5)cc34)n2)cc1 ; c1ccc(-c2nc(-c3ccccc3)nc(-c3cc4c5ccccc5oc4c4ccc(N(c5ccccc5)c5ccc6c(c5)sc5ccccc56)cc34)n2)cc1 ; c1ccc(-c2nc(-c3ccccc3)nc(-c3cc4c5ccccc5sc4c4ccc(-c5ccc(N(c6ccccc6)c6ccccc6)cc5)cc34)n2)cc1]

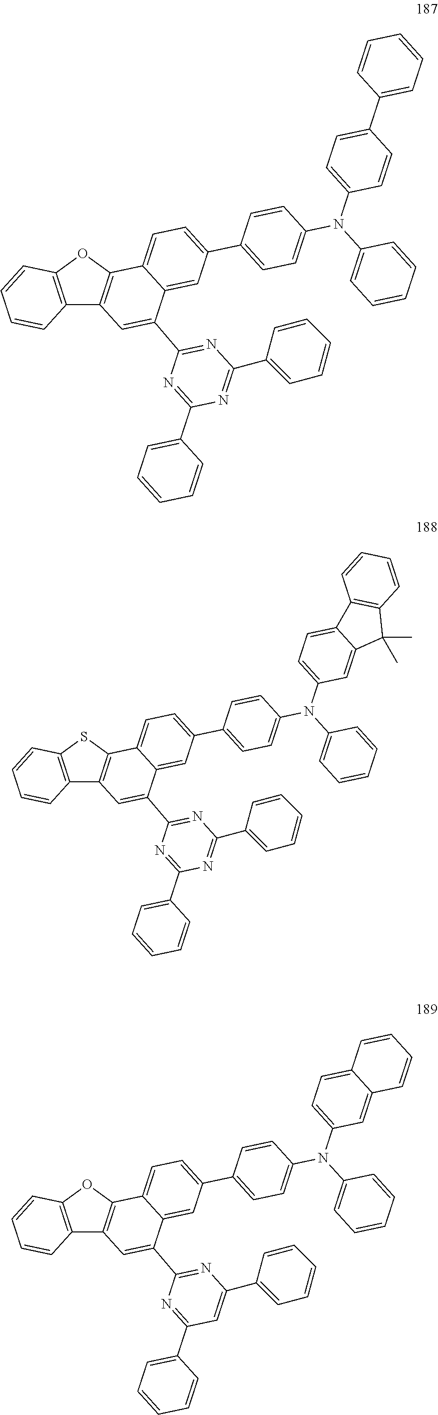 [SMILES: c1ccc(-c2ccc(N(c3ccccc3)c3ccc(-c4ccc5c(c4)c(-c4nc(-c6ccccc6)nc(-c6ccccc6)n4)cc4c6ccccc6oc54)cc3)cc2)cc1 ; CC1(C)c2ccccc2-c2ccc(N(c3ccccc3)c3ccc(-c4ccc5c(c4)c(-c4nc(-c6ccccc6)nc(-c6ccccc6)n4)cc4c6ccccc6sc54)cc3)cc21 ; c1ccc(-c2cc(-c3ccccc3)nc(-c3cc4c5ccccc5oc4c4ccc(-c5ccc(N(c6ccccc6)c6ccc7ccccc7c6)cc5)cc34)n2)cc1]

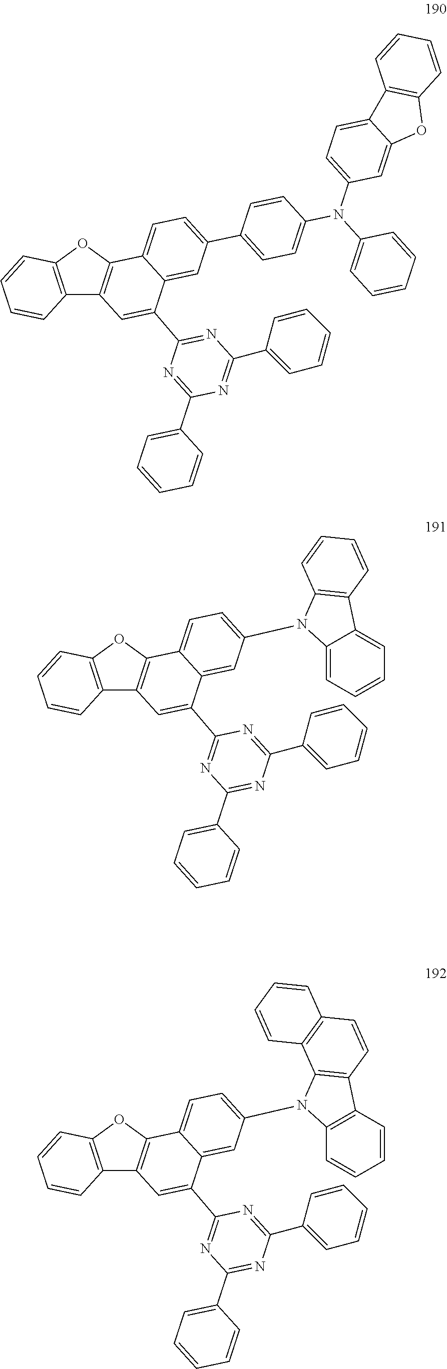 [SMILES: c1ccc(-c2nc(-c3ccccc3)nc(-c3cc4c5ccccc5oc4c4ccc(-c5ccc(N(c6ccccc6)c6ccc7c(c6)oc6ccccc67)cc5)cc34)n2)cc1 ; c1ccc(-c2nc(-c3ccccc3)nc(-c3cc4c5ccccc5oc4c4ccc(-n5c6ccccc6c6ccccc65)cc34)n2)cc1 ; c1ccc(-c2nc(-c3ccccc3)nc(-c3cc4c5ccccc5oc4c4ccc(-n5c6ccccc6c6ccc7ccccc7c65)cc34)n2)cc1]

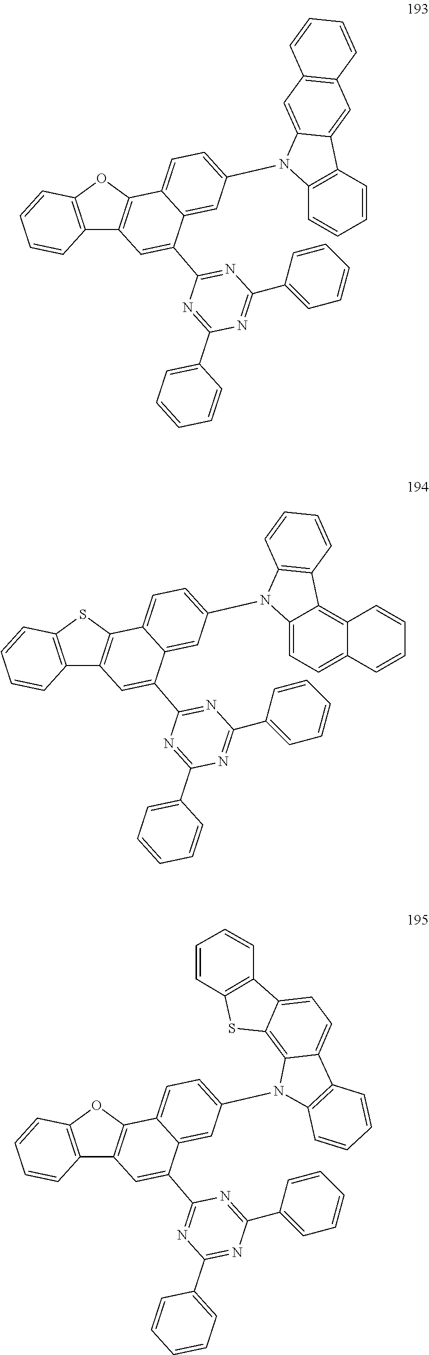 [SMILES: c1ccc(-c2nc(-c3ccccc3)nc(-c3cc4c5ccccc5oc4c4ccc(-n5c6ccccc6c6cc7ccccc7cc65)cc34)n2)cc1 ; c1ccc(-c2nc(-c3ccccc3)nc(-c3cc4c5ccccc5sc4c4ccc(-n5c6ccccc6c6c7ccccc7ccc65)cc34)n2)cc1 ; c1ccc(-c2nc(-c3ccccc3)nc(-c3cc4c5ccccc5oc4c4ccc(-n5c6ccccc6c6ccc7c8ccccc8sc7c65)cc34)n2)cc1]

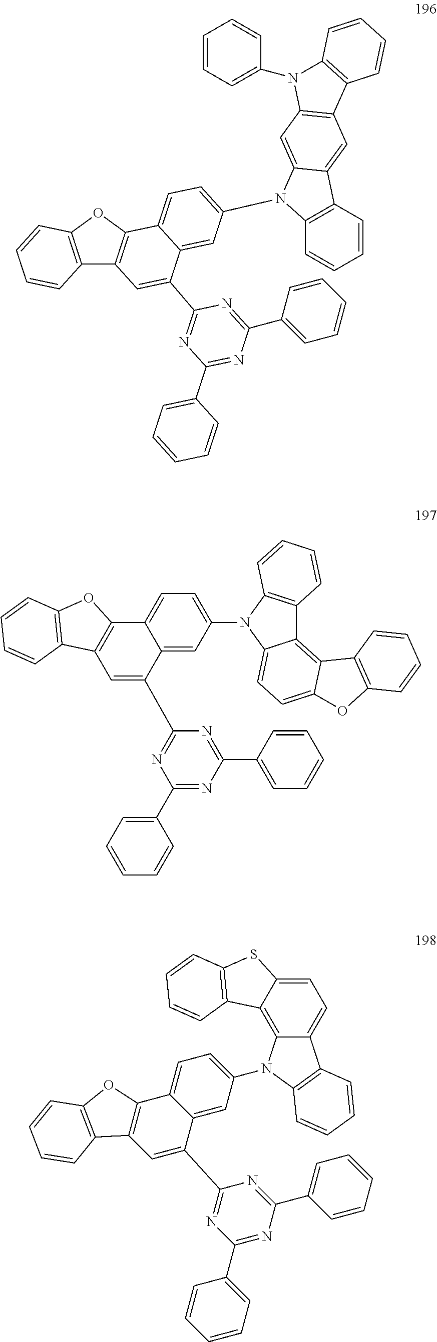 [SMILES: c1ccc(-c2nc(-c3ccccc3)nc(-c3cc4c5ccccc5oc4c4ccc(-n5c6ccccc6c6cc7c8ccccc8n(-c8ccccc8)c7cc65)cc34)n2)cc1 ; c1ccc(-c2nc(-c3ccccc3)nc(-c3cc4c5ccccc5oc4c4ccc(-n5c6ccccc6c6c7c(ccc65)oc5ccccc57)cc34)n2)cc1 ; c1ccc(-c2nc(-c3ccccc3)nc(-c3cc4c5ccccc5oc4c4ccc(-n5c6ccccc6c6ccc7sc8ccccc8c7c65)cc34)n2)cc1]

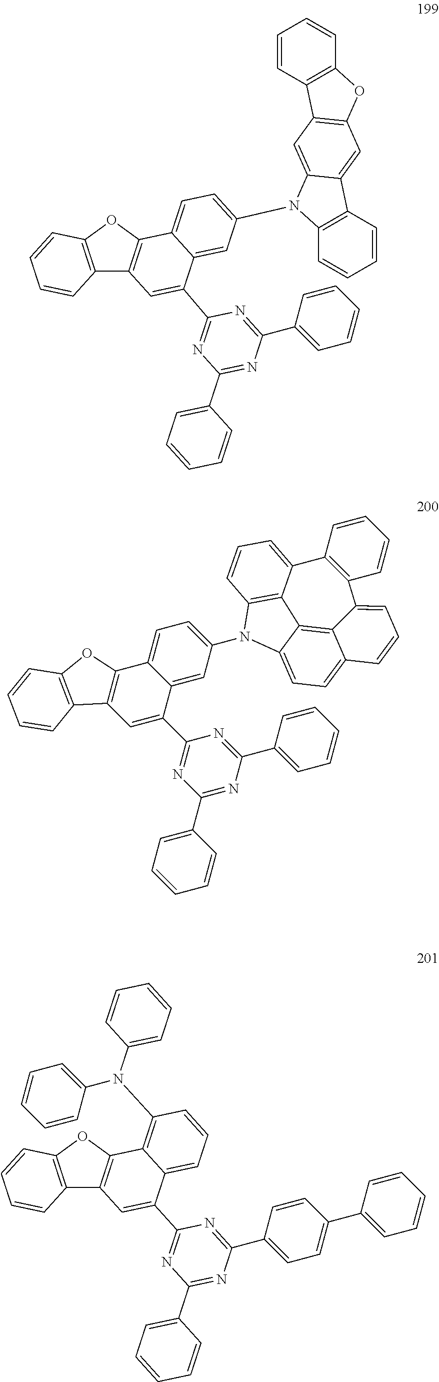 [SMILES: c1ccc(-c2nc(-c3ccccc3)nc(-c3cc4c5ccccc5oc4c4ccc(-n5c6ccccc6c6cc7oc8ccccc8c7cc65)cc34)n2)cc1 ; c1ccc(-c2nc(-c3ccccc3)nc(-c3cc4c5ccccc5oc4c4ccc(-n5c6cccc7c6c6c8c(cccc8ccc65)-c5ccccc5-7)cc34)n2)cc1 ; c1ccc(-c2ccc(-c3nc(-c4ccccc4)nc(-c4cc5c6ccccc6oc5c5c(N(c6ccccc6)c6ccccc6)cccc45)n3)cc2)cc1]

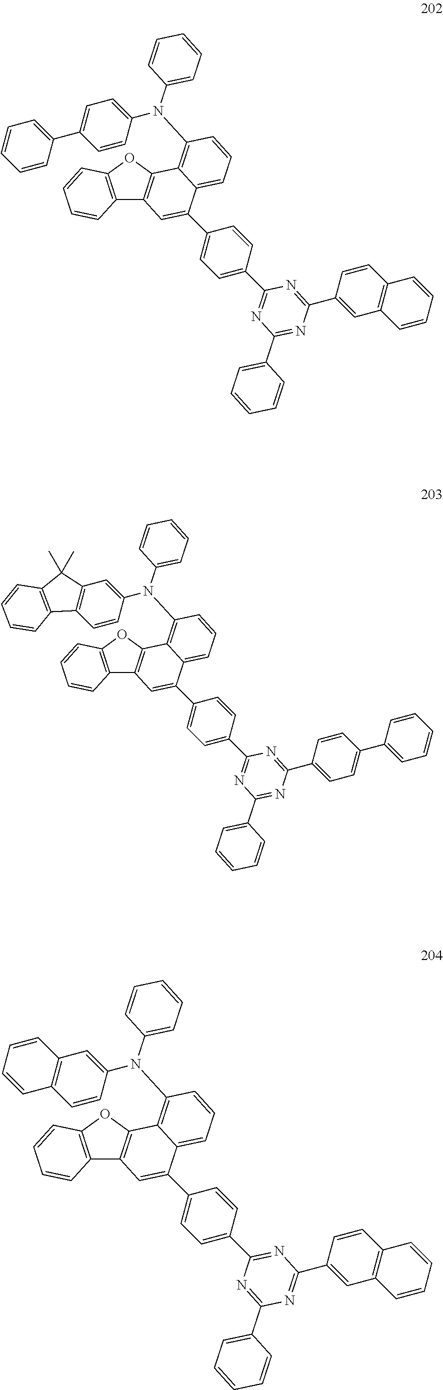 [SMILES: c1ccc(-c2ccc(N(c3ccccc3)c3cccc4c(-c5ccc(-c6nc(-c7ccccc7)nc(-c7ccc8ccccc8c7)n6)cc5)cc5c6ccccc6oc5c34)cc2)cc1 ; CC1(C)c2ccccc2-c2ccc(N(c3ccccc3)c3cccc4c(-c5ccc(-c6nc(-c7ccccc7)nc(-c7ccc(-c8ccccc8)cc7)n6)cc5)cc5c6ccccc6oc5c34)cc21 ; c1ccc(-c2nc(-c3ccc(-c4cc5c6ccccc6oc5c5c(N(c6ccccc6)c6ccc7ccccc7c6)cccc45)cc3)nc(-c3ccc4ccccc4c3)n2)cc1]

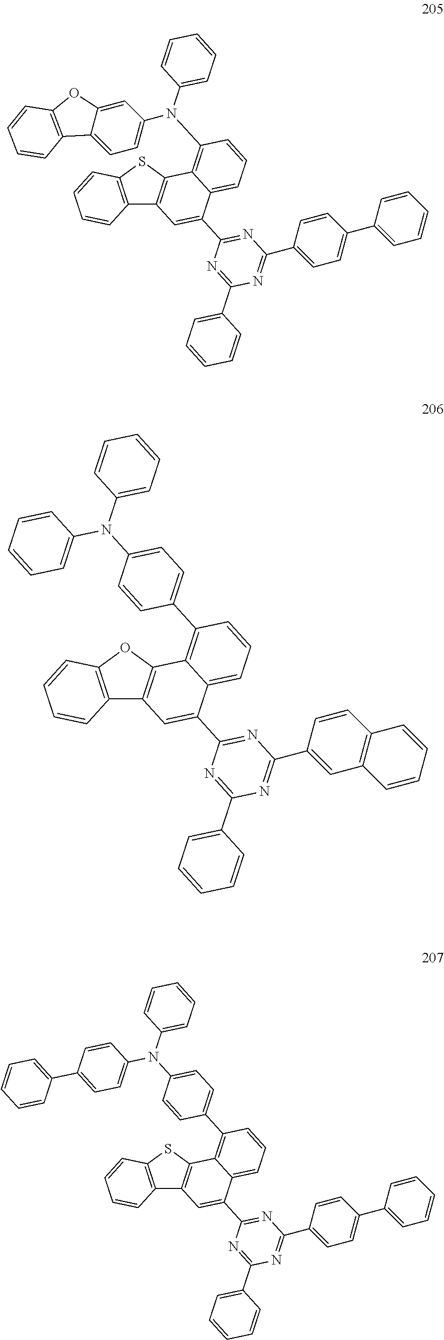 [SMILES: c1ccc(-c2ccc(-c3nc(-c4ccccc4)nc(-c4cc5c6ccccc6sc5c5c(N(c6ccccc6)c6ccc7c(c6)oc6ccccc67)cccc45)n3)cc2)cc1 ; c1ccc(-c2nc(-c3ccc4ccccc4c3)nc(-c3cc4c5ccccc5oc4c4c(-c5ccc(N(c6ccccc6)c6ccccc6)cc5)cccc34)n2)cc1 ; c1ccc(-c2ccc(-c3nc(-c4ccccc4)nc(-c4cc5c6ccccc6sc5c5c(-c6ccc(N(c7ccccc7)c7ccc(-c8ccccc8)cc7)cc6)cccc45)n3)cc2)cc1]

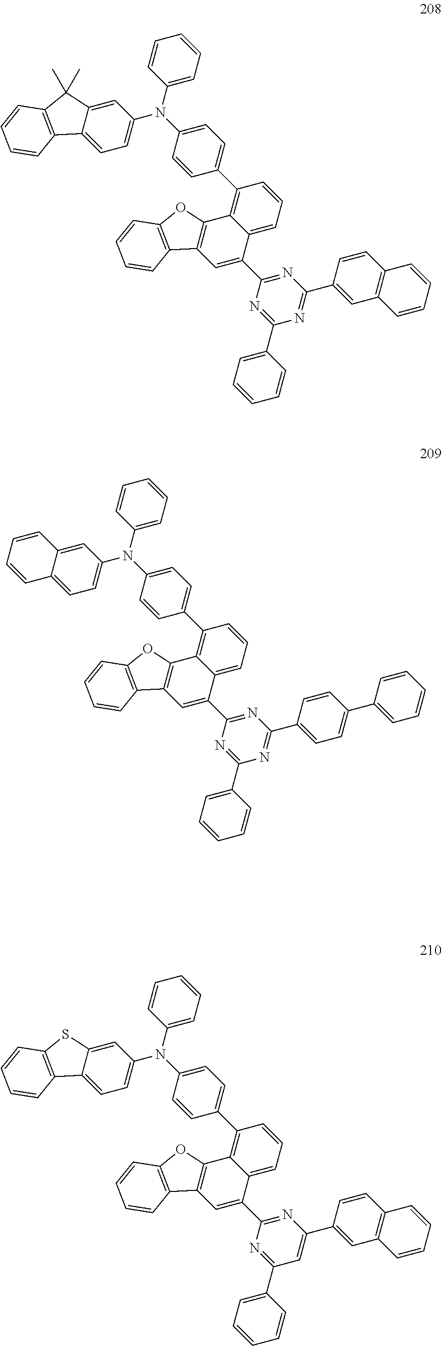 [SMILES: CC1(C)c2ccccc2-c2ccc(N(c3ccccc3)c3ccc(-c4cccc5c(-c6nc(-c7ccccc7)nc(-c7ccc8ccccc8c7)n6)cc6c7ccccc7oc6c45)cc3)cc21 ; c1ccc(-c2ccc(-c3nc(-c4ccccc4)nc(-c4cc5c6ccccc6oc5c5c(-c6ccc(N(c7ccccc7)c7ccc8ccccc8c7)cc6)cccc45)n3)cc2)cc1 ; c1ccc(-c2cc(-c3ccc4ccccc4c3)nc(-c3cc4c5ccccc5oc4c4c(-c5ccc(N(c6ccccc6)c6ccc7c(c6)sc6ccccc67)cc5)cccc34)n2)cc1]

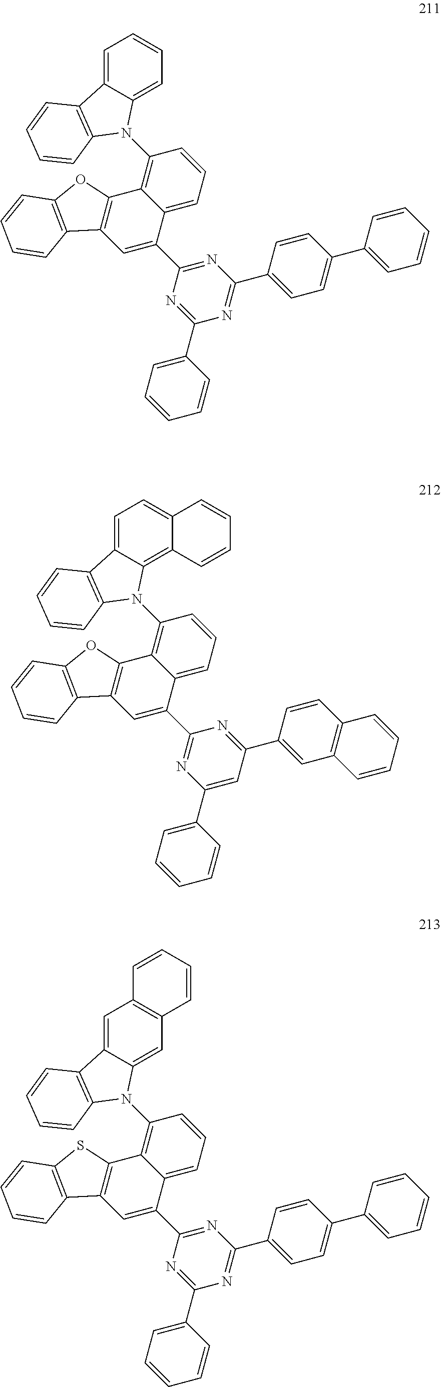 [SMILES: c1ccc(-c2ccc(-c3nc(-c4ccccc4)nc(-c4cc5c6ccccc6oc5c5c(-n6c7ccccc7c7ccccc76)cccc45)n3)cc2)cc1 ; c1ccc(-c2cc(-c3ccc4ccccc4c3)nc(-c3cc4c5ccccc5oc4c4c(-n5c6ccccc6c6ccc7ccccc7c65)cccc34)n2)cc1 ; c1ccc(-c2ccc(-c3nc(-c4ccccc4)nc(-c4cc5c6ccccc6sc5c5c(-n6c7ccccc7c7cc8ccccc8cc76)cccc45)n3)cc2)cc1]

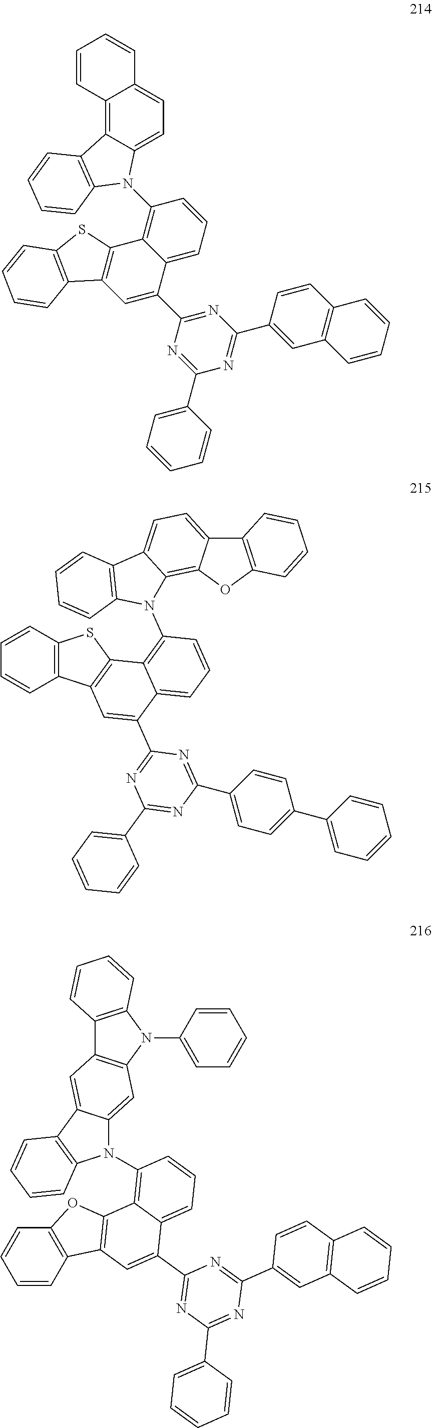

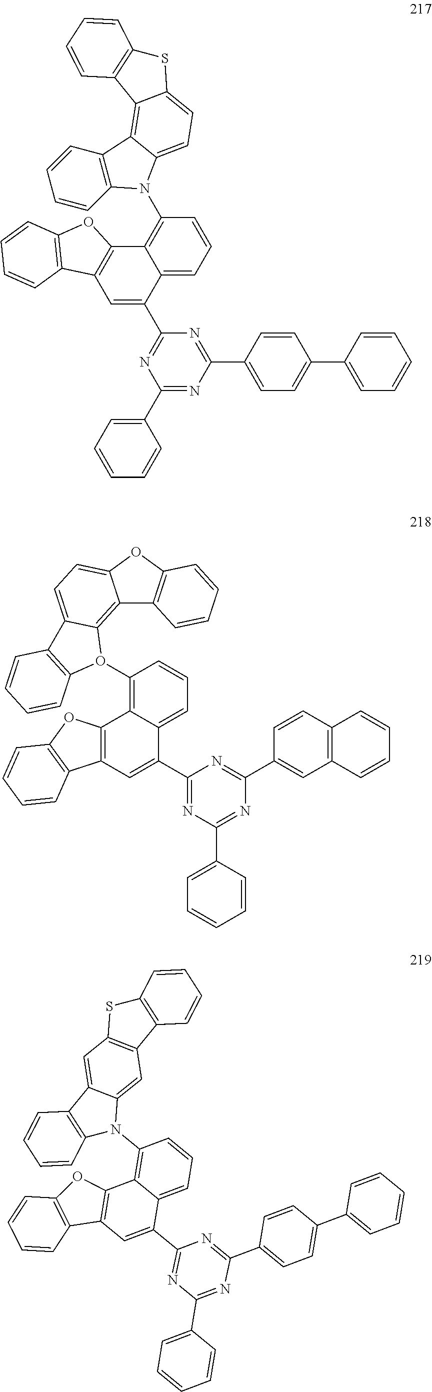

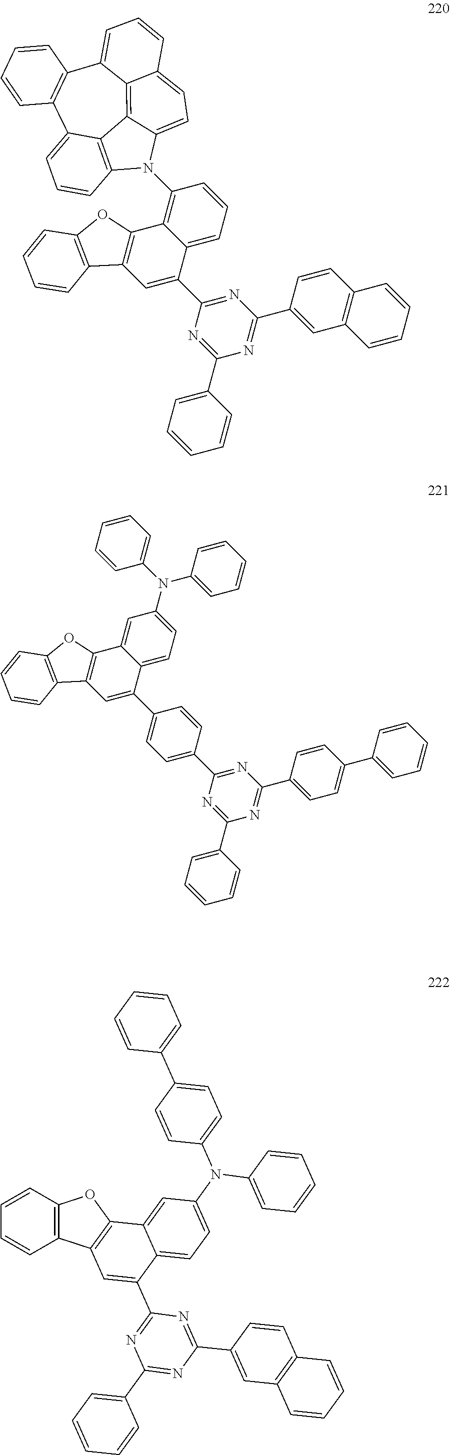 [SMILES: c1ccc(-c2nc(-c3ccc4ccccc4c3)nc(-c3cc4c5ccccc5oc4c4c(-n5c6cccc7c6c6c8c(cccc8ccc65)-c5ccccc5-7)cccc34)n2)cc1 ; c1ccc(-c2ccc(-c3nc(-c4ccccc4)nc(-c4ccc(-c5cc6c7ccccc7oc6c6cc(N(c7ccccc7)c7ccccc7)ccc56)cc4)n3)cc2)cc1 ; c1ccc(-c2ccc(N(c3ccccc3)c3ccc4c(-c5nc(-c6ccccc6)nc(-c6ccc7ccccc7c6)n5)cc5c6ccccc6oc5c4c3)cc2)cc1]

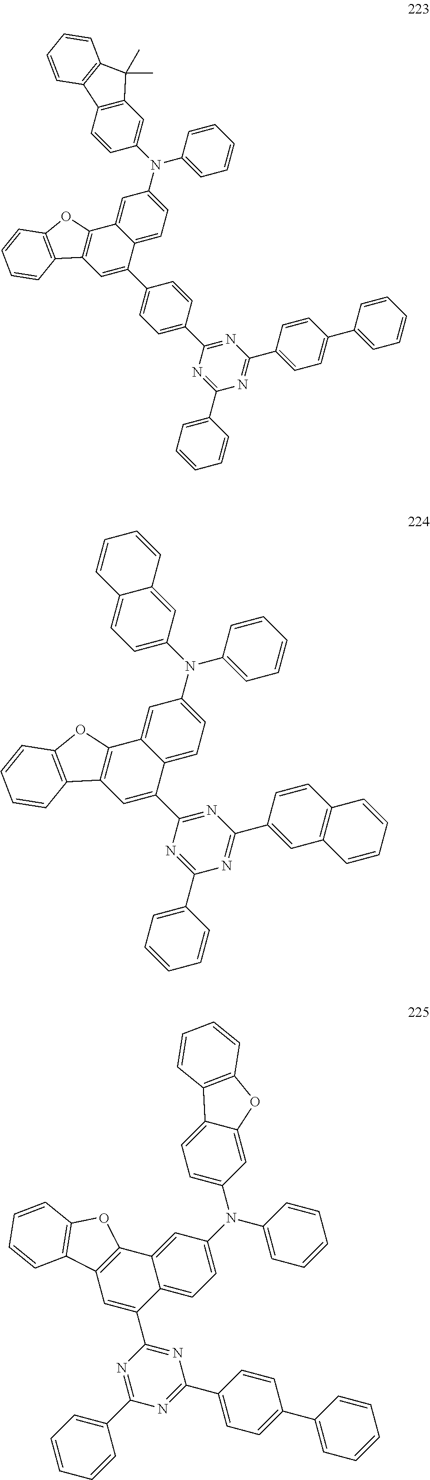 [SMILES: CC1(C)c2ccccc2-c2ccc(N(c3ccccc3)c3ccc4c(-c5ccc(-c6nc(-c7ccccc7)nc(-c7ccc(-c8ccccc8)cc7)n6)cc5)cc5c6ccccc6oc5c4c3)cc21 ; c1ccc(-c2nc(-c3ccc4ccccc4c3)nc(-c3cc4c5ccccc5oc4c4cc(N(c5ccccc5)c5ccc6ccccc6c5)ccc34)n2)cc1 ; c1ccc(-c2ccc(-c3nc(-c4ccccc4)nc(-c4cc5c6ccccc6oc5c5cc(N(c6ccccc6)c6ccc7c(c6)oc6ccccc67)ccc45)n3)cc2)cc1]

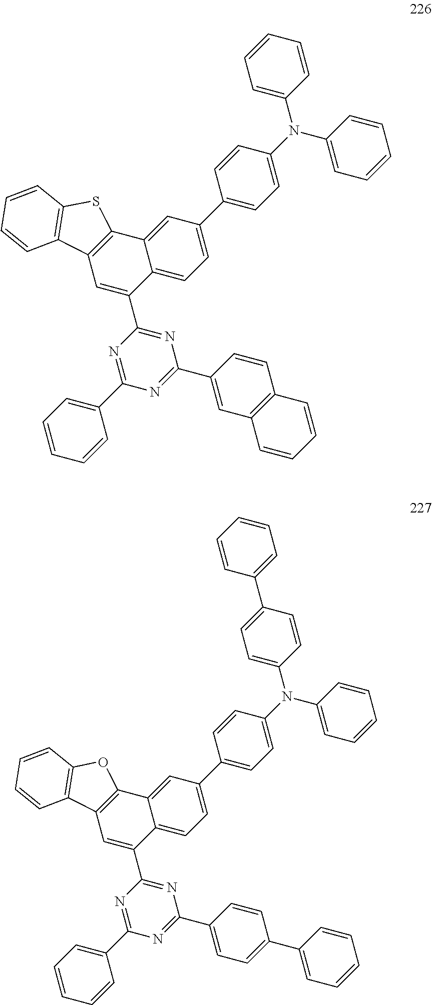 [SMILES: c1ccc(-c2nc(-c3ccc4ccccc4c3)nc(-c3cc4c5ccccc5sc4c4cc(-c5ccc(N(c6ccccc6)c6ccccc6)cc5)ccc34)n2)cc1 ; c1ccc(-c2ccc(-c3nc(-c4ccccc4)nc(-c4cc5c6ccccc6oc5c5cc(-c6ccc(N(c7ccccc7)c7ccc(-c8ccccc8)cc7)cc6)ccc45)n3)cc2)cc1]

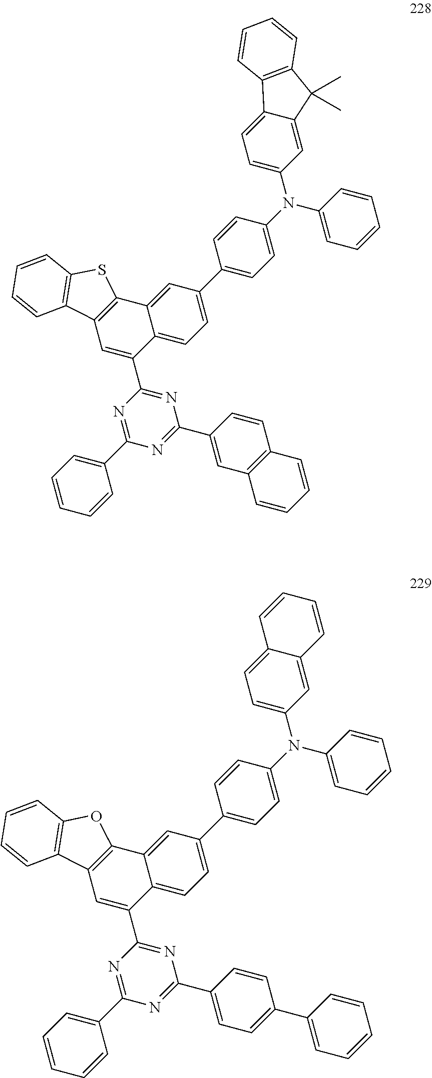 [SMILES: CC1(C)c2ccccc2-c2ccc(N(c3ccccc3)c3ccc(-c4ccc5c(-c6nc(-c7ccccc7)nc(-c7ccc8ccccc8c7)n6)cc6c7ccccc7sc6c5c4)cc3)cc21 ; c1ccc(-c2ccc(-c3nc(-c4ccccc4)nc(-c4cc5c6ccccc6oc5c5cc(-c6ccc(N(c7ccccc7)c7ccc8ccccc8c7)cc6)ccc45)n3)cc2)cc1]

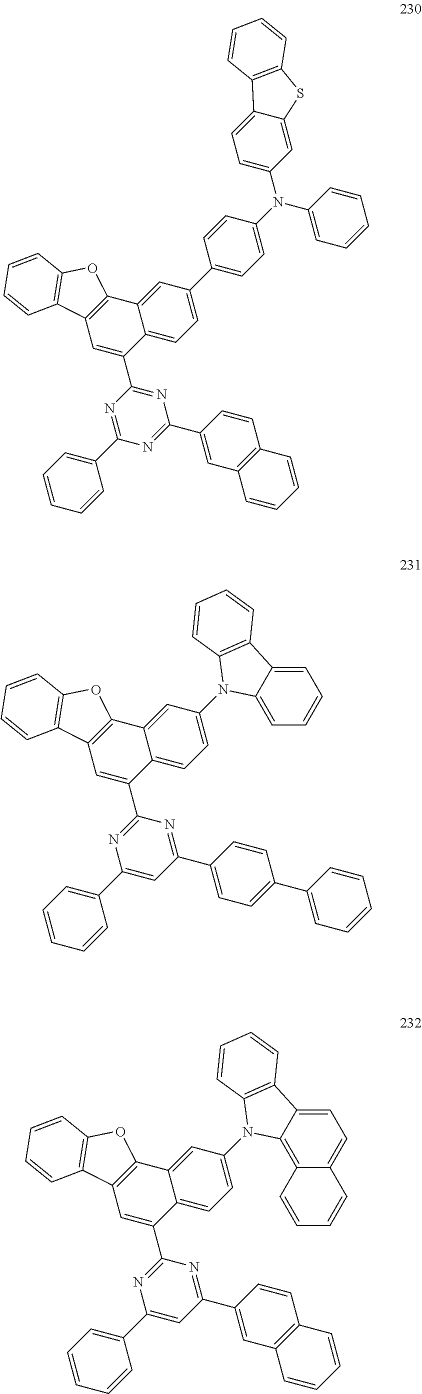 [SMILES: c1ccc(-c2nc(-c3ccc4ccccc4c3)nc(-c3cc4c5ccccc5oc4c4cc(-c5ccc(N(c6ccccc6)c6ccc7c(c6)sc6ccccc67)cc5)ccc34)n2)cc1 ; c1ccc(-c2ccc(-c3cc(-c4ccccc4)nc(-c4cc5c6ccccc6oc5c5cc(-n6c7ccccc7c7ccccc76)ccc45)n3)cc2)cc1 ; c1ccc(-c2cc(-c3ccc4ccccc4c3)nc(-c3cc4c5ccccc5oc4c4cc(-n5c6ccccc6c6ccc7ccccc7c65)ccc34)n2)cc1]

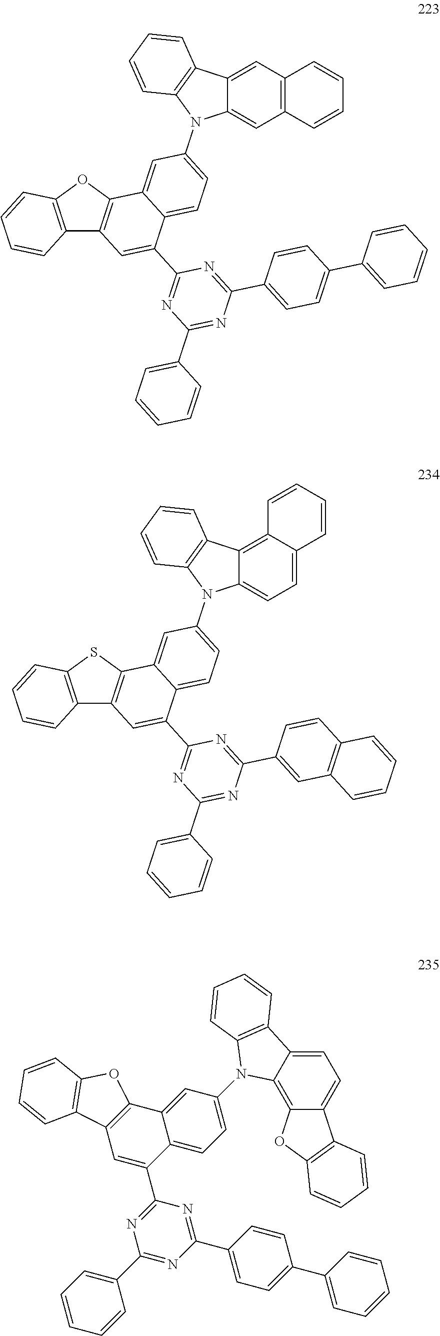 [SMILES: c1ccc(-c2ccc(-c3nc(-c4ccccc4)nc(-c4cc5c6ccccc6oc5c5cc(-n6c7ccccc7c7cc8ccccc8cc76)ccc45)n3)cc2)cc1 ; c1ccc(-c2nc(-c3ccc4ccccc4c3)nc(-c3cc4c5ccccc5sc4c4cc(-n5c6ccccc6c6c7ccccc7ccc65)ccc34)n2)cc1 ; c1ccc(-c2ccc(-c3nc(-c4ccccc4)nc(-c4cc5c6ccccc6oc5c5cc(-n6c7ccccc7c7ccc8c9ccccc9oc8c76)ccc45)n3)cc2)cc1]

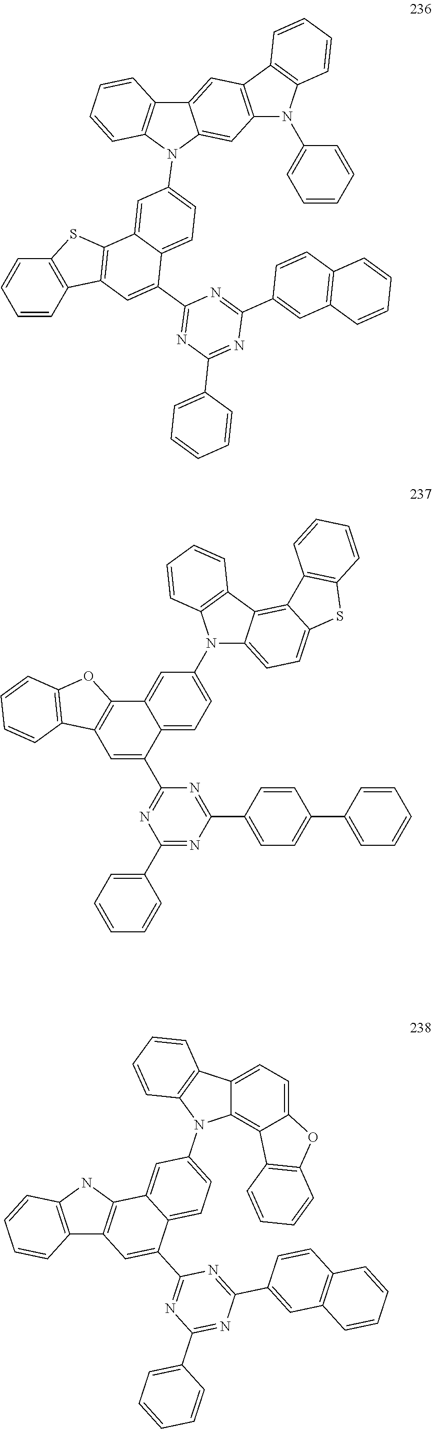 [SMILES: c1ccc(-c2nc(-c3ccc4ccccc4c3)nc(-c3cc4c5ccccc5sc4c4cc(-n5c6ccccc6c6cc7c8ccccc8n(-c8ccccc8)c7cc65)ccc34)n2)cc1 ; c1ccc(-c2ccc(-c3nc(-c4ccccc4)nc(-c4cc5c6ccccc6oc5c5cc(-n6c7ccccc7c7c8c(ccc76)sc6ccccc68)ccc45)n3)cc2)cc1 ; c1ccc(-c2nc(-c3ccc4ccccc4c3)nc(-c3cc4c5ccccc5[nH]c4c4cc(-n5c6ccccc6c6ccc7oc8ccccc8c7c65)ccc34)n2)cc1]

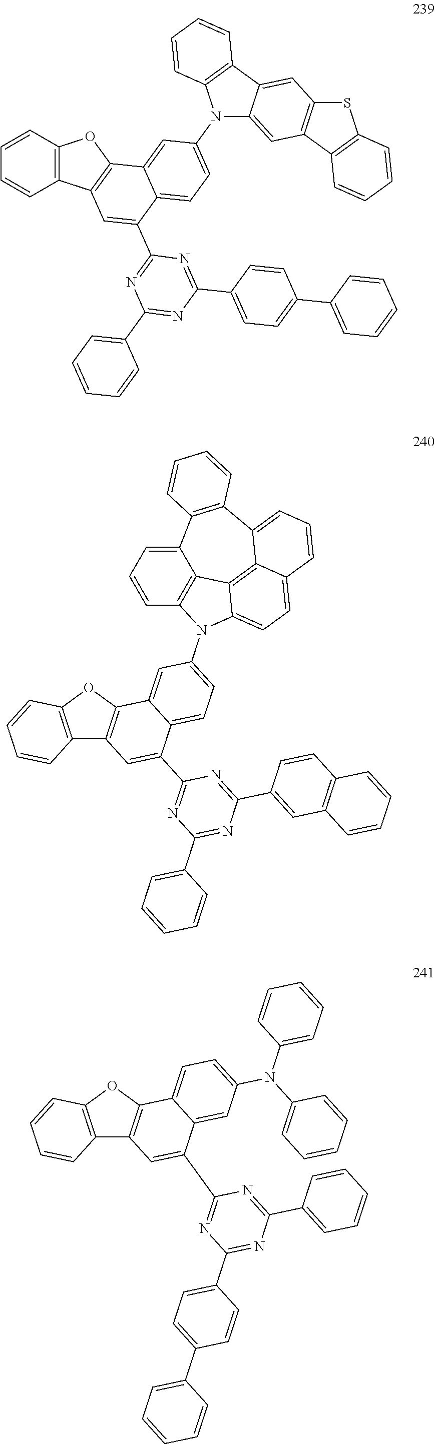 [SMILES: c1ccc(-c2ccc(-c3nc(-c4ccccc4)nc(-c4cc5c6ccccc6oc5c5cc(-n6c7ccccc7c7cc8sc9ccccc9c8cc76)ccc45)n3)cc2)cc1 ; c1ccc(-c2nc(-c3ccc4ccccc4c3)nc(-c3cc4c5ccccc5oc4c4cc(-n5c6cccc7c6c6c8c(cccc8ccc65)-c5ccccc5-7)ccc34)n2)cc1 ; c1ccc(-c2ccc(-c3nc(-c4ccccc4)nc(-c4cc5c6ccccc6oc5c5ccc(N(c6ccccc6)c6ccccc6)cc45)n3)cc2)cc1]

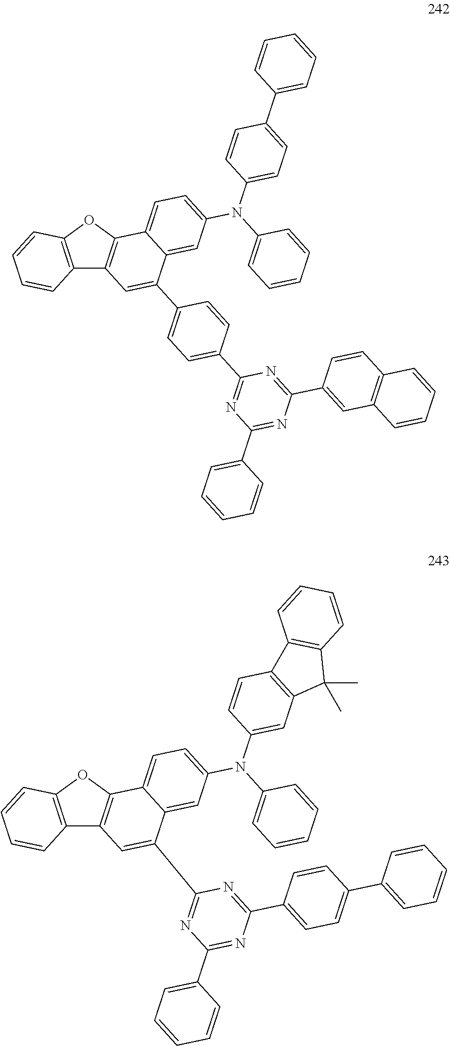 [SMILES: c1ccc(-c2ccc(N(c3ccccc3)c3ccc4c(c3)c(-c3ccc(-c5nc(-c6ccccc6)nc(-c6ccc7ccccc7c6)n5)cc3)cc3c5ccccc5oc43)cc2)cc1 ; CC1(C)c2ccccc2-c2ccc(N(c3ccccc3)c3ccc4c(c3)c(-c3nc(-c5ccccc5)nc(-c5ccc(-c6ccccc6)cc5)n3)cc3c5ccccc5oc43)cc21]

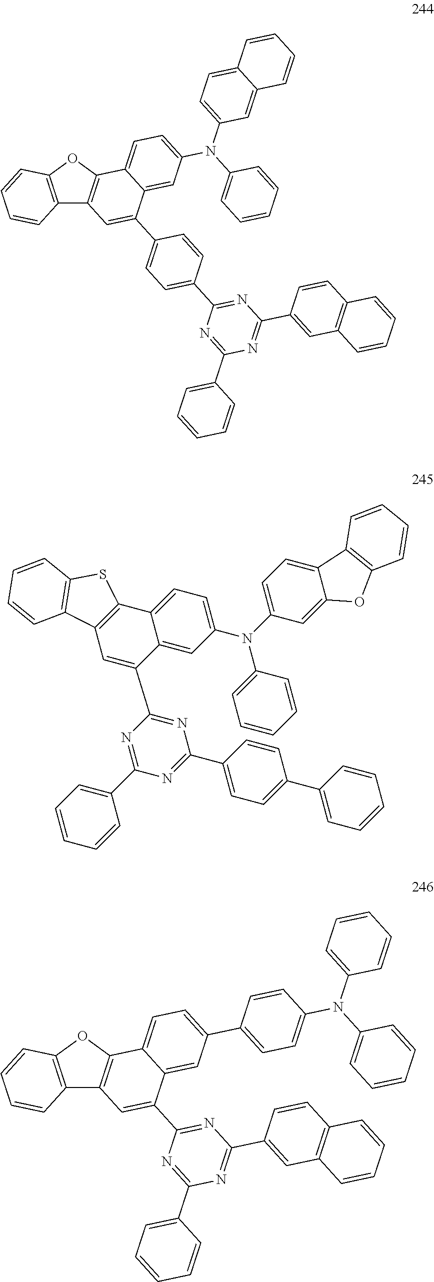 [SMILES: c1ccc(-c2nc(-c3ccc(-c4cc5c6ccccc6oc5c5ccc(N(c6ccccc6)c6ccc7ccccc7c6)cc45)cc3)nc(-c3ccc4ccccc4c3)n2)cc1 ; c1ccc(-c2ccc(-c3nc(-c4ccccc4)nc(-c4cc5c6ccccc6sc5c5ccc(N(c6ccccc6)c6ccc7c(c6)oc6ccccc67)cc45)n3)cc2)cc1 ; c1ccc(-c2nc(-c3ccc4ccccc4c3)nc(-c3cc4c5ccccc5oc4c4ccc(-c5ccc(N(c6ccccc6)c6ccccc6)cc5)cc34)n2)cc1]

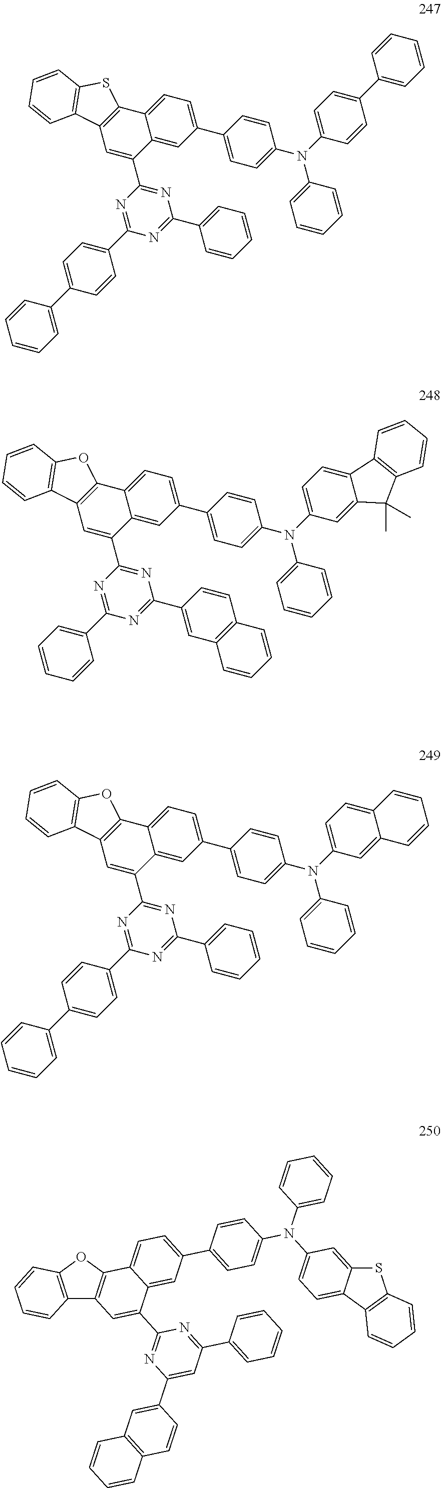 [SMILES: c1ccc(-c2ccc(-c3nc(-c4ccccc4)nc(-c4cc5c6ccccc6sc5c5ccc(-c6ccc(N(c7ccccc7)c7ccc(-c8ccccc8)cc7)cc6)cc45)n3)cc2)cc1 ; CC1(C)c2ccccc2-c2ccc(N(c3ccccc3)c3ccc(-c4ccc5c(c4)c(-c4nc(-c6ccccc6)nc(-c6ccc7ccccc7c6)n4)cc4c6ccccc6oc54)cc3)cc21 ; c1ccc(-c2ccc(-c3nc(-c4ccccc4)nc(-c4cc5c6ccccc6oc5c5ccc(-c6ccc(N(c7ccccc7)c7ccc8ccccc8c7)cc6)cc45)n3)cc2)cc1 ; c1ccc(-c2cc(-c3ccc4ccccc4c3)nc(-c3cc4c5ccccc5oc4c4ccc(-c5ccc(N(c6ccccc6)c6ccc7c(c6)sc6ccccc67)cc5)cc34)n2)cc1]

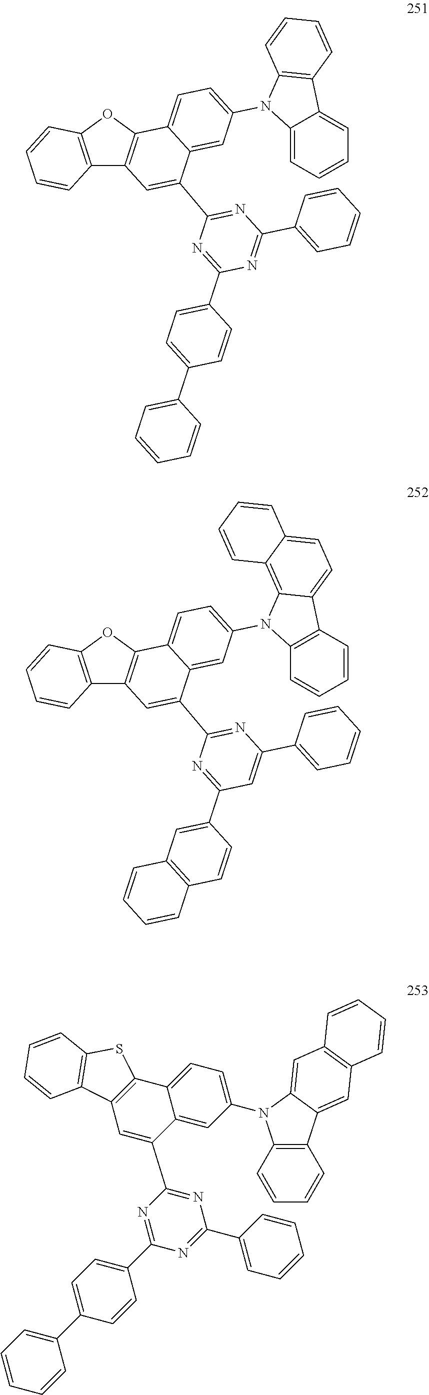 [SMILES: c1ccc(-c2ccc(-c3nc(-c4ccccc4)nc(-c4cc5c6ccccc6oc5c5ccc(-n6c7ccccc7c7ccccc76)cc45)n3)cc2)cc1 ; c1ccc(-c2cc(-c3ccc4ccccc4c3)nc(-c3cc4c5ccccc5oc4c4ccc(-n5c6ccccc6c6ccc7ccccc7c65)cc34)n2)cc1 ; c1ccc(-c2ccc(-c3nc(-c4ccccc4)nc(-c4cc5c6ccccc6sc5c5ccc(-n6c7ccccc7c7cc8ccccc8cc76)cc45)n3)cc2)cc1]

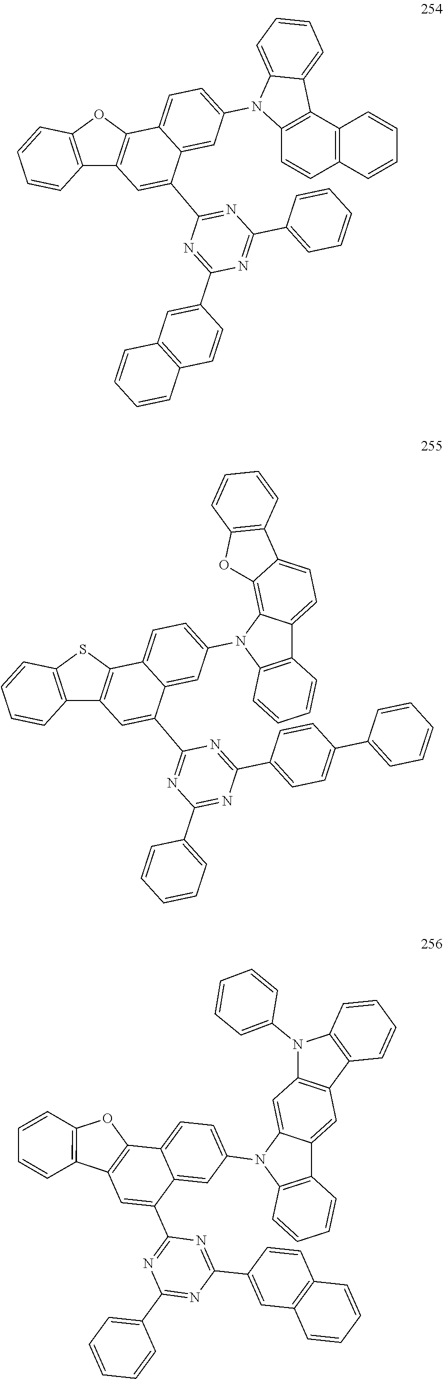 [SMILES: c1ccc(-c2nc(-c3ccc4ccccc4c3)nc(-c3cc4c5ccccc5oc4c4ccc(-n5c6ccccc6c6c7ccccc7ccc65)cc34)n2)cc1 ; c1ccc(-c2ccc(-c3nc(-c4ccccc4)nc(-c4cc5c6ccccc6sc5c5ccc(-n6c7ccccc7c7ccc8c9ccccc9oc8c76)cc45)n3)cc2)cc1 ; c1ccc(-c2nc(-c3ccc4ccccc4c3)nc(-c3cc4c5ccccc5oc4c4ccc(-n5c6ccccc6c6cc7c8ccccc8n(-c8ccccc8)c7cc65)cc34)n2)cc1]

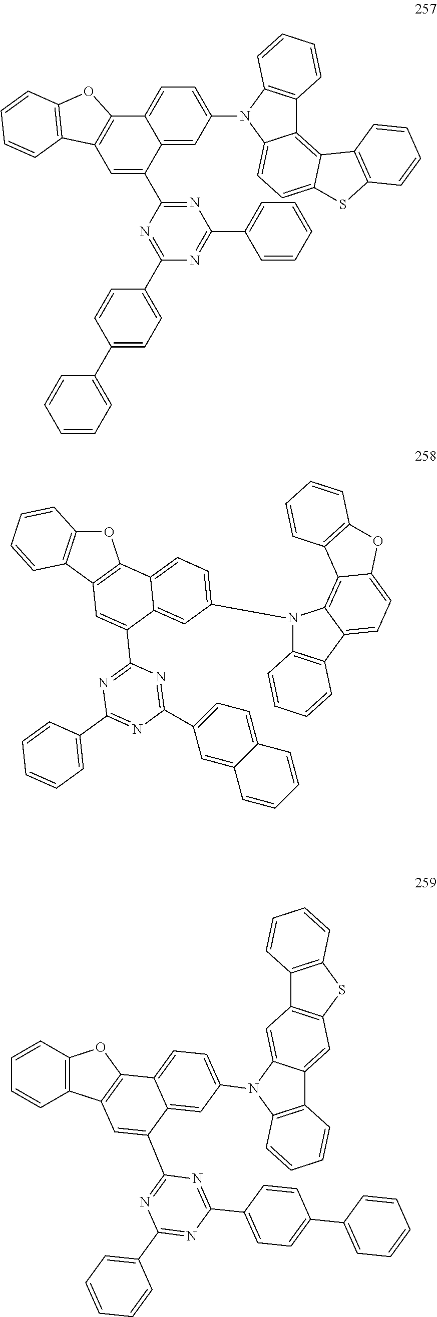 [SMILES: c1ccc(-c2ccc(-c3nc(-c4ccccc4)nc(-c4cc5c6ccccc6oc5c5ccc(-n6c7ccccc7c7c8c(ccc76)sc6ccccc68)cc45)n3)cc2)cc1 ; c1ccc(-c2nc(-c3ccc4ccccc4c3)nc(-c3cc4c5ccccc5oc4c4ccc(-n5c6ccccc6c6ccc7oc8ccccc8c7c65)cc34)n2)cc1 ; c1ccc(-c2ccc(-c3nc(-c4ccccc4)nc(-c4cc5c6ccccc6oc5c5ccc(-n6c7ccccc7c7cc8sc9ccccc9c8cc76)cc45)n3)cc2)cc1]

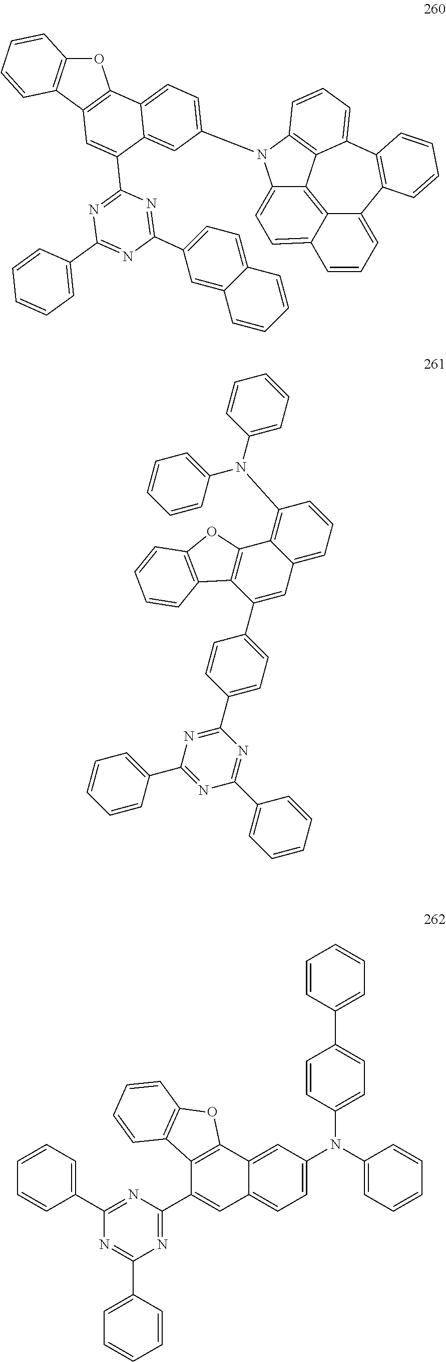

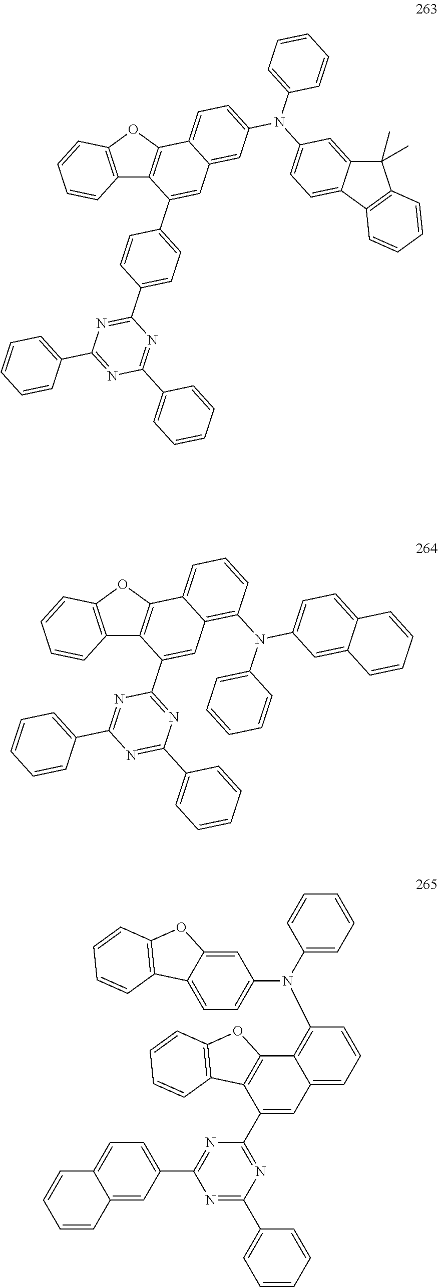 [SMILES: CC1(C)c2ccccc2-c2ccc(N(c3ccccc3)c3ccc4c(c3)cc(-c3ccc(-c5nc(-c6ccccc6)nc(-c6ccccc6)n5)cc3)c3c5ccccc5oc43)cc21 ; c1ccc(-c2nc(-c3ccccc3)nc(-c3cc4c(N(c5ccccc5)c5ccc6ccccc6c5)cccc4c4oc5ccccc5c34)n2)cc1 ; c1ccc(-c2nc(-c3ccc4ccccc4c3)nc(-c3cc4cccc(N(c5ccccc5)c5ccc6c(c5)oc5ccccc56)c4c4oc5ccccc5c34)n2)cc1]

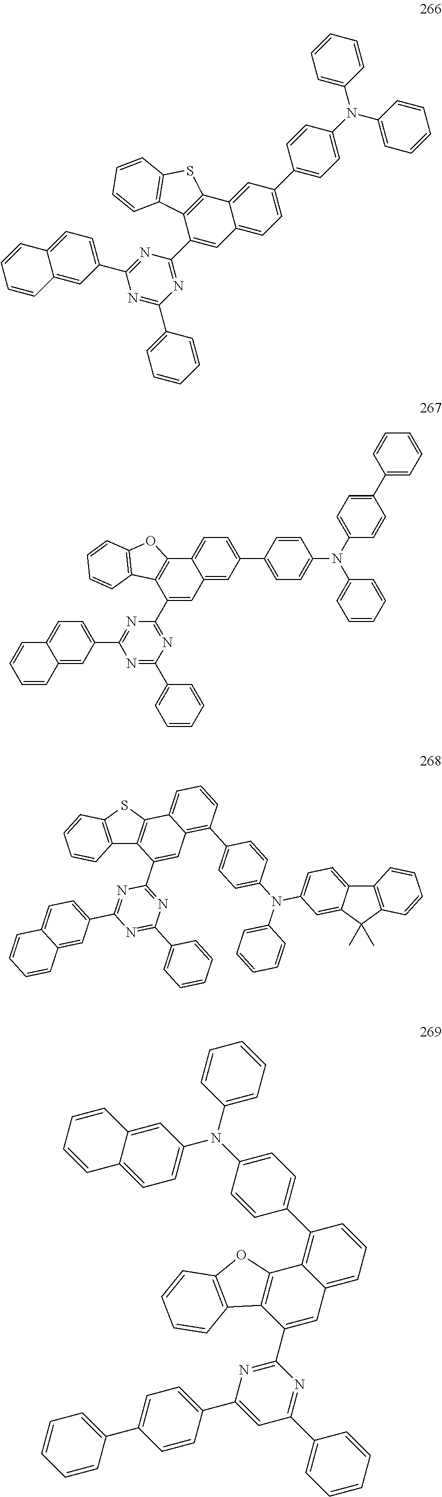 [SMILES: c1ccc(-c2nc(-c3ccc4ccccc4c3)nc(-c3cc4ccc(-c5ccc(N(c6ccccc6)c6ccccc6)cc5)cc4c4sc5ccccc5c34)n2)cc1 ; c1ccc(-c2ccc(N(c3ccccc3)c3ccc(-c4ccc5c(c4)cc(-c4nc(-c6ccccc6)nc(-c6ccc7ccccc7c6)n4)c4c6ccccc6oc54)cc3)cc2)cc1 ; CC1(C)c2ccccc2-c2ccc(N(c3ccccc3)c3ccc(-c4cccc5c4cc(-c4nc(-c6ccccc6)nc(-c6ccc7ccccc7c6)n4)c4c6ccccc6sc54)cc3)cc21 ; c1ccc(-c2ccc(-c3cc(-c4ccccc4)nc(-c4cc5cccc(-c6ccc(N(c7ccccc7)c7ccc8ccccc8c7)cc6)c5c5oc6ccccc6c45)n3)cc2)cc1]

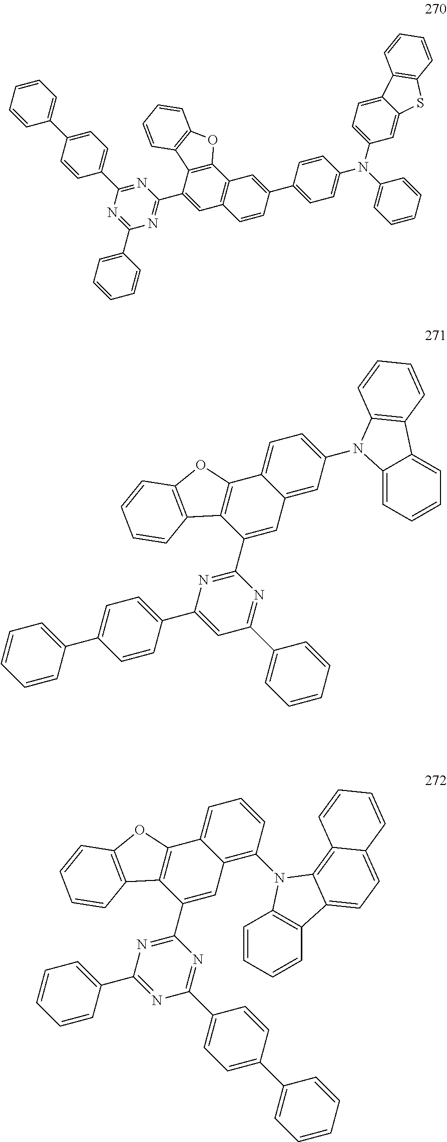 [SMILES: c1ccc(-c2ccc(-c3nc(-c4ccccc4)nc(-c4cc5ccc(-c6ccc(N(c7ccccc7)c7ccc8c(c7)sc7ccccc78)cc6)cc5c5oc6ccccc6c45)n3)cc2)cc1 ; c1ccc(-c2ccc(-c3cc(-c4ccccc4)nc(-c4cc5cc(-n6c7ccccc7c7ccccc76)ccc5c5oc6ccccc6c45)n3)cc2)cc1 ; c1ccc(-c2ccc(-c3nc(-c4ccccc4)nc(-c4cc5c(-n6c7ccccc7c7ccc8ccccc8c76)cccc5c5oc6ccccc6c45)n3)cc2)cc1]

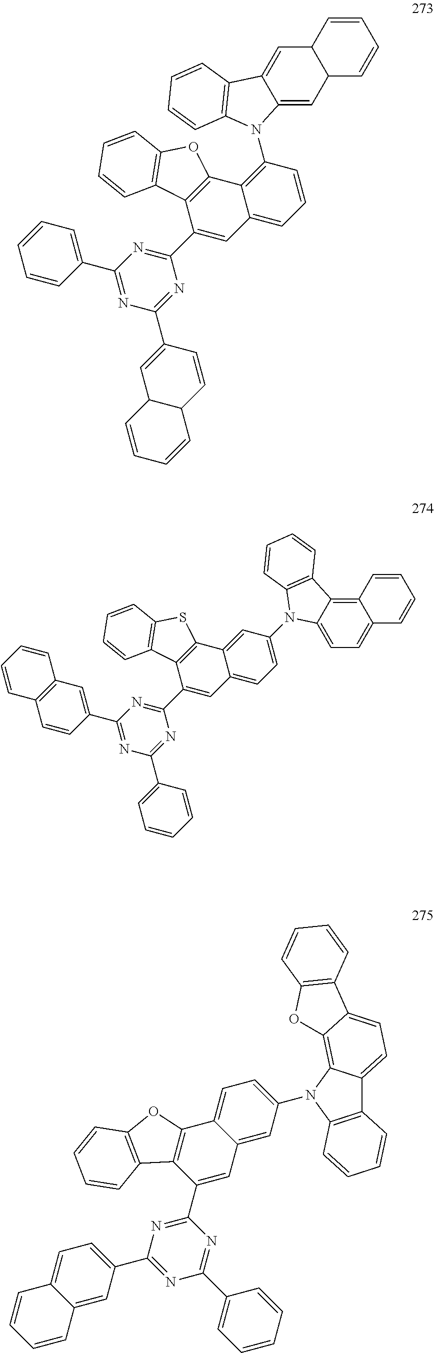 [SMILES: C1=CC2C=CC(c3nc(-c4ccccc4)nc(-c4cc5cccc(-n6c7c(c8ccccc86)=CC6C=CC=CC6C=7)c5c5oc6ccccc6c45)n3)=CC2C=C1 ; c1ccc(-c2nc(-c3ccc4ccccc4c3)nc(-c3cc4ccc(-n5c6ccccc6c6c7ccccc7ccc65)cc4c4sc5ccccc5c34)n2)cc1 ; c1ccc(-c2nc(-c3ccc4ccccc4c3)nc(-c3cc4cc(-n5c6ccccc6c6ccc7c8ccccc8oc7c65)ccc4c4oc5ccccc5c34)n2)cc1]

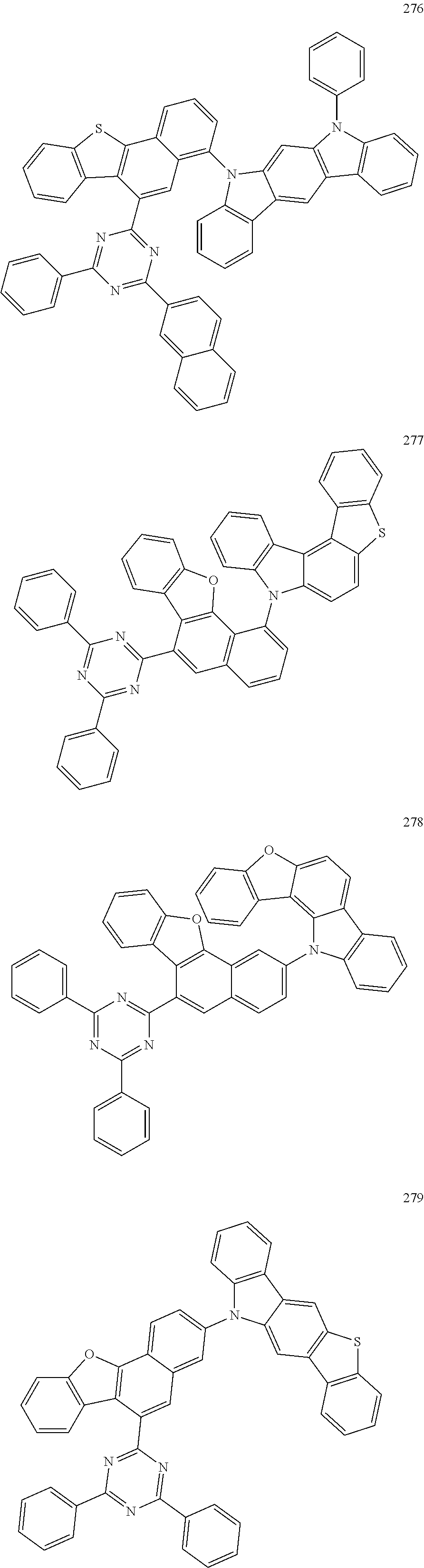 [SMILES: c1ccc(-c2nc(-c3ccc4ccccc4c3)nc(-c3cc4c(-n5c6ccccc6c6cc7c8ccccc8n(-c8ccccc8)c7cc65)cccc4c4sc5ccccc5c34)n2)cc1 ; c1ccc(-c2nc(-c3ccccc3)nc(-c3cc4cccc(-n5c6ccccc6c6c7c(ccc65)sc5ccccc57)c4c4oc5ccccc5c34)n2)cc1 ; c1ccc(-c2nc(-c3ccccc3)nc(-c3cc4ccc(-n5c6ccccc6c6ccc7oc8ccccc8c7c65)cc4c4oc5ccccc5c34)n2)cc1 ; c1ccc(-c2nc(-c3ccccc3)nc(-c3cc4cc(-n5c6ccccc6c6cc7sc8ccccc8c7cc65)ccc4c4oc5ccccc5c34)n2)cc1]

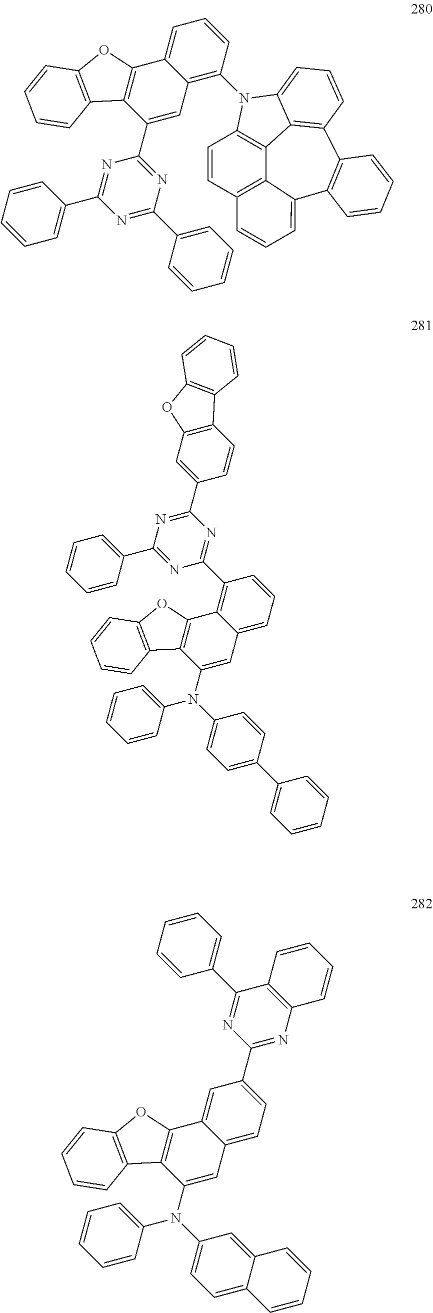

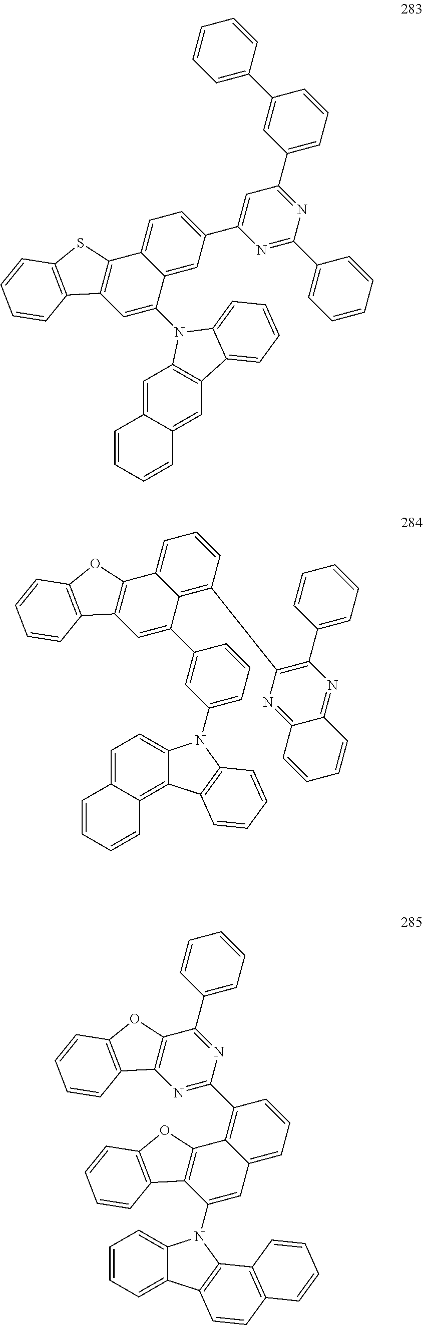 [SMILES: c1ccc(-c2cccc(-c3cc(-c4ccc5c(c4)c(-n4c6ccccc6c6cc7ccccc7cc64)cc4c6ccccc6sc54)nc(-c4ccccc4)n3)c2)cc1 ; c1ccc(-c2nc3ccccc3nc2-c2cccc3c2c(-c2cccc(-n4c5ccccc5c5c6ccccc6ccc54)c2)cc2c4ccccc4oc32)cc1 ; c1ccc(-c2nc(-c3cccc4cc(-n5c6ccccc6c6ccc7ccccc7c65)c5c6ccccc6oc5c34)nc3c2oc2ccccc23)cc1]

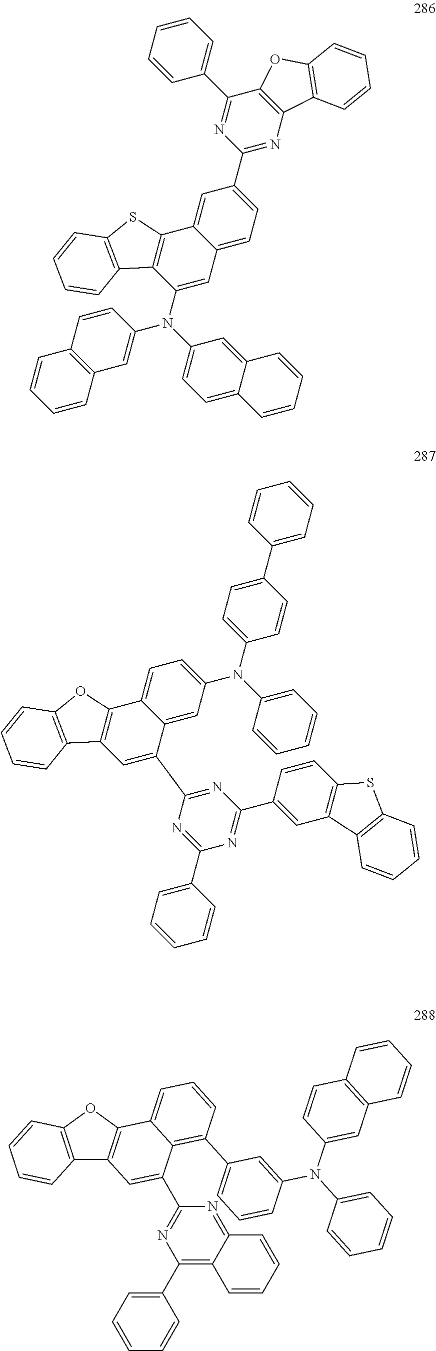 [SMILES: c1ccc(-c2nc(-c3ccc4cc(N(c5ccc6ccccc6c5)c5ccc6ccccc6c5)c5c6ccccc6sc5c4c3)nc3c2oc2ccccc23)cc1 ; c1ccc(-c2ccc(N(c3ccccc3)c3ccc4c(c3)c(-c3nc(-c5ccccc5)nc(-c5ccc6sc7ccccc7c6c5)n3)cc3c5ccccc5oc43)cc2)cc1 ; c1ccc(-c2nc(-c3cc4c5ccccc5oc4c4cccc(-c5cccc(N(c6ccccc6)c6ccc7ccccc7c6)c5)c34)nc3ccccc23)cc1]

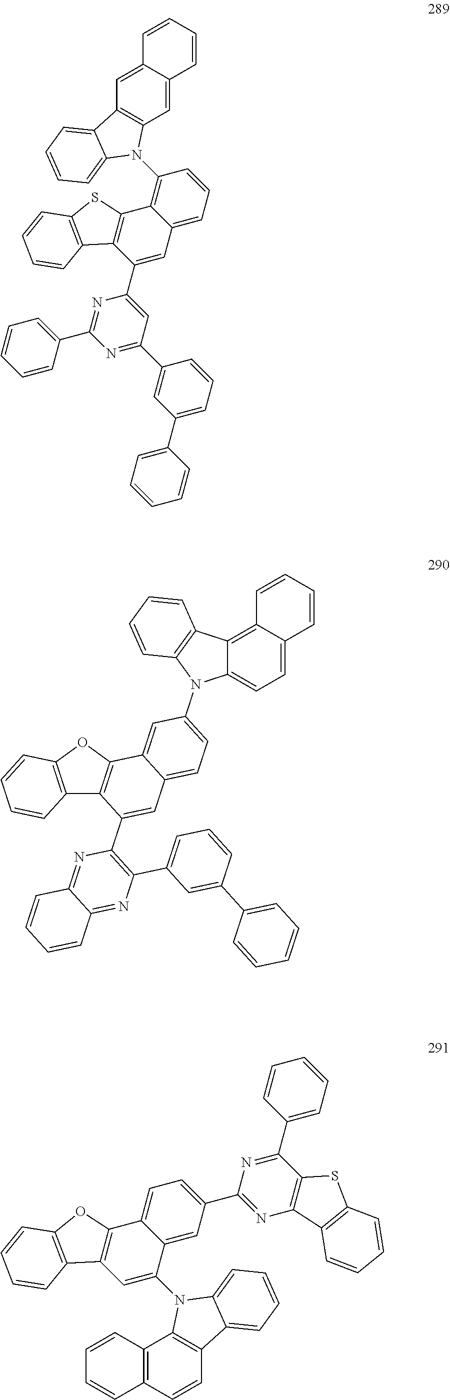 [SMILES: c1ccc(-c2cccc(-c3cc(-c4cc5cccc(-n6c7ccccc7c7cc8ccccc8cc76)c5c5sc6ccccc6c45)nc(-c4ccccc4)n3)c2)cc1 ; c1ccc(-c2cccc(-c3nc4ccccc4nc3-c3cc4ccc(-n5c6ccccc6c6c7ccccc7ccc65)cc4c4oc5ccccc5c34)c2)cc1 ; c1ccc(-c2nc(-c3ccc4c(c3)c(-n3c5ccccc5c5ccc6ccccc6c53)cc3c5ccccc5oc43)nc3c2sc2ccccc23)cc1]

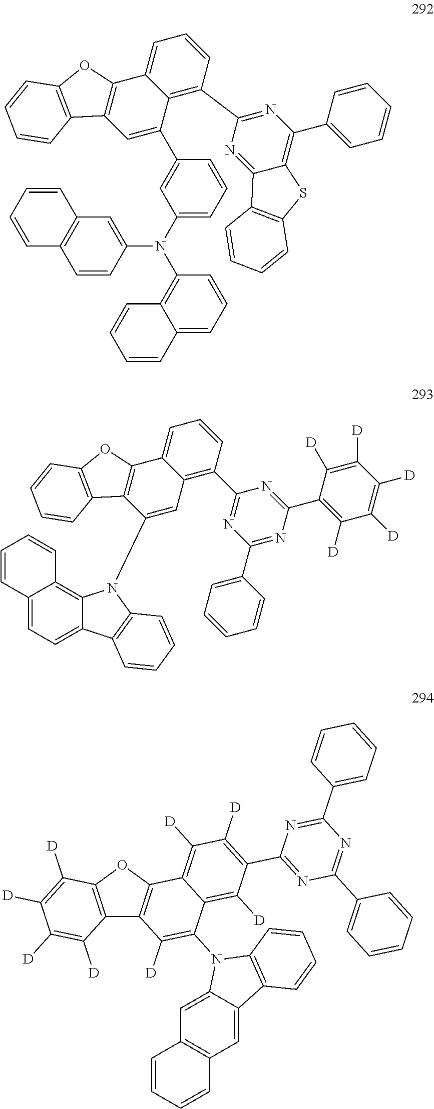 [SMILES: c1ccc(-c2nc(-c3cccc4c3c(-c3cccc(N(c5ccc6ccccc6c5)c5cccc6ccccc56)c3)cc3c5ccccc5oc43)nc3c2sc2ccccc23)cc1 ; [2H]c1c([2H])c([2H])c(-c2nc(-c3ccccc3)nc(-c3cccc4c3cc(-n3c5ccccc5c5ccc6ccccc6c53)c3c5ccccc5oc43)n2)c([2H])c1[2H] ; [2H]c1c([2H])c([2H])c2c(oc3c4c([2H])c([2H])c(-c5nc(-c6ccccc6)nc(-c6ccccc6)n5)c([2H])c4c(-n4c5ccccc5c5cc6ccccc6cc54)c([2H])c32)c1[2H]]

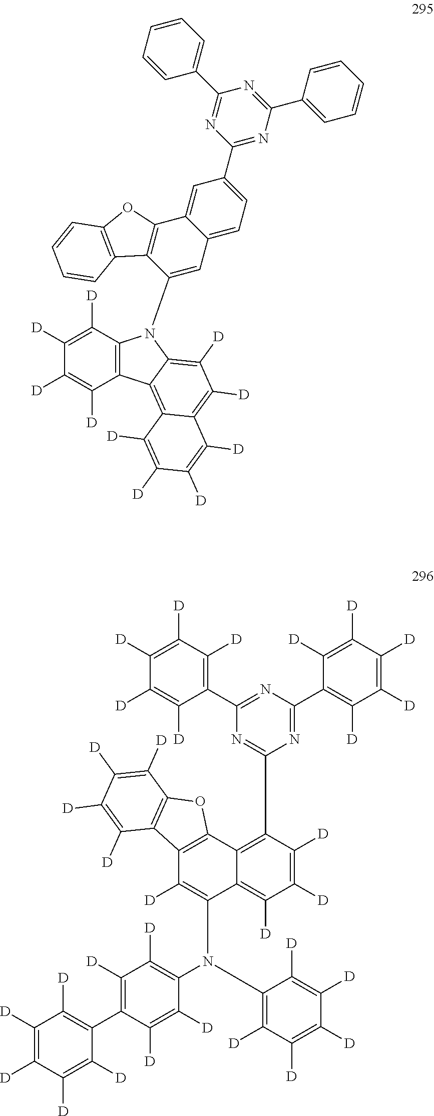 [SMILES: [2H]c1c([2H])c([2H])c2c(c1[2H])c([2H])c([2H])c1c2c2c([2H])c([2H])c([2H])c([2H])c2n1-c1cc2ccc(-c3nc(-c4ccccc4)nc(-c4ccccc4)n3)cc2c2oc3ccccc3c12 ; [2H]c1c([2H])c([2H])c(-c2nc(-c3c([2H])c([2H])c([2H])c([2H])c3[2H])nc(-c3c([2H])c([2H])c([2H])c4c(N(c5c([2H])c([2H])c([2H])c([2H])c5[2H])c5c([2H])c([2H])c(-c6c([2H])c([2H])c([2H])c([2H])c6[2H])c([2H])c5[2H])c([2H])c5c(oc6c([2H])c([2H])c([2H])c([2H])c65)c34)n2)c([2H])c1[2H]]

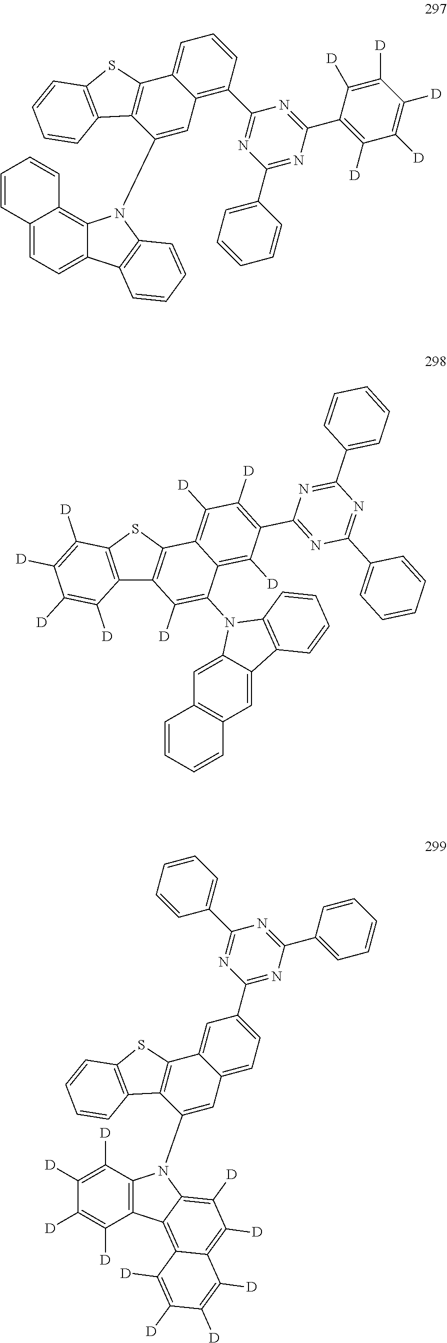 [SMILES: [2H]c1c([2H])c([2H])c(-c2nc(-c3ccccc3)nc(-c3cccc4c3cc(-n3c5ccccc5c5ccc6ccccc6c53)c3c5ccccc5sc43)n2)c([2H])c1[2H] ; [2H]c1c([2H])c([2H])c2c(sc3c4c([2H])c([2H])c(-c5nc(-c6ccccc6)nc(-c6ccccc6)n5)c([2H])c4c(-n4c5ccccc5c5cc6ccccc6cc54)c([2H])c32)c1[2H] ; [2H]c1c([2H])c([2H])c2c(c1[2H])c([2H])c([2H])c1c2c2c([2H])c([2H])c([2H])c([2H])c2n1-c1cc2ccc(-c3nc(-c4ccccc4)nc(-c4ccccc4)n3)cc2c2sc3ccccc3c12]

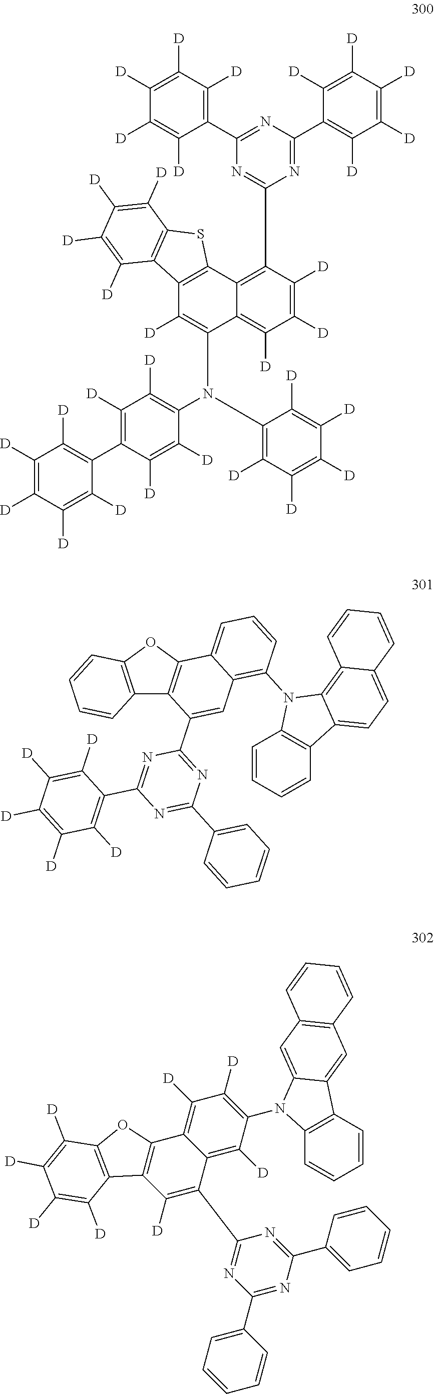

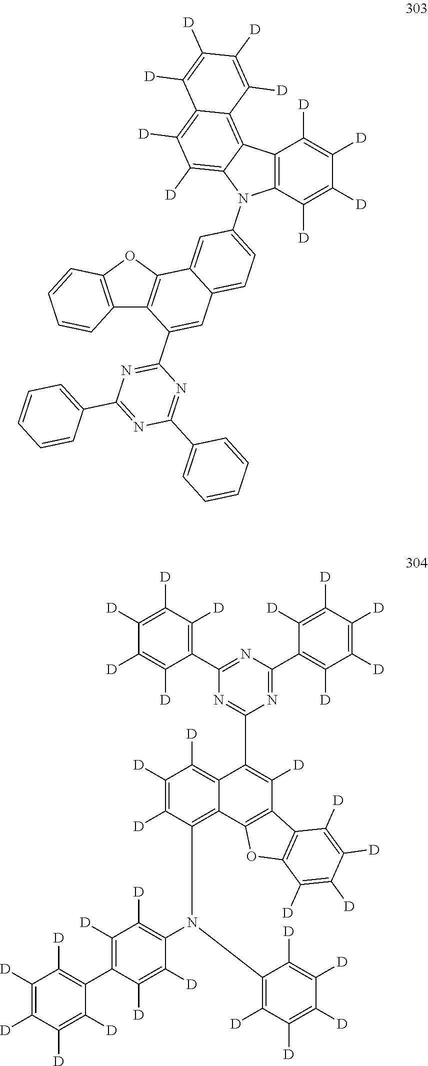 [SMILES: [2H]c1c([2H])c([2H])c2c(c1[2H])c([2H])c([2H])c1c2c2c([2H])c([2H])c([2H])c([2H])c2n1-c1ccc2cc(-c3nc(-c4ccccc4)nc(-c4ccccc4)n3)c3c4ccccc4oc3c2c1 ; [2H]c1c([2H])c([2H])c(-c2nc(-c3c([2H])c([2H])c([2H])c([2H])c3[2H])nc(-c3c([2H])c4c(oc5c([2H])c([2H])c([2H])c([2H])c54)c4c(N(c5c([2H])c([2H])c([2H])c([2H])c5[2H])c5c([2H])c([2H])c(-c6c([2H])c([2H])c([2H])c([2H])c6[2H])c([2H])c5[2H])c([2H])c([2H])c([2H])c34)n2)c([2H])c1[2H]]

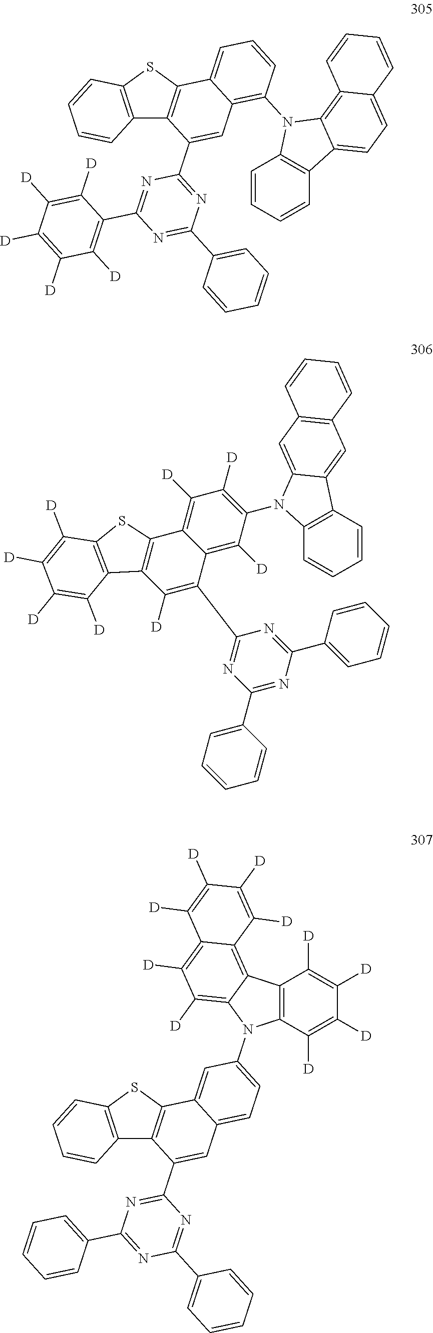 [SMILES: [2H]c1c([2H])c([2H])c(-c2nc(-c3ccccc3)nc(-c3cc4c(-n5c6ccccc6c6ccc7ccccc7c65)cccc4c4sc5ccccc5c34)n2)c([2H])c1[2H] ; [2H]c1c([2H])c([2H])c2c(sc3c4c([2H])c([2H])c(-n5c6ccccc6c6cc7ccccc7cc65)c([2H])c4c(-c4nc(-c5ccccc5)nc(-c5ccccc5)n4)c([2H])c32)c1[2H] ; [2H]c1c([2H])c([2H])c2c(c1[2H])c([2H])c([2H])c1c2c2c([2H])c([2H])c([2H])c([2H])c2n1-c1ccc2cc(-c3nc(-c4ccccc4)nc(-c4ccccc4)n3)c3c4ccccc4sc3c2c1]

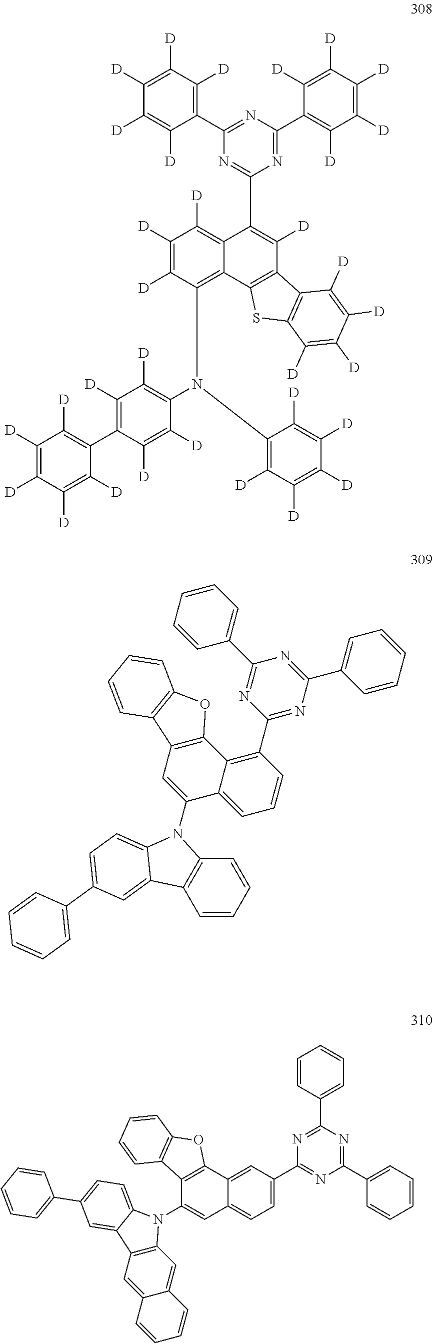 [SMILES: [2H]c1c([2H])c([2H])c(-c2nc(-c3c([2H])c([2H])c([2H])c([2H])c3[2H])nc(-c3c([2H])c4c(sc5c([2H])c([2H])c([2H])c([2H])c54)c4c(N(c5c([2H])c([2H])c([2H])c([2H])c5[2H])c5c([2H])c([2H])c(-c6c([2H])c([2H])c([2H])c([2H])c6[2H])c([2H])c5[2H])c([2H])c([2H])c([2H])c34)n2)c([2H])c1[2H] ; c1ccc(-c2ccc3c(c2)c2ccccc2n3-c2cc3c4ccccc4oc3c3c(-c4nc(-c5ccccc5)nc(-c5ccccc5)n4)cccc23)cc1 ; c1ccc(-c2ccc3c(c2)c2cc4ccccc4cc2n3-c2cc3ccc(-c4nc(-c5ccccc5)nc(-c5ccccc5)n4)cc3c3oc4ccccc4c23)cc1]

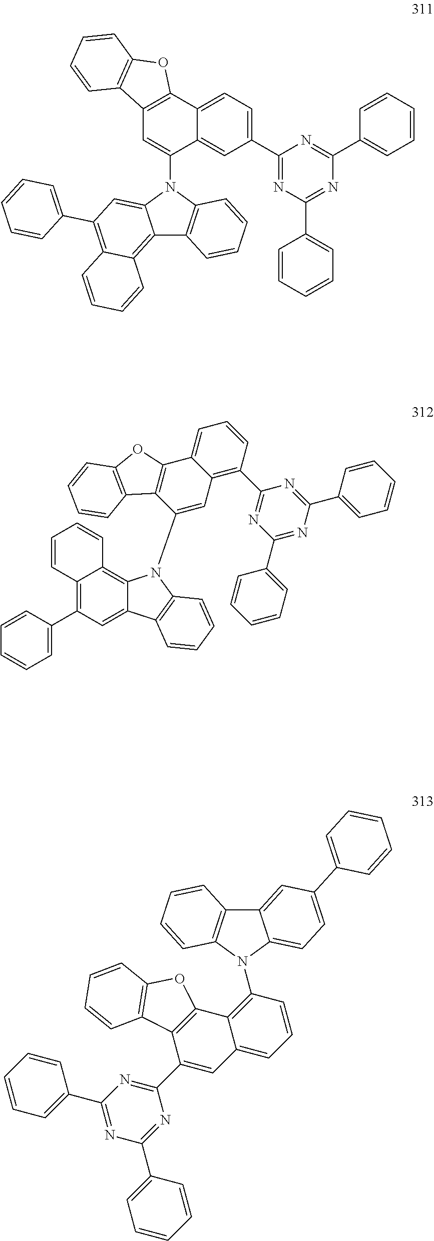 [SMILES: c1ccc(-c2nc(-c3ccccc3)nc(-c3ccc4c(c3)c(-n3c5ccccc5c5c6ccccc6c(-c6ccccc6)cc53)cc3c5ccccc5oc43)n2)cc1 ; c1ccc(-c2nc(-c3ccccc3)nc(-c3cccc4c3cc(-n3c5ccccc5c5cc(-c6ccccc6)c6ccccc6c53)c3c5ccccc5oc43)n2)cc1 ; c1ccc(-c2ccc3c(c2)c2ccccc2n3-c2cccc3cc(-c4nc(-c5ccccc5)nc(-c5ccccc5)n4)c4c5ccccc5oc4c23)cc1]

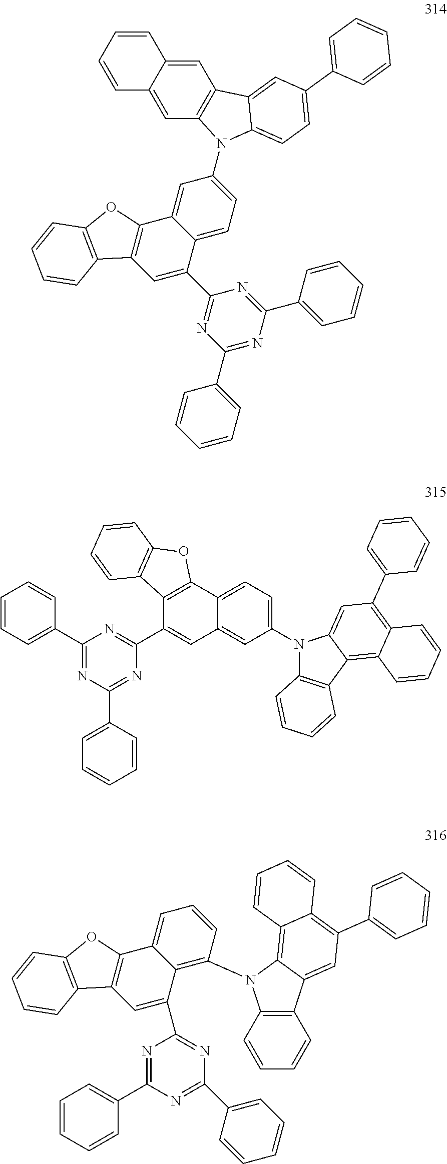 [SMILES: c1ccc(-c2ccc3c(c2)c2cc4ccccc4cc2n3-c2ccc3c(-c4nc(-c5ccccc5)nc(-c5ccccc5)n4)cc4c5ccccc5oc4c3c2)cc1 ; c1ccc(-c2nc(-c3ccccc3)nc(-c3cc4cc(-n5c6ccccc6c6c7ccccc7c(-c7ccccc7)cc65)ccc4c4oc5ccccc5c34)n2)cc1 ; c1ccc(-c2nc(-c3ccccc3)nc(-c3cc4c5ccccc5oc4c4cccc(-n5c6ccccc6c6cc(-c7ccccc7)c7ccccc7c65)c34)n2)cc1]

In addition, by introducing various substituents to the structure of Chemical Formula 1, compounds having unique properties of the introduced substituents may be synthesized. For example, by introducing substituents normally used as a hole injection layer material, a hole transport layer material, an electron blocking layer material, a light emitting layer material, an electron transport layer material, a hole blocking layer material and an electron injection layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

In addition, one embodiment of the present disclosure provides an organic light emitting device comprising: a first electrode; a second electrode provided opposite to the first electrode; and one or more organic material layers provided between the first electrode and the second electrode, wherein one or more layers of the organic material layers include the heterocyclic compound of any one of claims 1 to 7.

In one embodiment of the present disclosure, the first electrode may be a positive electrode, and the second electrode may be a negative electrode.

In another embodiment of the present disclosure, the first electrode may be a negative electrode, and the second electrode may be a positive electrode.

In one embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a green organic light emitting device, and the heterocyclic compound represented by Chemical Formula 1 may be used as a material of the green organic light emitting device.

In another embodiment of the present disclosure, the organic light emitting device may be a blue organic light emitting device, and the heterocyclic compound represented by Chemical Formula 1 may be used as a material of the blue organic light emitting device.

Specific descriptions on the heterocyclic compound represented by Chemical Formula 1 are the same as the descriptions provided above.

In one embodiment of the present disclosure, the organic light emitting device may be a red organic light emitting device, and the heterocyclic compound represented by Chemical Formula 1 may be used as a light emitting layer material of the red organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may form an organic material layer using a solution coating method as well as a vacuum deposition method when manufacturing the organic light emitting device. Herein, the solution coating method means spin coating, dip coating, inkjet printing, screen printing, a spray method, roll coating or the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron injection layer, an electron transport layer, an electron blocking layer, a hole blocking layer and the like as the organic material layers. However, the structure of the organic light emitting device is not limited thereto, and may include a smaller number of organic material layers.

In one embodiment of the present disclosure, the organic light emitting device may include one or more organic material layers, the organic material layer may include a light emitting layer, and the light emitting layer may include the heterocyclic compound represented by Chemical Formula 1.

In another embodiment of the present disclosure, the organic material layer may include a light emitting layer, the light emitting layer may include a host material, and the host material may include the heterocyclic compound.

In another embodiment of the present disclosure, the organic light emitting device may further include one, two or more layers selected from the group consisting of a light emitting layer, a hole injection layer, a hole transport layer, an electron injection layer, an electron transport layer, an electron blocking layer and a hole blocking layer.

In one embodiment of the present disclosure, the organic light emitting device may comprise the heterocyclic compound represented by Chemical Formula 1, and may further comprise a heterocyclic compound represented by the following Chemical Formula 2-1 or the following Chemical Formula 2-2:

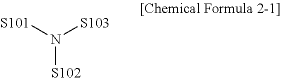

in Chemical Formula 2-1,
S101 to S103 are the same as or different from each other, and are each independently a substituted or unsubstituted C6 to C60 aryl group; or a substituted or unsubstituted C2 to C60 heteroaryl group,

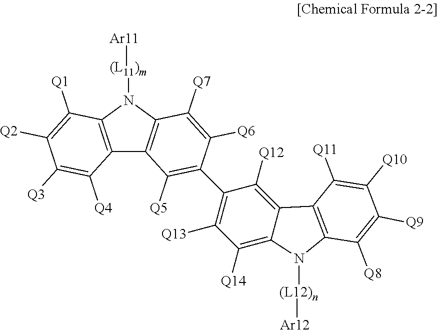

in Chemical Formula 2-2,
L11 and L12 are the same as or different from each other, and are each independently a single bond; a substituted or unsubstituted C6 to C60 arylene group; or a substituted or unsubstituted C2 to C60 heteroarylene group,
Ar11 and Ar12 are the same as or different from each other, and are each independently a substituted or unsubstituted C6 to C60 aryl group; or a substituted or unsubstituted C2 to C60 heteroaryl group,
Q1 to Q14 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or to C60 alkynyl group; a substituted or unsubstituted C2 unsubstituted C1 to C60 alkoxy group; a substituted or unsubstituted C3 to C60 cycloalkyl group; and a substituted or unsubstituted C2 to C60 heterocycloalkyl group, and
m and n are the same as or different from each other, and are each independently an integer of 0 to 3.

In another embodiment of the present disclosure, in Chemical Formula 2-1, S101 to S103 are the same as or different from each other, and may be each independently a substituted or unsubstituted C6 to C40 aryl group; or a substituted or unsubstituted C2 to C40 heteroaryl group.

In another embodiment of the present disclosure, in Chemical Formula 2-1, S101 to S103 are the same as or different from each other, and may be each independently a substituted or unsubstituted C6 to C30 aryl group; or a substituted or unsubstituted C2 to C30 heteroaryl group.

In another embodiment of the present disclosure, in Chemical Formula 2-2, L11 and L12 are the same as or different from each other, and may be each independently a single bond; or a substituted or unsubstituted C6 to C30 arylene group.

In another embodiment of the present disclosure, in Chemical Formula 2-2, Ar11 and Ar12 are the same as or different from each other, and may be each independently a substituted or unsubstituted C6 to C20 aryl group.

In another embodiment of the present disclosure, in Chemical Formula 2-2, 01 to Q14 are the same as or different from each other, and may be each independently hydrogen; deuterium; halogen or a cyano group.

In one embodiment of the present disclosure, the heterocyclic compound represented by Chemical Formula 2-1 or Chemical Formula 2-2 does not include deuterium as a substituent, or the content of deuterium based on the total number of hydrogen atoms and deuterium atoms may be 1% to 100%.

In another embodiment of the present disclosure, the heterocyclic compound represented by Chemical Formula 2-1 or Chemical Formula 2-2 does not include deuterium as a substituent, or the content of deuterium based on the total number of hydrogen atoms and deuterium atoms may be 10% to 90%.

In another embodiment of the present disclosure, the heterocyclic compound represented by Chemical Formula 2-1 or Chemical Formula 2-2 does not include deuterium as a substituent, or the content of deuterium based on the total number of hydrogen atoms and deuterium atoms may be 20% to 80%.

In another embodiment of the present disclosure, the heterocyclic compound represented by Chemical Formula 2-1 or Chemical Formula 2-2 does not include deuterium as a substituent, or the content of deuterium based on the total number of hydrogen atoms and deuterium atoms may be 30% to 70%.

For example, the heterocyclic compound represented by Chemical Formula 2-1 or Chemical Formula 2-2 does not include deuterium as a substituent, or the content of deuterium may be more than 0%, 1% or more, 5% or more, 10% or more, 15% or more, 20% or more, 25% or more, 30% or more, 35% or more, 40% or more, 45% or more or 50% or more, and 100% or less, 95% or less, 90% or less, 85% or less, 80% or less, 75% or less, 70% or less, 65% or less or 60% or less based on the total number of hydrogen atoms and deuterium atoms.

In one embodiment of the present disclosure, the heterocyclic compound represented by Chemical Formula 2-1 or Chemical Formula 2-2 may be any one selected from the group consisting of the following compounds.

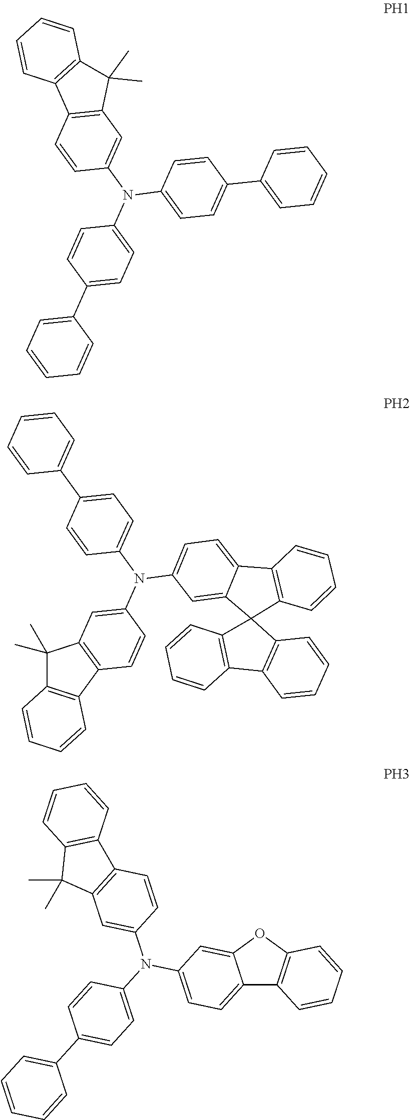

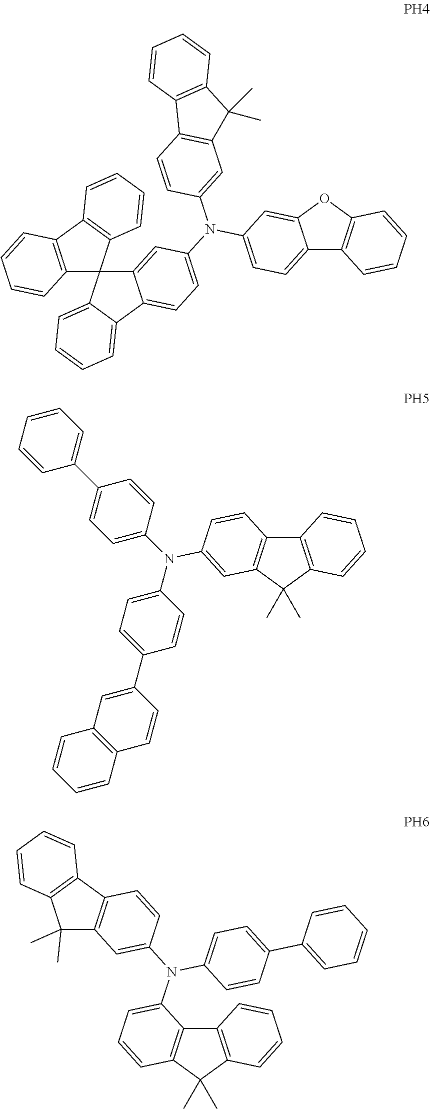

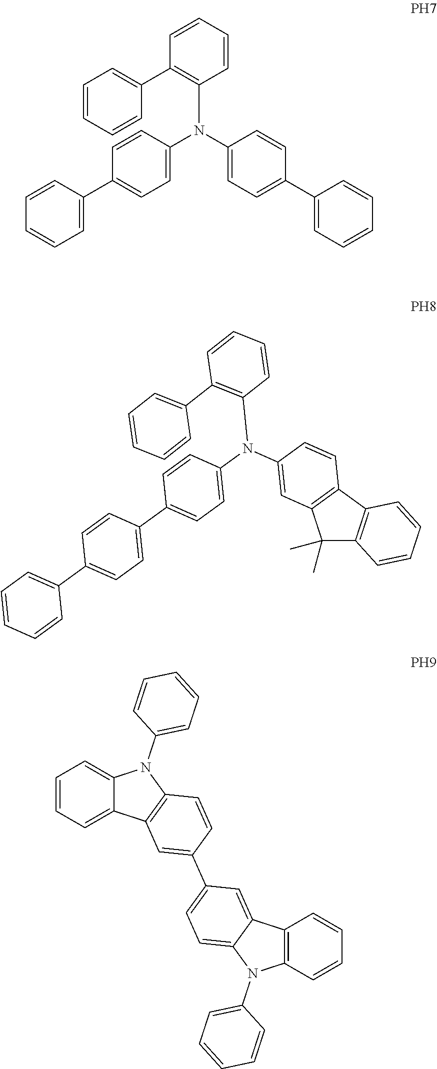 [SMILES: c1ccc(-c2ccc(N(c3ccc(-c4ccccc4)cc3)c3ccccc3-c3ccccc3)cc2)cc1 ; CC1(C)c2ccccc2-c2ccc(N(c3ccc(-c4ccc(-c5ccccc5)cc4)cc3)c3ccccc3-c3ccccc3)cc21 ; c1ccc(-n2c3ccccc3c3cc(-c4ccc5c(c4)c4ccccc4n5-c4ccccc4)ccc32)cc1]

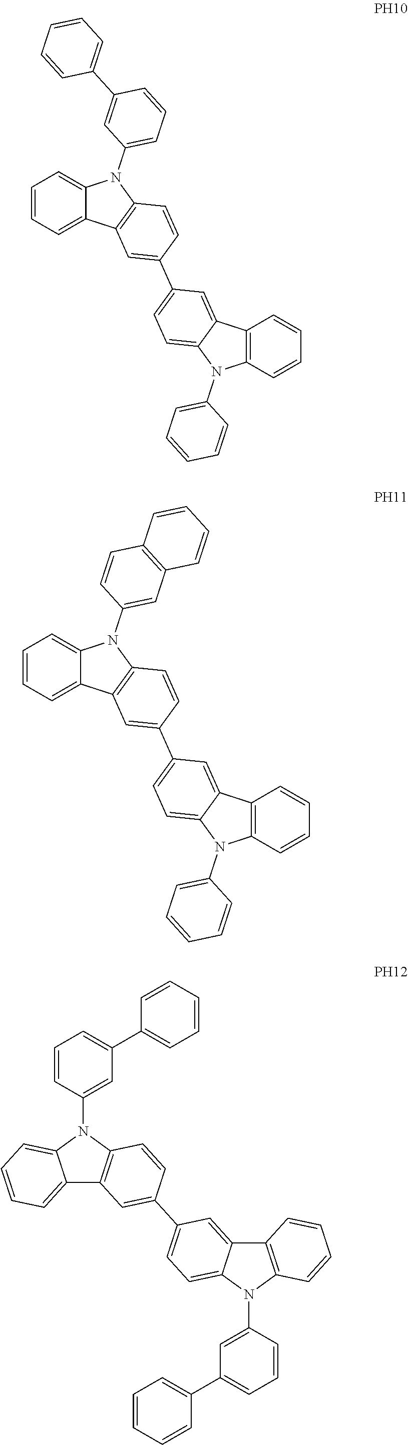

In manufacturing the organic light emitting device of the present disclosure, the heterocyclic compound represented by Chemical Formula 1 and the heterocyclic compound represented by Chemical Formula 2-1 or Chemical Formula 2-2 may be included.

In manufacturing the organic light emitting device of the present disclosure, when comprising the heterocyclic compound represented by Chemical Formula 1 and the heterocyclic compound represented by Chemical Formula 2-1, or the heterocyclic compound represented by Chemical Formula 1 and the heterocyclic compound represented by Chemical Formula 2-2 as a mixture, the organic light emitting device may have superior driving voltage and light emission efficiency, and improved lifetime. This may lead to a forecast that an exciplex phenomenon occurs when comprising the two heterocyclic compounds at the same time as a mixture.

The exciplex phenomenon is a phenomenon of releasing an energy difference between a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and a driving voltage may be lowered, which resultantly helps with enhancement in the lifetime. In other words, when using the compound represented by Chemical Formula 1 as the acceptor and using the compound represented by Chemical Formula 2-1 or Chemical Formula 2-2 as the donor, excellent organic light emitting device properties are obtained.

In one embodiment of the present disclosure, when the organic light emitting device includes the heterocyclic compound represented by Chemical Formula 1 and the heterocyclic compound represented by Chemical Formula 2-1 or Chemical Formula 2-2 as a mixture, at least one of the heterocyclic compounds does not include deuterium as a substituent, or the content of deuterium may be 1% to 100%.

In another embodiment of the present disclosure, when the organic light emitting device includes the heterocyclic compound represented by Chemical Formula 1 and the heterocyclic compound represented by Chemical Formula 2-1 or Chemical Formula 2-2 as a mixture, at least one of the heterocyclic compounds does not include deuterium as a substituent, or the content of deuterium may be 10% to 90%.

In another embodiment of the present disclosure, when the organic light emitting device includes the heterocyclic compound represented by Chemical Formula 1 and the heterocyclic compound represented by Chemical Formula 2-1 or Chemical Formula 2-2 as a mixture, at least one of the heterocyclic compounds does not include deuterium as a substituent, or the content of deuterium may be 20% to 80%.

In another embodiment of the present disclosure, when the organic light emitting device includes the heterocyclic compound represented by Chemical Formula 1 and the heterocyclic compound represented by Chemical Formula 2-1 or Chemical Formula 2-2 as a mixture, at least one of the heterocyclic compounds does not include deuterium as a substituent, or the content of deuterium may be 30% to 70%.

For example, when the organic light emitting device includes the heterocyclic compound represented by Chemical Formula 1 and the heterocyclic compound represented by Chemical Formula 2-1 or Chemical Formula 2-2 as a mixture, at least one of the heterocyclic compounds does not include deuterium as a substituent, or the content of deuterium may be more than 0%, 1% or more, 5% or more, 10% or more, 15% or more, 20% or more, 25% or more, 30% or more, 35% or more, 40% or more, 45% or more or 50% or more, and 100% or less, 95% or less, 90% or less, 85% or less, 80% or less, 75% or less, 70% or less, 65% or less or 60% or less.

In addition, one embodiment of the present disclosure provides a composition for an organic material layer of an organic light emitting device, the composition comprising the heterocyclic compound represented by Chemical Formula 1 and the heterocyclic compound represented by Chemical Formula 2-1 or Chemical Formula 2-2.

Specific descriptions on the heterocyclic compound represented by Chemical Formula 1 and the heterocyclic compound represented by Chemical Formula 2-1 or Chemical Formula 2-2 are the same as the descriptions provided above.

In one embodiment of the present disclosure, the heterocyclic compound represented by Chemical Formula 1 and the heterocyclic compound represented by Chemical Formula 2-1 or Chemical Formula 2-2 in the composition for an organic material layer of an organic light emitting device may have a weight ratio of 1:10 to 10:1, 1:8 to 8:1, 1:6 to 6:1, 1:4 to 4:1, 1:3 to 3:1, or 1:2 to 2:1, however, the weight ratio is not limited thereto.

The composition for an organic material layer of an organic light emitting device may be used when forming an organic material layer of an organic light emitting device, and particularly, may be more preferably used when forming a host of a light emitting layer.

In one embodiment of the present disclosure, the organic material layer includes the heterocyclic compound represented by Chemical Formula 1 and the heterocyclic compound represented by Chemical Formula 2-1 or Chemical Formula 2-2, and a phosphorescent dopant may be used therewith.

As the phosphorescent dopant material, those known in the art may be used. For example, phosphorescent dopant materials represented by LL′MX′, LL′L″M, LMX′X″, L2MX′ and L3M may be used, however, the scope of the present disclosure is not limited to these examples.

M may be iridium, platinum, osmium or the like.

L is an anionic bidentate ligand coordinated to M by sp2 carbon and heteroatom, and X may function to trap electrons or holes. Nonlimiting examples of L, L′ and L″ may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

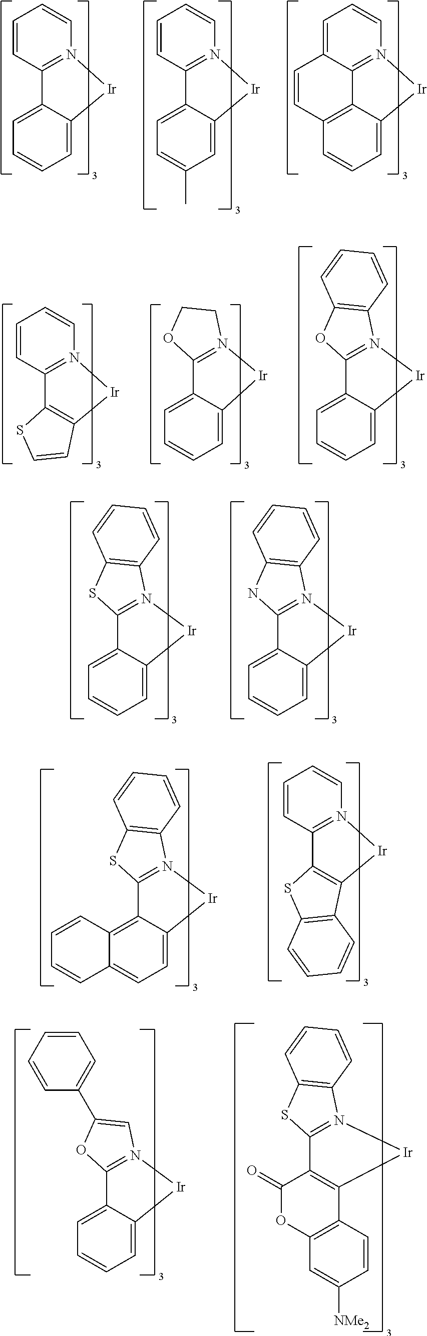

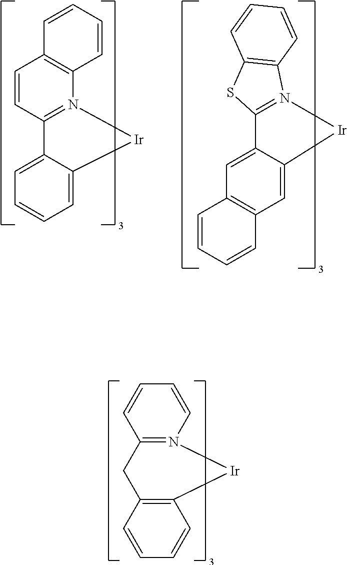

In one embodiment of the present disclosure, the organic material layer includes the heterocyclic compound represented by Chemical Formula 1 and the heterocyclic compound represented by Chemical Formula 2-1 or Chemical Formula 2-2, and an iridium-based dopant may be used therewith.

In one embodiment of the present disclosure, as the iridium-based dopant, Ir(ppy)₃ may be used as a green phosphorescent dopant.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the organic material layer includes an electron injection layer or an electron transport layer, and the electron injection layer or the electron transport layer may include the heterocyclic compound represented by Chemical Formula 1.

In the organic light emitting device according to another embodiment of the present disclosure, the organic material layer includes an electron blocking layer or a hole blocking layer, and the electron blocking layer or the hole blocking layer may include the heterocyclic compound represented by Chemical Formula 1.

In the organic light emitting device according to another embodiment of the present disclosure, the organic material layer includes an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment of the present disclosure, the organic material layer includes a light emitting layer, and the light emitting layer may include the heterocyclic compound represented by Chemical Formula 1.

In the organic light emitting device according to another embodiment of the present disclosure, the organic material layer includes a light emitting layer, the light emitting layer includes a host material, and the host material may include the heterocyclic compound represented by Chemical Formula 1.

The organic light emitting device according to one embodiment of the present disclosure may further include one, two or more layers selected from the group consisting of a light emitting layer, a hole injection layer, a hole transport layer, an electron injection layer, an electron blocking layer and a hole blocking layer.

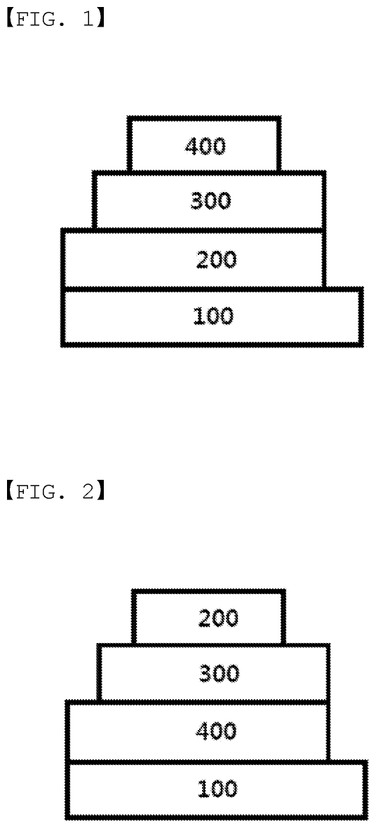

FIGS. 1 to 3 illustrate a lamination order of electrodes and organic material layers of the organic light emitting device according to one embodiment of the present disclosure. However, the scope of the present application is not limited to these diagrams, and structures of organic light emitting devices known in the art may also be used in the present application.

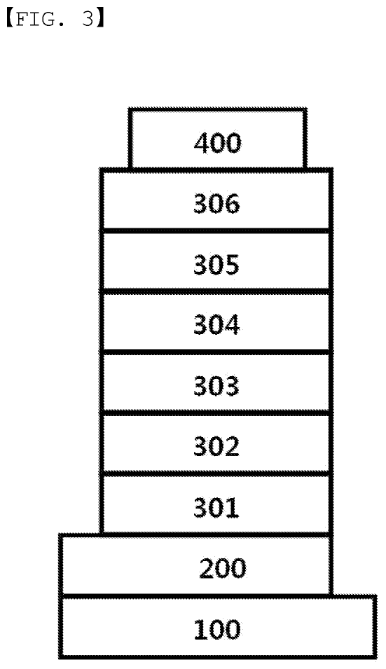

FIG. 1 illustrates an organic light emitting device in which a positive electrode 200, an organic material layer 300 and a negative electrode 400 are sequentially laminated on a substrate 100. However, the structure of the organic light emitting device is not limited to such a structure, and as illustrated in FIG. 2, an organic light emitting device in which a negative electrode, an organic material layer and a positive electrode are sequentially laminated on a substrate may also be obtained.

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited to such a lamination structure, and as necessary, the layers other than the light emitting layer may not be included, and other necessary functional layers may be further added.

One embodiment of the present disclosure provides a method for manufacturing an organic light emitting device, the method comprising: preparing a substrate; forming a first electrode on the substrate; forming one or more organic material layers on the first electrode; and forming a second electrode on the organic material layers, wherein the forming of organic material layers includes forming one or more organic material layers using the composition for an organic material layer according to one embodiment of the present disclosure.

In one embodiment of the present disclosure, the forming of organic material layers may be forming using a thermal vacuum deposition method after pre-mixing the heterocyclic compound represented by Chemical Formula 1 and the heterocyclic compound represented by Chemical Formula 2-1 or Chemical Formula 2-2.

The pre-mixing means mixing the heterocyclic compound represented by Chemical Formula 1 and the heterocyclic compound represented by Chemical Formula 2-1 or Chemical Formula 2-2 first in one source of supply before depositing on the organic material layer.

The pre-mixed material may be referred to as the composition for an organic material layer according to one embodiment of the present application.

The organic material layer comprising the heterocyclic compound represented by Chemical Formula 1 may further comprise other materials as necessary.

The organic material layer comprising the heterocyclic compound represented by Chemical Formula 1 and the heterocyclic compound represented by Chemical Formula 2-1 or Chemical Formula 2-2 at the same time may further comprise other materials as necessary.

In the organic light emitting device according to one embodiment of the present disclosure, materials other than the heterocyclic compound represented by Chemical Formula 1 or the heterocyclic compound represented by Chemical Formula 2-1 or Chemical Formula 2-2 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as Zno: Al or SnO₂: Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino] triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involving together in light emission may also be used.

When mixing hosts of the light emitting layer material, same series hosts may be mixed, or different series hosts may be mixed. For example, any two or more types of materials among n-type host materials or p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a similar principle used in the organic light emitting device.

Hereinafter, preferred examples are provided to help to understand the present disclosure, however, the following examples are only provided to more readily understand the present disclosure, and the present disclosure is not limited thereto.

### Preparation Example

### Preparation Example 1: Preparation of Compound 2

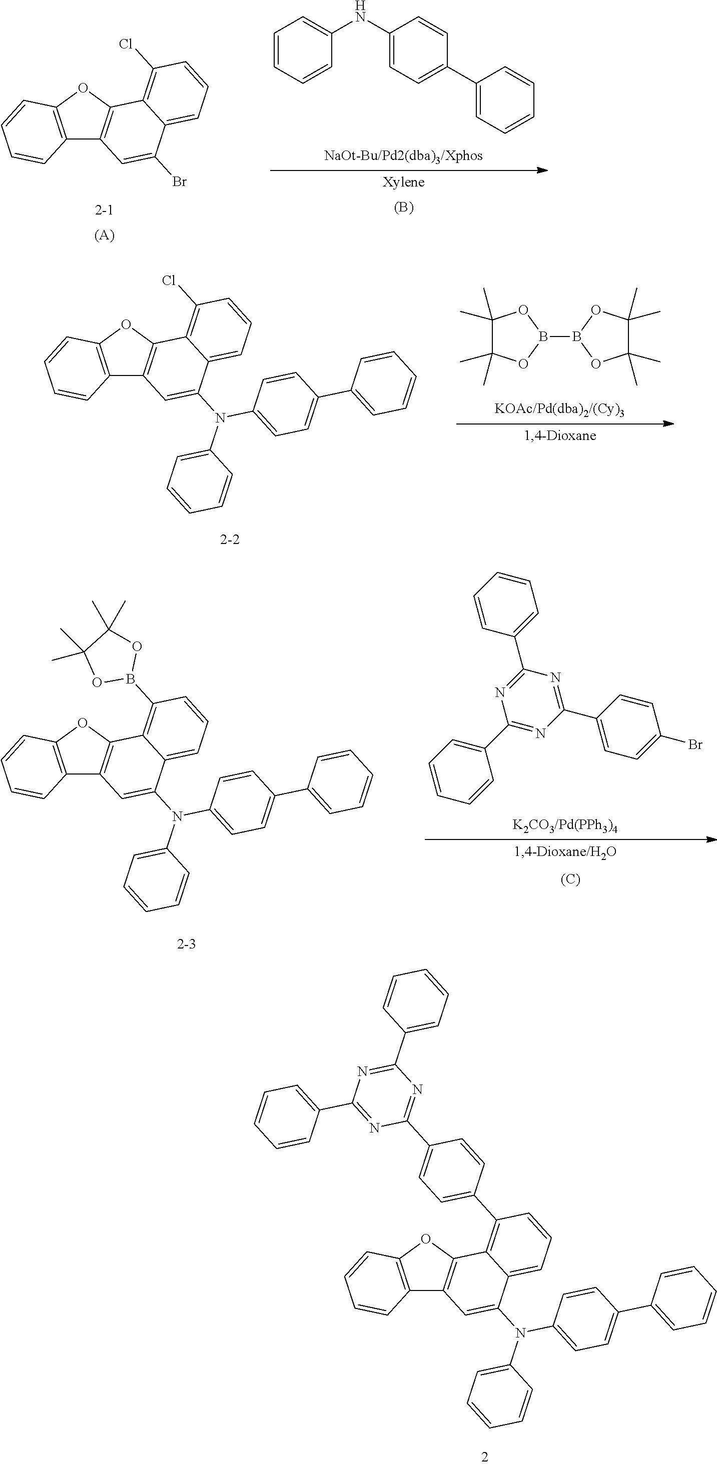

### 1) Preparation of Compound 2-2

Compound 2-1 (A) (10 g, 0.030 mol, 1 eq.), N-phenyl-[1,1′-biphenyl]-4-amine (B) (8.1 g, 0.033 mol, 1.1 eq.), tris(dibenzylideneacetone) dipalladium (Pd₂(dba)₃) (1.4 g, 0.0015 mol, 0.05 eq.) and XPhos (1.4 g, 0.0030 mol, 0.1 eq.) were introduced to xylene (150 ml), and the mixture was stirred for 6 hours at 140° C. The reaction was terminated by introducing water thereto, and then the result was extracted using methylene chloride (MC) and water. After that, moisture was removed using magnesium sulfate (MgSO₄), and then the result was separated using a silica gel column to obtain Compound 2-2 (8 g, yield 54%).

### 2) Preparation of Compound 2-3

Compound 2-2 (8 g, 0.016 mol, 1 eq.), 4, 4,4′,4′,5,5,5′,5′-octamethyl-2,2′-bi (1,3,2-dioxaborolane) (6.1 g, 0.024 mol, 1.5 eq.), bis(dibenzylideneacetone) palladium (Pd(dba)₂) (0.46 g, 0.0008 mol, 0.05 eq.) and tricyclohexylphosphine (P(Cy)₃) (0.45 g, 0.0016 mol, 0.1 eq.) were introduced to 1,4-dioxane (80 ml), and the mixture was stirred for 12 hours at 100° C. The reaction was terminated by introducing water thereto, and then the result was extracted using methylene chloride (MC) and water. After that, moisture was removed using magnesium sulfate (MgSO₄), and then the result was separated using a silica gel column to obtain Compound 2-3 (8 g, yield 84%).

### 3) Preparation of Compound 2

Compound 2-3 (8 g, 0.014 mol, 1 eq.), 2-(4-bromophenyl)-4,6-diphenyl-1,3,5-triazine (C) (5.8 g, 0.015 mol, 1.1 eq.), potassium carbonate (K₂CO₃) (4.7 g, 0.034 mol, 2.5 eq.) and tetrakis(triphenylphosphine) palladium (Pd(PPh₃)₄) (0.8 g, 0.0007 mol, 0.05 eq.) were introduced to 1,4-dioxane (240 ml) and water (60 ml), and the mixture was stirred for 8 hours at 90° C. The reaction was terminated by introducing water thereto, and then the result was extracted using methylene chloride (MC) and water. After that, moisture was removed using magnesium sulfate (MgSO₄), and the result was separated using a silica gel column to obtain Compound 2 (7 g, yield 66%).

Compounds were synthesized in the same manner as in Preparation Example 1 except that Intermediate A, Intermediate B and Intermediate C of the following Table 1 were respectively used instead of Compounds (A), (B) and (C).

TABLE 1

| Com- | | | |
|---|---|---|---|
| pound | | | |
| No. | Intermediate A | Intermediate B | Intermediate C |
| 24 | 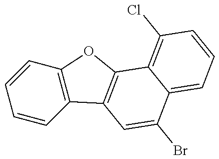 [SMILES: Clc1cccc2c(Br)cc3c4ccccc4oc3c12] | 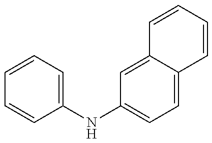 [SMILES: c1ccc(Nc2ccc3ccccc3c2)cc1] | 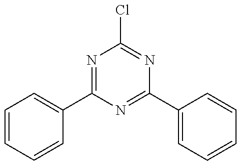 [SMILES: Clc1nc(-c2ccccc2)nc(-c2ccccc2)n1] |
| 53 | 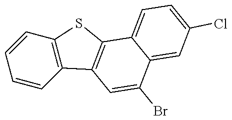 [SMILES: Clc1ccc2c(c1)c(Br)cc1c3ccccc3sc21] | 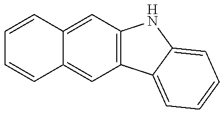 [SMILES: c1ccc2cc3c(cc2c1)[nH]c1ccccc13] | 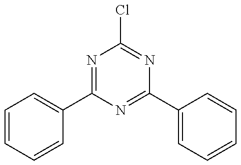 [SMILES: Clc1nc(-c2ccccc2)nc(-c2ccccc2)n1] |
| 71 | 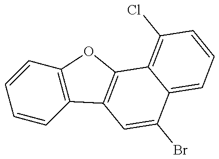 [SMILES: Clc1cccc2c(Br)cc3c4ccccc4oc3c12] | 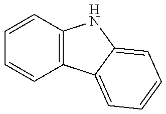 [SMILES: c1ccc2c(c1)[nH]c1ccccc12] | 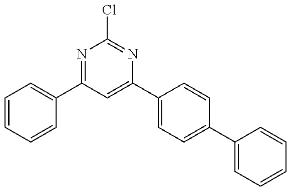 [SMILES: Clc1nc(-c2ccccc2)cc(-c2ccc(-c3ccccc3)cc2)n1] |
| 83 | 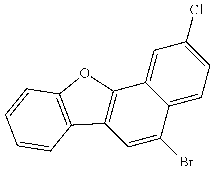 [SMILES: Clc1ccc2c(Br)cc3c4ccccc4oc3c2c1] | 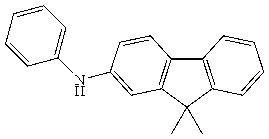 [SMILES: CC1(C)c2ccccc2-c2ccc(Nc3ccccc3)cc21] | 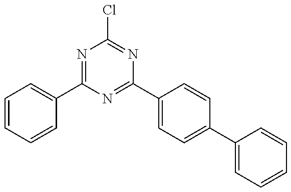 [SMILES: Clc1nc(-c2ccccc2)nc(-c2ccc(-c3ccccc3)cc2)n1] |
| 114 | 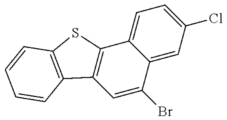 [SMILES: Clc1ccc2c(c1)c(Br)cc1c3ccccc3sc21] | 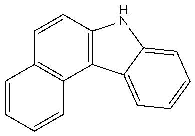 [SMILES: c1ccc2c(c1)ccc1[nH]c3ccccc3c12] | 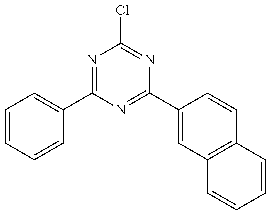 [SMILES: Clc1nc(-c2ccccc2)nc(-c2ccc3ccccc3c2)n1] |
| 123 | 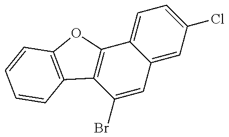 [SMILES: Clc1ccc2c(c1)cc(Br)c1c3ccccc3oc21] | 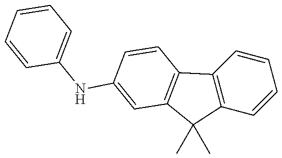 [SMILES: CC1(C)c2ccccc2-c2ccc(Nc3ccccc3)cc21] | 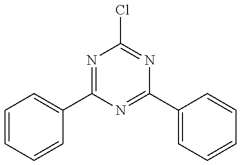 [SMILES: Clc1nc(-c2ccccc2)nc(-c2ccccc2)n1] |
| 152 | 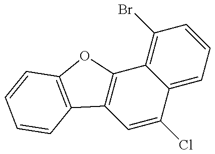 [SMILES: Clc1cc2c3ccccc3oc2c2c(Br)cccc12] | 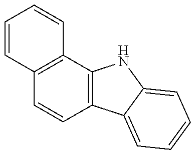 [SMILES: c1ccc2c(c1)ccc1c3ccccc3[nH]c21] | 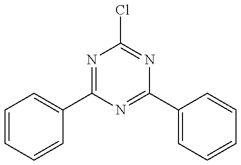 [SMILES: Clc1nc(-c2ccccc2)nc(-c2ccccc2)n1] |
| 180 | 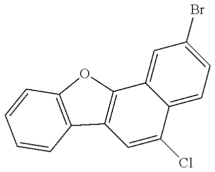 [SMILES: Clc1cc2c3ccccc3oc2c2cc(Br)ccc12] | 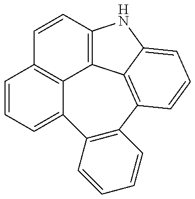 [SMILES: c1ccc2c(c1)-c1cccc3ccc4[nH]c5cccc-2c5c4c13] | 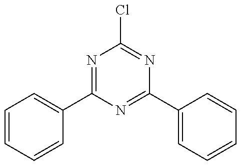 [SMILES: Clc1nc(-c2ccccc2)nc(-c2ccccc2)n1] |
| 193 | 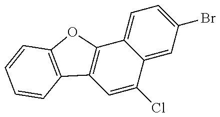 [SMILES: Clc1cc2c3ccccc3oc2c2ccc(Br)cc12] | 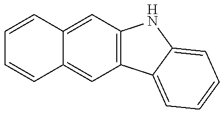 [SMILES: c1ccc2cc3c(cc2c1)[nH]c1ccccc13] | 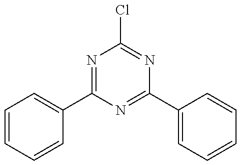 [SMILES: Clc1nc(-c2ccccc2)nc(-c2ccccc2)n1] |
| 201 | 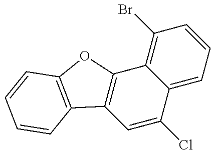 [SMILES: Clc1cc2c3ccccc3oc2c2c(Br)cccc12] | 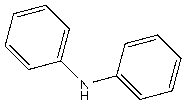 [SMILES: c1ccc(Nc2ccccc2)cc1] | 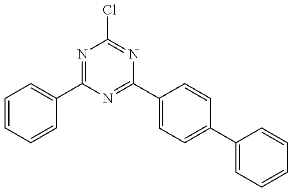 [SMILES: Clc1nc(-c2ccccc2)nc(-c2ccc(-c3ccccc3)cc2)n1] |
| 237 | 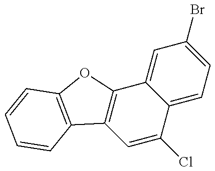 [SMILES: Clc1cc2c3ccccc3oc2c2cc(Br)ccc12] | 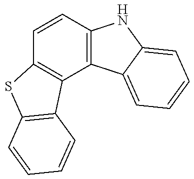 [SMILES: c1ccc2c(c1)[nH]c1ccc3sc4ccccc4c3c12] | 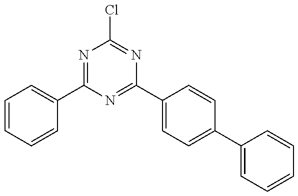 [SMILES: Clc1nc(-c2ccccc2)nc(-c2ccc(-c3ccccc3)cc2)n1] |
| 244 | 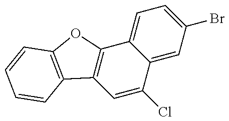 [SMILES: Clc1cc2c3ccccc3oc2c2ccc(Br)cc12] | 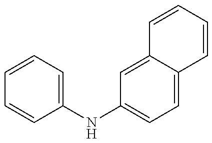 [SMILES: c1ccc(Nc2ccc3ccccc3c2)cc1] | 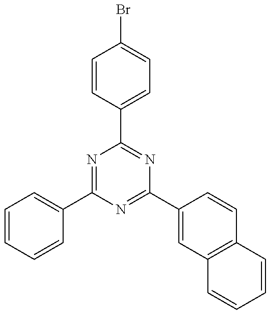 [SMILES: Brc1ccc(-c2nc(-c3ccccc3)nc(-c3ccc4ccccc4c3)n2)cc1] |
| 271 | 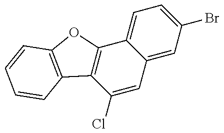 [SMILES: Clc1cc2cc(Br)ccc2c2oc3ccccc3c12] | 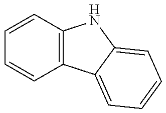 [SMILES: c1ccc2c(c1)[nH]c1ccccc12] | 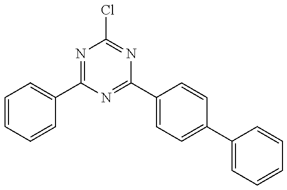 [SMILES: Clc1nc(-c2ccccc2)nc(-c2ccc(-c3ccccc3)cc2)n1] |
| 295 | 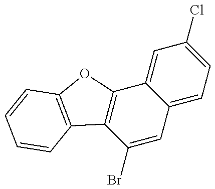 [SMILES: Clc1ccc2cc(Br)c3c4ccccc4oc3c2c1] | 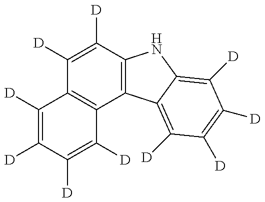 [SMILES: [2H]c1c([2H])c([2H])c2c([nH]c3c([2H])c([2H])c4c([2H])c([2H])c([2H])c([2H])c4c32)c1[2H]] | 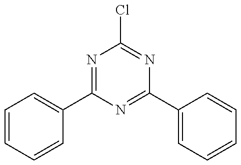 [SMILES: Clc1nc(-c2ccccc2)nc(-c2ccccc2)n1] |
| 296 | 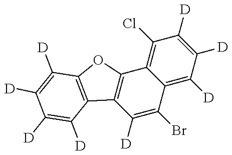 [SMILES: [2H]c1c([2H])c([2H])c2c(oc3c4c(Cl)c([2H])c([2H])c([2H])c4c(Br)c([2H])c32)c1[2H]] | 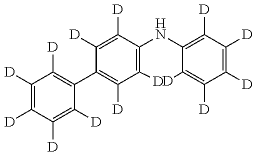 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 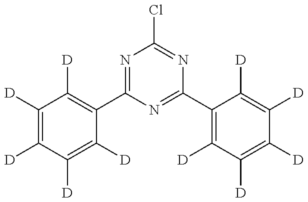 [SMILES: [2H]c1c([2H])c([2H])c(-c2nc(Cl)nc(-c3c([2H])c([2H])c([2H])c([2H])c3[2H])n2)c([2H])c1[2H]] |

The rest of compounds other than the compounds described in Preparation Example 1 and Table 1 were also prepared in the same manner as in the preparation example described above, and the synthesis results are shown in the following Table 2 and Table 3. The following Table 2 shows measurement values of 1H NMR (CDCl₃, 200 MHZ), and the following Table 3 shows measurement values of FD-mass spectrometry (FD-MS: field desorption mass spectrometry).

TABLE 2

| Compound | ¹H NMR (CDCl₃, 200 MHz) |
|---|---|
| 2 | δ = 8.42(1H, d), 8.28(4H, m), 8.09(1H, d), 7.85(3H, m), |
| | 7.66~7.25(22H, m), 6.81(1H, t), 6.69(4H, m) |
| 24 | δ = 8.28(4H, m), 8.14(1H, d), 7.89(7H, m), 7.66(2H, m), |
| | 7.51~7.32(11H, m), 7.20(2H, m), 6.81(1H, t), 6.63(2H, m) |
| 53 | δ = 8.45(1H, d), 8.28(4H, m), 8.16(4H, m), 7.94(4H, m), |
| | 7.67(3H, m), 7.50(11H, m), 7.29(1H, t) |
| 71 | δ = 8.55(1H, d), 8.30(2H, m), 8.23(1H, s), 8.12(1H, d), |
| | 8.04(1H, d), 7.89(8H, m), 7.63~7.29(17H, m) |
| 83 | δ = 8.28(2H, m), 8.58(1H, d), 7.87(6H, m), |
| | 7.66~7.20(20H, m), 6.81(1H, t), 6.75(1H, s), 6.63(3H, m), |
| | 1.72(6H, s) |
| 114 | δ = 9.09(1H, s), 8.45(3H, m), 8.28(2H, m), 8.14(3H, m), |
| | 7.98(9H, m), 7.59(11H, m), 7.29(1H, t) |
| 123 | δ = 9.09(1H, s), 8.49(1H, d), 8.28(4H, m), 8.06(1H, d), |
| | 7.87(2H, m), 7.66~7.20(16H, m), 6.81(1H, t), 6.75(1H, s), |
| | 6.58(3H, m), 1.72(6H, s) |
| 152 | δ = 8.55(3H, m), 8.28(4H, m), 8.12(2H, m), 7.86(2H, m), |
| | 7.67~7.29(17H, m) |
| 180 | δ = 8.42(2H, m), 8.16(9H, m), 7.95(4H, m), |
| | 7.67~7.32(15H, m) |
| 193 | δ = 8.28(4H, m), 8.08(4H, m), 7.95(1H, s), 7.89(1H, d), |
| | 7.67~7.29(18H, m) |
| 201 | δ = 8.28(2H, m), 8.04(1H, d), 7.85(3H, m), 7.71(2H, m), |
| | 7.52~7.20(17H, m), 6.98(1H, d), 6.81(2H, m), 6.63(4H, m) |
| 237 | δ = 8.45(1H, d), 7.28(2H, m), 8.12(2H, m), 7.85(6H, m), |
| | 7.66~7.25(21H, m) |
| 244 | δ = 9.09(1H, s), 8.49(1H, d), 8.28(2H, m), |
| | 8.00~7.19(28H, m), 6.81(2H, m), 6.63(2H, m) |
| 271 | δ = 8.55(1H, d), 8.30(2H, m), 8.23(1H, s), 8.12(1H, d), |
| | 7.83(8H, m), 7.66(2H, m), 7.52~7.25(16H, m) |
| 295 | δ = 8.49(1H, d), 8.34(5H, m), 7.92(2H, m), 7.66(1H, d), |
| | 7.44(9H, m) |
| 296 | — |

TABLE 3

| Compound | FD-MS |
|---|---|
| 2 | m/z = 768.29 |
| 24 | m/z = 666.24 |
| 53 | m/z = 680.20 |
| 71 | m/z = 689.25 |
| 83 | m/z = 808.32 |
| 114 | m/z = 730.22 |
| 123 | m/z = 732.29 |
| 152 | m/z = 664.23 |
| 180 | m/z = 738.24 |
| 193 | m/z = 664.23 |
| 201 | m/z = 692.26 |
| 237 | m/z = 796.23 |
| 244 | m/z = 792.29 |
| 271 | m/z = 689.25 |
| 295 | m/z = 674.29 |
| 296 | m/z = 724.46 |

### Experimental Example

### Experimental Example 1

### (1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and then subject to UVO (ultraviolet ozone) treatment for 5 minutes using UV in a UV (ultraviolet) cleaner. After that, the substrate was transferred to a plasma cleaner (PT), and after subjected to plasma treatment under vacuum for ITO work function increase and residual film removal, the substrate was transferred to a thermal deposition apparatus for organic deposition.

On the transparent ITO electrode (positive electrode), as common layers, 4,4′,4″-tris[2-naphthyl(phenyl)amino]triphenylamine (2-TNATA) was deposited as a hole injection layer, and N, N′-di(1-naphthyl)-N, N′-diphenyl-(1,1′-biphenyl)-4,4′-diamine (NPB) was deposited as a hole transport layer.

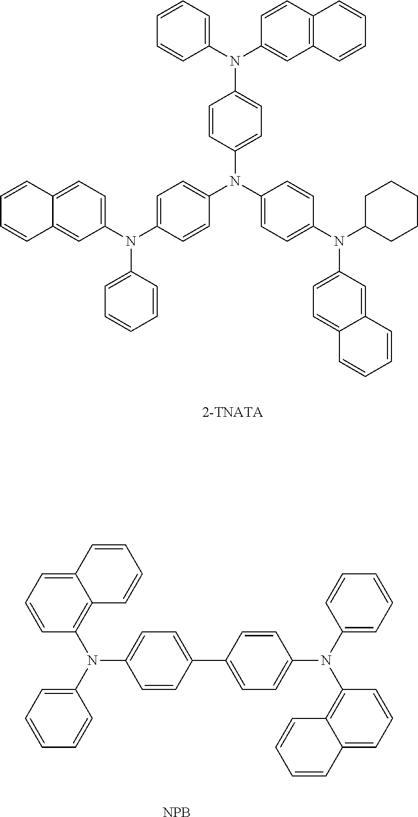 [SMILES: c1ccc(N(c2ccc(N(c3ccc(N(c4ccccc4)c4ccc5ccccc5c4)cc3)c3ccc(N(c4ccc5ccccc5c4)C4CCCCC4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

After the hole injection layer and the hole transport layer were formed as above, a light emitting layer was thermal vacuum deposited thereon as follows. The light emitting layer was deposited to a thickness of 400 Å by depositing one or two types of compounds described in the following Table 4 in one source of supply as a red host, and, using (piq)₂(Ir) (acac) as a red phosphorescent dopant, doping the host with 3 wt % of the (piq)₂(Ir) (acac).

After that, Bphen was deposited to a thickness of 30 Å as a hole blocking layer, and tris(8-hydroxyquinolinato)aluminum (Alq₃) was deposited to a thickness of 250 Å thereon as an electron transport layer.

Lastly, an electron injection layer was formed on the electron transport layer by depositing lithium fluoride (LiF) to a thickness of 10 Å, and then a negative electrode was formed on the electron injection layer by depositing an aluminum (Al) negative electrode to a thickness of 1,200 Å, and as a result, an organic light emitting device was manufactured.

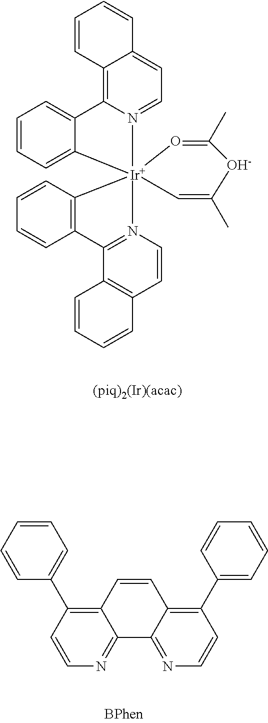

Meanwhile, all the organic compounds required to manufacture the organic light emitting device were vacuum sublimation purified under 10-8 torr to 10-6 torr for each material to be used in the organic light emitting device manufacture.

### (2) Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are as shown in Table 4.

TABLE 4

| | | | Light | | |
|---|---|---|---|---|---|
| | | Driving | Emission | | Life |
| | | Voltage | Efficiency | | time |
| | Compound | (V) | (cd/A) | CIE (x, y) | (T₉₀) |
| Example 1 | 2 | 5.11 | 54.8 | (0.680, 0.321) | 124 |
| Example 2 | 24 | 5.10 | 54.5 | (0.681, 0.319) | 125 |
| Example 3 | 53 | 5.17 | 55.1 | (0.679, 0.323) | 129 |
| Example 4 | 71 | 5.41 | 50.3 | (0.681, 0.322) | 99 |
| Example 5 | 83 | 5.15 | 54.4 | (0.678, 0.319) | 118 |
| Example 6 | 114 | 5.09 | 53.7 | (0.681, 0.322) | 122 |
| Example 7 | 123 | 5.16 | 53.5 | (0.678, 0.321) | 120 |
| Example 8 | 152 | 5.13 | 54.4 | (0.680, 0.320) | 129 |
| Example 9 | 180 | 5.10 | 53.1 | (0.681, 0.324) | 122 |
| Example 10 | 193 | 5.17 | 53.8 | (0.683, 0.323) | 117 |
| Example 11 | 201 | 5.14 | 54.2 | (0.681, 0.322) | 128 |
| Example 12 | 237 | 5.18 | 54.8 | (0.679, 0.322) | 121 |
| Example 13 | 244 | 5.13 | 53.9 | (0.680, 0.321) | 119 |
| Example 14 | 271 | 5.54 | 50.1 | (0.679, 0.322) | 98 |
| Example 15 | 295 | 5.17 | 54.0 | (0.682, 0.322) | 126 |
| Example 16 | 296 | 5.20 | 53.7 | (0.681, 0.321) | 130 |
| Comparative | H1 | 5.76 | 46.3 | (0.678, 0.324) | 89 |
| Example 1 | | | | | |
| Comparative | H2 | 6.28 | 40.2 | (0.674, 0.325) | 52 |
| Example 2 | | | | | |
| Comparative | H3 | 5.74 | 46.6 | (0.679, 0.324) | 83 |
| Example 3 | | | | | |
| Comparative | H4 | 5.79 | 46.5 | (0.680, 0.323) | 86 |
| Example 4 | | | | | |

[Comparative Compounds H1 to H4]

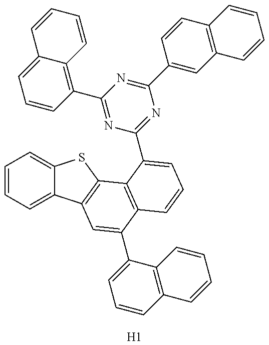

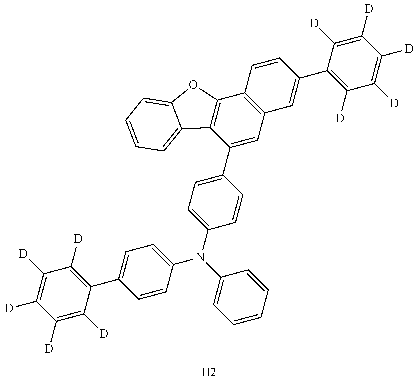 [SMILES: [2H]c1c([2H])c([2H])c(-c2ccc(N(c3ccccc3)c3ccc(-c4cc5cc(-c6c([2H])c([2H])c([2H])c([2H])c6[2H])ccc5c5oc6ccccc6c45)cc3)cc2)c([2H])c1[2H] ; "H2"]

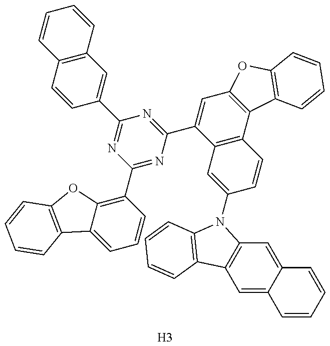 [SMILES: c1ccc2cc(-c3nc(-c4cc5oc6ccccc6c5c5ccc(-n6c7ccccc7c7cc8ccccc8cc76)cc45)nc(-c4cccc5c4oc4ccccc45)n3)ccc2c1 ; "H3"]

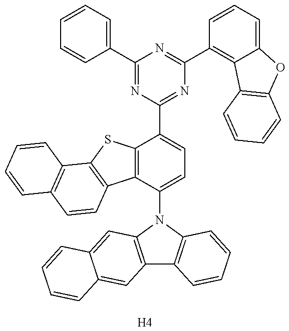 [SMILES: c1ccc(-c2nc(-c3ccc(-n4c5ccccc5c5cc6ccccc6cc54)c4c3sc3c5ccccc5ccc34)nc(-c3cccc4oc5ccccc5c34)n2)cc1 ; "H4"]

From the results of Table 4, it was identified that the organic light emitting devices of Examples 1 to 15 using the heterocyclic compound of the present disclosure as a host material had lower driving voltage and significantly improved light emission efficiency and lifetime compared to the organic light emitting devices of Comparative Examples 1 to 4.

The heterocyclic compound according to the present disclosure has, while having high thermal stability, molecular weight (m/z=500 to m/z=850) and band gap (2.4 eV to 3.2 eV) proper to be used in a light emitting layer of an organic light emitting device. The proper molecular weight facilitates formation of a light emitting layer of an organic light emitting device, and the proper band gap prevents loss of electrons and holes in the light emitting layer helping with effective formation a of recombination zone. In addition, the heterocyclic compound having electron transfer properties and substituted at a proper position may resolve a hole blocking phenomenon occurring in a dopant compared to compounds substituted at other positions. As seen from the results of Table 4, it can be seen that the heterocyclic compound of the present disclosure is superior in all aspects of driving voltage, light emission efficiency and lifetime compared to the compounds of the comparative examples.

### Experimental Example 2

### (1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and was subjected to UVO (ultraviolet ozone) treatment for 5 minutes using UV in a UV cleaner. After that, the substrate was transferred to a plasma cleaner (PT), and after subjected to plasma treatment under vacuum for ITO work function increase and residual film removal, the substrate was transferred to a thermal deposition apparatus for organic deposition. On the transparent ITO electrode (positive electrode), as common layers, 4,4′,4″-tris[2-naphthyl(phenyl)amino] triphenylamine (2-TNATA) was deposited as a hole injection layer, N, N′-di(1-naphthyl)-N, N′-diphenyl-(1,1′-biphenyl)-4,4′-diamine (NPB) was deposited as a hole transport layer, and cyclohexylidenebis[N, N-bis(4-methylphenyl)benzenamine] (TAPC) was deposited as an electron blocking layer or tris(4-carbazoyl-9-ylphenyl)amine (TCTA) was deposited as an exciton blocking layer.

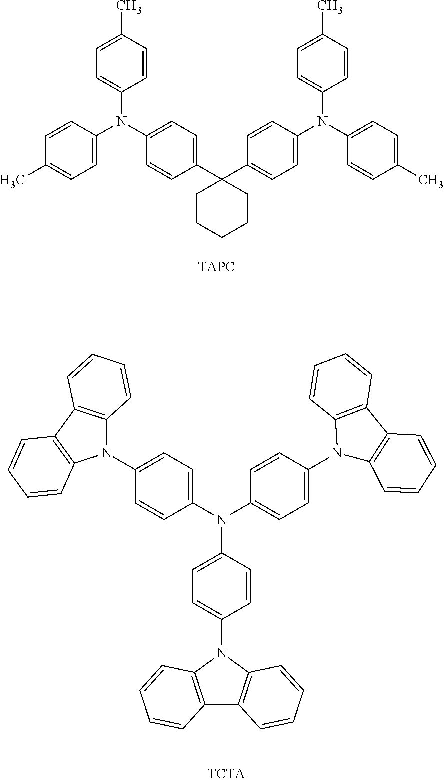

After the hole injection layer, the hole transport layer and the electron blocking layer or the exciton blocking layer were formed as above, a light emitting layer was thermal vacuum deposited thereon as follows. The light emitting layer was deposited to a thickness of 400 Å by depositing two types of compounds described in the following Table 5 in one source of supply as a red host, and, using (piq)₂(Ir) (acac) as a red phosphorescent dopant, doping the host with 3 wt % of the (piq)₂(Ir) (acac).

After that, Bphen was deposited to a thickness of 30 Å as a hole blocking layer, and TPBI was deposited to a thickness of 250 Å thereon as an electron transport layer. Lastly, an electron injection layer was formed on the electron transport layer by depositing lithium fluoride (LiF) to a thickness of 10 Å, and then a negative electrode was formed on the electron injection layer by depositing an aluminum (Al) negative electrode to a thickness of 1,200 Å, and as a result, an organic light emitting device was manufactured.

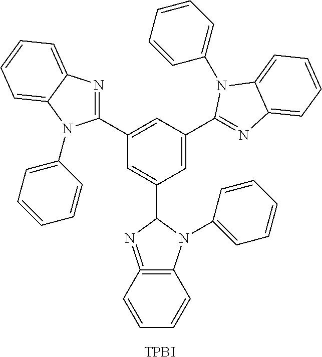

Meanwhile, all the organic compounds required to manufacture the organic light emitting device were vacuum sublimation purified under 10-8 torr to 10-6 torr for each material to be used in the organic light emitting device manufacture.

### (2) Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are as shown in Table 5.

TABLE 5

| | | | Driv- | Light | | |
|---|---|---|---|---|---|---|
| | | Ra- | ing | Emission | | |
| | | tio | Vol- | Effici- | | Life- |
| | Com- | (P/ | tage | ency | | time |
| | pound | N) | (V) | (cd/A) | CIE (x, y) | (T₉₀) |
| Example 17 | PH1:2 | 1:3 | 4.85 | 55.1 | (0.681, 0.321) | 132 |
| Example 18 | PH1:2 | 1:2 | 4.82 | 55.7 | (0.679, 0.320) | 134 |
| Example 19 | PH1:2 | 1:1 | 4.80 | 56.3 | (0.681, 0.322) | 140 |
| Example 20 | PH1:2 | 2:1 | 4.84 | 55.8 | (0.679, 0.321) | 128 |
| Example 21 | PH1:2 | 3:1 | 4.87 | 55.0 | (0.679, 0.322) | 124 |
| Example 22 | PH1:24 | 1:1 | 4.83 | 56.4 | (0.679, 0.321) | 129 |
| Example 23 | PH2:53 | 1:1 | 4.80 | 55.2 | (0.678, 0.323) | 127 |
| Example 24 | PH3:71 | 1:1 | 5.10 | 50.9 | (0.682, 0.322) | 100 |
| Example 25 | PH4:83 | 1:1 | 4.76 | 55.6 | (0.680, 0.320) | 135 |
| Example 26 | PH5:114 | 1:1 | 4.81 | 54.8 | (0.679, 0.319) | 139 |
| Example 27 | PH6:123 | 1:1 | 4.80 | 54.2 | (0.678, 0.320) | 128 |
| Example 28 | PH7:152 | 1:1 | 4.78 | 53.7 | (0.679, 0.323) | 130 |
| Example 29 | PH8:180 | 1:1 | 4.83 | 54.9 | (0.681, 0.322) | 137 |
| Example 30 | PH9:193 | 1:1 | 4.79 | 54.8 | (0.682, 0.321) | 135 |
| Example 31 | PH10:201 | 1:1 | 4.77 | 55.9 | (0.680, 0.319) | 133 |
| Example 32 | PH11:237 | 1:1 | 4.86 | 55.2 | (0.681, 0.322) | 131 |
| Example 33 | PH12:244 | 1:1 | 4.82 | 56.0 | (0.683, 0.322) | 127 |
| Example 34 | PH1:271 | 1:1 | 5.11 | 50.8 | (0.681, 0.321) | 102 |
| Example 35 | PH4:295 | 1:1 | 4.79 | 54.3 | (0.681, 0.321) | 138 |
| Example 36 | PH7:296 | 1:1 | 4.71 | 54.6 | (0.681, 0.322) | 140 |
| Compara- | PH7:H1 | 1:1 | 5.42 | 47.1 | (0.679, 0.321) | 82 |
| tive | | | | | | |
| Example 5 | | | | | | |
| Compara- | PH10:H2 | 1:1 | 6.19 | 40.8 | (0.676, 0.324) | 58 |
| tive | | | | | | |
| Example 6 | | | | | | |
| Compara- | PH2:H3 | 1:1 | 5.40 | 47.5 | (0.679, 0.322) | 81 |
| tive | | | | | | |
| Example 7 | | | | | | |
| Compara- | PH6:H4 | 1:1 | 5.45 | 47.3 | (0.680, 0.321) | 84 |
| tive | | | | | | |
| Example 8 | | | | | | |

[Comparative Compounds H1 to H4]

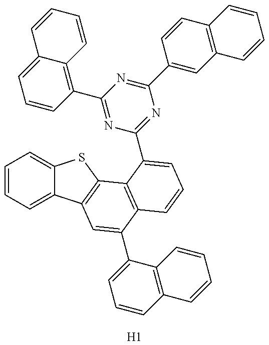

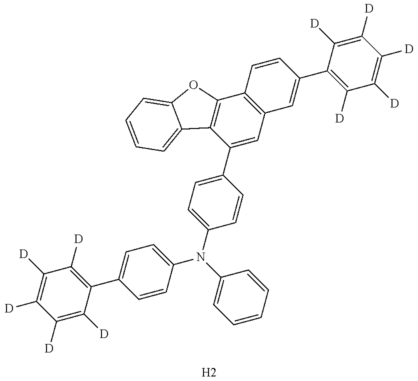 [SMILES: [2H]c1c([2H])c([2H])c(-c2ccc(N(c3ccccc3)c3ccc(-c4cc5cc(-c6c([2H])c([2H])c([2H])c([2H])c6[2H])ccc5c5oc6ccccc6c45)cc3)cc2)c([2H])c1[2H] ; "H2"]

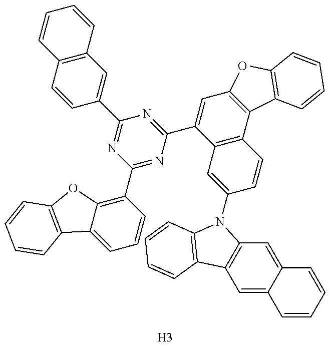 [SMILES: c1ccc2cc(-c3nc(-c4cc5oc6ccccc6c5c5ccc(-n6c7ccccc7c7cc8ccccc8cc76)cc45)nc(-c4cccc5c4oc4ccccc45)n3)ccc2c1 ; "H3"]

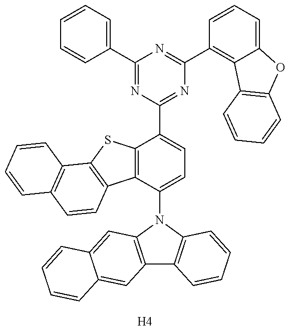

Compounds used as the P-type host (PH) of Table 5 are as follows.

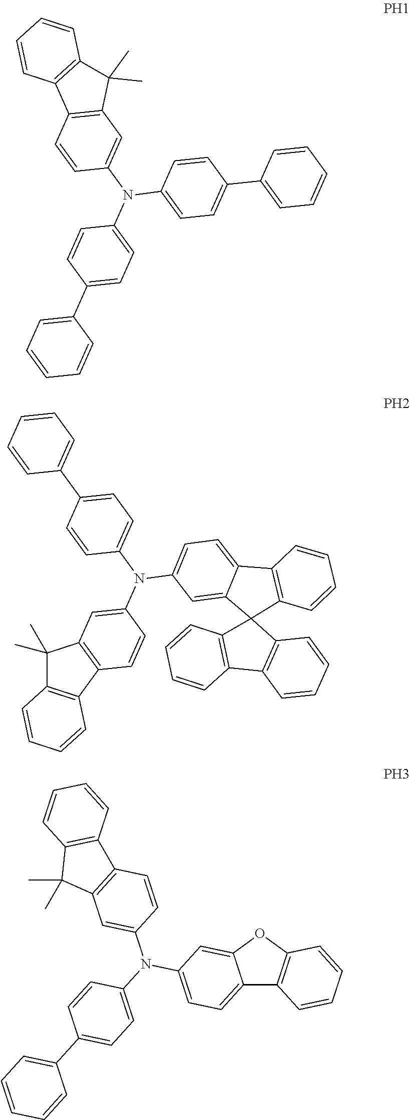

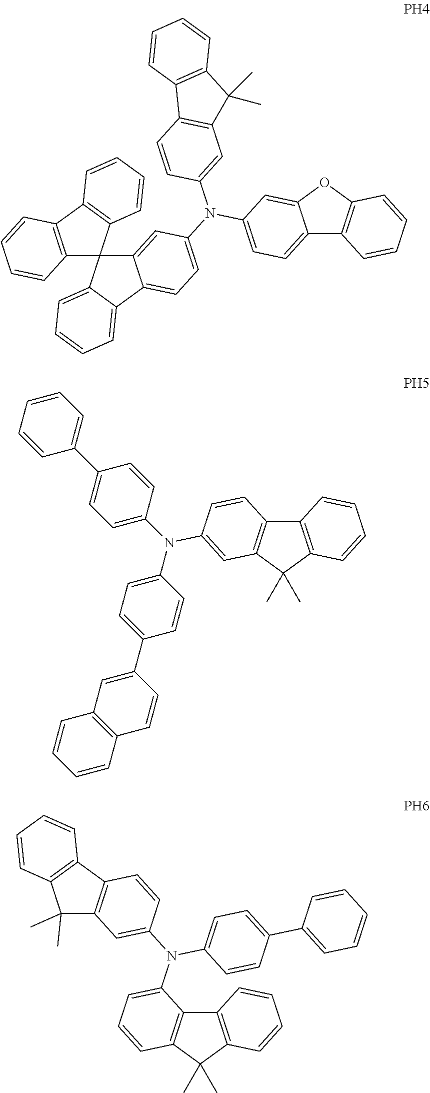

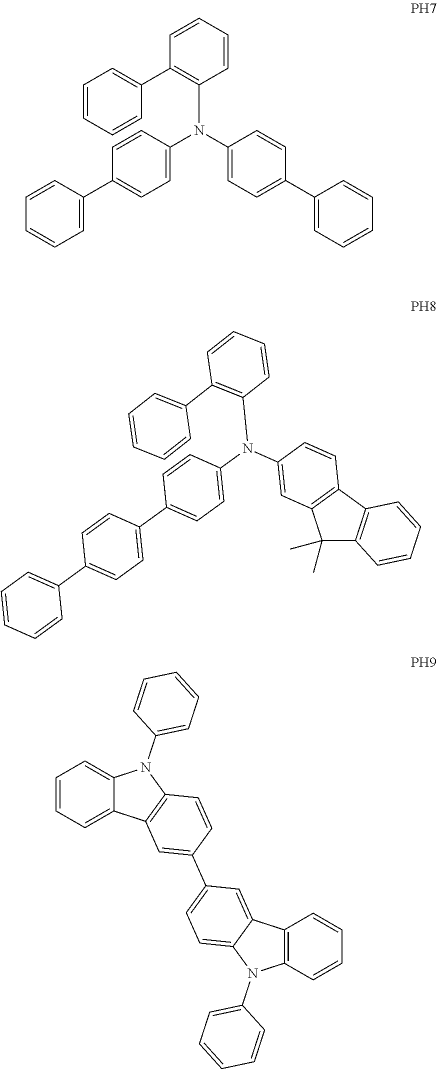 [SMILES: c1ccc(-c2ccc(N(c3ccc(-c4ccccc4)cc3)c3ccccc3-c3ccccc3)cc2)cc1 ; CC1(C)c2ccccc2-c2ccc(N(c3ccc(-c4ccc(-c5ccccc5)cc4)cc3)c3ccccc3-c3ccccc3)cc21 ; c1ccc(-n2c3ccccc3c3cc(-c4ccc5c(c4)c4ccccc4n5-c4ccccc4)ccc32)cc1]

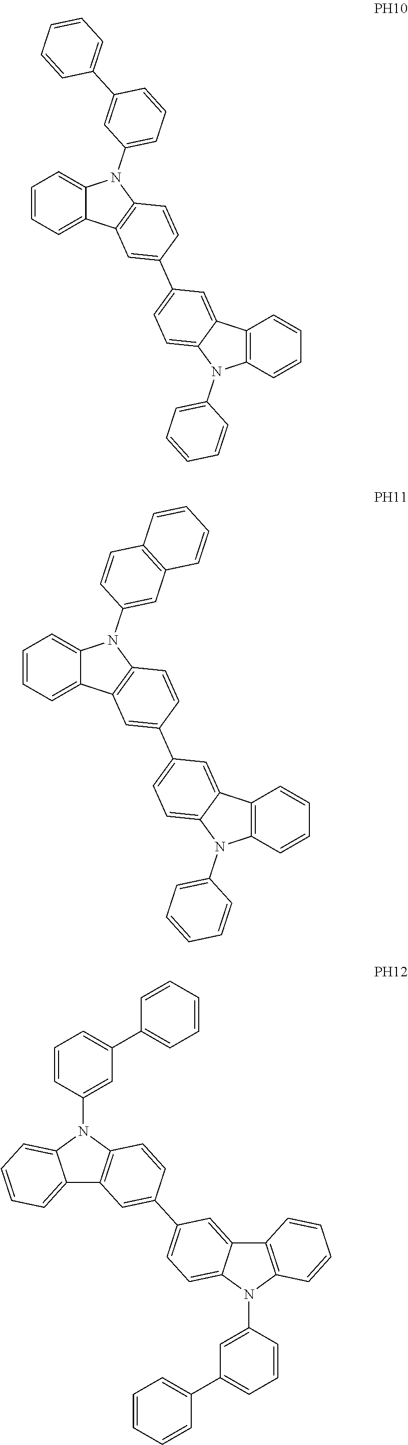

From the results of Table 5, it was identified that, when the heterocyclic compound of the present disclosure was used as an N-type host and deposited after mixed with a P-type host, the organic light emitting device had improved driving voltage, light emission efficiency and lifetime.

When a donor (p-host) having a favorable hole transport ability and an acceptor (n-host) having a favorable electron transport ability are used as a host of a light emitting layer, holes are injected to the p-host and electrons are injected to the n-host due to an exciplex phenomenon of the N+P compound, which realizes a charge balance in a device. Through this, it was seen that combining the N-type host compound having excellent electron transfer properties and the P-type host compound having excellent hole transfer properties in a proper ratio was able to help with enhancement in driving voltage, light emission efficiency and lifetime.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 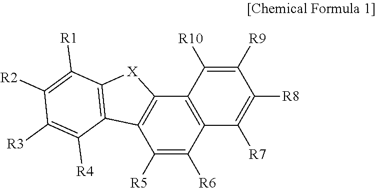
wherein,
X is O or S;
R1 to R10 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 1-1; and a group represented by the following Chemical Formula 1-2; and
at least one of R5 and R6 is the group represented by the following Chemical Formula 1-1, and at least one of R7 to R10 is the group represented by the following Chemical Formula 1-2, 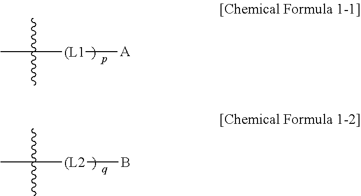
wherein,
L1 and L2 are the same as or different from each other, and are each independently a single bond; a substituted or unsubstituted C6 to C60 arylene group; or a substituted or unsubstituted C2 to C60 heteroarylene group;
A and B are different from each other, and are each independently selected from the group consisting of NA101A102; and groups represented by the following Chemical Formula 1-3 and the following Chemical Formula 1-4, and any one of A and B is the group represented by the following Chemical Formula 1-3;
A101 and A102 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group; and
p and q are the same as or different from each other, and are each independently an integer of 0 to 3, 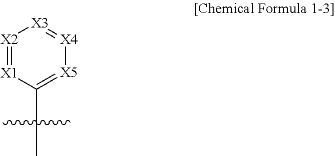
wherein,
X1 is N or CRa;
X2 is N or CRb;
X3 is N or CRc;
X4 is N or CRd;
X5 is N or CRe;
at least one of X1 to X5 is N; and
Ra to Re are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heterocycle, 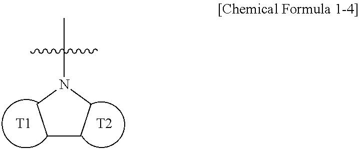
wherein,
T1 ring and T2 ring are the same as or different from each other, and are each independently a substituted or unsubstituted C6 to C60 aromatic ring; or a substituted or unsubstituted C2 to C60 hetero aromatic ring.

2. The heterocyclic compound according to claim 1, wherein the group represented by Chemical Formula 1-3 is any one of the following Chemical Formulae 1-3-1, 1-3-2 and 1-3-3: 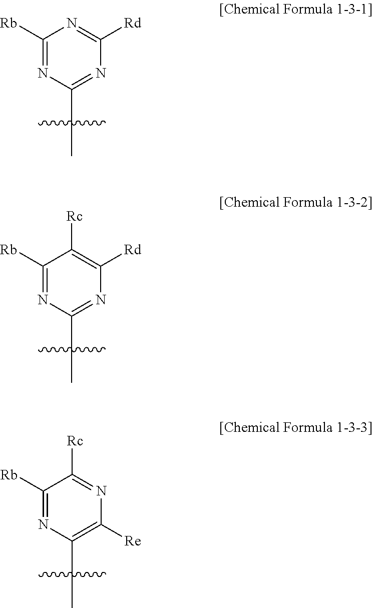
wherein,
Rb to Re are the same as defined in Chemical Formula 1-3 above.

3. The heterocyclic compound according to claim 1, wherein the group represented by Chemical Formula 1-4 is any one of the following Chemical Formulae 1-4-1, 1-4-2 and 1-4-3: 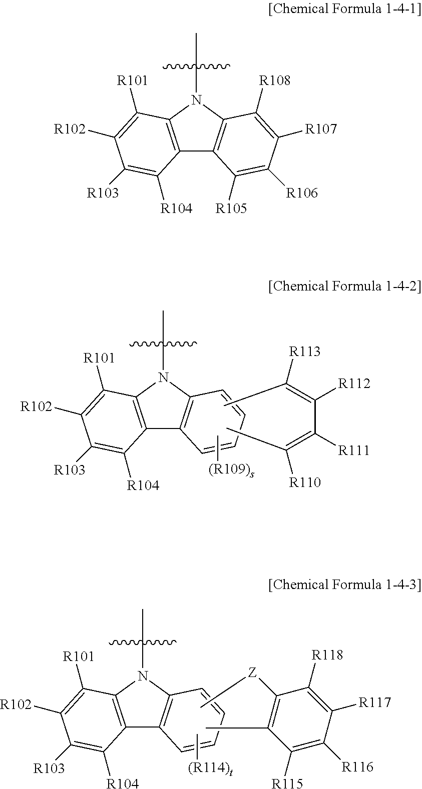
wherein,
R101 to R118 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic ring; or a substituted or unsubstituted C2 to C60 heterocycle;
Z is NR120, O or S;
R120 is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group; and
s and t are the same as or different from each other, and are each independently an integer of 1 or 2.

4. The heterocyclic compound according to claim 3, wherein the group represented by Chemical Formula 1-4-2 is any one of the following Chemical Formulae 1-4-2-a, 1-4-2-b and 1-4-2-c: 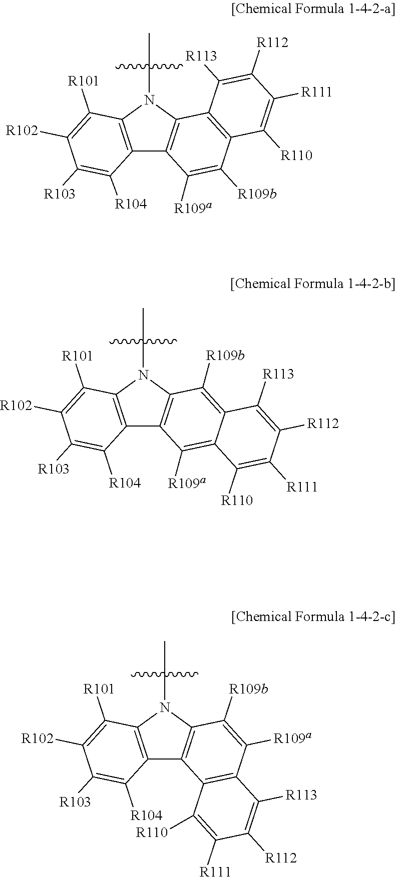
wherein,
R109a and R109b are the same as defined in R109; and
R101 to R104 and R109 to R113 are the same as defined in Chemical Formula 1-4-2.

5. The heterocyclic compound according to claim 3, wherein the group represented by Chemical Formula 1-4-3 is any one of the following Chemical Formulae 1-4-3-a to 1-4-3-f: 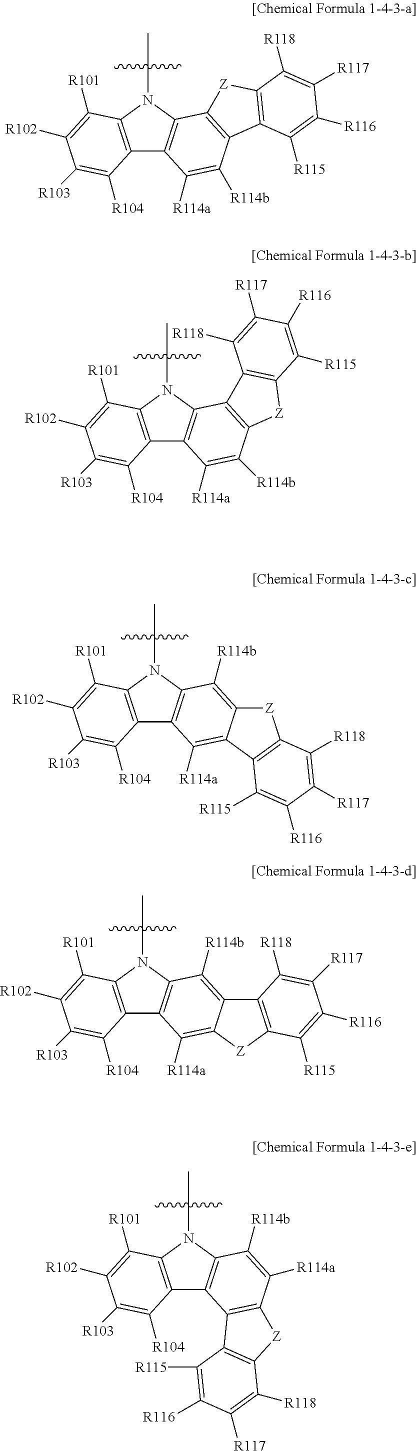 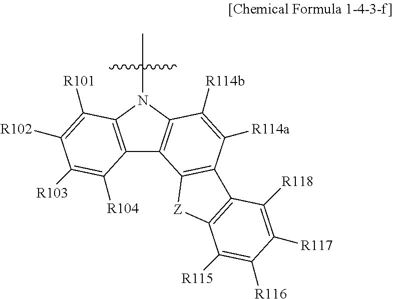
wherein,
R114a and R114b are the same as defined in R114; and
R101 to R104 and R114 to R118 are the same as defined in Chemical Formula 1-4-3.

6. The heterocyclic compound according to claim 1, wherein the heterocyclic compound represented by Chemical Formula 1 does not contain deuterium as a substituent, or the content of deuterium based on a total number of hydrogen atoms and deuterium atoms is 1% to 100%.

7. The heterocyclic compound according to claim 1, wherein the heterocyclic compound represented by Chemical Formula 1 is any one selected from the group consisting of the following compounds:

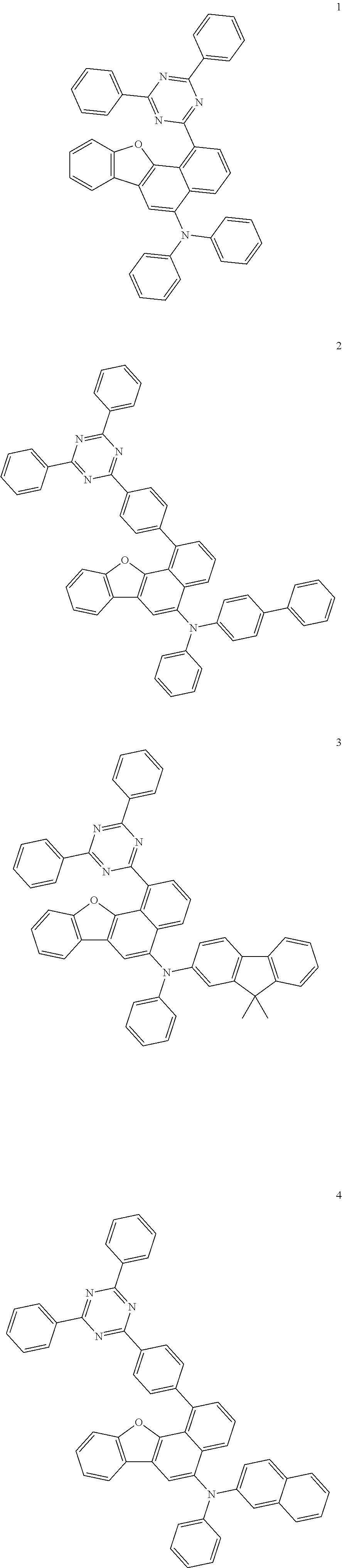 [SMILES: c1ccc(-c2nc(-c3ccccc3)nc(-c3cccc4c(N(c5ccccc5)c5ccccc5)cc5c6ccccc6oc5c34)n2)cc1 ; c1ccc(-c2ccc(N(c3ccccc3)c3cc4c5ccccc5oc4c4c(-c5ccc(-c6nc(-c7ccccc7)nc(-c7ccccc7)n6)cc5)cccc34)cc2)cc1 ; CC1(C)c2ccccc2-c2ccc(N(c3ccccc3)c3cc4c5ccccc5oc4c4c(-c5nc(-c6ccccc6)nc(-c6ccccc6)n5)cccc34)cc21 ; c1ccc(-c2nc(-c3ccccc3)nc(-c3ccc(-c4cccc5c(N(c6ccccc6)c6ccc7ccccc7c6)cc6c7ccccc7oc6c45)cc3)n2)cc1]

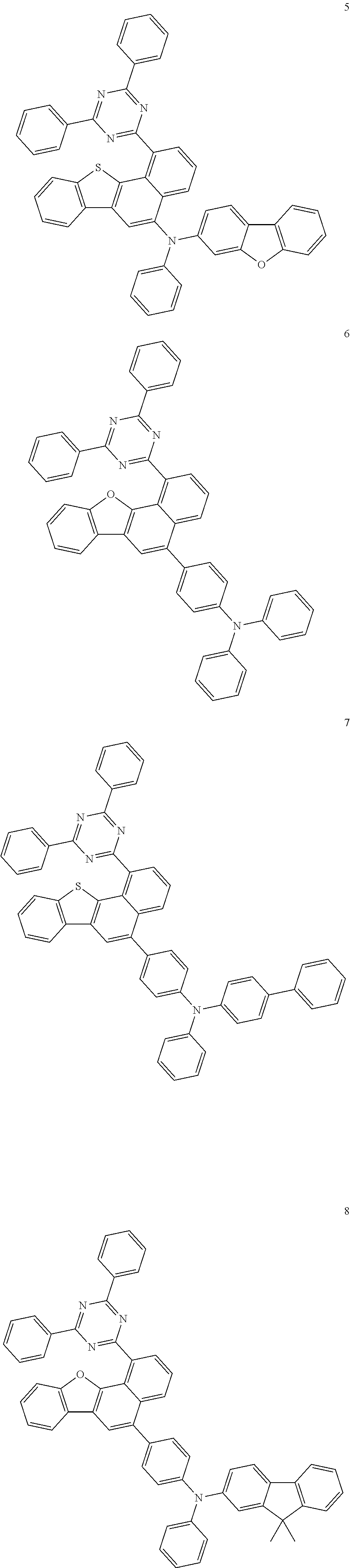

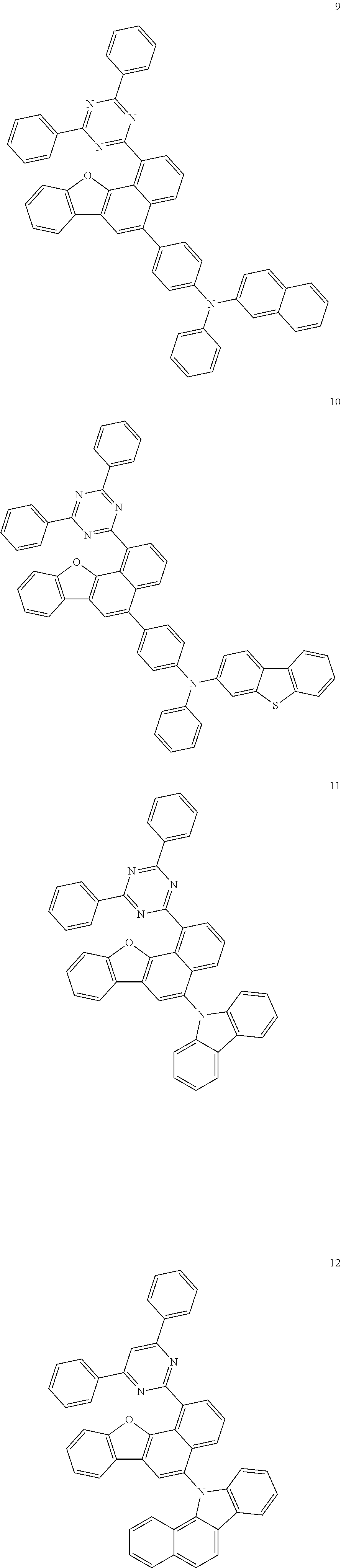 [SMILES: c1ccc(-c2nc(-c3ccccc3)nc(-c3cccc4c(-c5ccc(N(c6ccccc6)c6ccc7ccccc7c6)cc5)cc5c6ccccc6oc5c34)n2)cc1 ; c1ccc(-c2nc(-c3ccccc3)nc(-c3cccc4c(-c5ccc(N(c6ccccc6)c6ccc7c(c6)sc6ccccc67)cc5)cc5c6ccccc6oc5c34)n2)cc1 ; c1ccc(-c2nc(-c3ccccc3)nc(-c3cccc4c(-n5c6ccccc6c6ccccc65)cc5c6ccccc6oc5c34)n2)cc1 ; c1ccc(-c2cc(-c3ccccc3)nc(-c3cccc4c(-n5c6ccccc6c6ccc7ccccc7c65)cc5c6ccccc6oc5c34)n2)cc1]

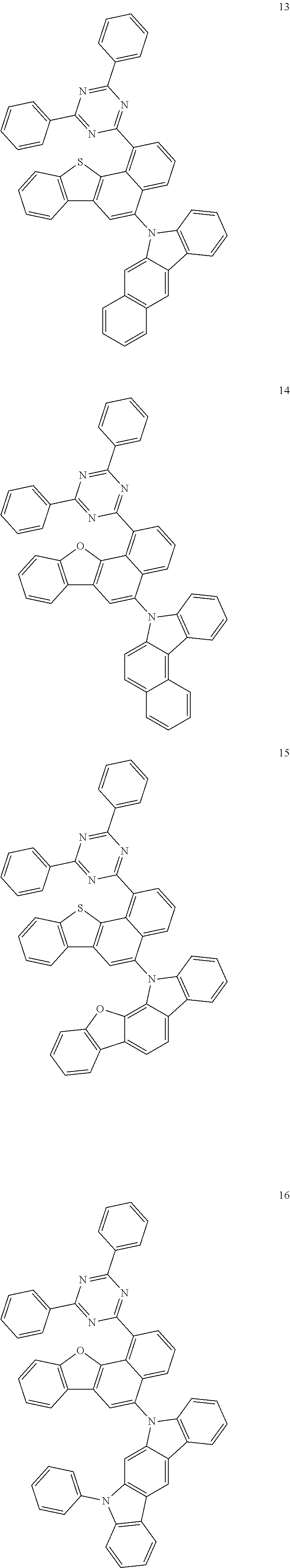 [SMILES: c1ccc(-c2nc(-c3ccccc3)nc(-c3cccc4c(-n5c6ccccc6c6cc7ccccc7cc65)cc5c6ccccc6sc5c34)n2)cc1 ; c1ccc(-c2nc(-c3ccccc3)nc(-c3cccc4c(-n5c6ccccc6c6c7ccccc7ccc65)cc5c6ccccc6oc5c34)n2)cc1 ; c1ccc(-c2nc(-c3ccccc3)nc(-c3cccc4c(-n5c6ccccc6c6ccc7c8ccccc8oc7c65)cc5c6ccccc6sc5c34)n2)cc1 ; c1ccc(-c2nc(-c3ccccc3)nc(-c3cccc4c(-n5c6ccccc6c6cc7c8ccccc8n(-c8ccccc8)c7cc65)cc5c6ccccc6oc5c34)n2)cc1]

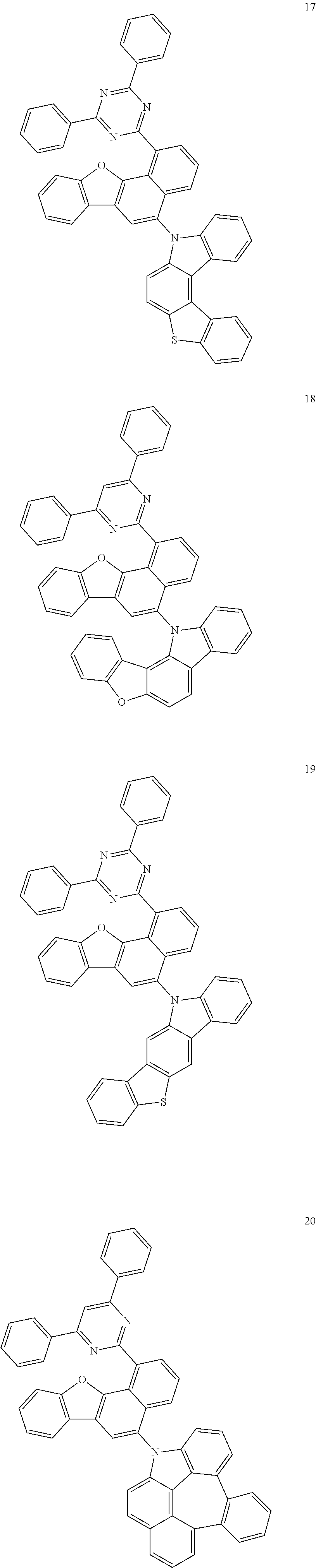 [SMILES: c1ccc(-c2nc(-c3ccccc3)nc(-c3cccc4c(-n5c6ccccc6c6c7c(ccc65)sc5ccccc57)cc5c6ccccc6oc5c34)n2)cc1 ; c1ccc(-c2cc(-c3ccccc3)nc(-c3cccc4c(-n5c6ccccc6c6ccc7oc8ccccc8c7c65)cc5c6ccccc6oc5c34)n2)cc1 ; c1ccc(-c2nc(-c3ccccc3)nc(-c3cccc4c(-n5c6ccccc6c6cc7sc8ccccc8c7cc65)cc5c6ccccc6oc5c34)n2)cc1 ; c1ccc(-c2cc(-c3ccccc3)nc(-c3cccc4c(-n5c6cccc7c6c6c8c(cccc8ccc65)-c5ccccc5-7)cc5c6ccccc6oc5c34)n2)cc1]

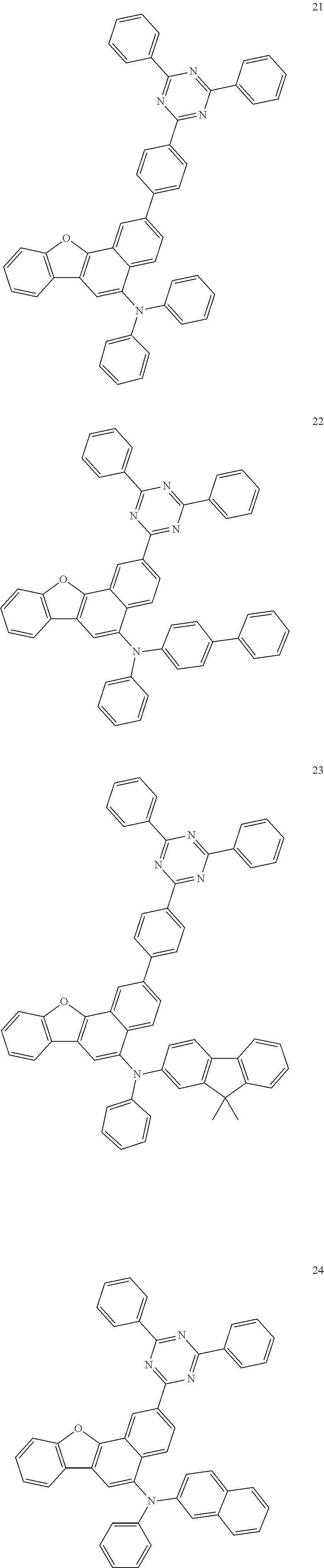 [SMILES: c1ccc(-c2nc(-c3ccccc3)nc(-c3ccc(-c4ccc5c(N(c6ccccc6)c6ccccc6)cc6c7ccccc7oc6c5c4)cc3)n2)cc1 ; c1ccc(-c2ccc(N(c3ccccc3)c3cc4c5ccccc5oc4c4cc(-c5nc(-c6ccccc6)nc(-c6ccccc6)n5)ccc34)cc2)cc1 ; CC1(C)c2ccccc2-c2ccc(N(c3ccccc3)c3cc4c5ccccc5oc4c4cc(-c5ccc(-c6nc(-c7ccccc7)nc(-c7ccccc7)n6)cc5)ccc34)cc21 ; c1ccc(-c2nc(-c3ccccc3)nc(-c3ccc4c(N(c5ccccc5)c5ccc6ccccc6c5)cc5c6ccccc6oc5c4c3)n2)cc1]

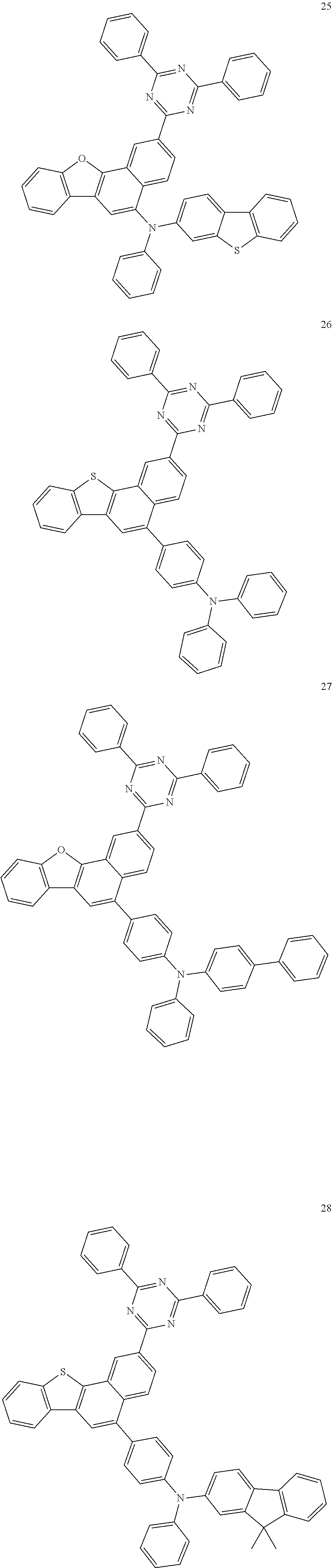 [SMILES: c1ccc(-c2nc(-c3ccccc3)nc(-c3ccc4c(N(c5ccccc5)c5ccc6c(c5)sc5ccccc56)cc5c6ccccc6oc5c4c3)n2)cc1 ; c1ccc(-c2nc(-c3ccccc3)nc(-c3ccc4c(-c5ccc(N(c6ccccc6)c6ccccc6)cc5)cc5c6ccccc6sc5c4c3)n2)cc1 ; c1ccc(-c2ccc(N(c3ccccc3)c3ccc(-c4cc5c6ccccc6oc5c5cc(-c6nc(-c7ccccc7)nc(-c7ccccc7)n6)ccc45)cc3)cc2)cc1 ; CC1(C)c2ccccc2-c2ccc(N(c3ccccc3)c3ccc(-c4cc5c6ccccc6sc5c5cc(-c6nc(-c7ccccc7)nc(-c7ccccc7)n6)ccc45)cc3)cc21]

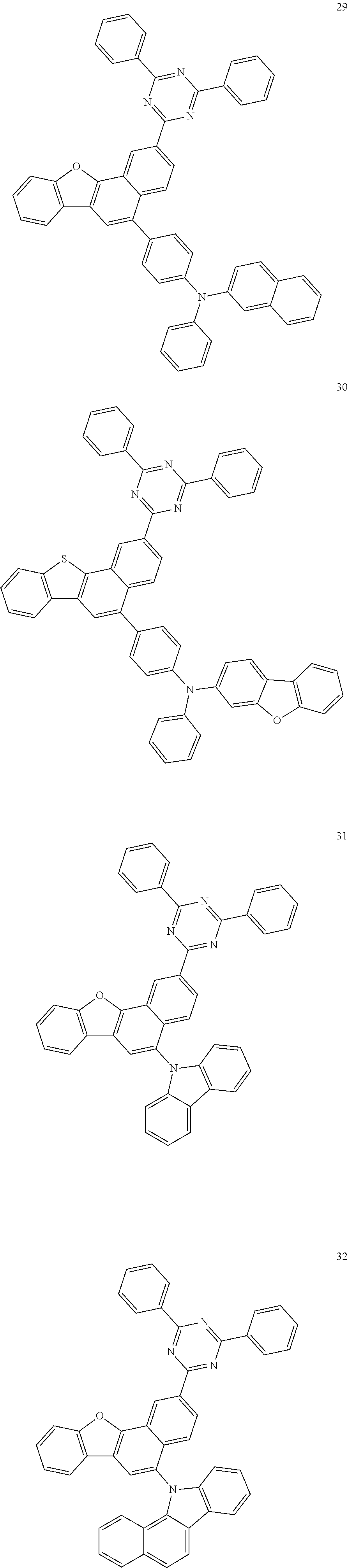 [SMILES: c1ccc(-c2nc(-c3ccccc3)nc(-c3ccc4c(-c5ccc(N(c6ccccc6)c6ccc7ccccc7c6)cc5)cc5c6ccccc6oc5c4c3)n2)cc1 ; c1ccc(-c2nc(-c3ccccc3)nc(-c3ccc4c(-c5ccc(N(c6ccccc6)c6ccc7c(c6)oc6ccccc67)cc5)cc5c6ccccc6sc5c4c3)n2)cc1 ; c1ccc(-c2nc(-c3ccccc3)nc(-c3ccc4c(-n5c6ccccc6c6ccccc65)cc5c6ccccc6oc5c4c3)n2)cc1 ; c1ccc(-c2nc(-c3ccccc3)nc(-c3ccc4c(-n5c6ccccc6c6ccc7ccccc7c65)cc5c6ccccc6oc5c4c3)n2)cc1]

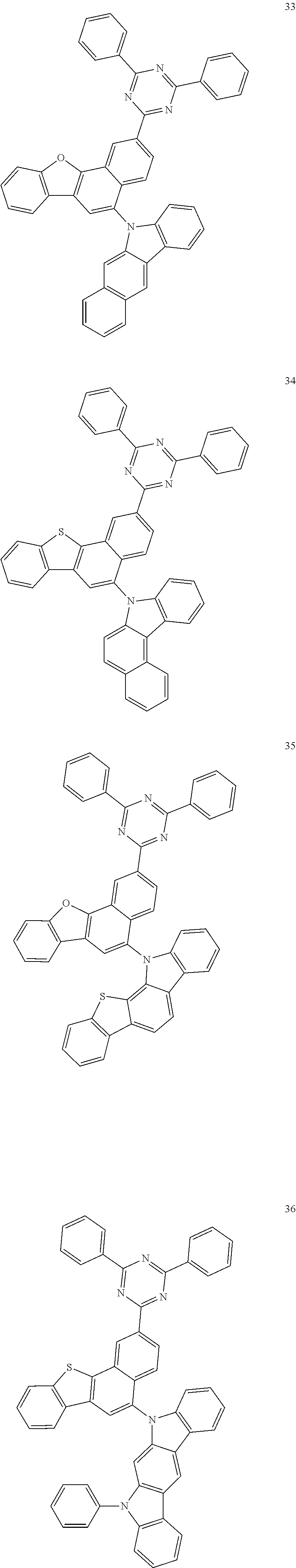 [SMILES: c1ccc(-c2nc(-c3ccccc3)nc(-c3ccc4c(-n5c6ccccc6c6cc7ccccc7cc65)cc5c6ccccc6oc5c4c3)n2)cc1 ; c1ccc(-c2nc(-c3ccccc3)nc(-c3ccc4c(-n5c6ccccc6c6c7ccccc7ccc65)cc5c6ccccc6sc5c4c3)n2)cc1 ; c1ccc(-c2nc(-c3ccccc3)nc(-c3ccc4c(-n5c6ccccc6c6ccc7c8ccccc8sc7c65)cc5c6ccccc6oc5c4c3)n2)cc1 ; c1ccc(-c2nc(-c3ccccc3)nc(-c3ccc4c(-n5c6ccccc6c6cc7c8ccccc8n(-c8ccccc8)c7cc65)cc5c6ccccc6sc5c4c3)n2)cc1]

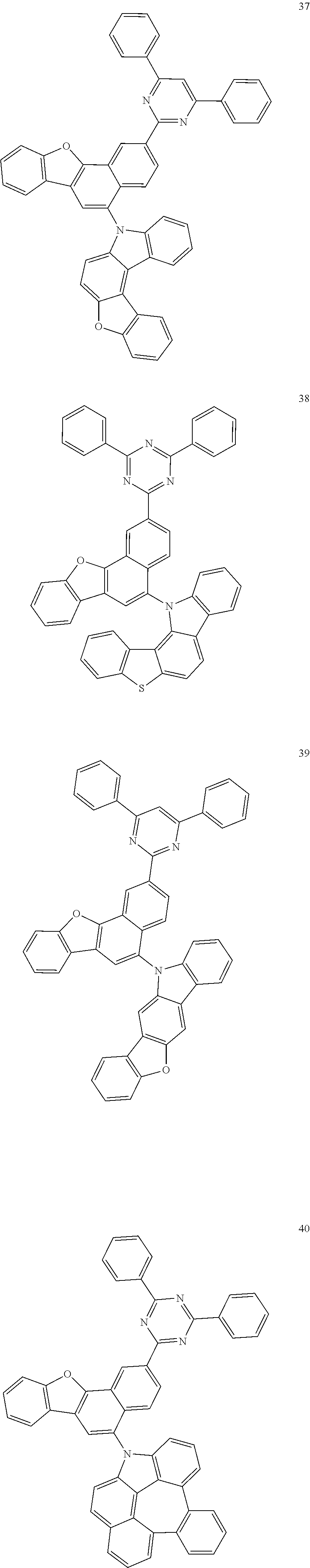 [SMILES: c1ccc(-c2cc(-c3ccccc3)nc(-c3ccc4c(-n5c6ccccc6c6c7c(ccc65)oc5ccccc57)cc5c6ccccc6oc5c4c3)n2)cc1 ; c1ccc(-c2nc(-c3ccccc3)nc(-c3ccc4c(-n5c6ccccc6c6ccc7sc8ccccc8c7c65)cc5c6ccccc6oc5c4c3)n2)cc1 ; c1ccc(-c2cc(-c3ccccc3)nc(-c3ccc4c(-n5c6ccccc6c6cc7oc8ccccc8c7cc65)cc5c6ccccc6oc5c4c3)n2)cc1 ; c1ccc(-c2nc(-c3ccccc3)nc(-c3ccc4c(-n5c6cccc7c6c6c8c(cccc8ccc65)-c5ccccc5-7)cc5c6ccccc6oc5c4c3)n2)cc1]

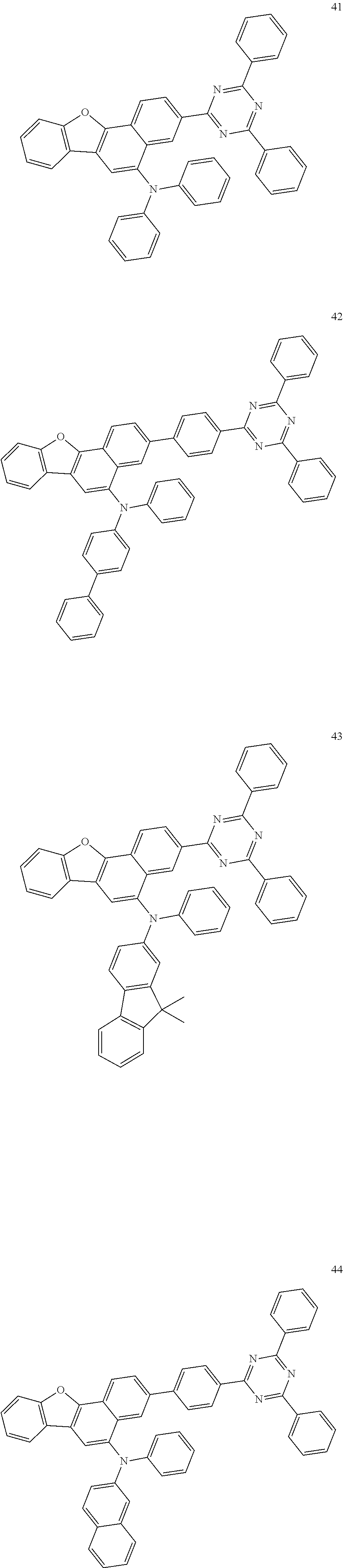 [SMILES: c1ccc(-c2nc(-c3ccccc3)nc(-c3ccc4c(c3)c(N(c3ccccc3)c3ccccc3)cc3c5ccccc5oc43)n2)cc1 ; c1ccc(-c2ccc(N(c3ccccc3)c3cc4c5ccccc5oc4c4ccc(-c5ccc(-c6nc(-c7ccccc7)nc(-c7ccccc7)n6)cc5)cc34)cc2)cc1 ; CC1(C)c2ccccc2-c2ccc(N(c3ccccc3)c3cc4c5ccccc5oc4c4ccc(-c5nc(-c6ccccc6)nc(-c6ccccc6)n5)cc34)cc21 ; c1ccc(-c2nc(-c3ccccc3)nc(-c3ccc(-c4ccc5c(c4)c(N(c4ccccc4)c4ccc6ccccc6c4)cc4c6ccccc6oc54)cc3)n2)cc1]

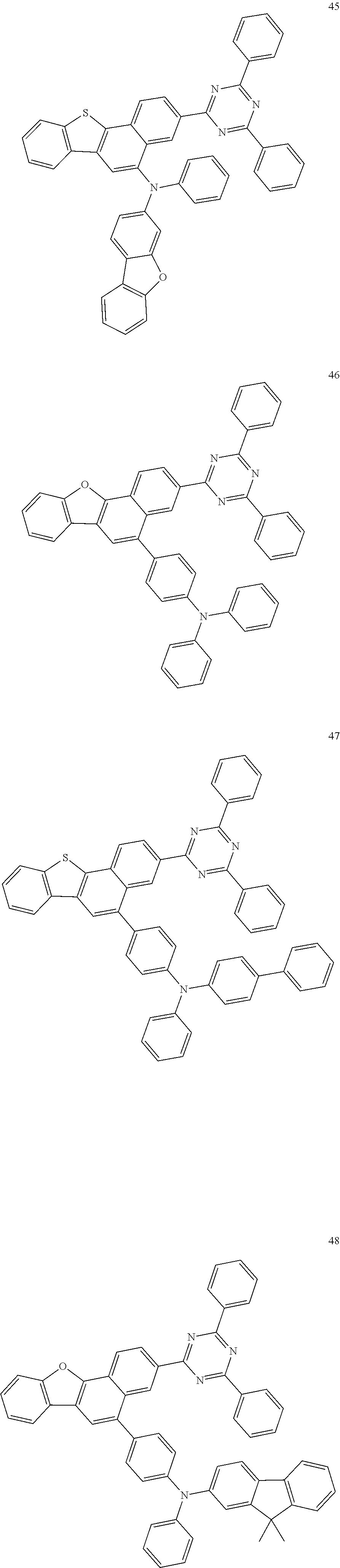 [SMILES: c1ccc(-c2nc(-c3ccccc3)nc(-c3ccc4c(c3)c(N(c3ccccc3)c3ccc5c(c3)oc3ccccc35)cc3c5ccccc5sc43)n2)cc1 ; c1ccc(-c2nc(-c3ccccc3)nc(-c3ccc4c(c3)c(-c3ccc(N(c5ccccc5)c5ccccc5)cc3)cc3c5ccccc5oc43)n2)cc1 ; c1ccc(-c2ccc(N(c3ccccc3)c3ccc(-c4cc5c6ccccc6sc5c5ccc(-c6nc(-c7ccccc7)nc(-c7ccccc7)n6)cc45)cc3)cc2)cc1 ; CC1(C)c2ccccc2-c2ccc(N(c3ccccc3)c3ccc(-c4cc5c6ccccc6oc5c5ccc(-c6nc(-c7ccccc7)nc(-c7ccccc7)n6)cc45)cc3)cc21]

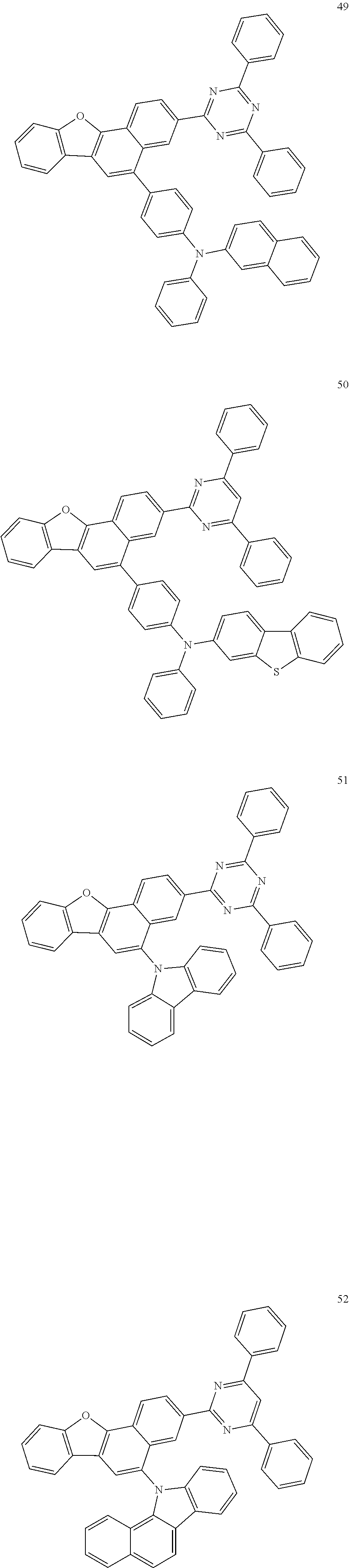 [SMILES: c1ccc(-c2nc(-c3ccccc3)nc(-c3ccc4c(c3)c(-c3ccc(N(c5ccccc5)c5ccc6ccccc6c5)cc3)cc3c5ccccc5oc43)n2)cc1 ; c1ccc(-c2cc(-c3ccccc3)nc(-c3ccc4c(c3)c(-c3ccc(N(c5ccccc5)c5ccc6c(c5)sc5ccccc56)cc3)cc3c5ccccc5oc43)n2)cc1 ; c1ccc(-c2nc(-c3ccccc3)nc(-c3ccc4c(c3)c(-n3c5ccccc5c5ccccc53)cc3c5ccccc5oc43)n2)cc1 ; c1ccc(-c2cc(-c3ccccc3)nc(-c3ccc4c(c3)c(-n3c5ccccc5c5ccc6ccccc6c53)cc3c5ccccc5oc43)n2)cc1]

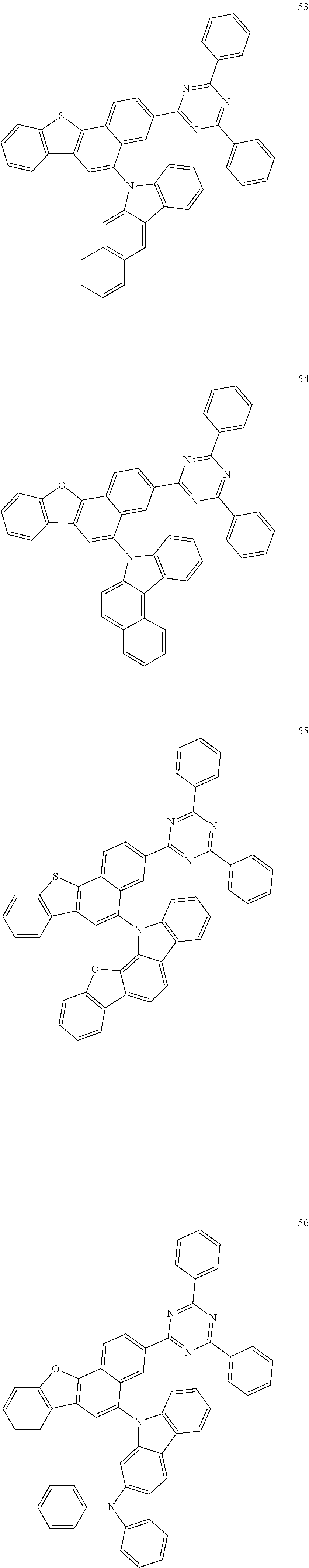 [SMILES: c1ccc(-c2nc(-c3ccccc3)nc(-c3ccc4c(c3)c(-n3c5ccccc5c5cc6ccccc6cc53)cc3c5ccccc5sc43)n2)cc1 ; c1ccc(-c2nc(-c3ccccc3)nc(-c3ccc4c(c3)c(-n3c5ccccc5c5c6ccccc6ccc53)cc3c5ccccc5oc43)n2)cc1 ; c1ccc(-c2nc(-c3ccccc3)nc(-c3ccc4c(c3)c(-n3c5ccccc5c5ccc6c7ccccc7oc6c53)cc3c5ccccc5sc43)n2)cc1 ; c1ccc(-c2nc(-c3ccccc3)nc(-c3ccc4c(c3)c(-n3c5ccccc5c5cc6c7ccccc7n(-c7ccccc7)c6cc53)cc3c5ccccc5oc43)n2)cc1]

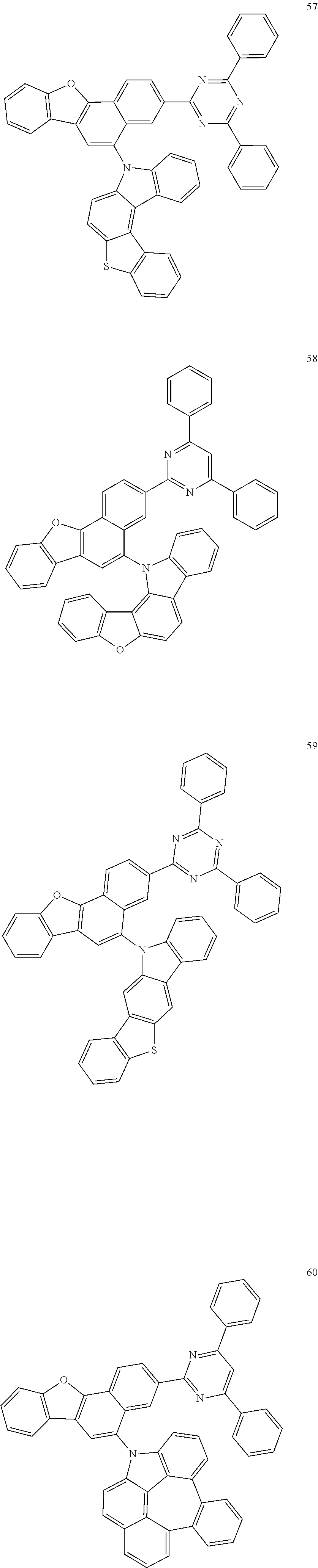 [SMILES: c1ccc(-c2nc(-c3ccccc3)nc(-c3ccc4c(c3)c(-n3c5ccccc5c5c6c(ccc53)sc3ccccc36)cc3c5ccccc5oc43)n2)cc1 ; c1ccc(-c2cc(-c3ccccc3)nc(-c3ccc4c(c3)c(-n3c5ccccc5c5ccc6oc7ccccc7c6c53)cc3c5ccccc5oc43)n2)cc1 ; c1ccc(-c2nc(-c3ccccc3)nc(-c3ccc4c(c3)c(-n3c5ccccc5c5cc6sc7ccccc7c6cc53)cc3c5ccccc5oc43)n2)cc1 ; c1ccc(-c2cc(-c3ccccc3)nc(-c3ccc4c(c3)c(-n3c5cccc6c5c5c7c(cccc7ccc53)-c3ccccc3-6)cc3c5ccccc5oc43)n2)cc1]

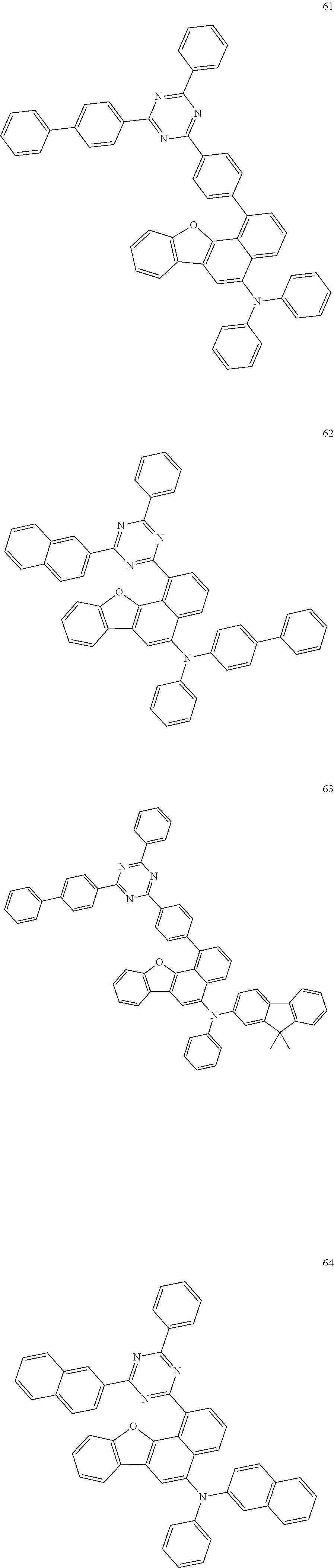 [SMILES: c1ccc(-c2ccc(-c3nc(-c4ccccc4)nc(-c4ccc(-c5cccc6c(N(c7ccccc7)c7ccccc7)cc7c8ccccc8oc7c56)cc4)n3)cc2)cc1 ; c1ccc(-c2ccc(N(c3ccccc3)c3cc4c5ccccc5oc4c4c(-c5nc(-c6ccccc6)nc(-c6ccc7ccccc7c6)n5)cccc34)cc2)cc1 ; CC1(C)c2ccccc2-c2ccc(N(c3ccccc3)c3cc4c5ccccc5oc4c4c(-c5ccc(-c6nc(-c7ccccc7)nc(-c7ccc(-c8ccccc8)cc7)n6)cc5)cccc34)cc21 ; c1ccc(-c2nc(-c3ccc4ccccc4c3)nc(-c3cccc4c(N(c5ccccc5)c5ccc6ccccc6c5)cc5c6ccccc6oc5c34)n2)cc1]

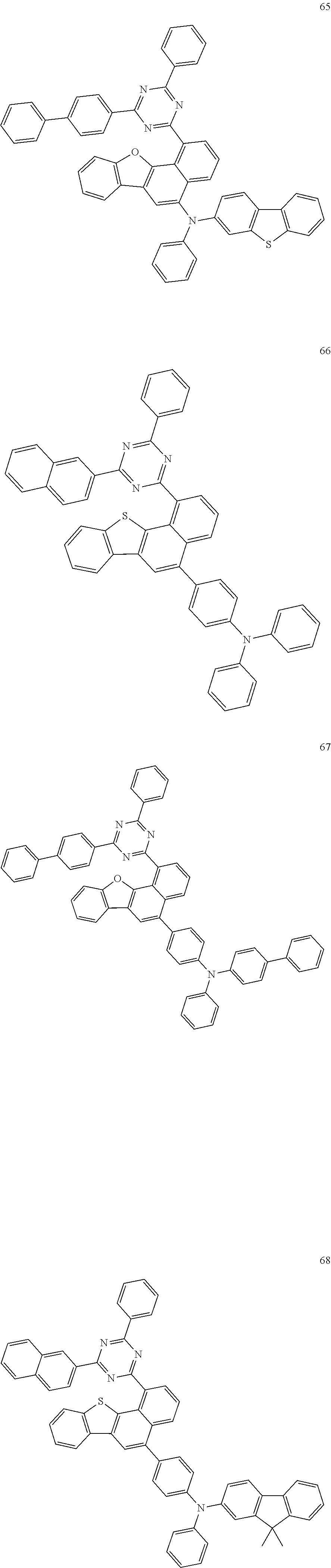 [SMILES: c1ccc(-c2ccc(-c3nc(-c4ccccc4)nc(-c4cccc5c(N(c6ccccc6)c6ccc7c(c6)sc6ccccc67)cc6c7ccccc7oc6c45)n3)cc2)cc1 ; c1ccc(-c2nc(-c3ccc4ccccc4c3)nc(-c3cccc4c(-c5ccc(N(c6ccccc6)c6ccccc6)cc5)cc5c6ccccc6sc5c34)n2)cc1 ; c1ccc(-c2ccc(-c3nc(-c4ccccc4)nc(-c4cccc5c(-c6ccc(N(c7ccccc7)c7ccc(-c8ccccc8)cc7)cc6)cc6c7ccccc7oc6c45)n3)cc2)cc1 ; CC1(C)c2ccccc2-c2ccc(N(c3ccccc3)c3ccc(-c4cc5c6ccccc6sc5c5c(-c6nc(-c7ccccc7)nc(-c7ccc8ccccc8c7)n6)cccc45)cc3)cc21]

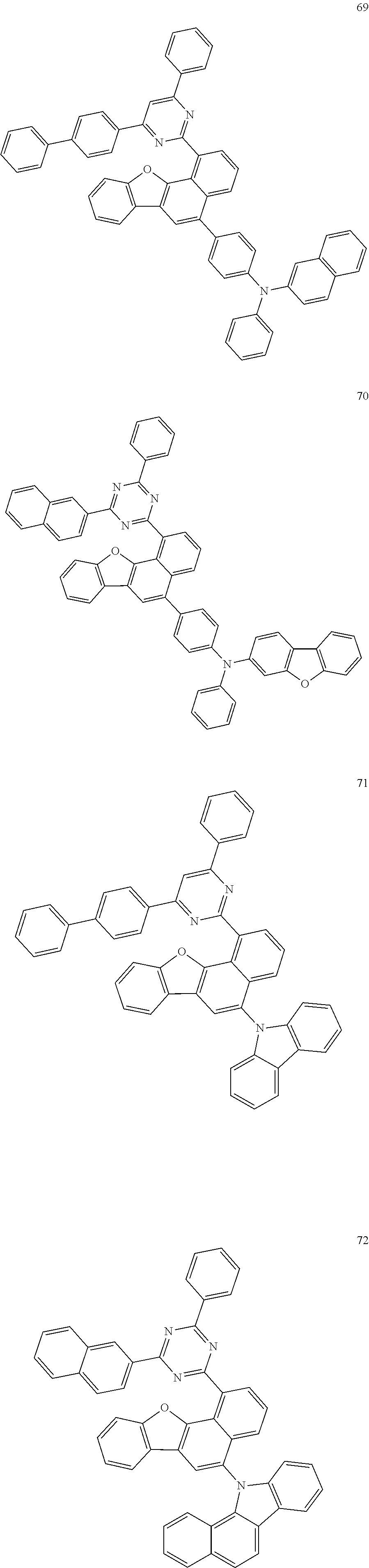 [SMILES: c1ccc(-c2ccc(-c3cc(-c4ccccc4)nc(-c4cccc5c(-c6ccc(N(c7ccccc7)c7ccc8ccccc8c7)cc6)cc6c7ccccc7oc6c45)n3)cc2)cc1 ; c1ccc(-c2nc(-c3ccc4ccccc4c3)nc(-c3cccc4c(-c5ccc(N(c6ccccc6)c6ccc7c(c6)oc6ccccc67)cc5)cc5c6ccccc6oc5c34)n2)cc1 ; c1ccc(-c2ccc(-c3cc(-c4ccccc4)nc(-c4cccc5c(-n6c7ccccc7c7ccccc76)cc6c7ccccc7oc6c45)n3)cc2)cc1 ; c1ccc(-c2nc(-c3ccc4ccccc4c3)nc(-c3cccc4c(-n5c6ccccc6c6ccc7ccccc7c65)cc5c6ccccc6oc5c34)n2)cc1]

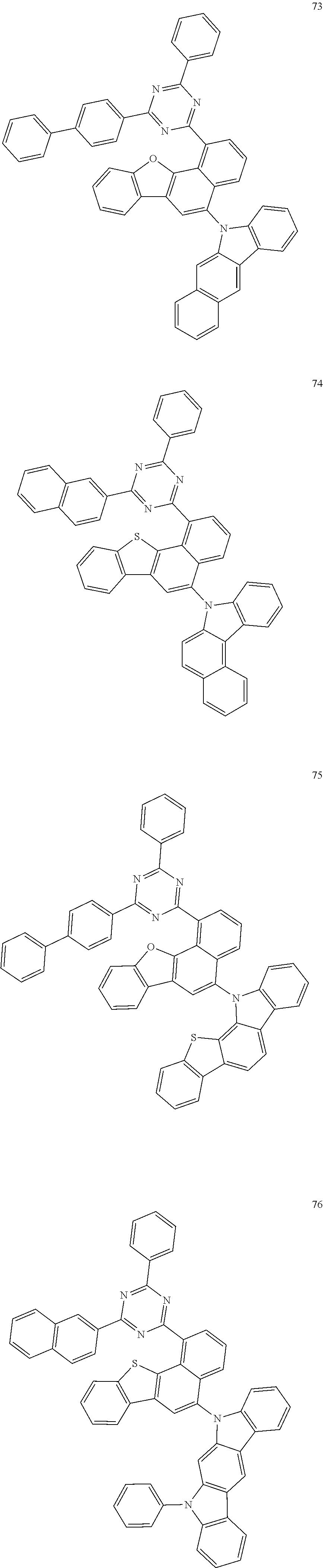 [SMILES: c1ccc(-c2ccc(-c3nc(-c4ccccc4)nc(-c4cccc5c(-n6c7ccccc7c7cc8ccccc8cc76)cc6c7ccccc7oc6c45)n3)cc2)cc1 ; c1ccc(-c2nc(-c3ccc4ccccc4c3)nc(-c3cccc4c(-n5c6ccccc6c6c7ccccc7ccc65)cc5c6ccccc6sc5c34)n2)cc1 ; c1ccc(-c2ccc(-c3nc(-c4ccccc4)nc(-c4cccc5c(-n6c7ccccc7c7ccc8c9ccccc9sc8c76)cc6c7ccccc7oc6c45)n3)cc2)cc1 ; c1ccc(-c2nc(-c3ccc4ccccc4c3)nc(-c3cccc4c(-n5c6ccccc6c6cc7c8ccccc8n(-c8ccccc8)c7cc65)cc5c6ccccc6sc5c34)n2)cc1]

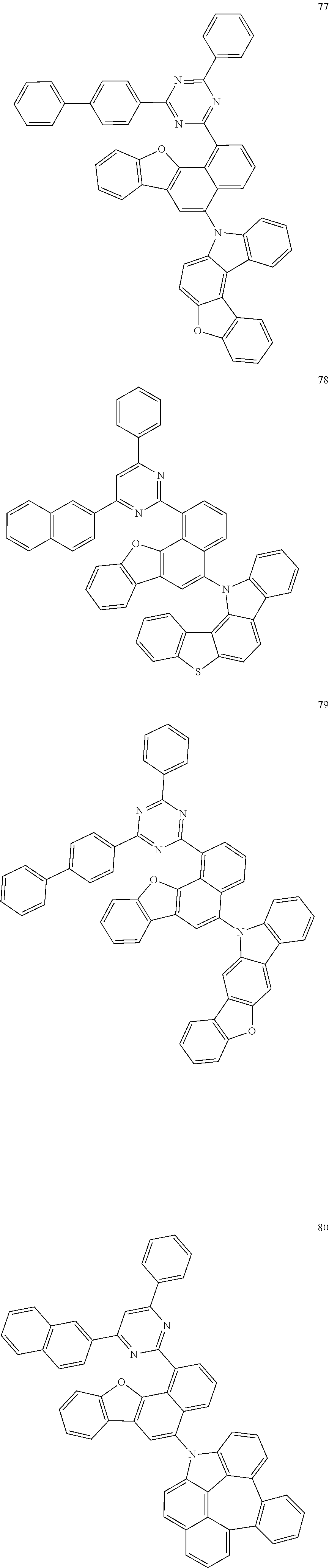 [SMILES: c1ccc(-c2ccc(-c3nc(-c4ccccc4)nc(-c4cccc5c(-n6c7ccccc7c7c8c(ccc76)oc6ccccc68)cc6c7ccccc7oc6c45)n3)cc2)cc1 ; c1ccc(-c2cc(-c3ccc4ccccc4c3)nc(-c3cccc4c(-n5c6ccccc6c6ccc7sc8ccccc8c7c65)cc5c6ccccc6oc5c34)n2)cc1 ; c1ccc(-c2ccc(-c3nc(-c4ccccc4)nc(-c4cccc5c(-n6c7ccccc7c7cc8oc9ccccc9c8cc76)cc6c7ccccc7oc6c45)n3)cc2)cc1 ; c1ccc(-c2cc(-c3ccc4ccccc4c3)nc(-c3cccc4c(-n5c6cccc7c6c6c8c(cccc8ccc65)-c5ccccc5-7)cc5c6ccccc6oc5c34)n2)cc1]

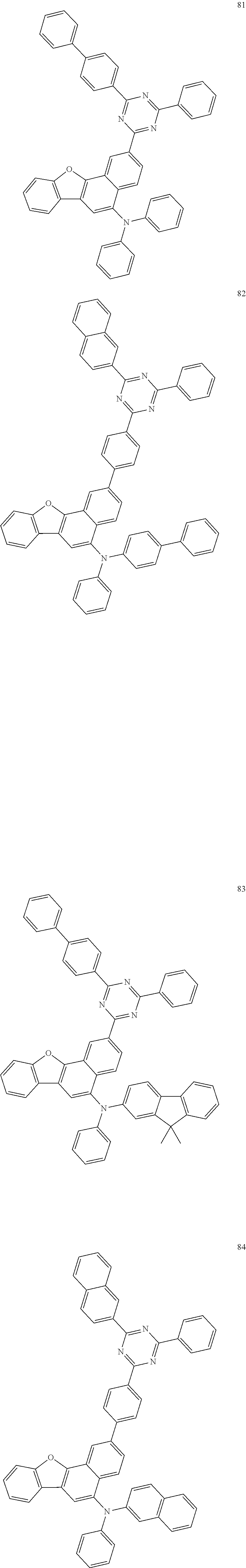

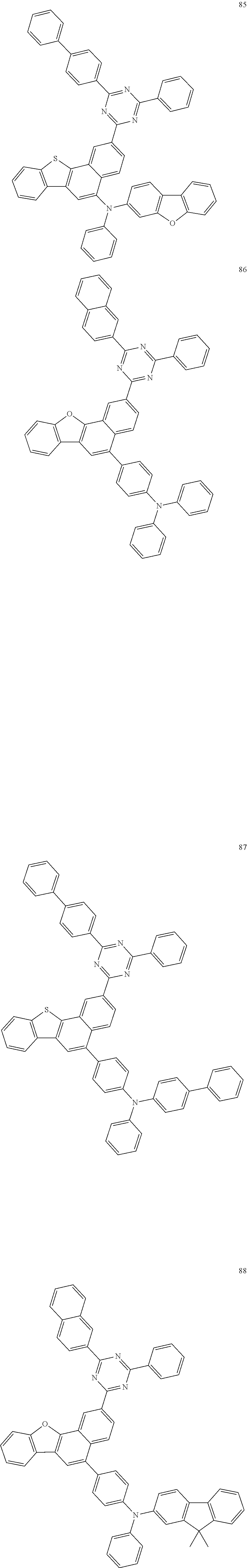

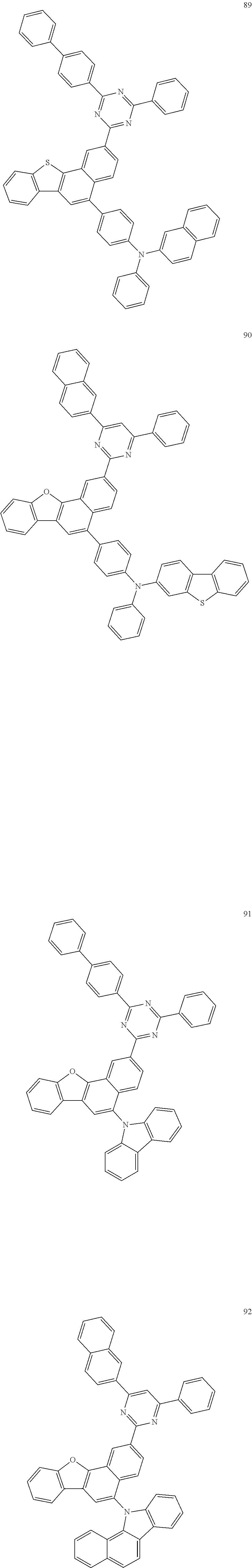 [SMILES: c1ccc(-c2ccc(-c3nc(-c4ccccc4)nc(-c4ccc5c(-c6ccc(N(c7ccccc7)c7ccc8ccccc8c7)cc6)cc6c7ccccc7sc6c5c4)n3)cc2)cc1 ; c1ccc(-c2cc(-c3ccc4ccccc4c3)nc(-c3ccc4c(-c5ccc(N(c6ccccc6)c6ccc7c(c6)sc6ccccc67)cc5)cc5c6ccccc6oc5c4c3)n2)cc1 ; c1ccc(-c2ccc(-c3nc(-c4ccccc4)nc(-c4ccc5c(-n6c7ccccc7c7ccccc76)cc6c7ccccc7oc6c5c4)n3)cc2)cc1 ; c1ccc(-c2cc(-c3ccc4ccccc4c3)nc(-c3ccc4c(-n5c6ccccc6c6ccc7ccccc7c65)cc5c6ccccc6oc5c4c3)n2)cc1]

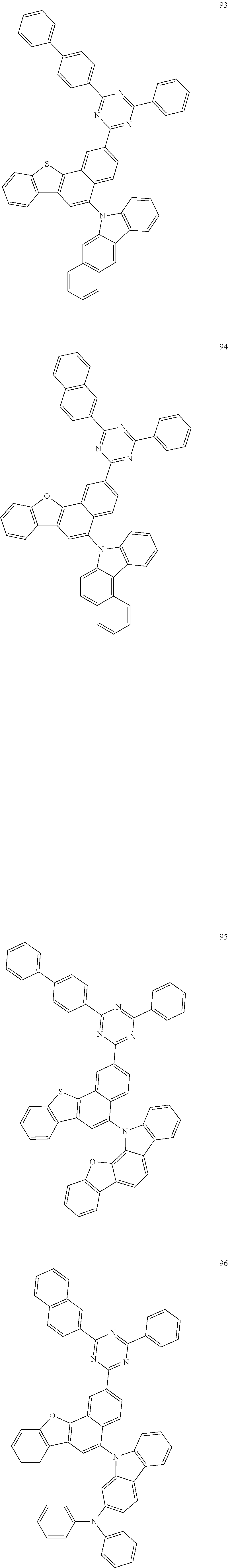 [SMILES: c1ccc(-c2ccc(-c3nc(-c4ccccc4)nc(-c4ccc5c(-n6c7ccccc7c7cc8ccccc8cc76)cc6c7ccccc7sc6c5c4)n3)cc2)cc1 ; c1ccc(-c2nc(-c3ccc4ccccc4c3)nc(-c3ccc4c(-n5c6ccccc6c6c7ccccc7ccc65)cc5c6ccccc6oc5c4c3)n2)cc1 ; c1ccc(-c2ccc(-c3nc(-c4ccccc4)nc(-c4ccc5c(-n6c7ccccc7c7ccc8c9ccccc9oc8c76)cc6c7ccccc7sc6c5c4)n3)cc2)cc1 ; c1ccc(-c2nc(-c3ccc4ccccc4c3)nc(-c3ccc4c(-n5c6ccccc6c6cc7c8ccccc8n(-c8ccccc8)c7cc65)cc5c6ccccc6oc5c4c3)n2)cc1]

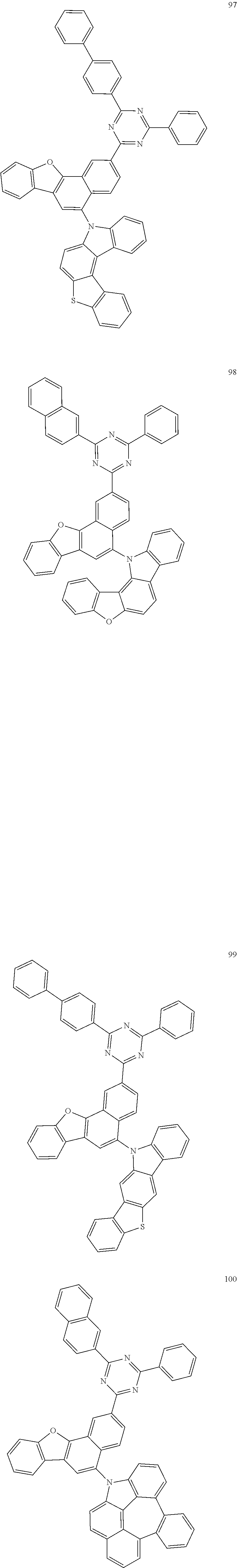 [SMILES: c1ccc(-c2ccc(-c3nc(-c4ccccc4)nc(-c4ccc5c(-n6c7ccccc7c7c8c(ccc76)sc6ccccc68)cc6c7ccccc7oc6c5c4)n3)cc2)cc1 ; c1ccc(-c2nc(-c3ccc4ccccc4c3)nc(-c3ccc4c(-n5c6ccccc6c6ccc7oc8ccccc8c7c65)cc5c6ccccc6oc5c4c3)n2)cc1 ; c1ccc(-c2ccc(-c3nc(-c4ccccc4)nc(-c4ccc5c(-n6c7ccccc7c7cc8sc9ccccc9c8cc76)cc6c7ccccc7oc6c5c4)n3)cc2)cc1 ; c1ccc(-c2nc(-c3ccc4ccccc4c3)nc(-c3ccc4c(-n5c6cccc7c6c6c8c(cccc8ccc65)-c5ccccc5-7)cc5c6ccccc6oc5c4c3)n2)cc1]

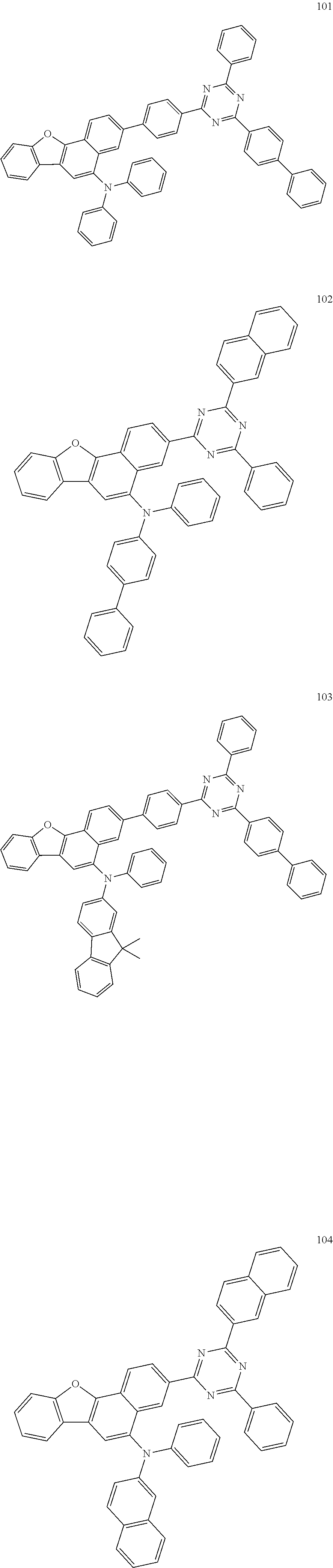 [SMILES: c1ccc(-c2ccc(-c3nc(-c4ccccc4)nc(-c4ccc(-c5ccc6c(c5)c(N(c5ccccc5)c5ccccc5)cc5c7ccccc7oc65)cc4)n3)cc2)cc1 ; c1ccc(-c2ccc(N(c3ccccc3)c3cc4c5ccccc5oc4c4ccc(-c5nc(-c6ccccc6)nc(-c6ccc7ccccc7c6)n5)cc34)cc2)cc1 ; CC1(C)c2ccccc2-c2ccc(N(c3ccccc3)c3cc4c5ccccc5oc4c4ccc(-c5ccc(-c6nc(-c7ccccc7)nc(-c7ccc(-c8ccccc8)cc7)n6)cc5)cc34)cc21 ; c1ccc(-c2nc(-c3ccc4ccccc4c3)nc(-c3ccc4c(c3)c(N(c3ccccc3)c3ccc5ccccc5c3)cc3c5ccccc5oc43)n2)cc1]

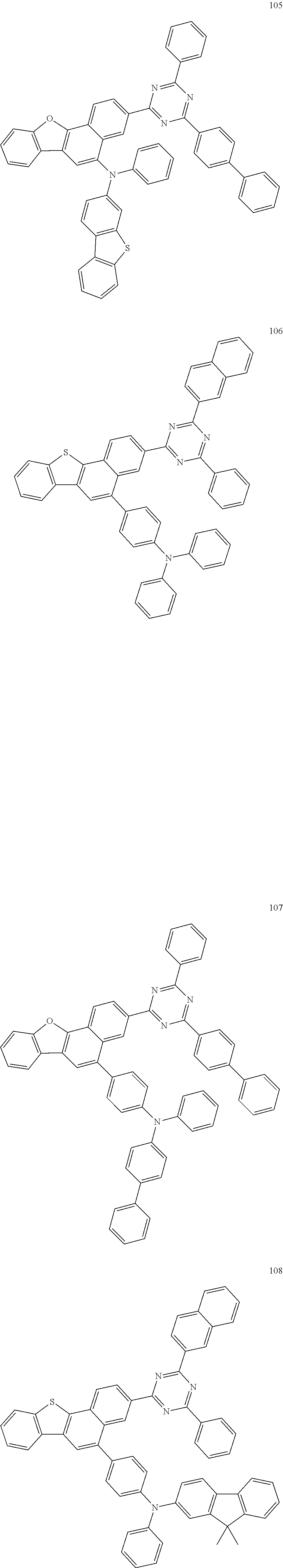 [SMILES: c1ccc(-c2ccc(-c3nc(-c4ccccc4)nc(-c4ccc5c(c4)c(N(c4ccccc4)c4ccc6c(c4)sc4ccccc46)cc4c6ccccc6oc54)n3)cc2)cc1 ; c1ccc(-c2nc(-c3ccc4ccccc4c3)nc(-c3ccc4c(c3)c(-c3ccc(N(c5ccccc5)c5ccccc5)cc3)cc3c5ccccc5sc43)n2)cc1 ; c1ccc(-c2ccc(-c3nc(-c4ccccc4)nc(-c4ccc5c(c4)c(-c4ccc(N(c6ccccc6)c6ccc(-c7ccccc7)cc6)cc4)cc4c6ccccc6oc54)n3)cc2)cc1 ; CC1(C)c2ccccc2-c2ccc(N(c3ccccc3)c3ccc(-c4cc5c6ccccc6sc5c5ccc(-c6nc(-c7ccccc7)nc(-c7ccc8ccccc8c7)n6)cc45)cc3)cc21]

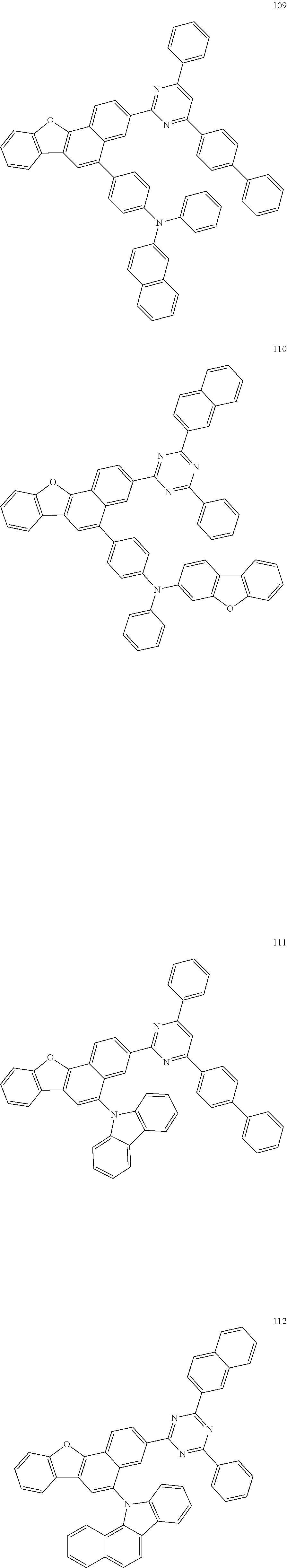 [SMILES: c1ccc(-c2ccc(-c3cc(-c4ccccc4)nc(-c4ccc5c(c4)c(-c4ccc(N(c6ccccc6)c6ccc7ccccc7c6)cc4)cc4c6ccccc6oc54)n3)cc2)cc1 ; c1ccc(-c2nc(-c3ccc4ccccc4c3)nc(-c3ccc4c(c3)c(-c3ccc(N(c5ccccc5)c5ccc6c(c5)oc5ccccc56)cc3)cc3c5ccccc5oc43)n2)cc1 ; c1ccc(-c2ccc(-c3cc(-c4ccccc4)nc(-c4ccc5c(c4)c(-n4c6ccccc6c6ccccc64)cc4c6ccccc6oc54)n3)cc2)cc1 ; c1ccc(-c2nc(-c3ccc4ccccc4c3)nc(-c3ccc4c(c3)c(-n3c5ccccc5c5ccc6ccccc6c53)cc3c5ccccc5oc43)n2)cc1]

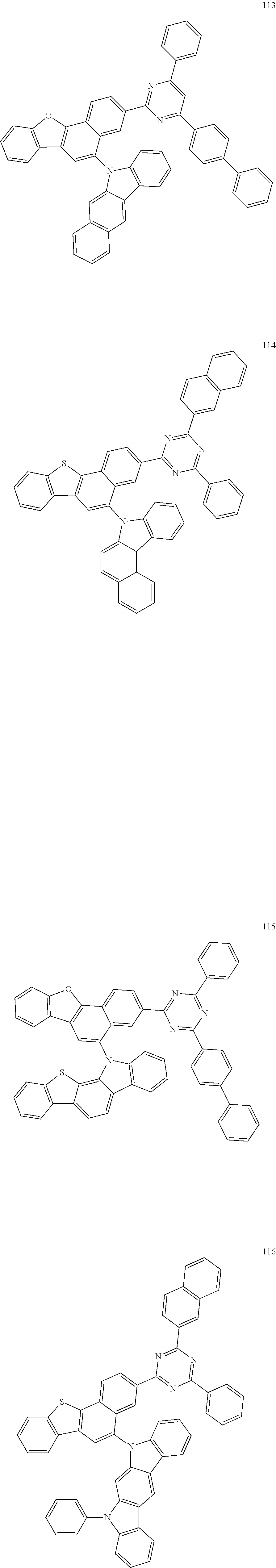 [SMILES: c1ccc(-c2ccc(-c3cc(-c4ccccc4)nc(-c4ccc5c(c4)c(-n4c6ccccc6c6cc7ccccc7cc64)cc4c6ccccc6oc54)n3)cc2)cc1 ; c1ccc(-c2nc(-c3ccc4ccccc4c3)nc(-c3ccc4c(c3)c(-n3c5ccccc5c5c6ccccc6ccc53)cc3c5ccccc5sc43)n2)cc1 ; c1ccc(-c2ccc(-c3nc(-c4ccccc4)nc(-c4ccc5c(c4)c(-n4c6ccccc6c6ccc7c8ccccc8sc7c64)cc4c6ccccc6oc54)n3)cc2)cc1 ; c1ccc(-c2nc(-c3ccc4ccccc4c3)nc(-c3ccc4c(c3)c(-n3c5ccccc5c5cc6c7ccccc7n(-c7ccccc7)c6cc53)cc3c5ccccc5sc43)n2)cc1]

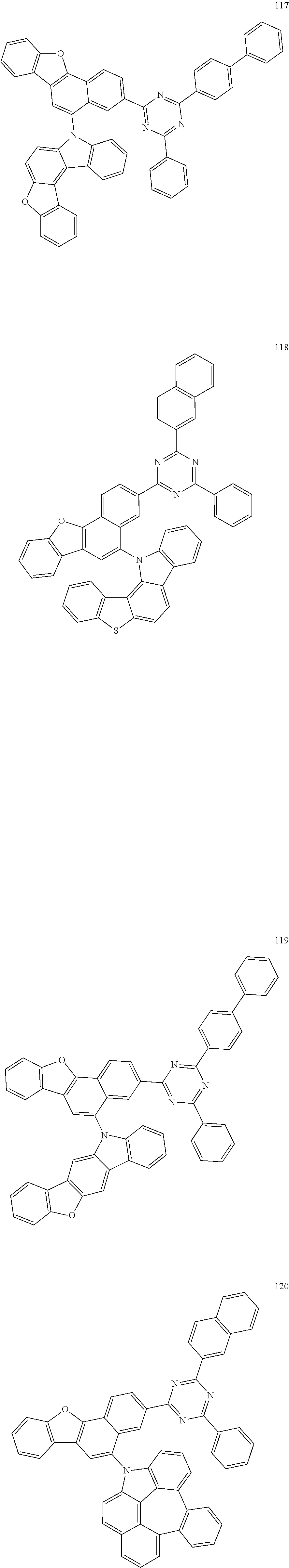 [SMILES: c1ccc(-c2ccc(-c3nc(-c4ccccc4)nc(-c4ccc5c(c4)c(-n4c6ccccc6c6c7c(ccc64)oc4ccccc47)cc4c6ccccc6oc54)n3)cc2)cc1 ; c1ccc(-c2nc(-c3ccc4ccccc4c3)nc(-c3ccc4c(c3)c(-n3c5ccccc5c5ccc6sc7ccccc7c6c53)cc3c5ccccc5oc43)n2)cc1 ; c1ccc(-c2ccc(-c3nc(-c4ccccc4)nc(-c4ccc5c(c4)c(-n4c6ccccc6c6cc7oc8ccccc8c7cc64)cc4c6ccccc6oc54)n3)cc2)cc1 ; c1ccc(-c2nc(-c3ccc4ccccc4c3)nc(-c3ccc4c(c3)c(-n3c5cccc6c5c5c7c(cccc7ccc53)-c3ccccc3-6)cc3c5ccccc5oc43)n2)cc1]

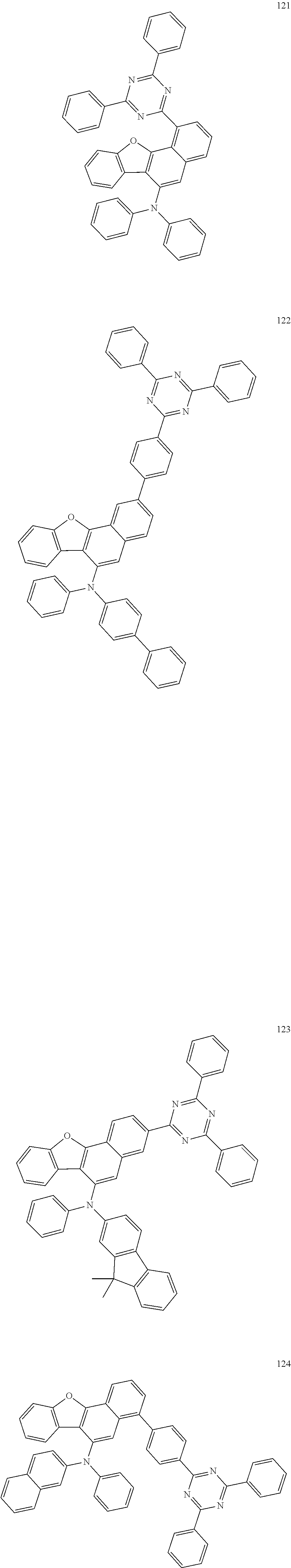 [SMILES: c1ccc(-c2nc(-c3ccccc3)nc(-c3cccc4cc(N(c5ccccc5)c5ccccc5)c5c6ccccc6oc5c34)n2)cc1 ; c1ccc(-c2ccc(N(c3ccccc3)c3cc4ccc(-c5ccc(-c6nc(-c7ccccc7)nc(-c7ccccc7)n6)cc5)cc4c4oc5ccccc5c34)cc2)cc1 ; CC1(C)c2ccccc2-c2ccc(N(c3ccccc3)c3cc4cc(-c5nc(-c6ccccc6)nc(-c6ccccc6)n5)ccc4c4oc5ccccc5c34)cc21 ; c1ccc(-c2nc(-c3ccccc3)nc(-c3ccc(-c4cccc5c4cc(N(c4ccccc4)c4ccc6ccccc6c4)c4c6ccccc6oc54)cc3)n2)cc1]

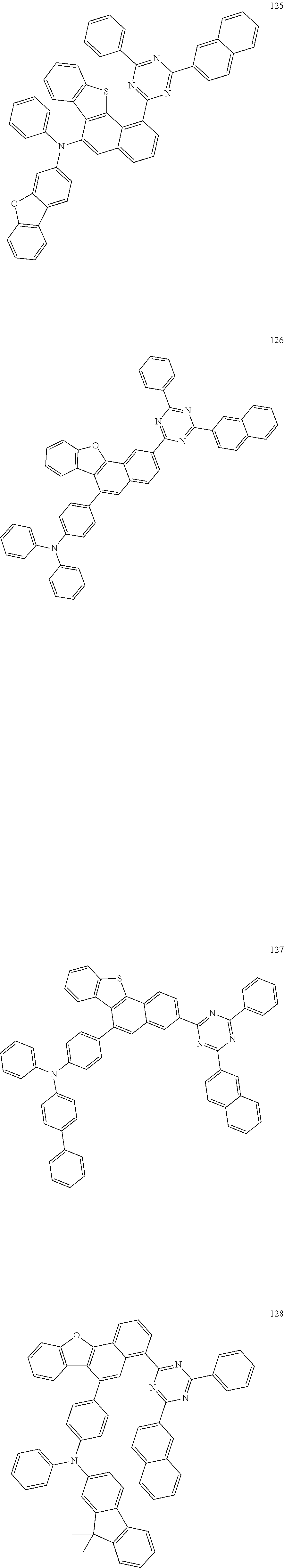 [SMILES: c1ccc(-c2nc(-c3ccc4ccccc4c3)nc(-c3cccc4cc(N(c5ccccc5)c5ccc6c(c5)oc5ccccc56)c5c6ccccc6sc5c34)n2)cc1 ; c1ccc(-c2nc(-c3ccc4ccccc4c3)nc(-c3ccc4cc(-c5ccc(N(c6ccccc6)c6ccccc6)cc5)c5c6ccccc6oc5c4c3)n2)cc1 ; c1ccc(-c2ccc(N(c3ccccc3)c3ccc(-c4cc5cc(-c6nc(-c7ccccc7)nc(-c7ccc8ccccc8c7)n6)ccc5c5sc6ccccc6c45)cc3)cc2)cc1 ; CC1(C)c2ccccc2-c2ccc(N(c3ccccc3)c3ccc(-c4cc5c(-c6nc(-c7ccccc7)nc(-c7ccc8ccccc8c7)n6)cccc5c5oc6ccccc6c45)cc3)cc21]

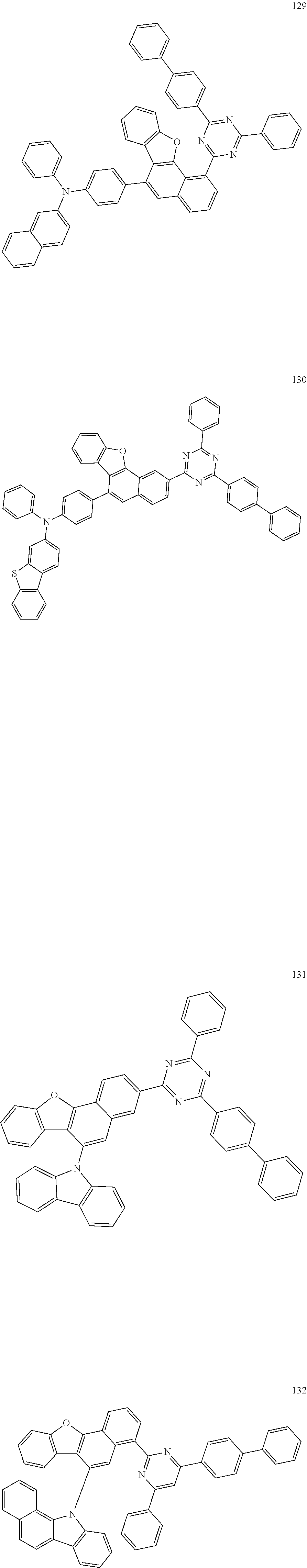 [SMILES: c1ccc(-c2ccc(-c3nc(-c4ccccc4)nc(-c4cccc5cc(-c6ccc(N(c7ccccc7)c7ccc8ccccc8c7)cc6)c6c7ccccc7oc6c45)n3)cc2)cc1 ; c1ccc(-c2ccc(-c3nc(-c4ccccc4)nc(-c4ccc5cc(-c6ccc(N(c7ccccc7)c7ccc8c(c7)sc7ccccc78)cc6)c6c7ccccc7oc6c5c4)n3)cc2)cc1 ; c1ccc(-c2ccc(-c3nc(-c4ccccc4)nc(-c4ccc5c(c4)cc(-n4c6ccccc6c6ccccc64)c4c6ccccc6oc54)n3)cc2)cc1 ; c1ccc(-c2ccc(-c3cc(-c4ccccc4)nc(-c4cccc5c4cc(-n4c6ccccc6c6ccc7ccccc7c64)c4c6ccccc6oc54)n3)cc2)cc1]

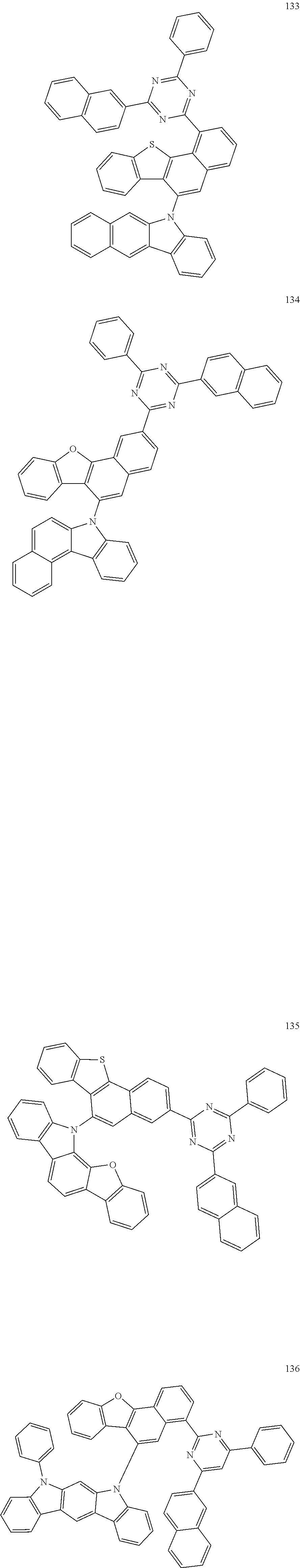 [SMILES: c1ccc(-c2nc(-c3ccc4ccccc4c3)nc(-c3cccc4cc(-n5c6ccccc6c6cc7ccccc7cc65)c5c6ccccc6sc5c34)n2)cc1 ; c1ccc(-c2nc(-c3ccc4ccccc4c3)nc(-c3ccc4cc(-n5c6ccccc6c6c7ccccc7ccc65)c5c6ccccc6oc5c4c3)n2)cc1 ; c1ccc(-c2nc(-c3ccc4ccccc4c3)nc(-c3ccc4c(c3)cc(-n3c5ccccc5c5ccc6c7ccccc7oc6c53)c3c5ccccc5sc43)n2)cc1 ; c1ccc(-c2cc(-c3ccc4ccccc4c3)nc(-c3cccc4c3cc(-n3c5ccccc5c5cc6c7ccccc7n(-c7ccccc7)c6cc53)c3c5ccccc5oc43)n2)cc1]

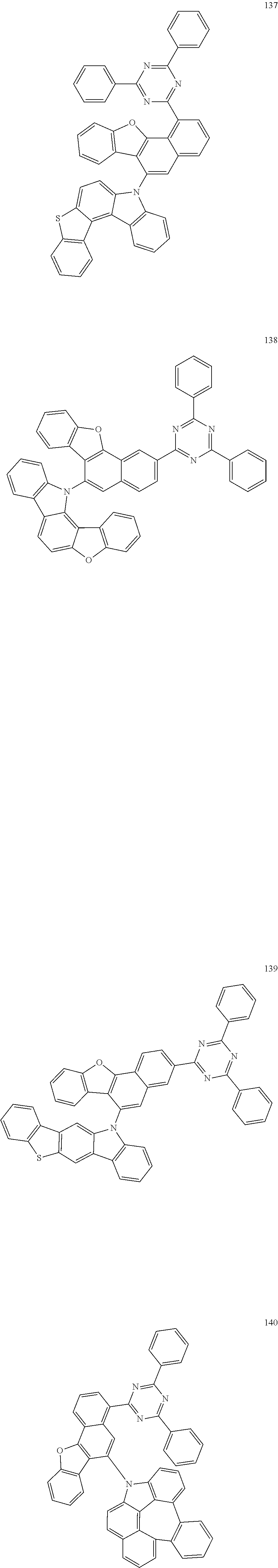 [SMILES: c1ccc(-c2nc(-c3ccccc3)nc(-c3cccc4cc(-n5c6ccccc6c6c7c(ccc65)sc5ccccc57)c5c6ccccc6oc5c34)n2)cc1 ; c1ccc(-c2nc(-c3ccccc3)nc(-c3ccc4cc(-n5c6ccccc6c6ccc7oc8ccccc8c7c65)c5c6ccccc6oc5c4c3)n2)cc1 ; c1ccc(-c2nc(-c3ccccc3)nc(-c3ccc4c(c3)cc(-n3c5ccccc5c5cc6sc7ccccc7c6cc53)c3c5ccccc5oc43)n2)cc1 ; c1ccc(-c2nc(-c3ccccc3)nc(-c3cccc4c3cc(-n3c5cccc6c5c5c7c(cccc7ccc53)-c3ccccc3-6)c3c5ccccc5oc43)n2)cc1]

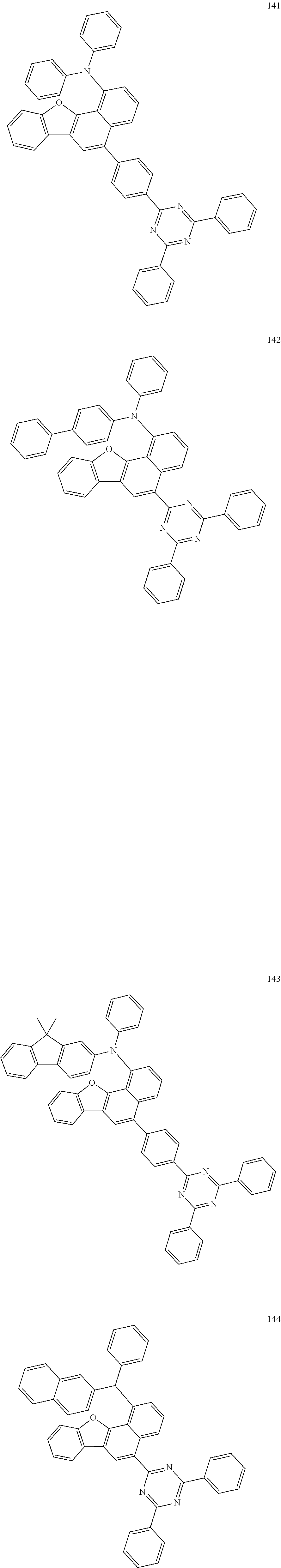 [SMILES: c1ccc(-c2nc(-c3ccccc3)nc(-c3ccc(-c4cc5c6ccccc6oc5c5c(N(c6ccccc6)c6ccccc6)cccc45)cc3)n2)cc1 ; c1ccc(-c2ccc(N(c3ccccc3)c3cccc4c(-c5nc(-c6ccccc6)nc(-c6ccccc6)n5)cc5c6ccccc6oc5c34)cc2)cc1 ; CC1(C)c2ccccc2-c2ccc(N(c3ccccc3)c3cccc4c(-c5ccc(-c6nc(-c7ccccc7)nc(-c7ccccc7)n6)cc5)cc5c6ccccc6oc5c34)cc21 ; c1ccc(-c2nc(-c3ccccc3)nc(-c3cc4c5ccccc5oc4c4c(C(c5ccccc5)c5ccc6ccccc6c5)cccc34)n2)cc1]

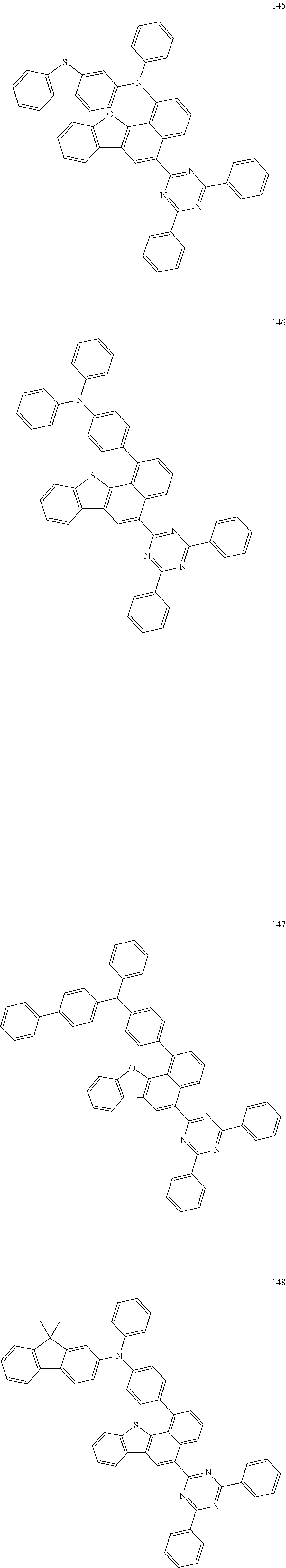 [SMILES: c1ccc(-c2nc(-c3ccccc3)nc(-c3cc4c5ccccc5oc4c4c(N(c5ccccc5)c5ccc6c(c5)sc5ccccc56)cccc34)n2)cc1 ; c1ccc(-c2nc(-c3ccccc3)nc(-c3cc4c5ccccc5sc4c4c(-c5ccc(N(c6ccccc6)c6ccccc6)cc5)cccc34)n2)cc1 ; c1ccc(-c2ccc(C(c3ccccc3)c3ccc(-c4cccc5c(-c6nc(-c7ccccc7)nc(-c7ccccc7)n6)cc6c7ccccc7oc6c45)cc3)cc2)cc1 ; CC1(C)c2ccccc2-c2ccc(N(c3ccccc3)c3ccc(-c4cccc5c(-c6nc(-c7ccccc7)nc(-c7ccccc7)n6)cc6c7ccccc7sc6c45)cc3)cc21]

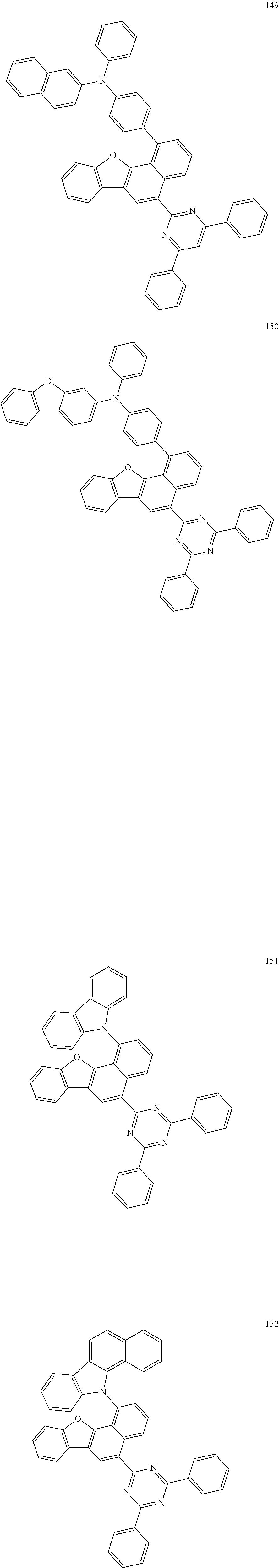 [SMILES: c1ccc(-c2cc(-c3ccccc3)nc(-c3cc4c5ccccc5oc4c4c(-c5ccc(N(c6ccccc6)c6ccc7ccccc7c6)cc5)cccc34)n2)cc1 ; c1ccc(-c2nc(-c3ccccc3)nc(-c3cc4c5ccccc5oc4c4c(-c5ccc(N(c6ccccc6)c6ccc7c(c6)oc6ccccc67)cc5)cccc34)n2)cc1 ; c1ccc(-c2nc(-c3ccccc3)nc(-c3cc4c5ccccc5oc4c4c(-n5c6ccccc6c6ccccc65)cccc34)n2)cc1 ; c1ccc(-c2nc(-c3ccccc3)nc(-c3cc4c5ccccc5oc4c4c(-n5c6ccccc6c6ccc7ccccc7c65)cccc34)n2)cc1]

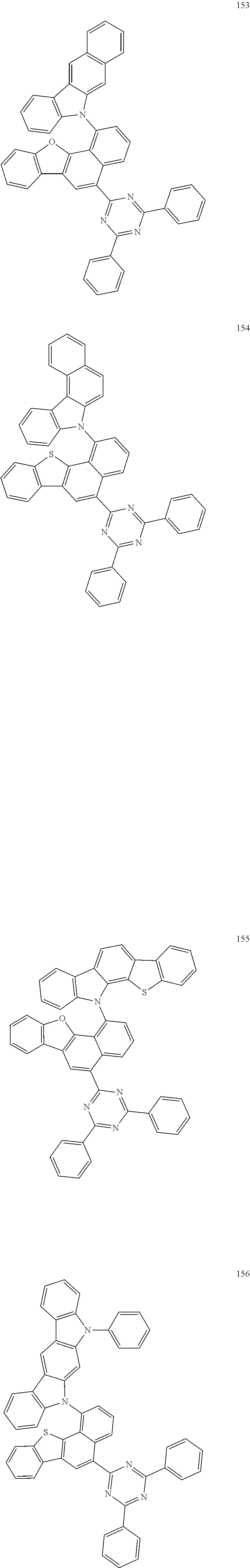 [SMILES: c1ccc(-c2nc(-c3ccccc3)nc(-c3cc4c5ccccc5oc4c4c(-n5c6ccccc6c6cc7ccccc7cc65)cccc34)n2)cc1 ; c1ccc(-c2nc(-c3ccccc3)nc(-c3cc4c5ccccc5sc4c4c(-n5c6ccccc6c6c7ccccc7ccc65)cccc34)n2)cc1 ; c1ccc(-c2nc(-c3ccccc3)nc(-c3cc4c5ccccc5oc4c4c(-n5c6ccccc6c6ccc7c8ccccc8sc7c65)cccc34)n2)cc1 ; c1ccc(-c2nc(-c3ccccc3)nc(-c3cc4c5ccccc5sc4c4c(-n5c6ccccc6c6cc7c8ccccc8n(-c8ccccc8)c7cc65)cccc34)n2)cc1]

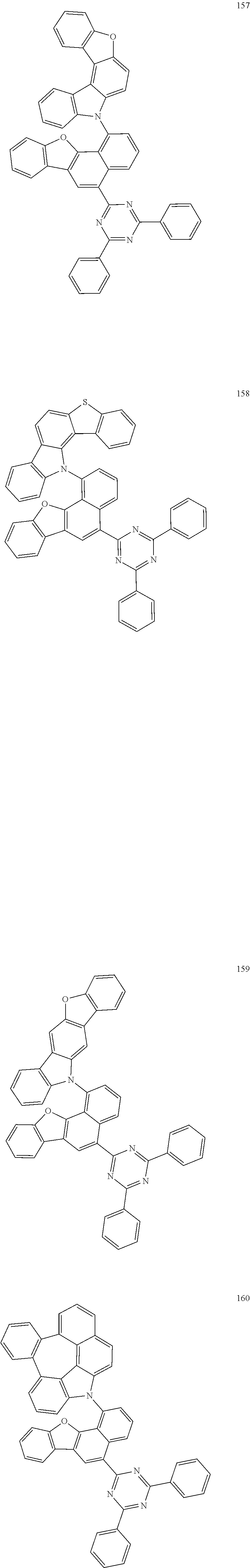 [SMILES: c1ccc(-c2nc(-c3ccccc3)nc(-c3cc4c5ccccc5oc4c4c(-n5c6ccccc6c6c7c(ccc65)oc5ccccc57)cccc34)n2)cc1 ; c1ccc(-c2nc(-c3ccccc3)nc(-c3cc4c5ccccc5oc4c4c(-n5c6ccccc6c6ccc7sc8ccccc8c7c65)cccc34)n2)cc1 ; c1ccc(-c2nc(-c3ccccc3)nc(-c3cc4c5ccccc5oc4c4c(-n5c6ccccc6c6cc7oc8ccccc8c7cc65)cccc34)n2)cc1 ; c1ccc(-c2nc(-c3ccccc3)nc(-c3cc4c5ccccc5oc4c4c(-n5c6cccc7c6c6c8c(cccc8ccc65)-c5ccccc5-7)cccc34)n2)cc1]

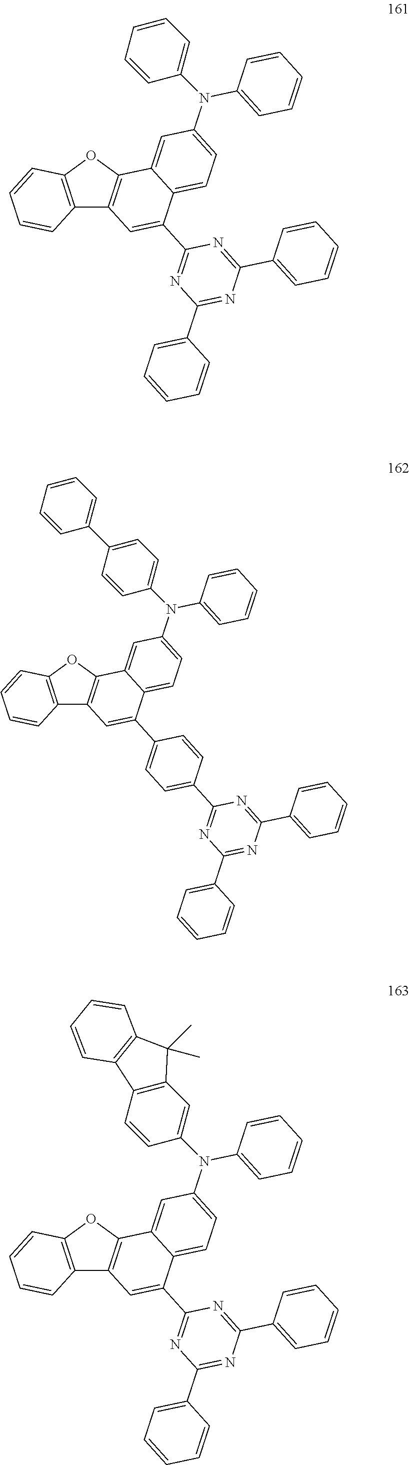 [SMILES: c1ccc(-c2nc(-c3ccccc3)nc(-c3cc4c5ccccc5oc4c4cc(N(c5ccccc5)c5ccccc5)ccc34)n2)cc1 ; c1ccc(-c2ccc(N(c3ccccc3)c3ccc4c(-c5ccc(-c6nc(-c7ccccc7)nc(-c7ccccc7)n6)cc5)cc5c6ccccc6oc5c4c3)cc2)cc1 ; CC1(C)c2ccccc2-c2ccc(N(c3ccccc3)c3ccc4c(-c5nc(-c6ccccc6)nc(-c6ccccc6)n5)cc5c6ccccc6oc5c4c3)cc21]

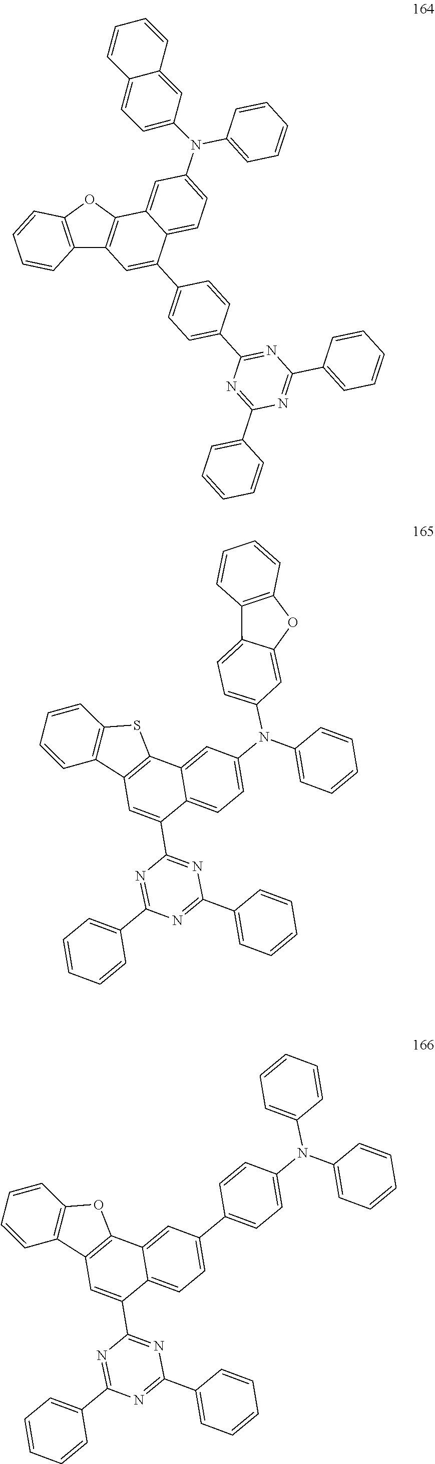 [SMILES: c1ccc(-c2nc(-c3ccccc3)nc(-c3ccc(-c4cc5c6ccccc6oc5c5cc(N(c6ccccc6)c6ccc7ccccc7c6)ccc45)cc3)n2)cc1 ; c1ccc(-c2nc(-c3ccccc3)nc(-c3cc4c5ccccc5sc4c4cc(N(c5ccccc5)c5ccc6c(c5)oc5ccccc56)ccc34)n2)cc1 ; c1ccc(-c2nc(-c3ccccc3)nc(-c3cc4c5ccccc5oc4c4cc(-c5ccc(N(c6ccccc6)c6ccccc6)cc5)ccc34)n2)cc1]

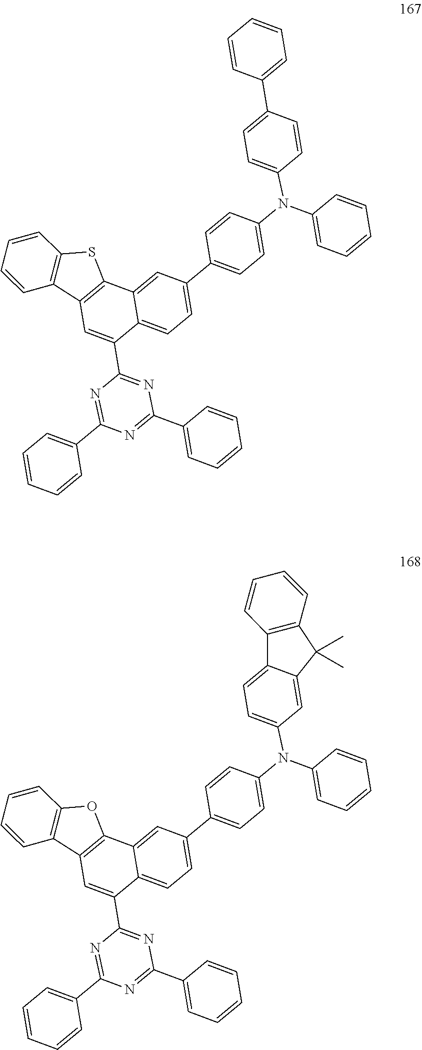 [SMILES: c1ccc(-c2ccc(N(c3ccccc3)c3ccc(-c4ccc5c(-c6nc(-c7ccccc7)nc(-c7ccccc7)n6)cc6c7ccccc7sc6c5c4)cc3)cc2)cc1 ; CC1(C)c2ccccc2-c2ccc(N(c3ccccc3)c3ccc(-c4ccc5c(-c6nc(-c7ccccc7)nc(-c7ccccc7)n6)cc6c7ccccc7oc6c5c4)cc3)cc21]

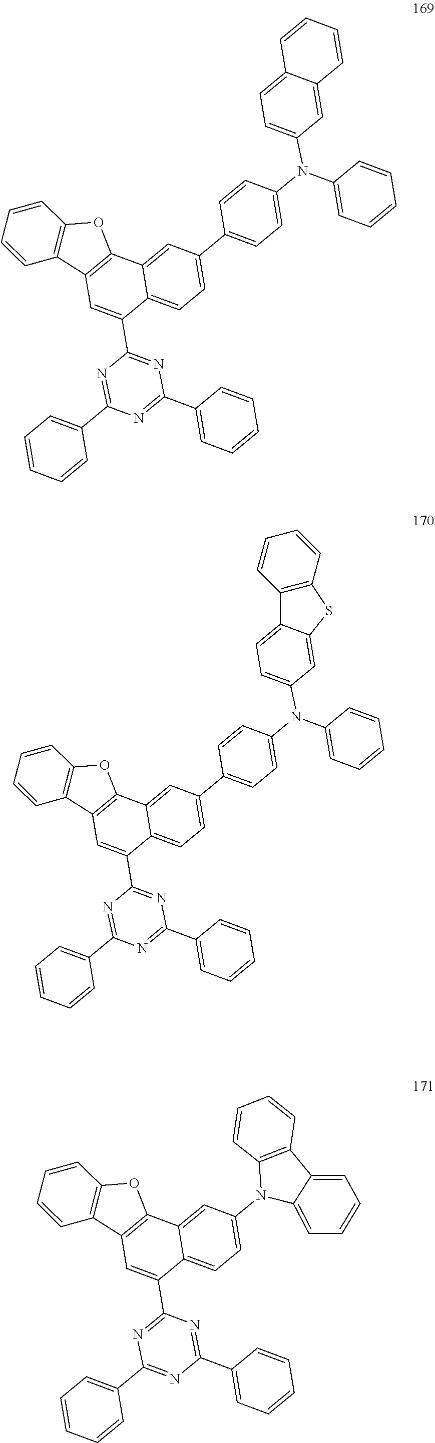 [SMILES: c1ccc(-c2nc(-c3ccccc3)nc(-c3cc4c5ccccc5oc4c4cc(-c5ccc(N(c6ccccc6)c6ccc7ccccc7c6)cc5)ccc34)n2)cc1 ; c1ccc(-c2nc(-c3ccccc3)nc(-c3cc4c5ccccc5oc4c4cc(-c5ccc(N(c6ccccc6)c6ccc7c(c6)sc6ccccc67)cc5)ccc34)n2)cc1 ; c1ccc(-c2nc(-c3ccccc3)nc(-c3cc4c5ccccc5oc4c4cc(-n5c6ccccc6c6ccccc65)ccc34)n2)cc1]

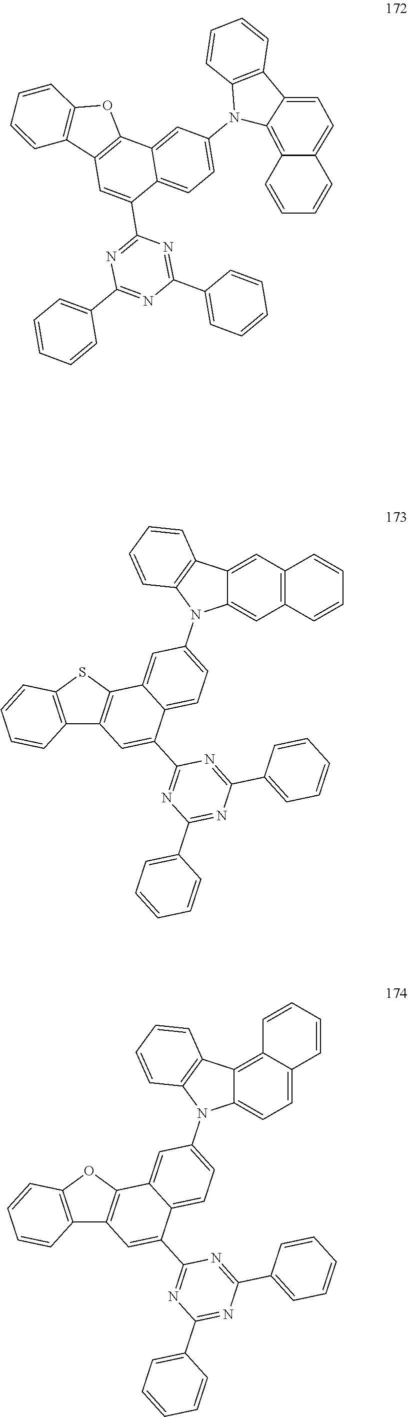 [SMILES: c1ccc(-c2nc(-c3ccccc3)nc(-c3cc4c5ccccc5oc4c4cc(-n5c6ccccc6c6ccc7ccccc7c65)ccc34)n2)cc1 ; c1ccc(-c2nc(-c3ccccc3)nc(-c3cc4c5ccccc5sc4c4cc(-n5c6ccccc6c6cc7ccccc7cc65)ccc34)n2)cc1 ; c1ccc(-c2nc(-c3ccccc3)nc(-c3cc4c5ccccc5oc4c4cc(-n5c6ccccc6c6c7ccccc7ccc65)ccc34)n2)cc1]

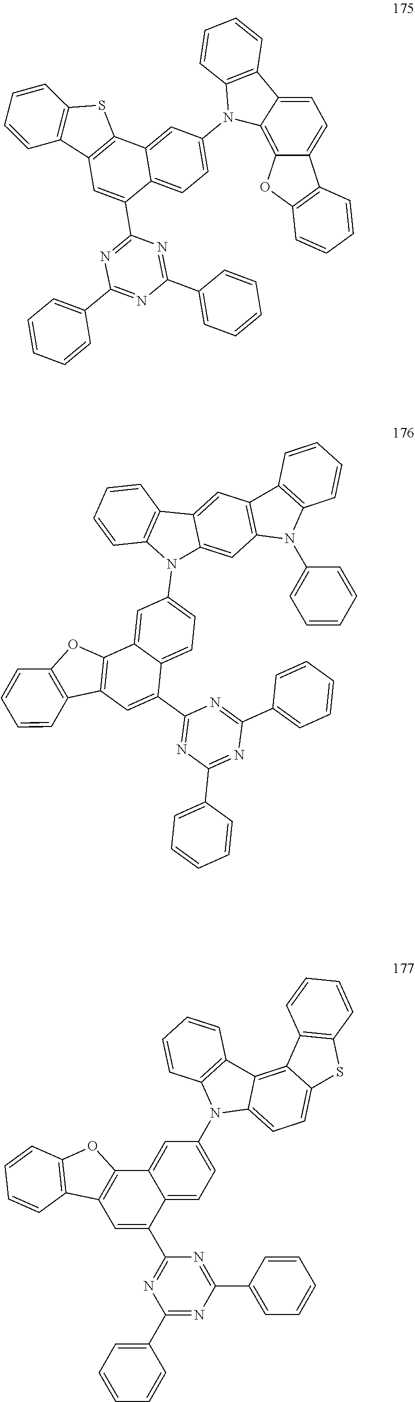 [SMILES: c1ccc(-c2nc(-c3ccccc3)nc(-c3cc4c5ccccc5sc4c4cc(-n5c6ccccc6c6ccc7c8ccccc8oc7c65)ccc34)n2)cc1 ; c1ccc(-c2nc(-c3ccccc3)nc(-c3cc4c5ccccc5oc4c4cc(-n5c6ccccc6c6cc7c8ccccc8n(-c8ccccc8)c7cc65)ccc34)n2)cc1 ; c1ccc(-c2nc(-c3ccccc3)nc(-c3cc4c5ccccc5oc4c4cc(-n5c6ccccc6c6c7c(ccc65)sc5ccccc57)ccc34)n2)cc1]

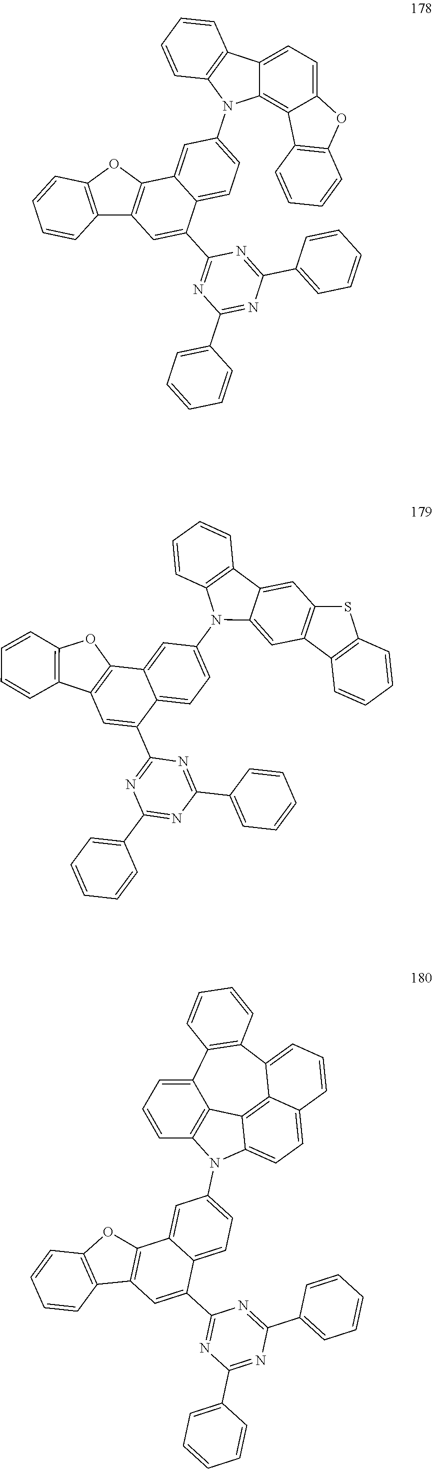 [SMILES: c1ccc(-c2nc(-c3ccccc3)nc(-c3cc4c5ccccc5oc4c4cc(-n5c6ccccc6c6ccc7oc8ccccc8c7c65)ccc34)n2)cc1 ; c1ccc(-c2nc(-c3ccccc3)nc(-c3cc4c5ccccc5oc4c4cc(-n5c6ccccc6c6cc7sc8ccccc8c7cc65)ccc34)n2)cc1 ; c1ccc(-c2nc(-c3ccccc3)nc(-c3cc4c5ccccc5oc4c4cc(-n5c6cccc7c6c6c8c(cccc8ccc65)-c5ccccc5-7)ccc34)n2)cc1]

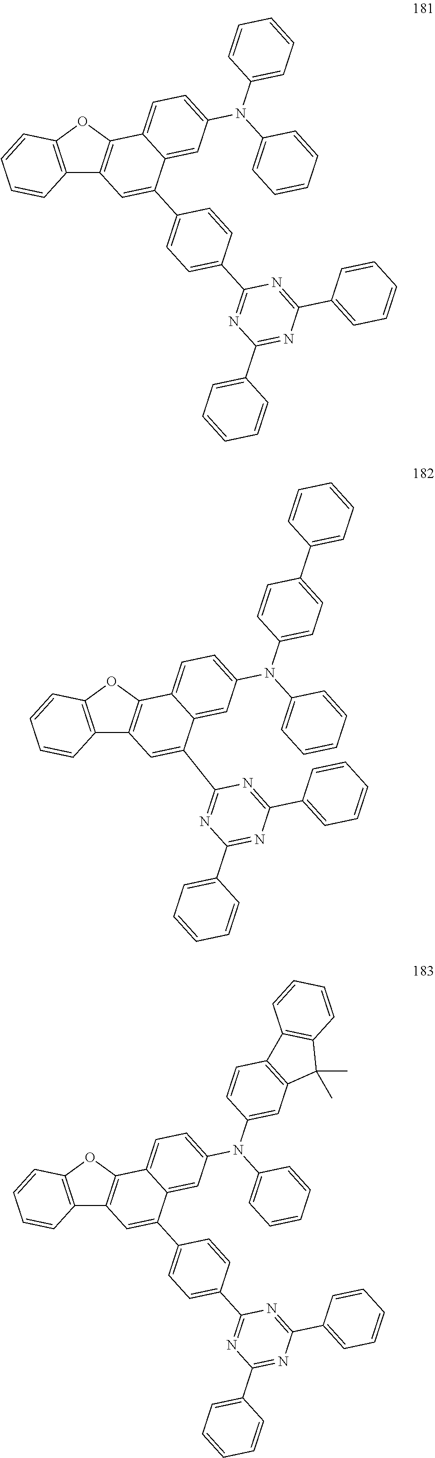 [SMILES: c1ccc(-c2nc(-c3ccccc3)nc(-c3ccc(-c4cc5c6ccccc6oc5c5ccc(N(c6ccccc6)c6ccccc6)cc45)cc3)n2)cc1 ; c1ccc(-c2ccc(N(c3ccccc3)c3ccc4c(c3)c(-c3nc(-c5ccccc5)nc(-c5ccccc5)n3)cc3c5ccccc5oc43)cc2)cc1 ; CC1(C)c2ccccc2-c2ccc(N(c3ccccc3)c3ccc4c(c3)c(-c3ccc(-c5nc(-c6ccccc6)nc(-c6ccccc6)n5)cc3)cc3c5ccccc5oc43)cc21]

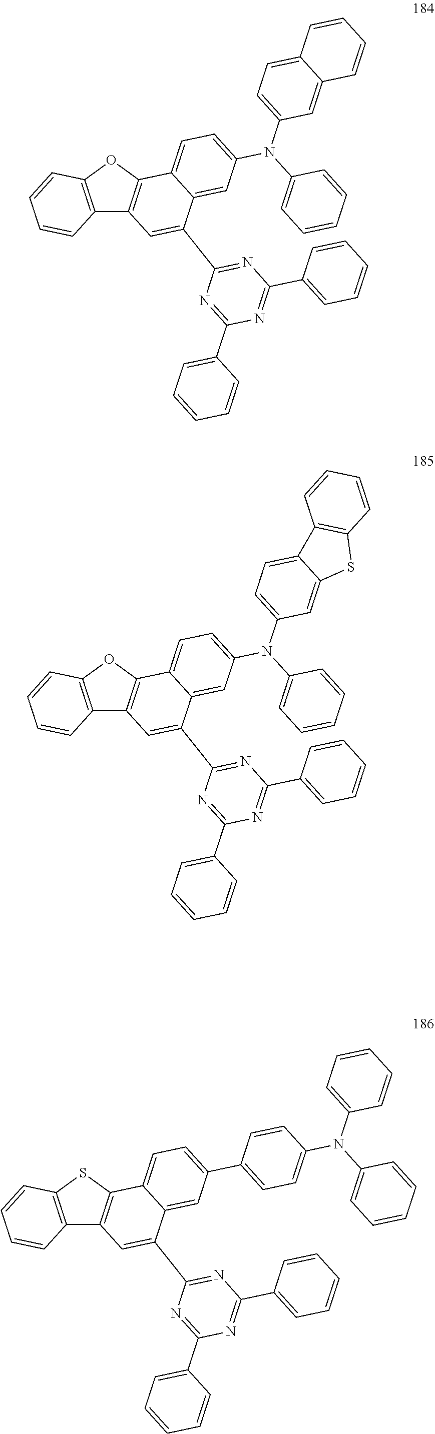 [SMILES: c1ccc(-c2nc(-c3ccccc3)nc(-c3cc4c5ccccc5oc4c4ccc(N(c5ccccc5)c5ccc6ccccc6c5)cc34)n2)cc1 ; c1ccc(-c2nc(-c3ccccc3)nc(-c3cc4c5ccccc5oc4c4ccc(N(c5ccccc5)c5ccc6c(c5)sc5ccccc56)cc34)n2)cc1 ; c1ccc(-c2nc(-c3ccccc3)nc(-c3cc4c5ccccc5sc4c4ccc(-c5ccc(N(c6ccccc6)c6ccccc6)cc5)cc34)n2)cc1]

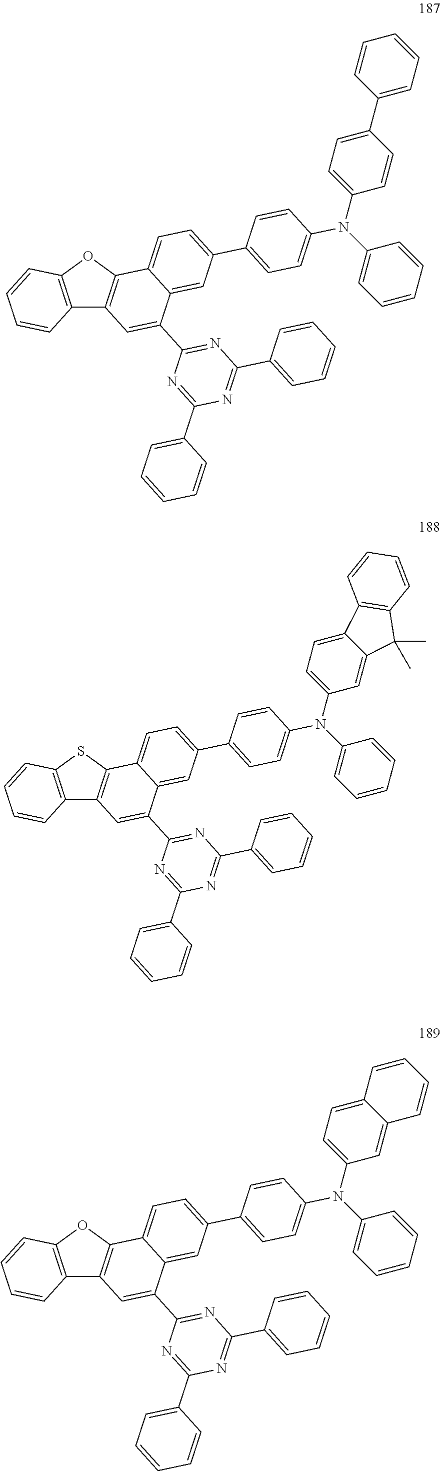 [SMILES: c1ccc(-c2ccc(N(c3ccccc3)c3ccc(-c4ccc5c(c4)c(-c4nc(-c6ccccc6)nc(-c6ccccc6)n4)cc4c6ccccc6oc54)cc3)cc2)cc1 ; CC1(C)c2ccccc2-c2ccc(N(c3ccccc3)c3ccc(-c4ccc5c(c4)c(-c4nc(-c6ccccc6)nc(-c6ccccc6)n4)cc4c6ccccc6sc54)cc3)cc21 ; c1ccc(-c2nc(-c3ccccc3)nc(-c3cc4c5ccccc5oc4c4ccc(-c5ccc(N(c6ccccc6)c6ccc7ccccc7c6)cc5)cc34)n2)cc1]

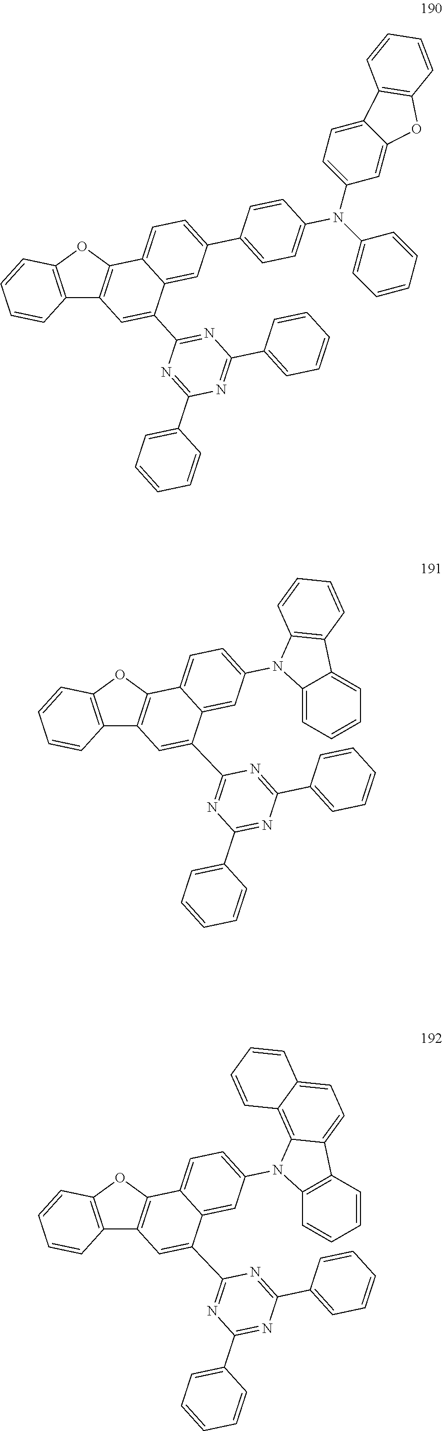 [SMILES: c1ccc(-c2nc(-c3ccccc3)nc(-c3cc4c5ccccc5oc4c4ccc(-c5ccc(N(c6ccccc6)c6ccc7c(c6)oc6ccccc67)cc5)cc34)n2)cc1 ; c1ccc(-c2nc(-c3ccccc3)nc(-c3cc4c5ccccc5oc4c4ccc(-n5c6ccccc6c6ccccc65)cc34)n2)cc1 ; c1ccc(-c2nc(-c3ccccc3)nc(-c3cc4c5ccccc5oc4c4ccc(-n5c6ccccc6c6ccc7ccccc7c65)cc34)n2)cc1]

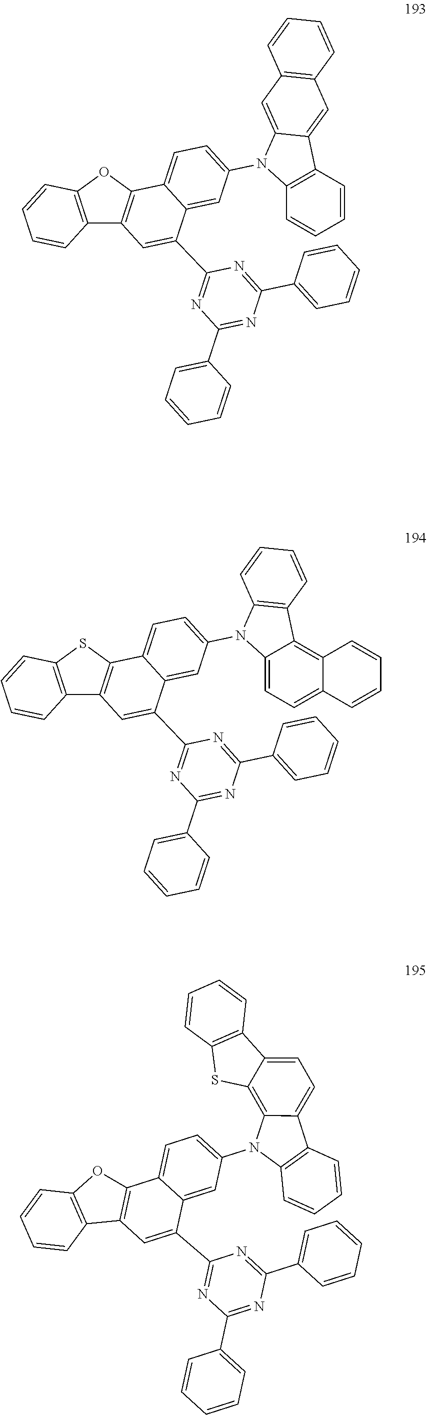 [SMILES: c1ccc(-c2nc(-c3ccccc3)nc(-c3cc4c5ccccc5oc4c4ccc(-n5c6ccccc6c6cc7ccccc7cc65)cc34)n2)cc1 ; c1ccc(-c2nc(-c3ccccc3)nc(-c3cc4c5ccccc5sc4c4ccc(-n5c6ccccc6c6c7ccccc7ccc65)cc34)n2)cc1 ; c1ccc(-c2nc(-c3ccccc3)nc(-c3cc4c5ccccc5oc4c4ccc(-n5c6ccccc6c6ccc7c8ccccc8sc7c65)cc34)n2)cc1]

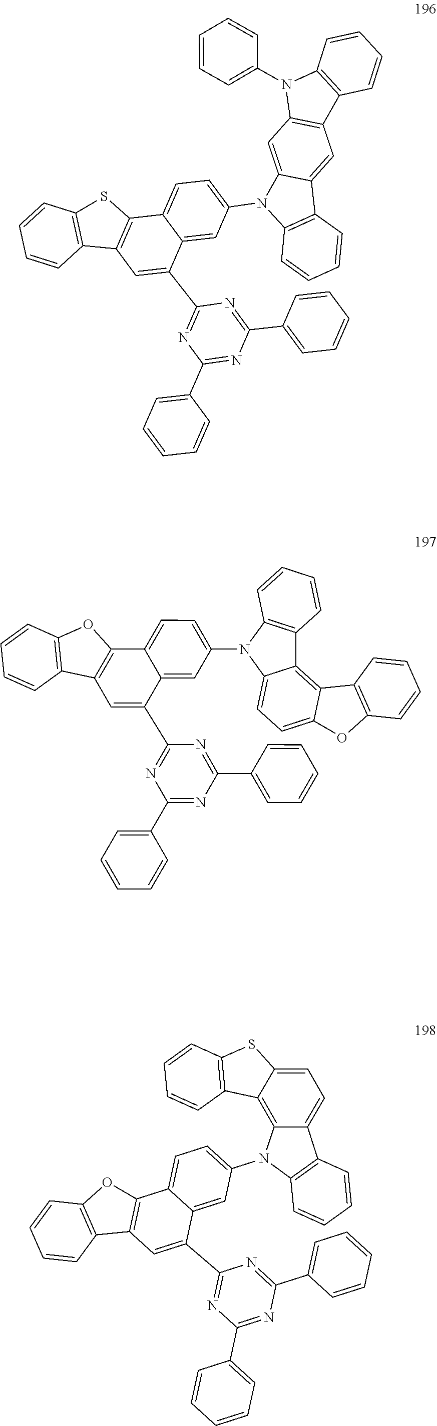 [SMILES: c1ccc(-c2nc(-c3ccccc3)nc(-c3cc4c5ccccc5sc4c4ccc(-n5c6ccccc6c6cc7c8ccccc8n(-c8ccccc8)c7cc65)cc34)n2)cc1 ; c1ccc(-c2nc(-c3ccccc3)nc(-c3cc4c5ccccc5oc4c4ccc(-n5c6ccccc6c6c7c(ccc65)oc5ccccc57)cc34)n2)cc1 ; c1ccc(-c2nc(-c3ccccc3)nc(-c3cc4c5ccccc5oc4c4ccc(-n5c6ccccc6c6ccc7sc8ccccc8c7c65)cc34)n2)cc1]

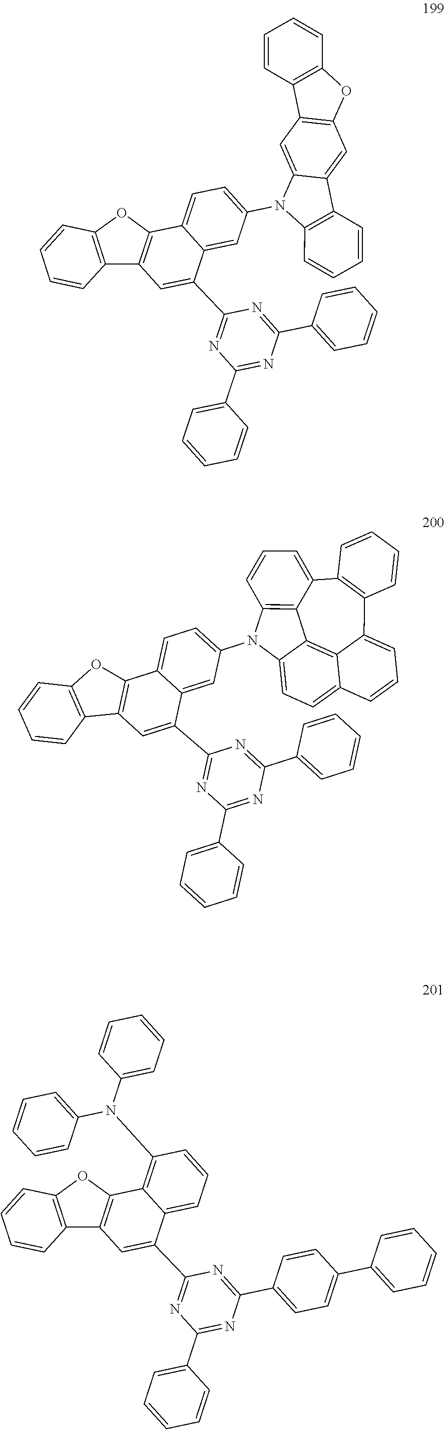 [SMILES: c1ccc(-c2nc(-c3ccccc3)nc(-c3cc4c5ccccc5oc4c4ccc(-n5c6ccccc6c6cc7oc8ccccc8c7cc65)cc34)n2)cc1 ; c1ccc(-c2nc(-c3ccccc3)nc(-c3cc4c5ccccc5oc4c4ccc(-n5c6cccc7c6c6c8c(cccc8ccc65)-c5ccccc5-7)cc34)n2)cc1 ; c1ccc(-c2ccc(-c3nc(-c4ccccc4)nc(-c4cc5c6ccccc6oc5c5c(N(c6ccccc6)c6ccccc6)cccc45)n3)cc2)cc1]

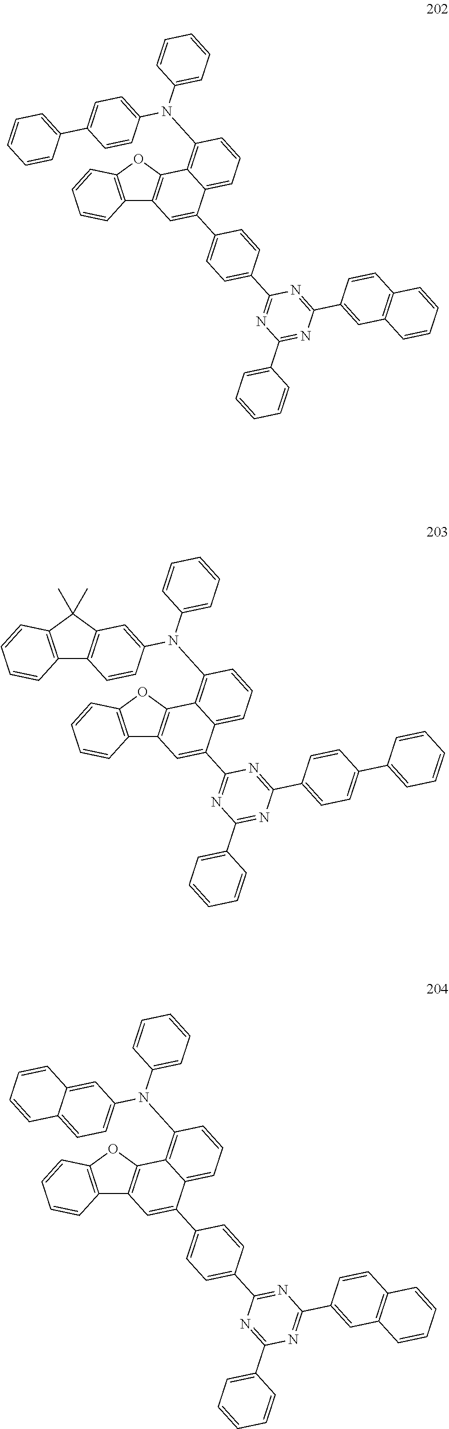 [SMILES: c1ccc(-c2ccc(N(c3ccccc3)c3cccc4c(-c5ccc(-c6nc(-c7ccccc7)nc(-c7ccc8ccccc8c7)n6)cc5)cc5c6ccccc6oc5c34)cc2)cc1 ; CC1(C)c2ccccc2-c2ccc(N(c3ccccc3)c3cccc4c(-c5nc(-c6ccccc6)nc(-c6ccc(-c7ccccc7)cc6)n5)cc5c6ccccc6oc5c34)cc21 ; c1ccc(-c2nc(-c3ccc(-c4cc5c6ccccc6oc5c5c(N(c6ccccc6)c6ccc7ccccc7c6)cccc45)cc3)nc(-c3ccc4ccccc4c3)n2)cc1]

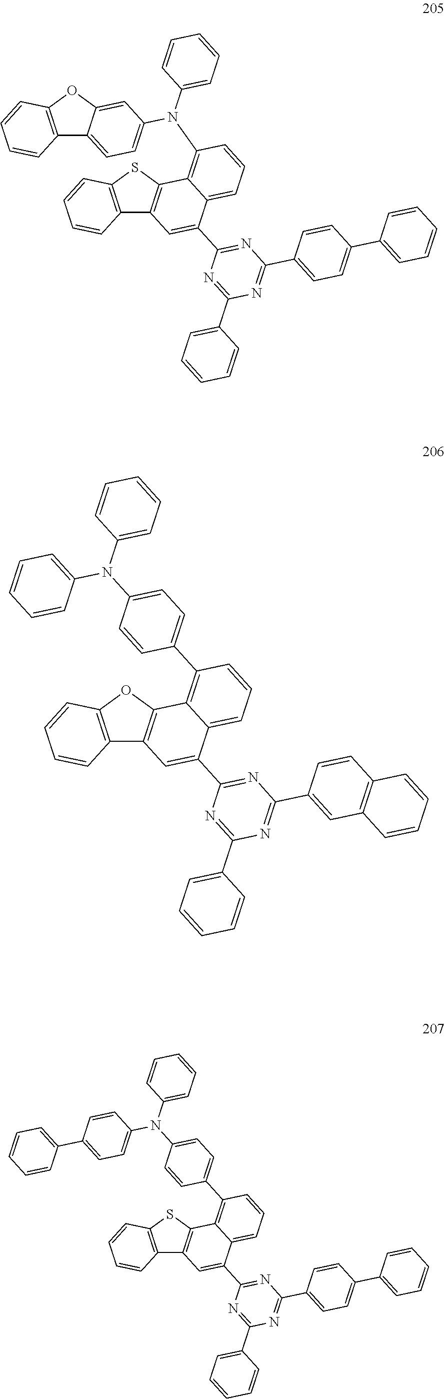 [SMILES: c1ccc(-c2ccc(-c3nc(-c4ccccc4)nc(-c4cc5c6ccccc6sc5c5c(N(c6ccccc6)c6ccc7c(c6)oc6ccccc67)cccc45)n3)cc2)cc1 ; c1ccc(-c2nc(-c3ccc4ccccc4c3)nc(-c3cc4c5ccccc5oc4c4c(-c5ccc(N(c6ccccc6)c6ccccc6)cc5)cccc34)n2)cc1 ; c1ccc(-c2ccc(-c3nc(-c4ccccc4)nc(-c4cc5c6ccccc6sc5c5c(-c6ccc(N(c7ccccc7)c7ccc(-c8ccccc8)cc7)cc6)cccc45)n3)cc2)cc1]

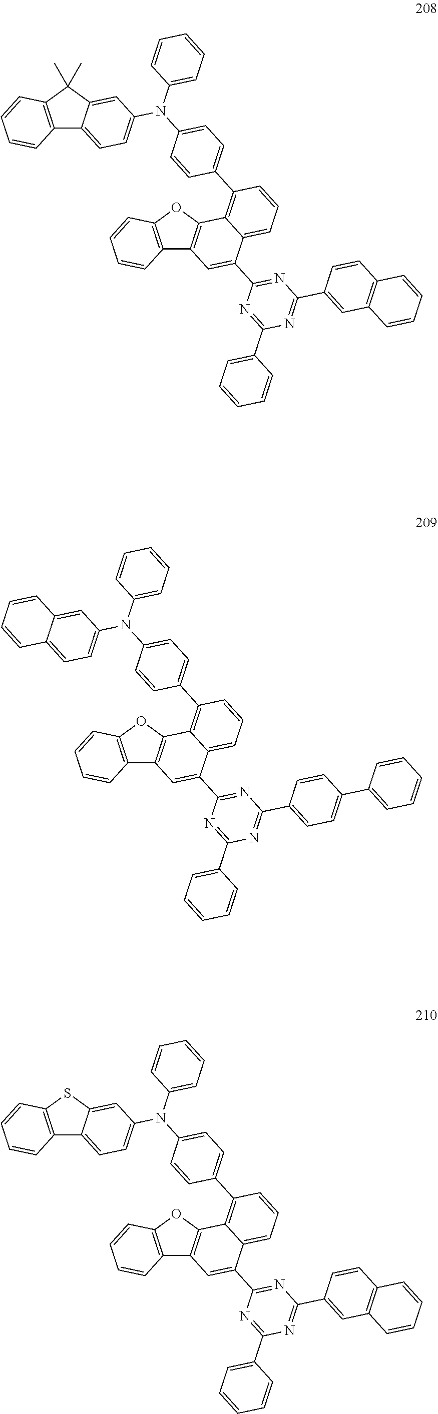 [SMILES: CC1(C)c2ccccc2-c2ccc(N(c3ccccc3)c3ccc(-c4cccc5c(-c6nc(-c7ccccc7)nc(-c7ccc8ccccc8c7)n6)cc6c7ccccc7oc6c45)cc3)cc21 ; c1ccc(-c2ccc(-c3nc(-c4ccccc4)nc(-c4cc5c6ccccc6oc5c5c(-c6ccc(N(c7ccccc7)c7ccc8ccccc8c7)cc6)cccc45)n3)cc2)cc1 ; c1ccc(-c2nc(-c3ccc4ccccc4c3)nc(-c3cc4c5ccccc5oc4c4c(-c5ccc(N(c6ccccc6)c6ccc7c(c6)sc6ccccc67)cc5)cccc34)n2)cc1]

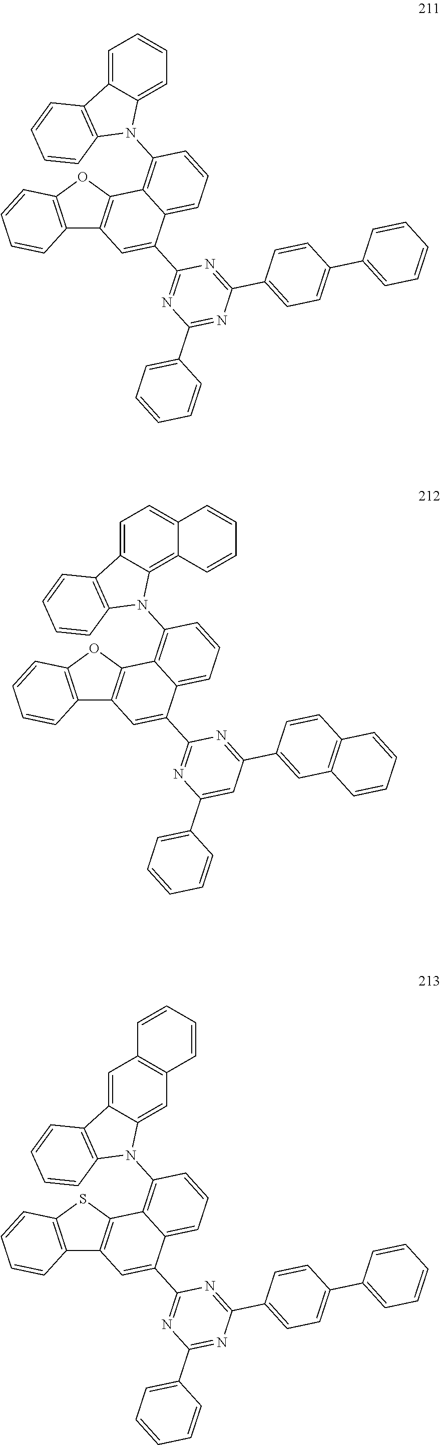 [SMILES: c1ccc(-c2ccc(-c3nc(-c4ccccc4)nc(-c4cc5c6ccccc6oc5c5c(-n6c7ccccc7c7ccccc76)cccc45)n3)cc2)cc1 ; c1ccc(-c2cc(-c3ccc4ccccc4c3)nc(-c3cc4c5ccccc5oc4c4c(-n5c6ccccc6c6ccc7ccccc7c65)cccc34)n2)cc1 ; c1ccc(-c2ccc(-c3nc(-c4ccccc4)nc(-c4cc5c6ccccc6sc5c5c(-n6c7ccccc7c7cc8ccccc8cc76)cccc45)n3)cc2)cc1]

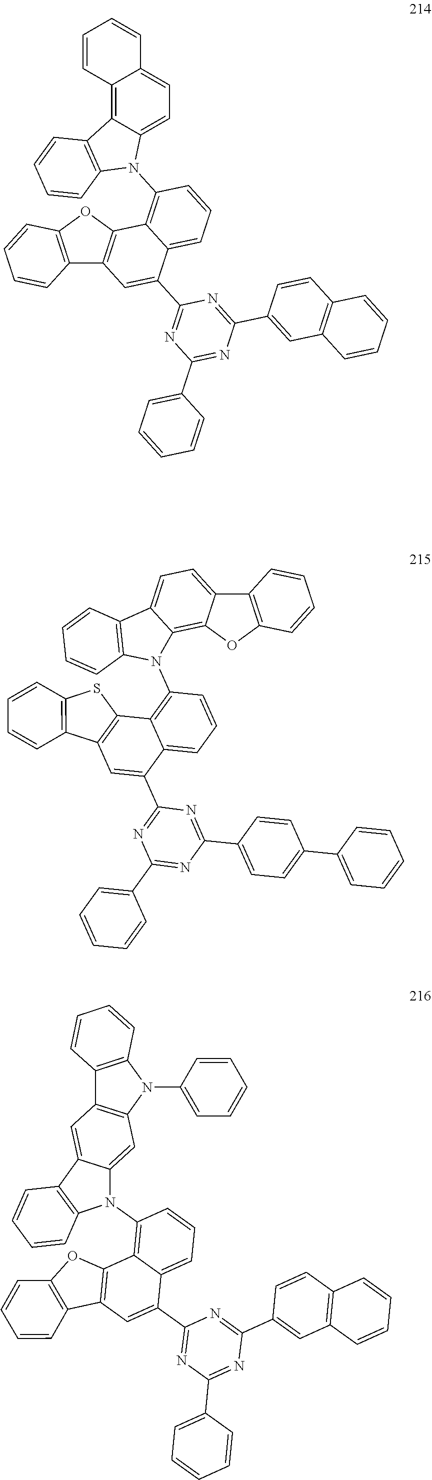 [SMILES: c1ccc(-c2nc(-c3ccc4ccccc4c3)nc(-c3cc4c5ccccc5oc4c4c(-n5c6ccccc6c6c7ccccc7ccc65)cccc34)n2)cc1 ; c1ccc(-c2ccc(-c3nc(-c4ccccc4)nc(-c4cc5c6ccccc6sc5c5c(-n6c7ccccc7c7ccc8c9ccccc9oc8c76)cccc45)n3)cc2)cc1 ; c1ccc(-c2nc(-c3ccc4ccccc4c3)nc(-c3cc4c5ccccc5oc4c4c(-n5c6ccccc6c6cc7c8ccccc8n(-c8ccccc8)c7cc65)cccc34)n2)cc1]

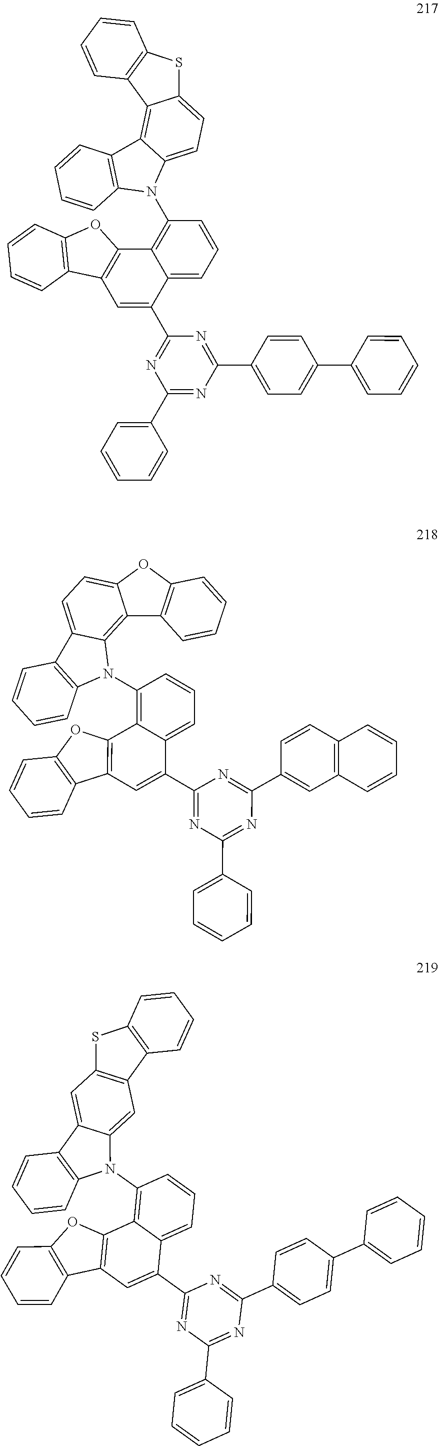 [SMILES: c1ccc(-c2ccc(-c3nc(-c4ccccc4)nc(-c4cc5c6ccccc6oc5c5c(-n6c7ccccc7c7c8c(ccc76)sc6ccccc68)cccc45)n3)cc2)cc1 ; c1ccc(-c2nc(-c3ccc4ccccc4c3)nc(-c3cc4c5ccccc5oc4c4c(-n5c6ccccc6c6ccc7oc8ccccc8c7c65)cccc34)n2)cc1 ; c1ccc(-c2ccc(-c3nc(-c4ccccc4)nc(-c4cc5c6ccccc6oc5c5c(-n6c7ccccc7c7cc8sc9ccccc9c8cc76)cccc45)n3)cc2)cc1]

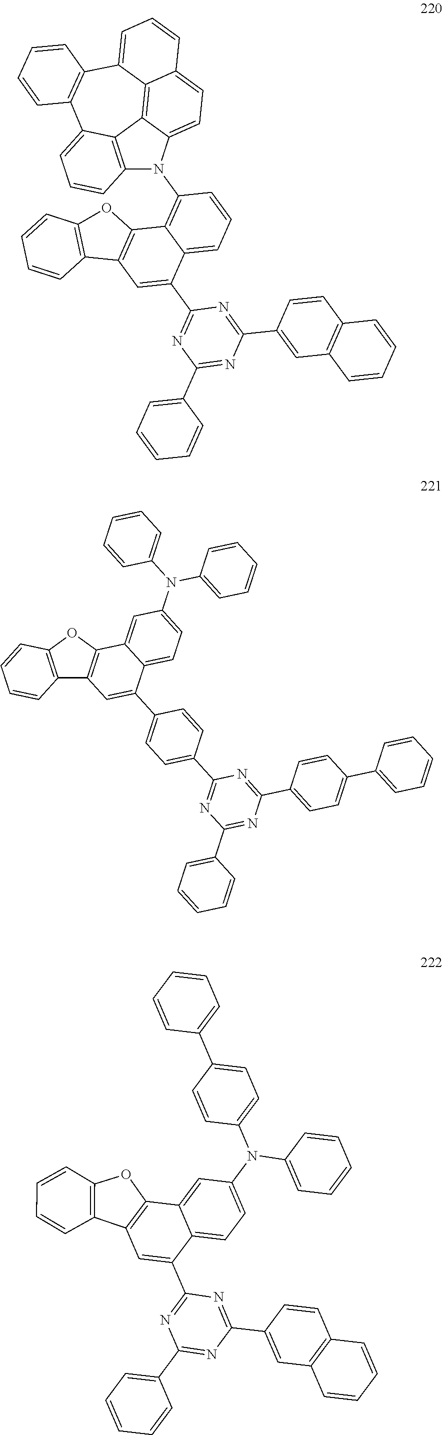 [SMILES: c1ccc(-c2nc(-c3ccc4ccccc4c3)nc(-c3cc4c5ccccc5oc4c4c(-n5c6cccc7c6c6c8c(cccc8ccc65)-c5ccccc5-7)cccc34)n2)cc1 ; c1ccc(-c2ccc(-c3nc(-c4ccccc4)nc(-c4ccc(-c5cc6c7ccccc7oc6c6cc(N(c7ccccc7)c7ccccc7)ccc56)cc4)n3)cc2)cc1 ; c1ccc(-c2ccc(N(c3ccccc3)c3ccc4c(-c5nc(-c6ccccc6)nc(-c6ccc7ccccc7c6)n5)cc5c6ccccc6oc5c4c3)cc2)cc1]

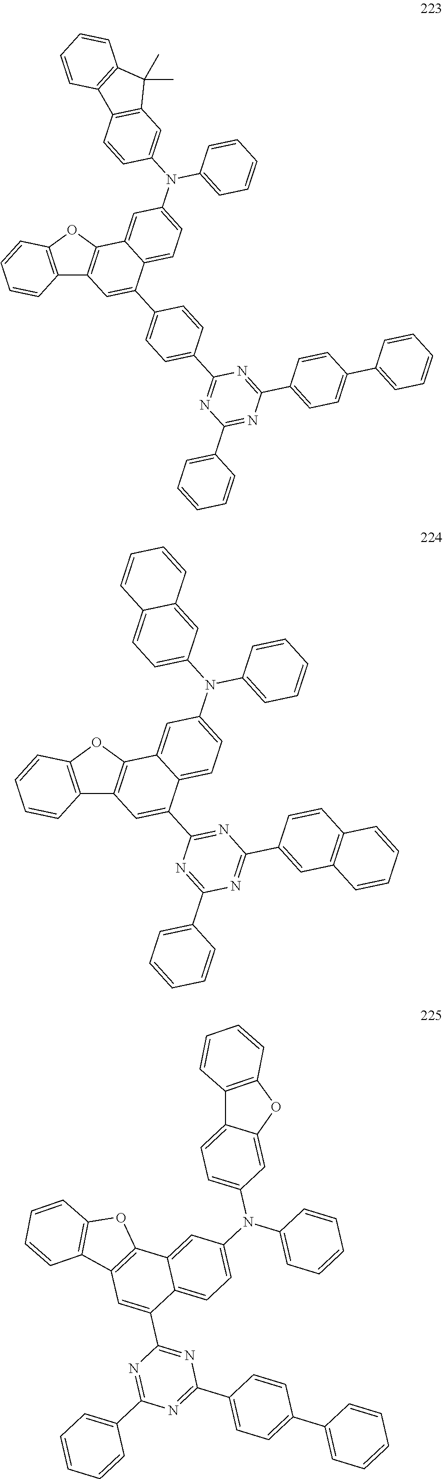 [SMILES: CC1(C)c2ccccc2-c2ccc(N(c3ccccc3)c3ccc4c(-c5ccc(-c6nc(-c7ccccc7)nc(-c7ccc(-c8ccccc8)cc7)n6)cc5)cc5c6ccccc6oc5c4c3)cc21 ; c1ccc(-c2nc(-c3ccc4ccccc4c3)nc(-c3cc4c5ccccc5oc4c4cc(N(c5ccccc5)c5ccc6ccccc6c5)ccc34)n2)cc1 ; c1ccc(-c2ccc(-c3nc(-c4ccccc4)nc(-c4cc5c6ccccc6oc5c5cc(N(c6ccccc6)c6ccc7c(c6)oc6ccccc67)ccc45)n3)cc2)cc1]

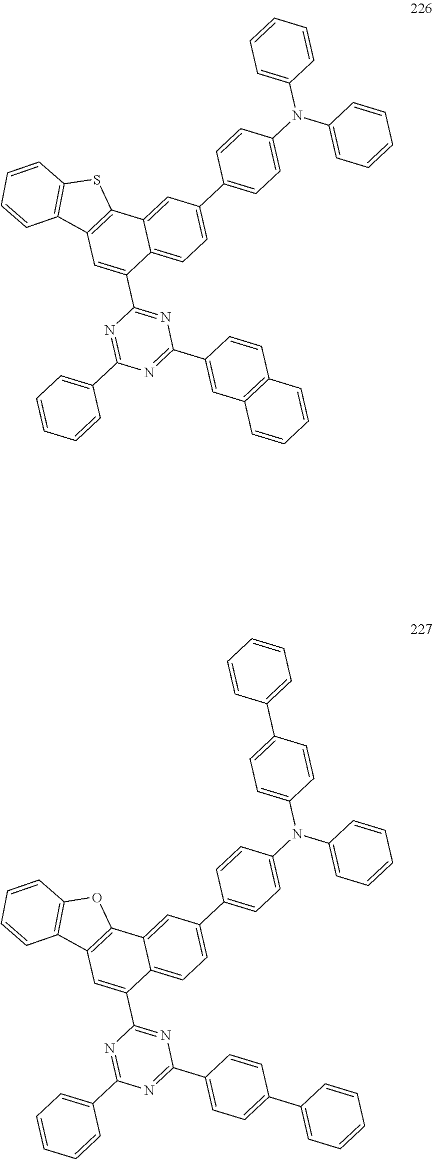 [SMILES: c1ccc(-c2nc(-c3ccc4ccccc4c3)nc(-c3cc4c5ccccc5sc4c4cc(-c5ccc(N(c6ccccc6)c6ccccc6)cc5)ccc34)n2)cc1 ; c1ccc(-c2ccc(-c3nc(-c4ccccc4)nc(-c4cc5c6ccccc6oc5c5cc(-c6ccc(N(c7ccccc7)c7ccc(-c8ccccc8)cc7)cc6)ccc45)n3)cc2)cc1]

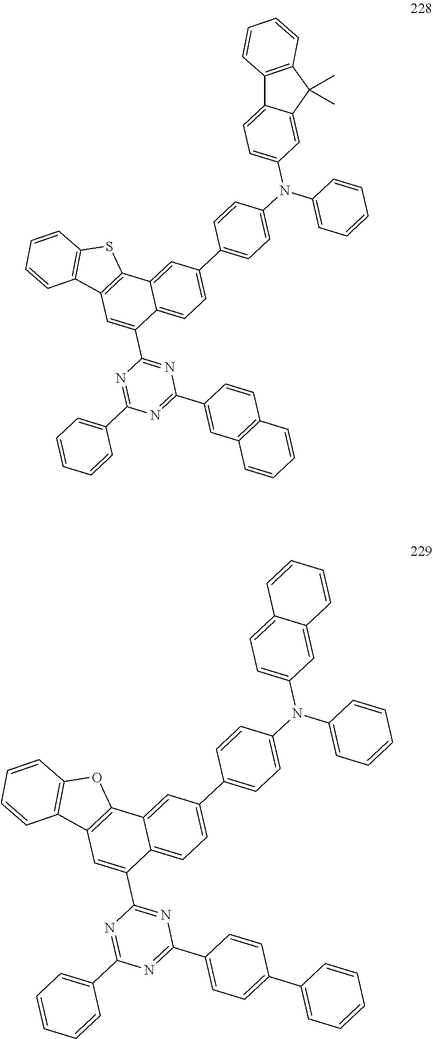 [SMILES: CC1(C)c2ccccc2-c2ccc(N(c3ccccc3)c3ccc(-c4ccc5c(-c6nc(-c7ccccc7)nc(-c7ccc8ccccc8c7)n6)cc6c7ccccc7sc6c5c4)cc3)cc21 ; c1ccc(-c2ccc(-c3nc(-c4ccccc4)nc(-c4cc5c6ccccc6oc5c5cc(-c6ccc(N(c7ccccc7)c7ccc8ccccc8c7)cc6)ccc45)n3)cc2)cc1]

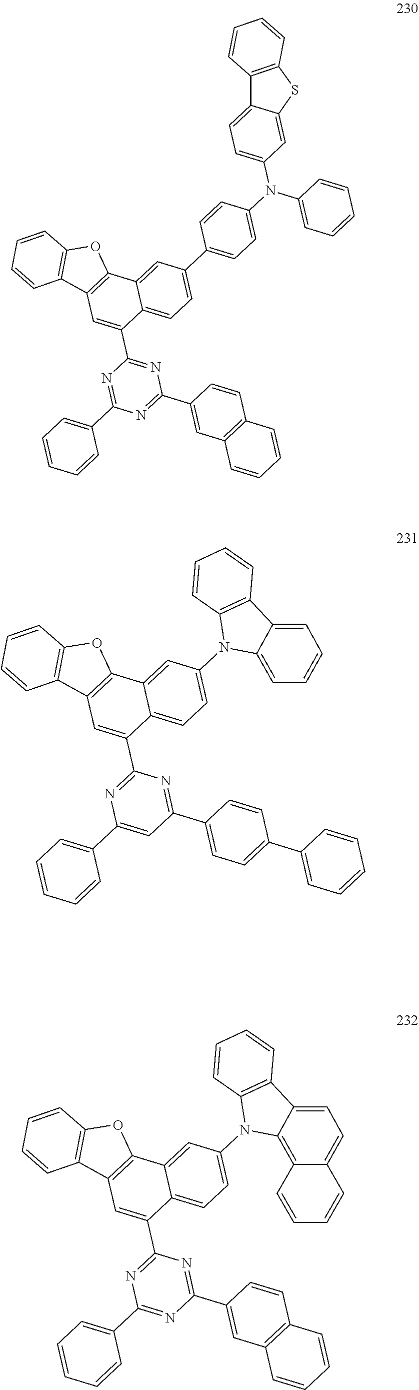 [SMILES: c1ccc(-c2nc(-c3ccc4ccccc4c3)nc(-c3cc4c5ccccc5oc4c4cc(-c5ccc(N(c6ccccc6)c6ccc7c(c6)sc6ccccc67)cc5)ccc34)n2)cc1 ; c1ccc(-c2ccc(-c3cc(-c4ccccc4)nc(-c4cc5c6ccccc6oc5c5cc(-n6c7ccccc7c7ccccc76)ccc45)n3)cc2)cc1 ; c1ccc(-c2nc(-c3ccc4ccccc4c3)nc(-c3cc4c5ccccc5oc4c4cc(-n5c6ccccc6c6ccc7ccccc7c65)ccc34)n2)cc1]

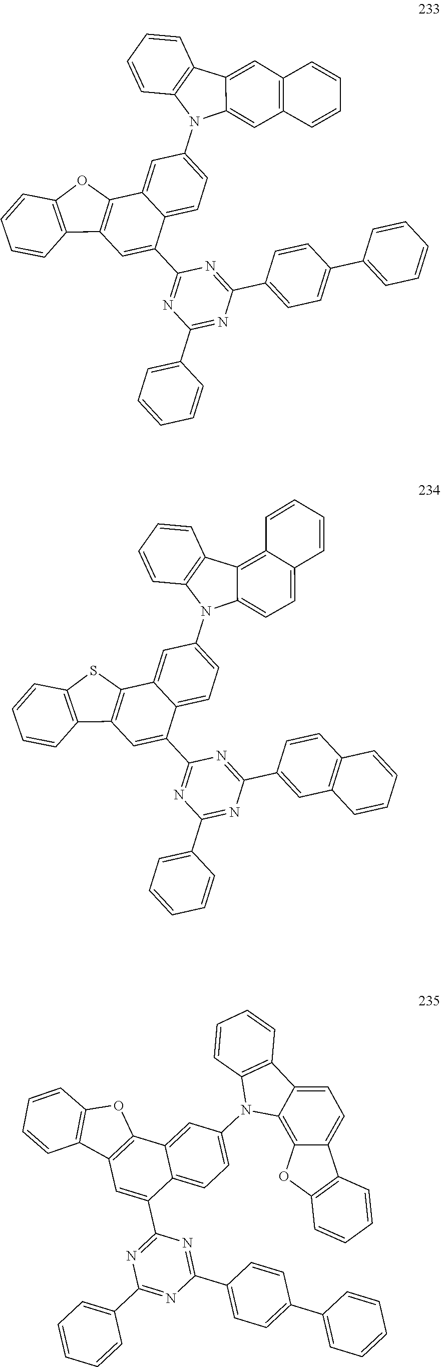 [SMILES: c1ccc(-c2ccc(-c3nc(-c4ccccc4)nc(-c4cc5c6ccccc6oc5c5cc(-n6c7ccccc7c7cc8ccccc8cc76)ccc45)n3)cc2)cc1 ; c1ccc(-c2nc(-c3ccc4ccccc4c3)nc(-c3cc4c5ccccc5sc4c4cc(-n5c6ccccc6c6c7ccccc7ccc65)ccc34)n2)cc1 ; c1ccc(-c2ccc(-c3nc(-c4ccccc4)nc(-c4cc5c6ccccc6oc5c5cc(-n6c7ccccc7c7ccc8c9ccccc9oc8c76)ccc45)n3)cc2)cc1]

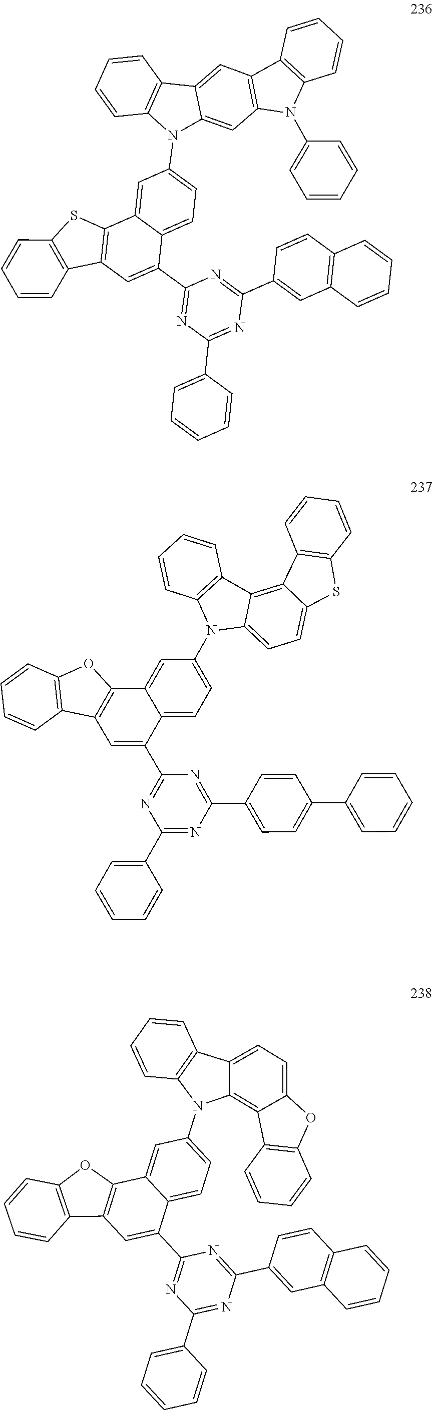 [SMILES: c1ccc(-c2nc(-c3ccc4ccccc4c3)nc(-c3cc4c5ccccc5sc4c4cc(-n5c6ccccc6c6cc7c8ccccc8n(-c8ccccc8)c7cc65)ccc34)n2)cc1 ; c1ccc(-c2ccc(-c3nc(-c4ccccc4)nc(-c4cc5c6ccccc6oc5c5cc(-n6c7ccccc7c7c8c(ccc76)sc6ccccc68)ccc45)n3)cc2)cc1 ; c1ccc(-c2nc(-c3ccc4ccccc4c3)nc(-c3cc4c5ccccc5oc4c4cc(-n5c6ccccc6c6ccc7oc8ccccc8c7c65)ccc34)n2)cc1]

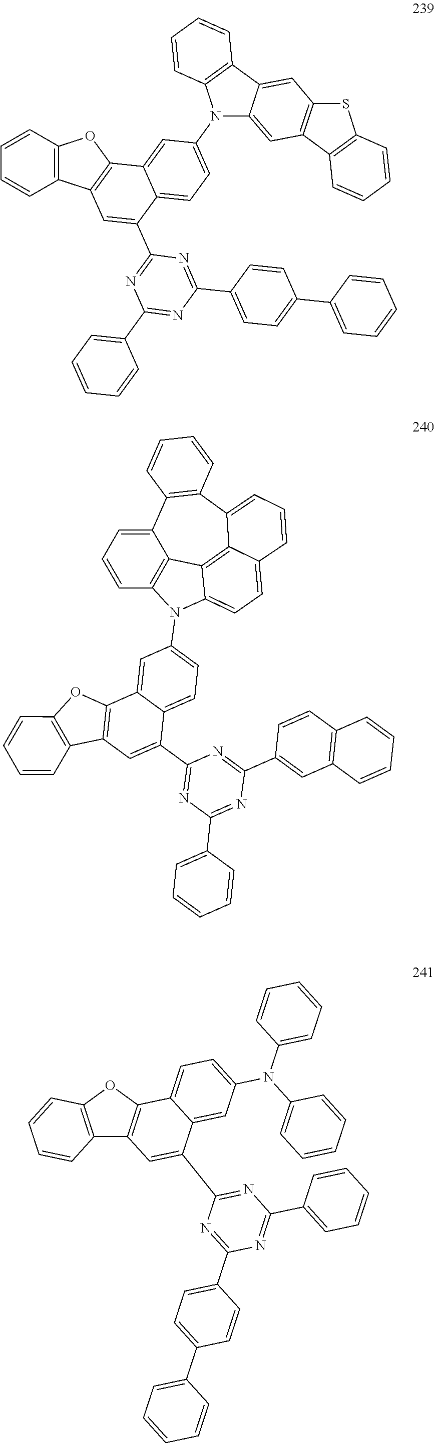 [SMILES: c1ccc(-c2ccc(-c3nc(-c4ccccc4)nc(-c4cc5c6ccccc6oc5c5cc(-n6c7ccccc7c7cc8sc9ccccc9c8cc76)ccc45)n3)cc2)cc1 ; c1ccc(-c2nc(-c3ccc4ccccc4c3)nc(-c3cc4c5ccccc5oc4c4cc(-n5c6cccc7c6c6c8c(cccc8ccc65)-c5ccccc5-7)ccc34)n2)cc1 ; c1ccc(-c2ccc(-c3nc(-c4ccccc4)nc(-c4cc5c6ccccc6oc5c5ccc(N(c6ccccc6)c6ccccc6)cc45)n3)cc2)cc1]

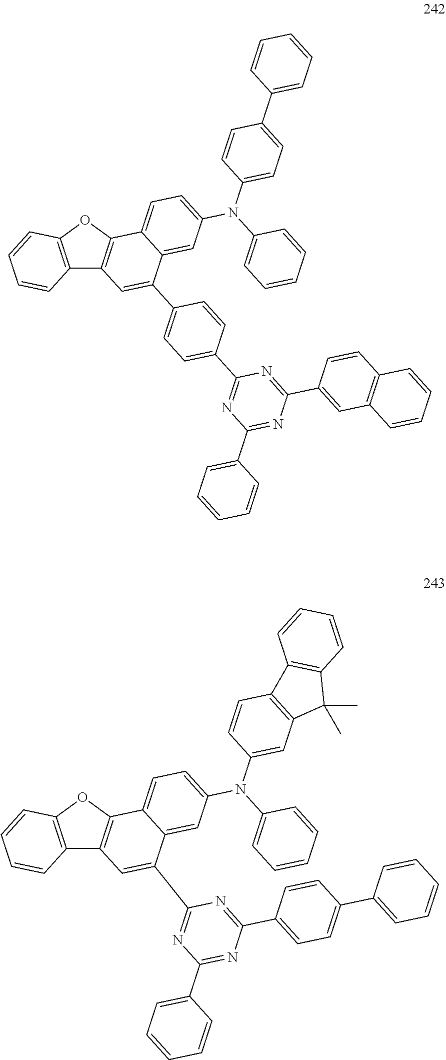 [SMILES: c1ccc(-c2ccc(N(c3ccccc3)c3ccc4c(c3)c(-c3ccc(-c5nc(-c6ccccc6)nc(-c6ccc7ccccc7c6)n5)cc3)cc3c5ccccc5oc43)cc2)cc1 ; CC1(C)c2ccccc2-c2ccc(N(c3ccccc3)c3ccc4c(c3)c(-c3nc(-c5ccccc5)nc(-c5ccc(-c6ccccc6)cc5)n3)cc3c5ccccc5oc43)cc21]

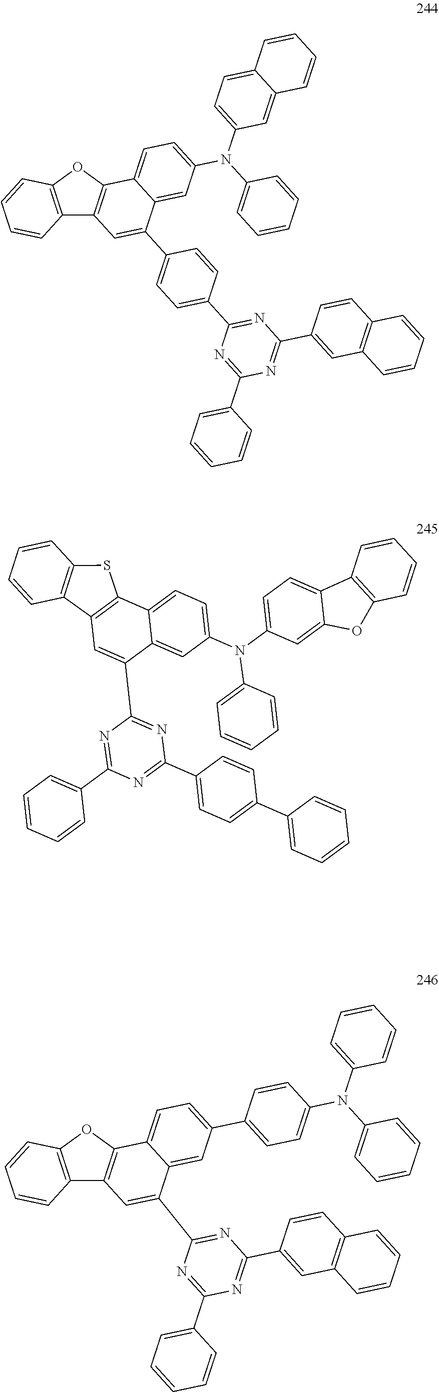 [SMILES: c1ccc(-c2nc(-c3ccc(-c4cc5c6ccccc6oc5c5ccc(N(c6ccccc6)c6ccc7ccccc7c6)cc45)cc3)nc(-c3ccc4ccccc4c3)n2)cc1 ; c1ccc(-c2ccc(-c3nc(-c4ccccc4)nc(-c4cc5c6ccccc6sc5c5ccc(N(c6ccccc6)c6ccc7c(c6)oc6ccccc67)cc45)n3)cc2)cc1 ; c1ccc(-c2nc(-c3ccc4ccccc4c3)nc(-c3cc4c5ccccc5oc4c4ccc(-c5ccc(N(c6ccccc6)c6ccccc6)cc5)cc34)n2)cc1]

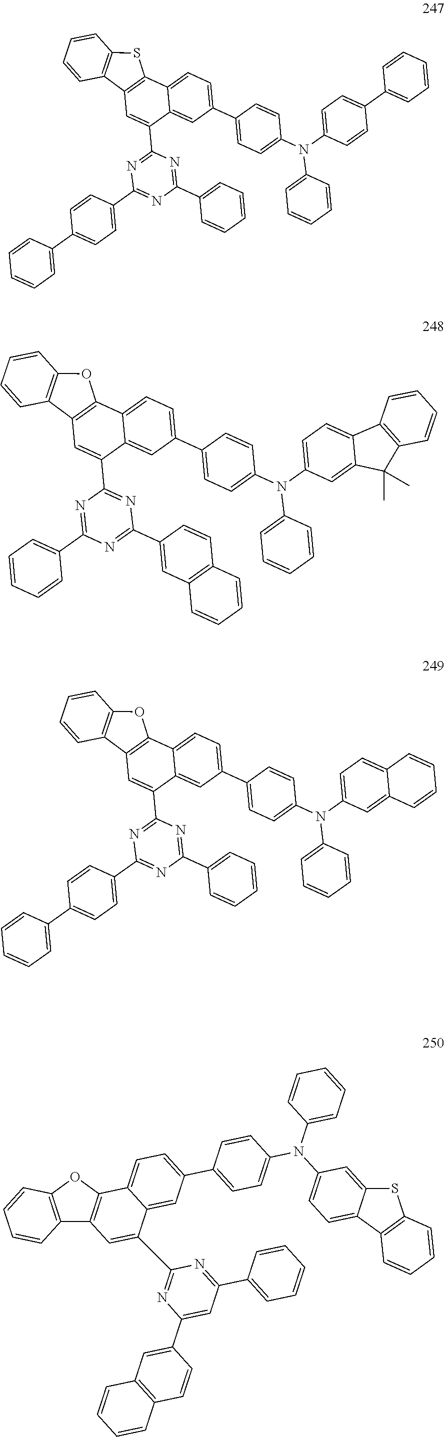 [SMILES: c1ccc(-c2ccc(-c3nc(-c4ccccc4)nc(-c4cc5c6ccccc6sc5c5ccc(-c6ccc(N(c7ccccc7)c7ccc(-c8ccccc8)cc7)cc6)cc45)n3)cc2)cc1 ; CC1(C)c2ccccc2-c2ccc(N(c3ccccc3)c3ccc(-c4ccc5c(c4)c(-c4nc(-c6ccccc6)nc(-c6ccc7ccccc7c6)n4)cc4c6ccccc6oc54)cc3)cc21 ; c1ccc(-c2ccc(-c3nc(-c4ccccc4)nc(-c4cc5c6ccccc6oc5c5ccc(-c6ccc(N(c7ccccc7)c7ccc8ccccc8c7)cc6)cc45)n3)cc2)cc1 ; c1ccc(-c2cc(-c3ccc4ccccc4c3)nc(-c3cc4c5ccccc5oc4c4ccc(-c5ccc(N(c6ccccc6)c6ccc7c(c6)sc6ccccc67)cc5)cc34)n2)cc1]

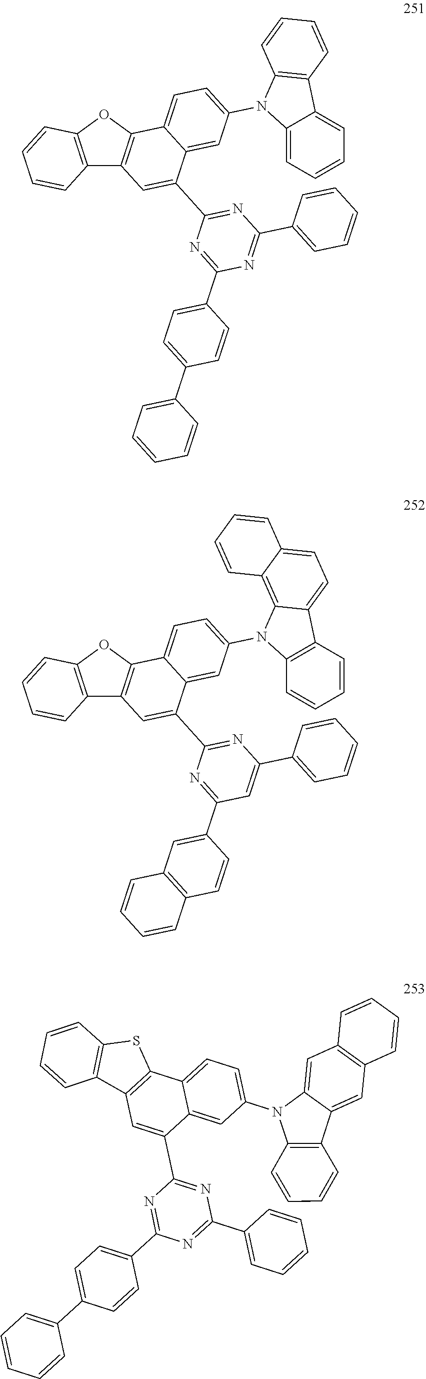 [SMILES: c1ccc(-c2ccc(-c3nc(-c4ccccc4)nc(-c4cc5c6ccccc6oc5c5ccc(-n6c7ccccc7c7ccccc76)cc45)n3)cc2)cc1 ; c1ccc(-c2cc(-c3ccc4ccccc4c3)nc(-c3cc4c5ccccc5oc4c4ccc(-n5c6ccccc6c6ccc7ccccc7c65)cc34)n2)cc1 ; c1ccc(-c2ccc(-c3nc(-c4ccccc4)nc(-c4cc5c6ccccc6sc5c5ccc(-n6c7ccccc7c7cc8ccccc8cc76)cc45)n3)cc2)cc1]

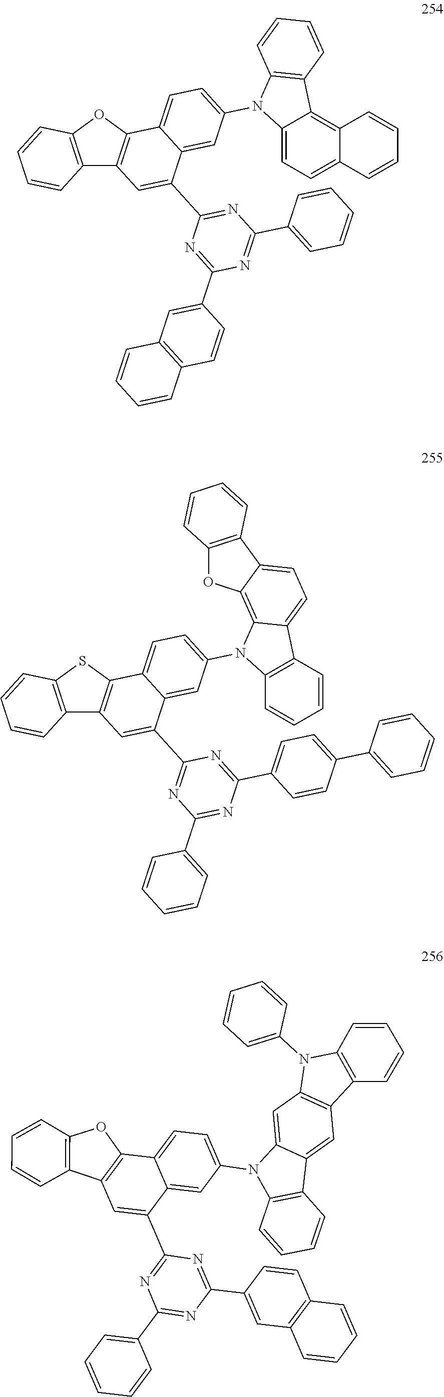 [SMILES: c1ccc(-c2nc(-c3ccc4ccccc4c3)nc(-c3cc4c5ccccc5oc4c4ccc(-n5c6ccccc6c6c7ccccc7ccc65)cc34)n2)cc1 ; c1ccc(-c2ccc(-c3nc(-c4ccccc4)nc(-c4cc5c6ccccc6sc5c5ccc(-n6c7ccccc7c7ccc8c9ccccc9oc8c76)cc45)n3)cc2)cc1 ; c1ccc(-c2nc(-c3ccc4ccccc4c3)nc(-c3cc4c5ccccc5oc4c4ccc(-n5c6ccccc6c6cc7c8ccccc8n(-c8ccccc8)c7cc65)cc34)n2)cc1]

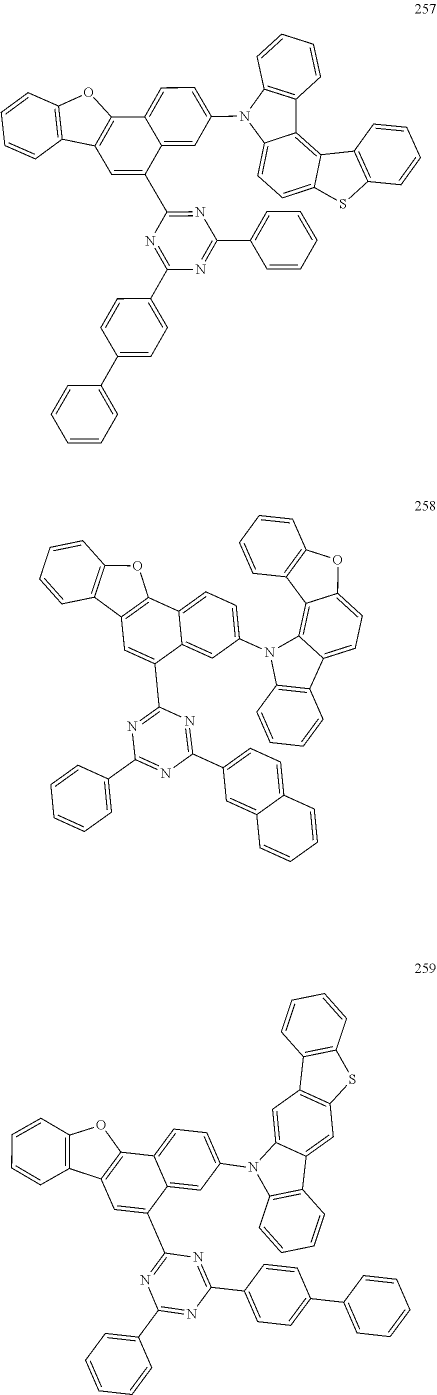 [SMILES: c1ccc(-c2ccc(-c3nc(-c4ccccc4)nc(-c4cc5c6ccccc6oc5c5ccc(-n6c7ccccc7c7c8c(ccc76)sc6ccccc68)cc45)n3)cc2)cc1 ; c1ccc(-c2nc(-c3ccc4ccccc4c3)nc(-c3cc4c5ccccc5oc4c4ccc(-n5c6ccccc6c6ccc7oc8ccccc8c7c65)cc34)n2)cc1 ; c1ccc(-c2ccc(-c3nc(-c4ccccc4)nc(-c4cc5c6ccccc6oc5c5ccc(-n6c7ccccc7c7cc8sc9ccccc9c8cc76)cc45)n3)cc2)cc1]

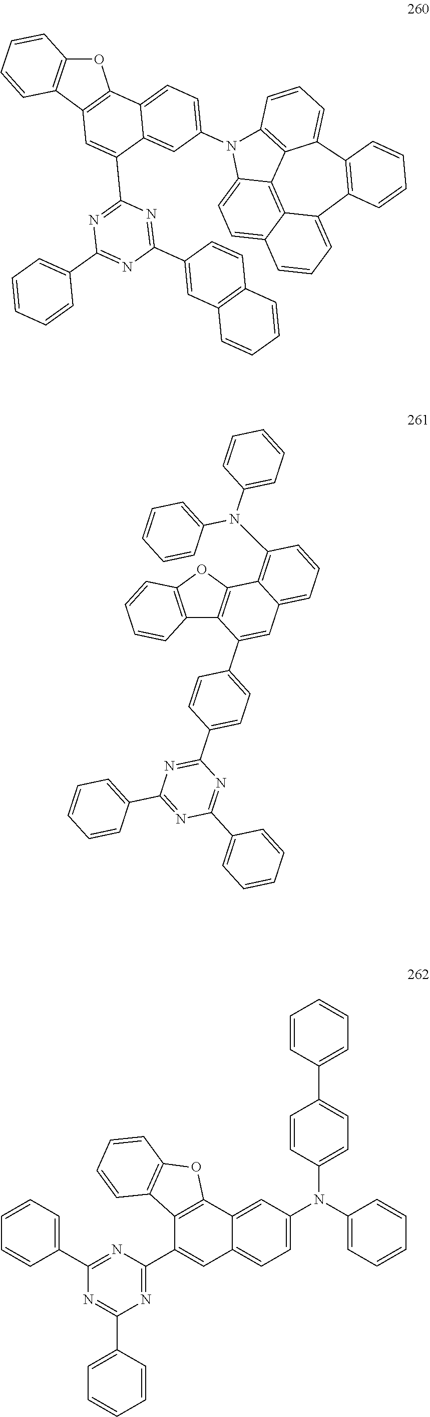 [SMILES: c1ccc(-c2nc(-c3ccc4ccccc4c3)nc(-c3cc4c5ccccc5oc4c4ccc(-n5c6cccc7c6c6c8c(cccc8ccc65)-c5ccccc5-7)cc34)n2)cc1 ; c1ccc(-c2nc(-c3ccccc3)nc(-c3ccc(-c4cc5cccc(N(c6ccccc6)c6ccccc6)c5c5oc6ccccc6c45)cc3)n2)cc1 ; c1ccc(-c2ccc(N(c3ccccc3)c3ccc4cc(-c5nc(-c6ccccc6)nc(-c6ccccc6)n5)c5c6ccccc6oc5c4c3)cc2)cc1]

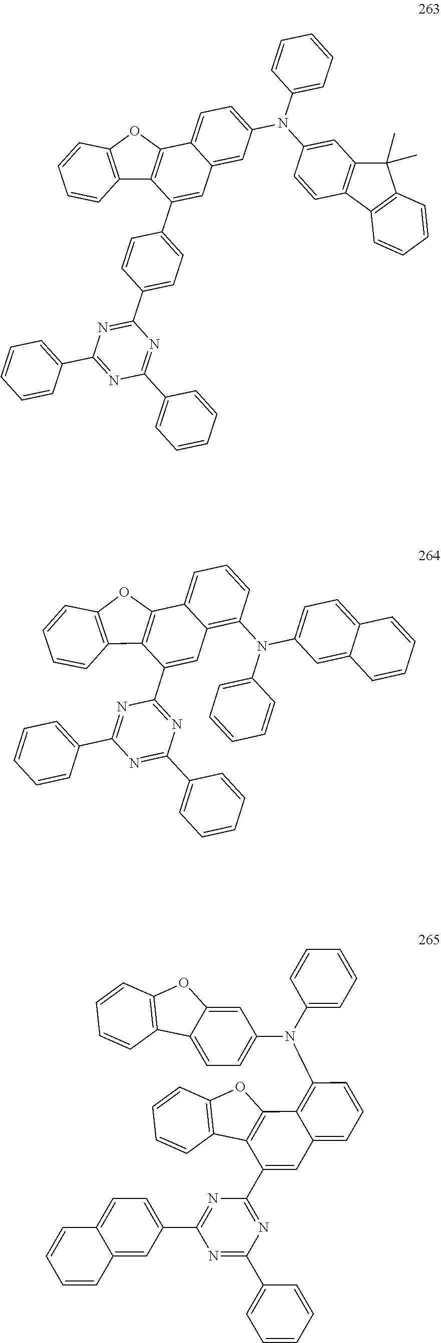 [SMILES: CC1(C)c2ccccc2-c2ccc(N(c3ccccc3)c3ccc4c(c3)cc(-c3ccc(-c5nc(-c6ccccc6)nc(-c6ccccc6)n5)cc3)c3c5ccccc5oc43)cc21 ; c1ccc(-c2nc(-c3ccccc3)nc(-c3cc4c(N(c5ccccc5)c5ccc6ccccc6c5)cccc4c4oc5ccccc5c34)n2)cc1 ; c1ccc(-c2nc(-c3ccc4ccccc4c3)nc(-c3cc4cccc(N(c5ccccc5)c5ccc6c(c5)oc5ccccc56)c4c4oc5ccccc5c34)n2)cc1]

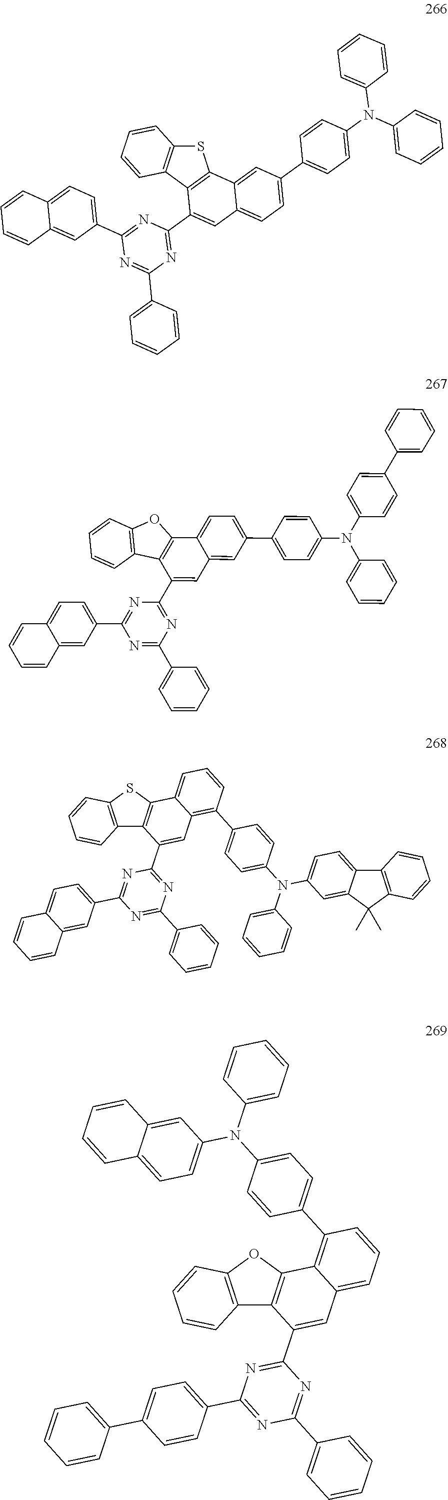 [SMILES: c1ccc(-c2nc(-c3ccc4ccccc4c3)nc(-c3cc4ccc(-c5ccc(N(c6ccccc6)c6ccccc6)cc5)cc4c4sc5ccccc5c34)n2)cc1 ; c1ccc(-c2ccc(N(c3ccccc3)c3ccc(-c4ccc5c(c4)cc(-c4nc(-c6ccccc6)nc(-c6ccc7ccccc7c6)n4)c4c6ccccc6oc54)cc3)cc2)cc1 ; CC1(C)c2ccccc2-c2ccc(N(c3ccccc3)c3ccc(-c4cccc5c4cc(-c4nc(-c6ccccc6)nc(-c6ccc7ccccc7c6)n4)c4c6ccccc6sc54)cc3)cc21 ; c1ccc(-c2ccc(-c3nc(-c4ccccc4)nc(-c4cc5cccc(-c6ccc(N(c7ccccc7)c7ccc8ccccc8c7)cc6)c5c5oc6ccccc6c45)n3)cc2)cc1]

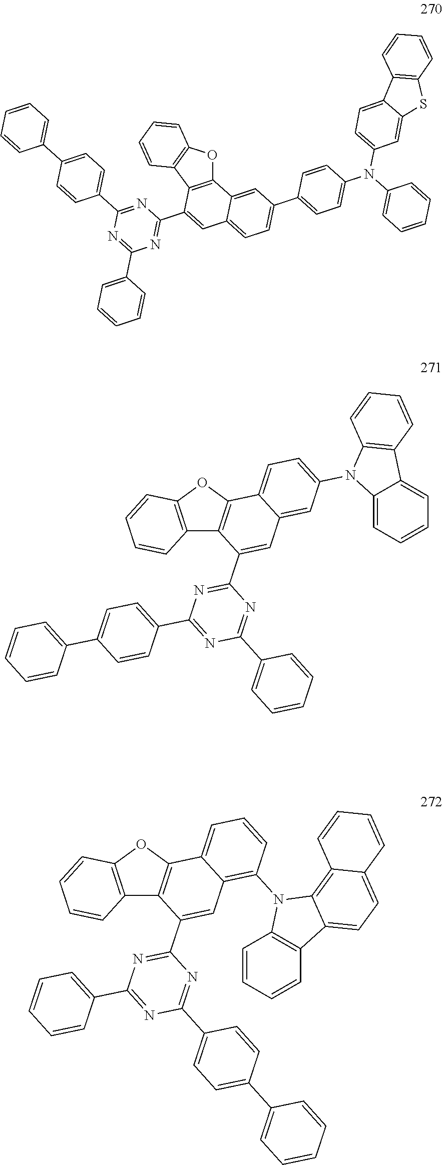 [SMILES: c1ccc(-c2ccc(-c3nc(-c4ccccc4)nc(-c4cc5ccc(-c6ccc(N(c7ccccc7)c7ccc8c(c7)sc7ccccc78)cc6)cc5c5oc6ccccc6c45)n3)cc2)cc1 ; c1ccc(-c2ccc(-c3nc(-c4ccccc4)nc(-c4cc5cc(-n6c7ccccc7c7ccccc76)ccc5c5oc6ccccc6c45)n3)cc2)cc1 ; c1ccc(-c2ccc(-c3nc(-c4ccccc4)nc(-c4cc5c(-n6c7ccccc7c7ccc8ccccc8c76)cccc5c5oc6ccccc6c45)n3)cc2)cc1]

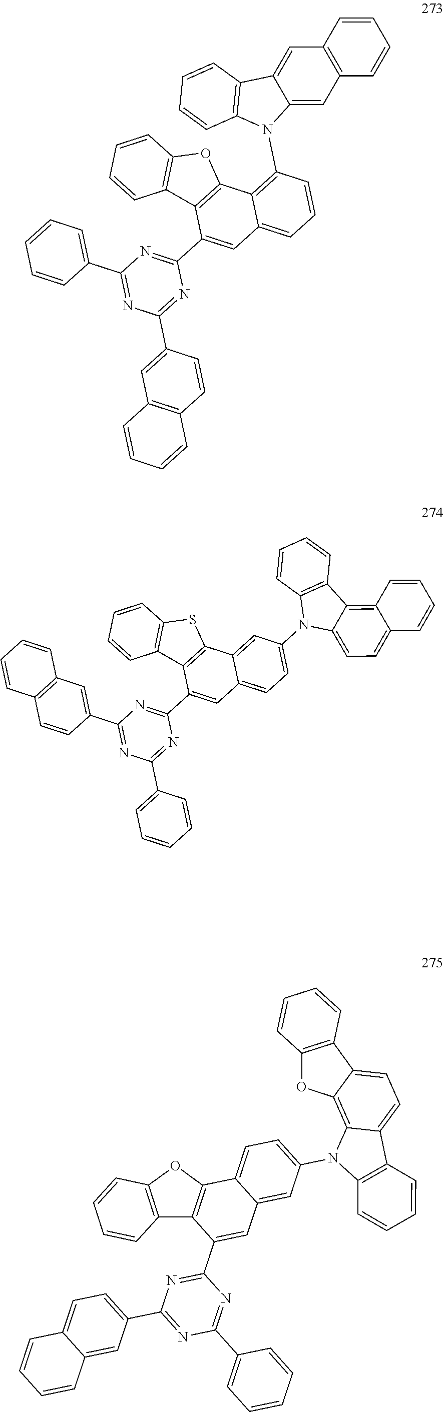 [SMILES: c1ccc(-c2nc(-c3ccc4ccccc4c3)nc(-c3cc4cccc(-n5c6ccccc6c6cc7ccccc7cc65)c4c4oc5ccccc5c34)n2)cc1 ; c1ccc(-c2nc(-c3ccc4ccccc4c3)nc(-c3cc4ccc(-n5c6ccccc6c6c7ccccc7ccc65)cc4c4sc5ccccc5c34)n2)cc1 ; c1ccc(-c2nc(-c3ccc4ccccc4c3)nc(-c3cc4cc(-n5c6ccccc6c6ccc7c8ccccc8oc7c65)ccc4c4oc5ccccc5c34)n2)cc1]

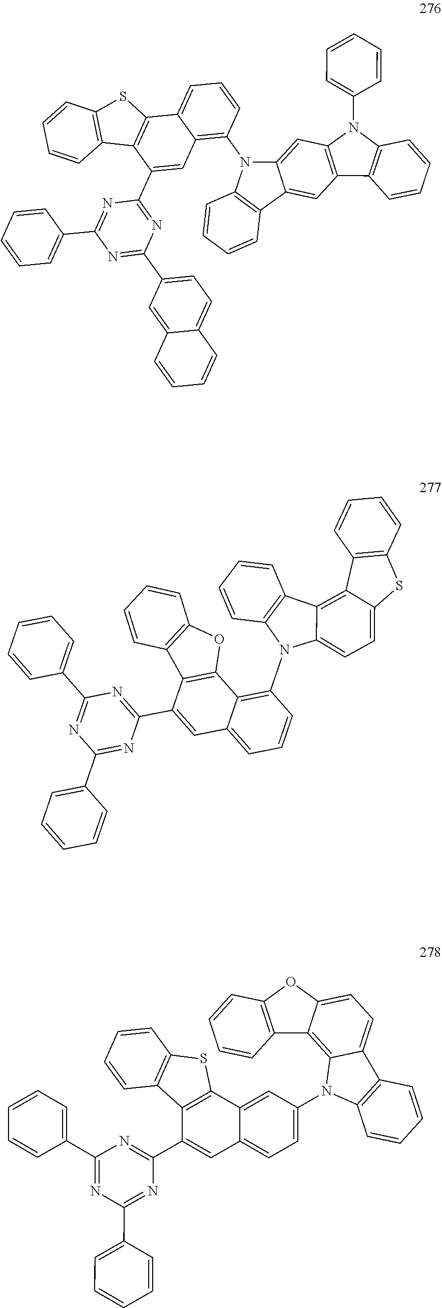 [SMILES: c1ccc(-c2nc(-c3ccc4ccccc4c3)nc(-c3cc4c(-n5c6ccccc6c6cc7c8ccccc8n(-c8ccccc8)c7cc65)cccc4c4sc5ccccc5c34)n2)cc1 ; c1ccc(-c2nc(-c3ccccc3)nc(-c3cc4cccc(-n5c6ccccc6c6c7c(ccc65)sc5ccccc57)c4c4oc5ccccc5c34)n2)cc1 ; c1ccc(-c2nc(-c3ccccc3)nc(-c3cc4ccc(-n5c6ccccc6c6ccc7oc8ccccc8c7c65)cc4c4sc5ccccc5c34)n2)cc1]

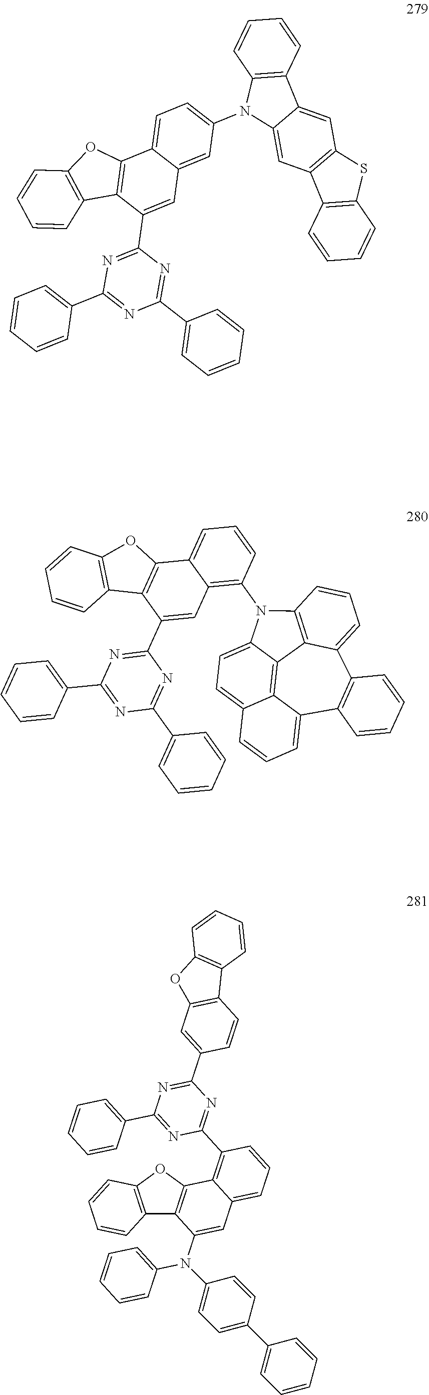 [SMILES: c1ccc(-c2nc(-c3ccccc3)nc(-c3cc4cc(-n5c6ccccc6c6cc7sc8ccccc8c7cc65)ccc4c4oc5ccccc5c34)n2)cc1 ; c1ccc(-c2nc(-c3ccccc3)nc(-c3cc4c(-n5c6cccc7c6c6c8c(cccc8ccc65)-c5ccccc5-7)cccc4c4oc5ccccc5c34)n2)cc1 ; c1ccc(-c2ccc(N(c3ccccc3)c3cc4cccc(-c5nc(-c6ccccc6)nc(-c6ccc7c(c6)oc6ccccc67)n5)c4c4oc5ccccc5c34)cc2)cc1]

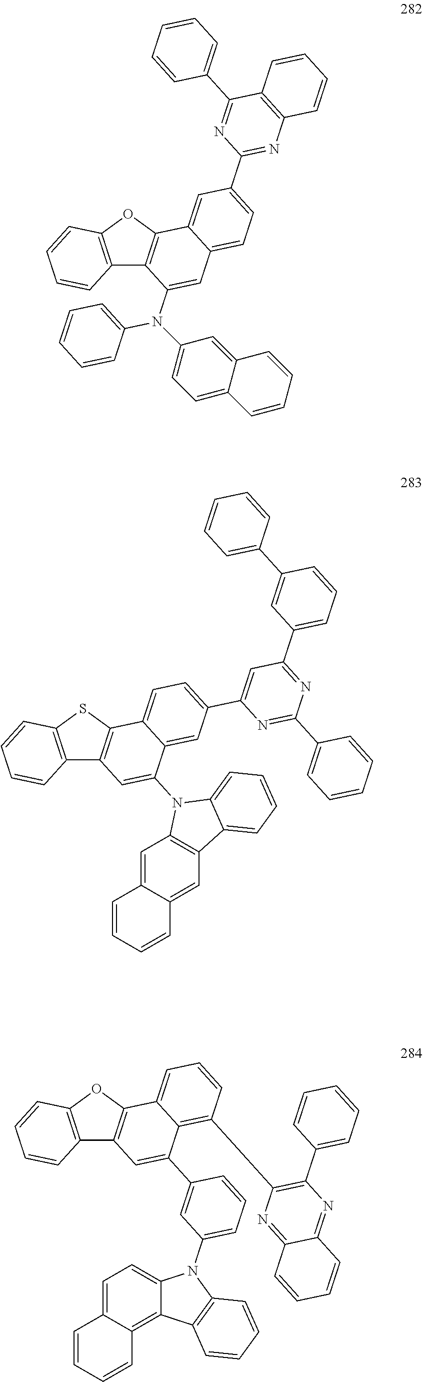 [SMILES: c1ccc(-c2nc(-c3ccc4cc(N(c5ccccc5)c5ccc6ccccc6c5)c5c6ccccc6oc5c4c3)nc3ccccc23)cc1 ; c1ccc(-c2cccc(-c3cc(-c4ccc5c(c4)c(-n4c6ccccc6c6cc7ccccc7cc64)cc4c6ccccc6sc54)nc(-c4ccccc4)n3)c2)cc1 ; c1ccc(-c2nc3ccccc3nc2-c2cccc3c2c(-c2cccc(-n4c5ccccc5c5c6ccccc6ccc54)c2)cc2c4ccccc4oc32)cc1]

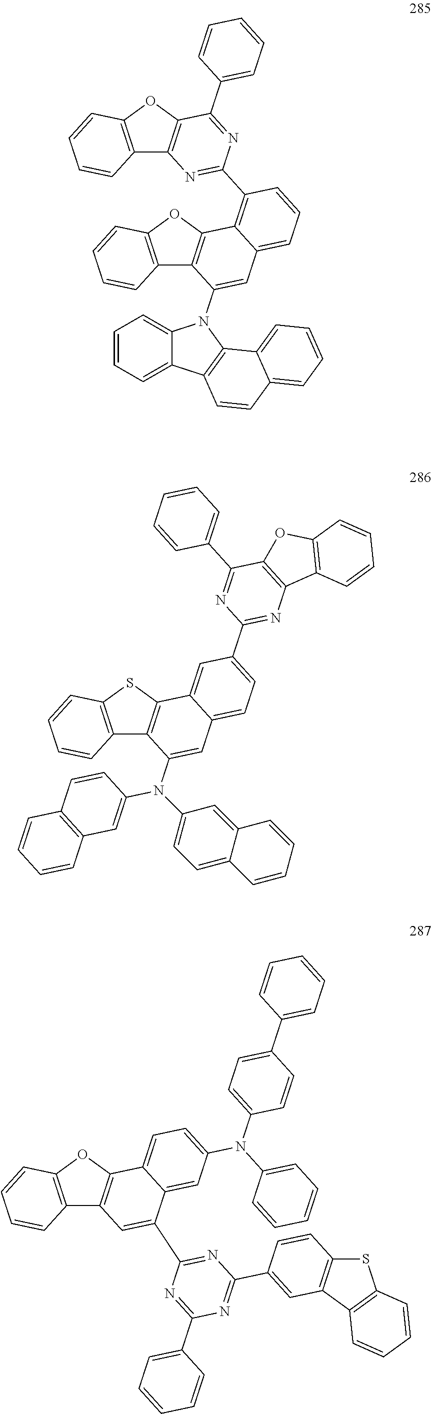 [SMILES: c1ccc(-c2nc(-c3cccc4cc(-n5c6ccccc6c6ccc7ccccc7c65)c5c6ccccc6oc5c34)nc3c2oc2ccccc23)cc1 ; c1ccc(-c2nc(-c3ccc4cc(N(c5ccc6ccccc6c5)c5ccc6ccccc6c5)c5c6ccccc6sc5c4c3)nc3c2oc2ccccc23)cc1 ; c1ccc(-c2ccc(N(c3ccccc3)c3ccc4c(c3)c(-c3nc(-c5ccccc5)nc(-c5ccc6sc7ccccc7c6c5)n3)cc3c5ccccc5oc43)cc2)cc1]

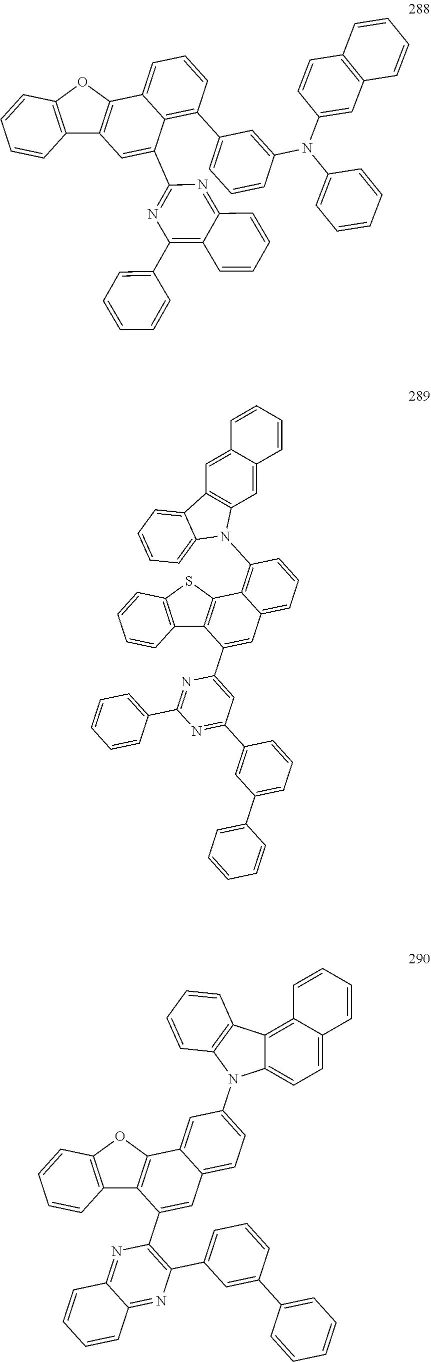 [SMILES: c1ccc(-c2nc(-c3cc4c5ccccc5oc4c4cccc(-c5cccc(N(c6ccccc6)c6ccc7ccccc7c6)c5)c34)nc3ccccc23)cc1 ; c1ccc(-c2cccc(-c3cc(-c4cc5cccc(-n6c7ccccc7c7cc8ccccc8cc76)c5c5sc6ccccc6c45)nc(-c4ccccc4)n3)c2)cc1 ; c1ccc(-c2cccc(-c3nc4ccccc4nc3-c3cc4ccc(-n5c6ccccc6c6c7ccccc7ccc65)cc4c4oc5ccccc5c34)c2)cc1]

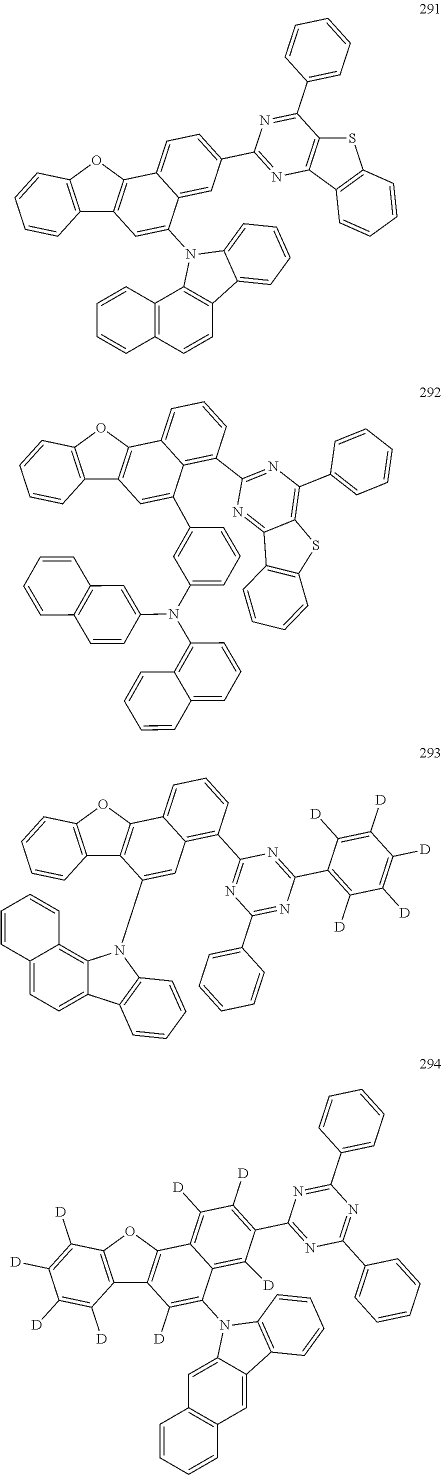 [SMILES: c1ccc(-c2nc(-c3ccc4c(c3)c(-n3c5ccccc5c5ccc6ccccc6c53)cc3c5ccccc5oc43)nc3c2sc2ccccc23)cc1 ; c1ccc(-c2nc(-c3cccc4c3c(-c3cccc(N(c5ccc6ccccc6c5)c5cccc6ccccc56)c3)cc3c5ccccc5oc43)nc3c2sc2ccccc23)cc1 ; [2H]c1c([2H])c([2H])c(-c2nc(-c3ccccc3)nc(-c3cccc4c3cc(-n3c5ccccc5c5ccc6ccccc6c53)c3c5ccccc5oc43)n2)c([2H])c1[2H] ; [2H]c1c([2H])c([2H])c2c(oc3c4c([2H])c([2H])c(-c5nc(-c6ccccc6)nc(-c6ccccc6)n5)c([2H])c4c(-n4c5ccccc5c5cc6ccccc6cc54)c([2H])c32)c1[2H]]

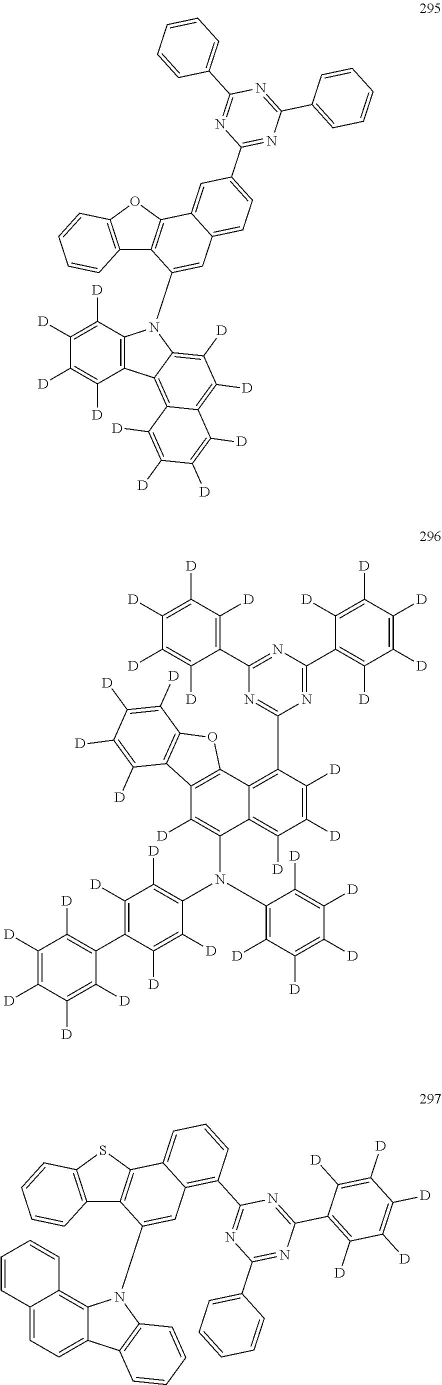

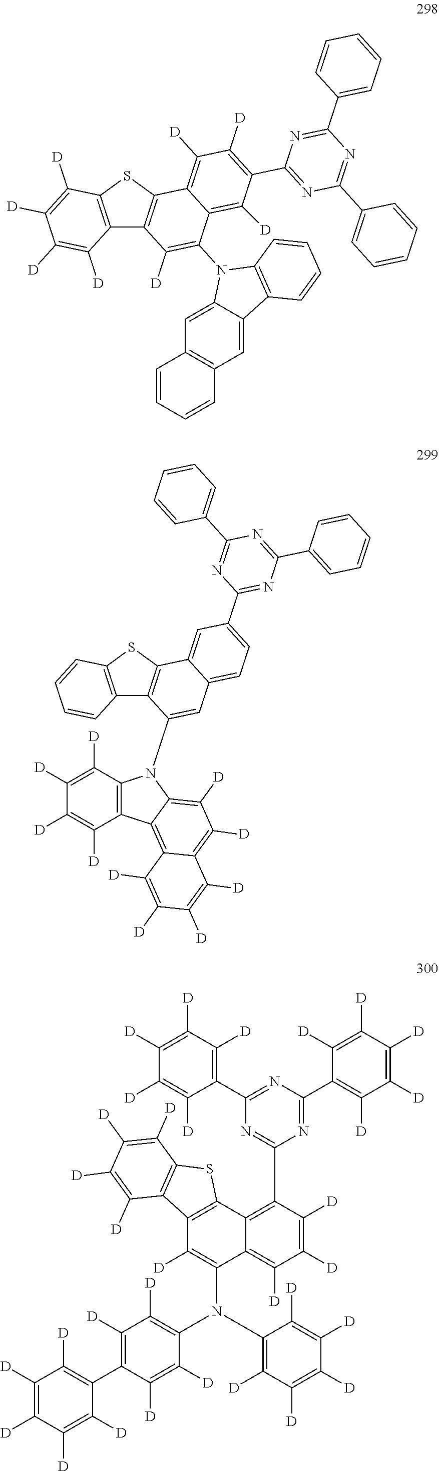

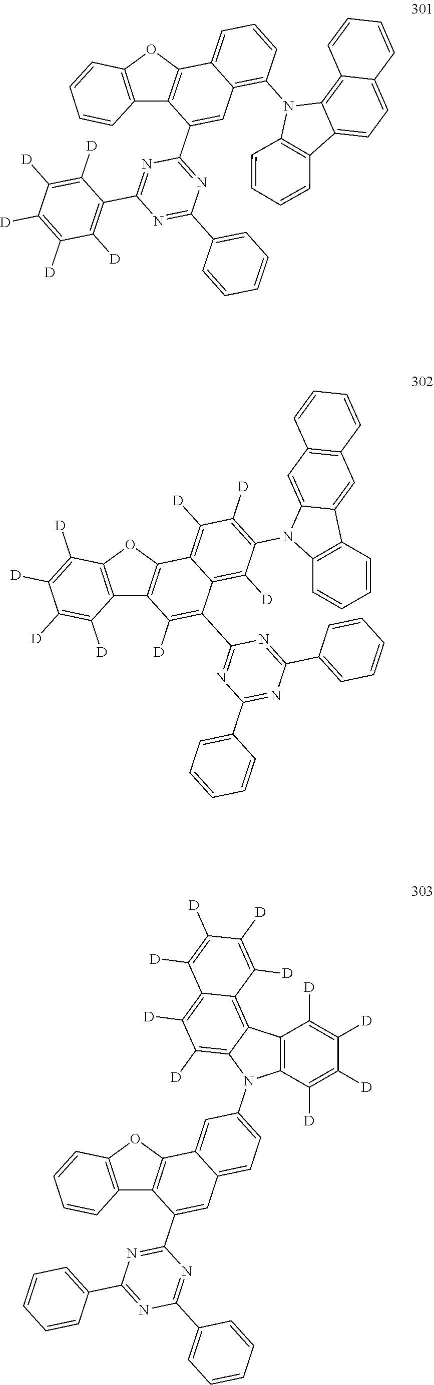 [SMILES: [2H]c1c([2H])c([2H])c(-c2nc(-c3ccccc3)nc(-c3cc4c(-n5c6ccccc6c6ccc7ccccc7c65)cccc4c4oc5ccccc5c34)n2)c([2H])c1[2H] ; [2H]c1c([2H])c([2H])c2c(oc3c4c([2H])c([2H])c(-n5c6ccccc6c6cc7ccccc7cc65)c([2H])c4c(-c4nc(-c5ccccc5)nc(-c5ccccc5)n4)c([2H])c32)c1[2H] ; [2H]c1c([2H])c([2H])c2c(c1[2H])c([2H])c([2H])c1c2c2c([2H])c([2H])c([2H])c([2H])c2n1-c1ccc2cc(-c3nc(-c4ccccc4)nc(-c4ccccc4)n3)c3c4ccccc4oc3c2c1]

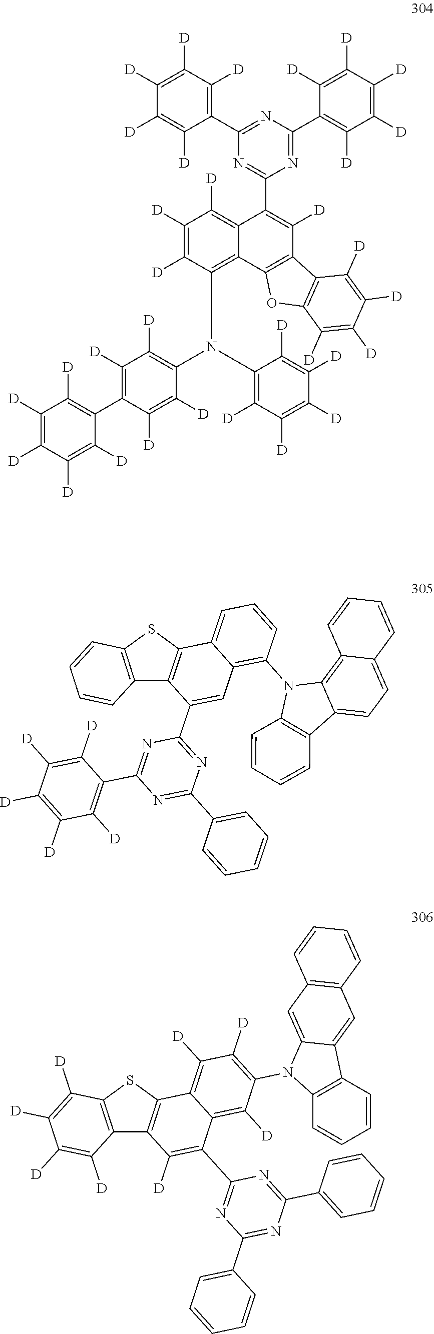

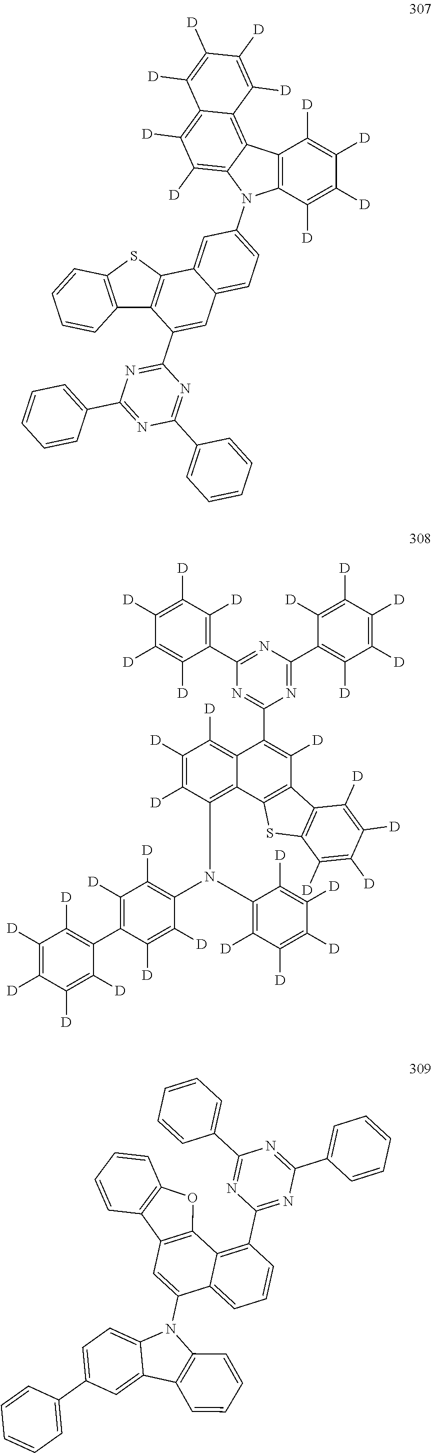

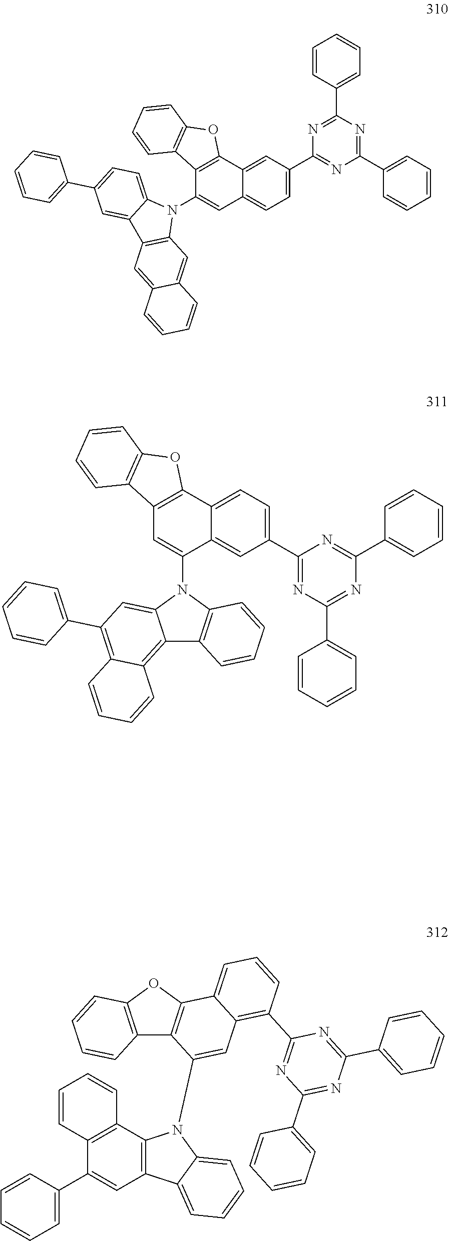 [SMILES: c1ccc(-c2ccc3c(c2)c2cc4ccccc4cc2n3-c2cc3ccc(-c4nc(-c5ccccc5)nc(-c5ccccc5)n4)cc3c3oc4ccccc4c23)cc1 ; c1ccc(-c2nc(-c3ccccc3)nc(-c3ccc4c(c3)c(-n3c5ccccc5c5c6ccccc6c(-c6ccccc6)cc53)cc3c5ccccc5oc43)n2)cc1 ; c1ccc(-c2nc(-c3ccccc3)nc(-c3cccc4c3cc(-n3c5ccccc5c5cc(-c6ccccc6)c6ccccc6c53)c3c5ccccc5oc43)n2)cc1]

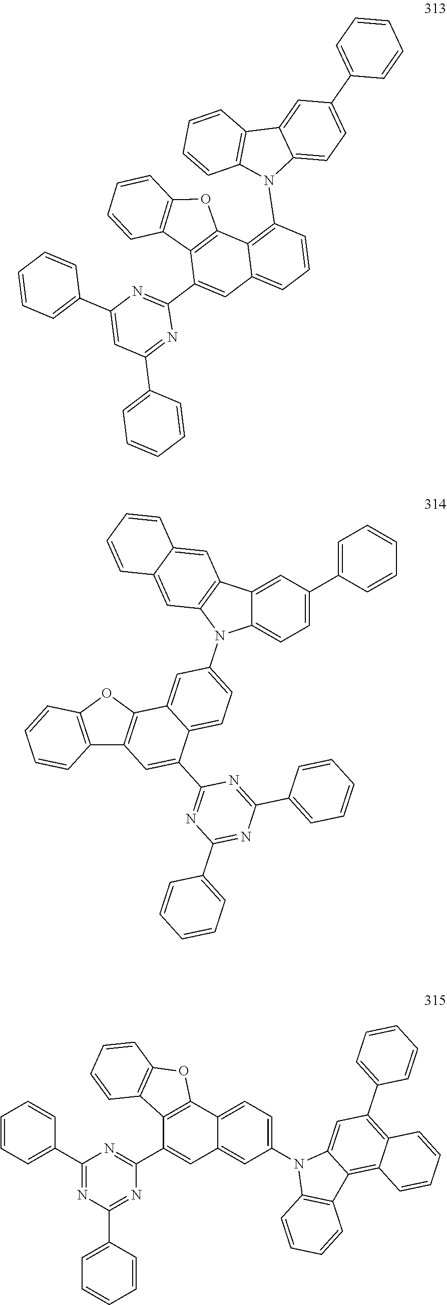 [SMILES: c1ccc(-c2ccc3c(c2)c2ccccc2n3-c2cccc3cc(-c4nc(-c5ccccc5)cc(-c5ccccc5)n4)c4c5ccccc5oc4c23)cc1 ; c1ccc(-c2ccc3c(c2)c2cc4ccccc4cc2n3-c2ccc3c(-c4nc(-c5ccccc5)nc(-c5ccccc5)n4)cc4c5ccccc5oc4c3c2)cc1 ; c1ccc(-c2nc(-c3ccccc3)nc(-c3cc4cc(-n5c6ccccc6c6c7ccccc7c(-c7ccccc7)cc65)ccc4c4oc5ccccc5c34)n2)cc1]

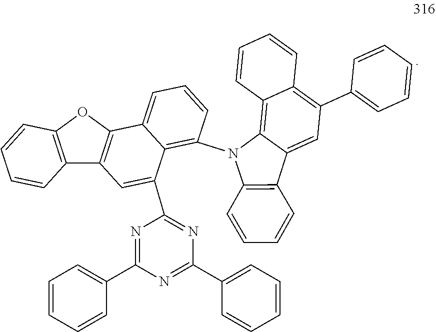 [SMILES: c1ccc(-c2nc(-c3ccccc3)nc(-c3cc4c5ccccc5oc4c4cccc(-n5c6ccccc6c6cc(-c7ccccc7)c7ccccc7c65)c34)n2)cc1]

8. An organic light emitting device comprising:
a first electrode;
a second electrode provided to face the first electrode; and
one or more organic material layers provided between the first electrode and the second electrode,
wherein one or more layers of the organic material layers comprise the heterocyclic compound of claim 1.

9. The organic light emitting device according to claim 8, wherein the organic material layer comprises a light emitting layer, and the light emitting layer comprises the heterocyclic compound.

10. The organic light emitting device according to claim 8, wherein the organic material layer comprises a light emitting layer, the light emitting layer comprises a host material, and the host material comprises the heterocyclic compound.

11. The organic light emitting device according to claim 8, wherein the organic light emitting device further comprises one or more layers selected from the group consisting of a light emitting layer, a hole injection layer, a hole transport layer, an electron injection layer, an electron transport layer, an electron blocking layer and a hole blocking layer.

12. The organic light emitting device according to claim 8, wherein the organic material layer further comprises a heterocyclic compound represented by the following Chemical Formula 2-1 or the following Chemical Formula 2-2: 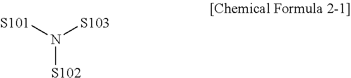
wherein,
S101 to S103 are the same as or different from each other, and are each independently a substituted or unsubstituted C6 to C60 aryl group; or a substituted or unsubstituted C2 to C60 heteroaryl group, 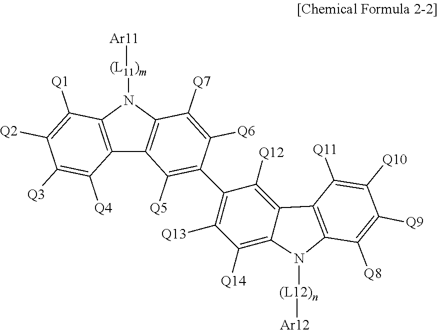
wherein,
L11 and L12 are the same as or different from each other, and are each independently a single bond; a substituted or unsubstituted C6 to C60 arylene group; or a substituted or unsubstituted C2 to C60 heteroarylene group;
Ar11 and Ar12 are the same as or different from each other, and are each independently a substituted or unsubstituted C6 to C60 aryl group; or a substituted or unsubstituted C2 to C60 heteroaryl group;
Q1 to Q14 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group; and
m and n are the same as or different from each other, and are each independently an integer of 0 to 3.

13. The organic light emitting device according to claim 12, wherein the heterocyclic compound represented by Chemical Formula 2-1 or Chemical Formula 2-2 does not contain deuterium as a substituent, or the content of deuterium based on a total number of hydrogen atoms and deuterium atoms is 1% to 100%.

14. The organic light emitting device according to claim 12, wherein the heterocyclic compound represented by Chemical Formula 2-1 or Chemical Formula 2-2 is any one selected from the group consisting of the following compounds:

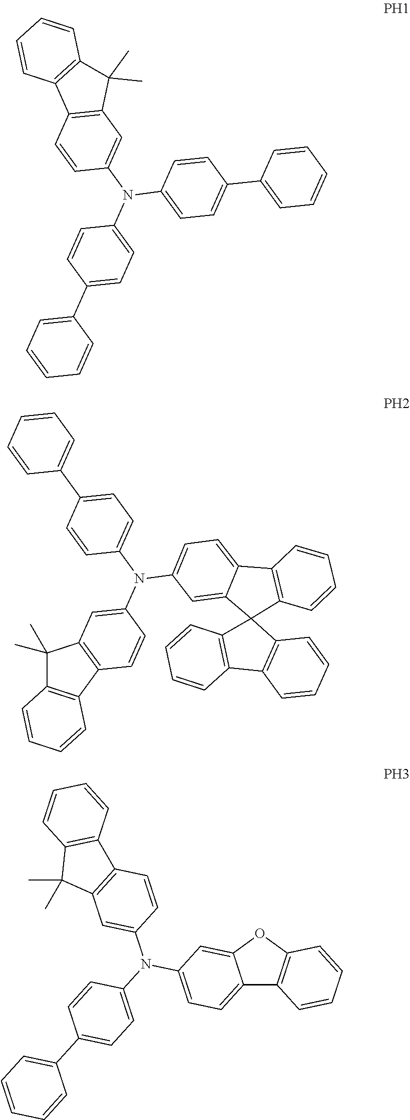

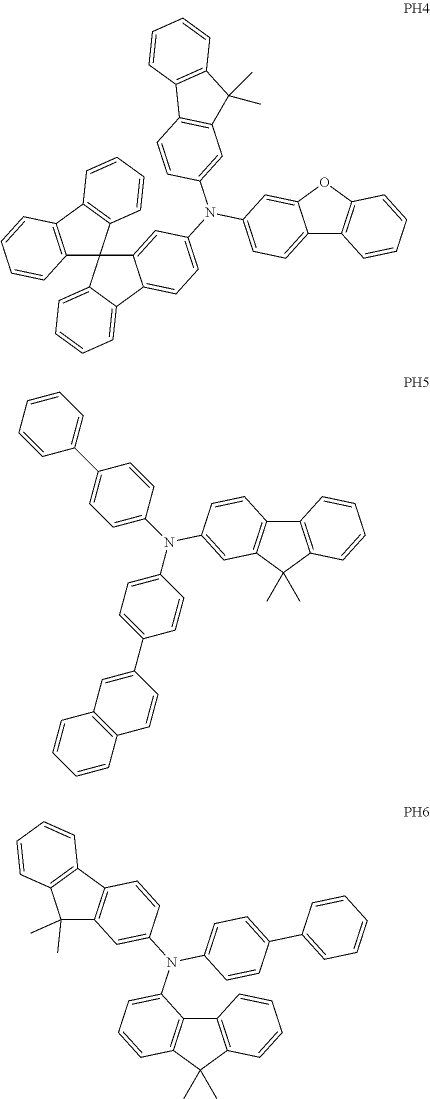

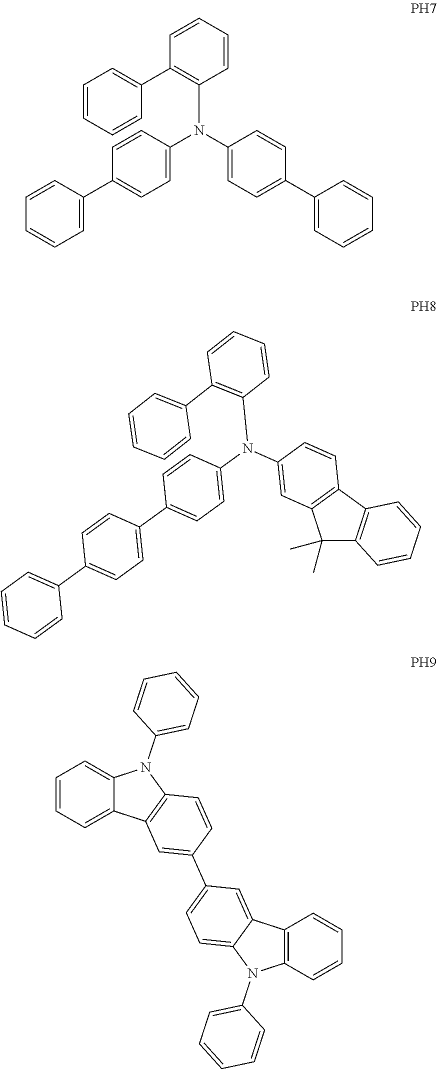 [SMILES: c1ccc(-c2ccc(N(c3ccc(-c4ccccc4)cc3)c3ccccc3-c3ccccc3)cc2)cc1 ; CC1(C)c2ccccc2-c2ccc(N(c3ccc(-c4ccc(-c5ccccc5)cc4)cc3)c3ccccc3-c3ccccc3)cc21 ; c1ccc(-n2c3ccccc3c3cc(-c4ccc5c(c4)c4ccccc4n5-c4ccccc4)ccc32)cc1]

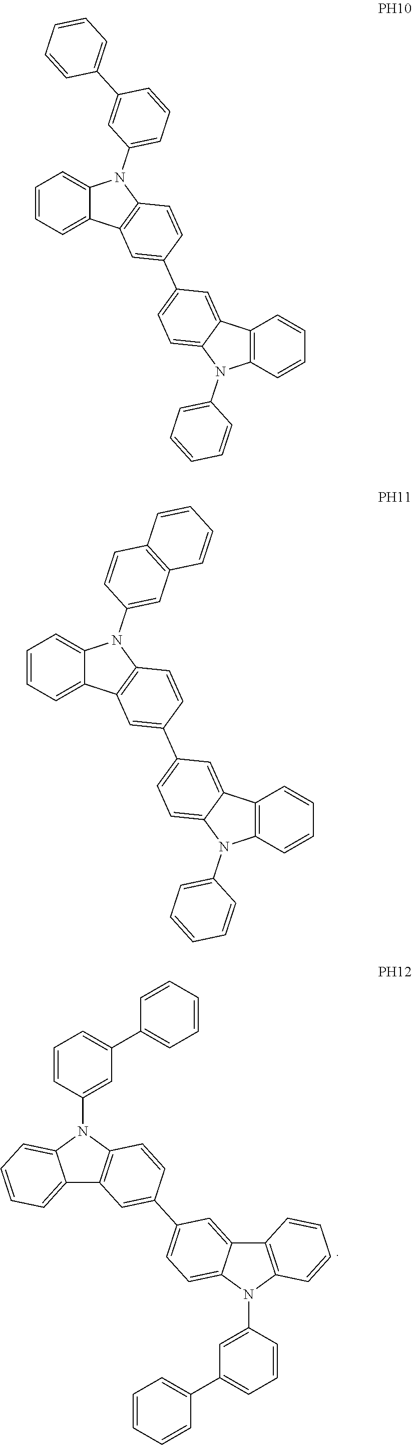

15. The organic light emitting device according to claim 12, wherein at least one of the heterocyclic compound represented by Chemical Formula 1 and the heterocyclic compound represented by Chemical Formula 2-1 or Chemical Formula 2-2 does not contain deuterium as a substituent, or the content of deuterium based on a total number of hydrogen atoms and deuterium atoms is 1% to 100%.

16. A composition for an organic material layer of an organic light emitting device, the composition comprising:
the heterocyclic compound represented by Chemical Formula 1 of claim 1; and
a heterocyclic compound represented by the following Chemical Formula 2-1 or Chemical Formula 2-2: 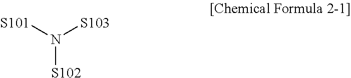
wherein,
S101 to S103 are the same as or different from each other, and are each independently a substituted or unsubstituted C6 to C60 aryl group; or a substituted or unsubstituted C2 to C60 heteroaryl group, 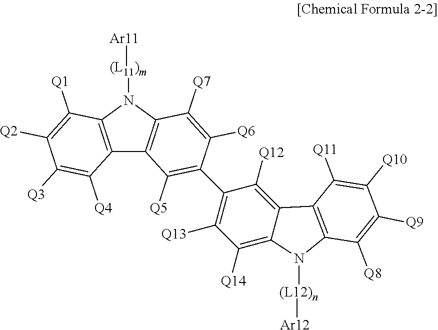
wherein,
L11 and L12 are the same as or different from each other, and are each independently a single bond; a substituted or unsubstituted C6 to C60 arylene group; or a substituted or unsubstituted C2 to C60 heteroarylene group;
Ar11 and Ar12 are the same as or different from each other, and are each independently a substituted or unsubstituted C6 to C60 aryl group; or a substituted or unsubstituted C2 to C60 heteroaryl group;
Q1 to Q14 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; and a substituted or unsubstituted C2 to C60 heterocycloalkyl group; and
m and n are the same as or different from each other, and are each independently an integer of 0 to 3.

17. The composition of claim 16, wherein the heterocyclic compound represented by Chemical Formula 1 and the heterocyclic compound represented by Chemical Formula 2-1 or Chemical Formula 2-2 have a weight ratio of 1:10 to 10:1.
